(19) United States
(12) Patent Application Publication
(10) Pub. No.: US 2012/0004157 A1
(43) Pub. Date: Jan. 5, 2012

(54) HYDROXYPROLINE COMPOSITIONS AND USES THEREOF

(75) Inventor: Anders Aksnes, Bergen (NO)

(73) Assignee: Bergen Teknologioverforing AS, Bergen (NO)

(21) Appl. No.: 12/451,940

(22) PCT Filed: Jun. 6, 2008
(86) PCT No.: PCT/EP2008/057084

§ 371 Date: Apr. 22, 2010

Related U.S. Application Data
(60) Provisional application No. 60/933,737, filed on Jun. 8, 2007.

(30) Foreign Application Priority Data
Jun. 8, 2007 (DK) PA 2007 00833

(51) Int. Cl. A61K 38/02; A61P 43/00; A61P 19/08; C07D 207/16; A61K 31/4015

(52) U.S. Cl. 514/1.1; 548/532; 514/423

(57) ABSTRACT

The invention concerns novel use for Hydroxyproline in a pharmaceutical or dietary food or feed compositions for promoting growth e.g. of fish and/or for treating, ameliorating, or curing certain disorders including osteoporosis, psoriasis, enteritis and gingivitis.

### FIELD OF INVENTION

The invention concerns novel use for Hydroxyproline in a pharmaceutical or dietary food or feed compositions for promoting growth and/or for treating, ameliorating, or curing certain disorders.

### BACKGROUND

Essential amino acids are designated according to classic nutritional studies as being either essential or non-essential. While non-essential amino acids are able to be synthesised by the organism, essential amino acids must be supplied via the diet. Which amino acids are essential or non-essential respectively, varies according the biochemical make-up of the organism in question.

To ensure the optimal growth of an organism, it is generally accepted that the amino acids essential to that organism should be provided in sufficient amounts in the food or feed. This can be achieved either by choosing a source rich in those amino acids, or by supplementing the diet with the essential amino acids. Concurrently, it is also accepted that those amino acids established as non-essential amino do not necessarily need to be provided via diet.

Hydroxyproline is regarded as a non-essential amino acid. Hydroxyproline can be synthesized in the organism, and the metabolic capacity is expected to meet the amount required by the organism. Because it is formed by post-transcriptional hydroxylation of Proline, dietary supplementation of Hydroxyproline is not been expected to influence processes in the organisms that require Hydroxyproline. Instead, efforts to influence the amount of Hydroxyproline available to an organism have been concerned with either supplementing the diet with Proline, precursor to Hydroxyproline, and/or stimulating the hydroxylation process, most notably by increasing availability of the hydroxylase co-factor, vitamin C.

The issue of which amino acids are essential or non-essential, and thus should be provided in the diet to ensure growth and health becomes particularly relevant in the commercial farming of animals, e.g. of fish and poultry.

In commercial fish farming, fish meal has traditionally been the base of fish feed for carnivorous species, but in order to support a growing global farming of shrimp and fish, there is an interest in replacing marine protein sources. However, use of plant-based protein sources has not yet proven to be able to match marine source aqua feed. Research is now directed to understanding in what ways plant protein sources may be supplemented to achieve the results corresponding to that of marine source fish feed.

### SUMMARY OF INVENTION

Disclosed is a composition, such as a food, feed or dietary supplement, comprising or consisting of Hydroxyproline. The invention is suitable for use in treating, ameliorating or curing disorders and for stimulating growth. Also disclosed are methods for making said composition, such as a food, feed or dietary supplement.

The present invention relates to a composition comprising hydroxyproline and optionally also a protein source, and the use of such a composition as a supplement, such as a dietary supplement. The invention further relates to a pharmaceutical composition comprising a therapeutically effective amount of hydroxyproline or hydroprolyl.

### BRIEF DESCRIPTION OF THE ILLUSTRATIONS

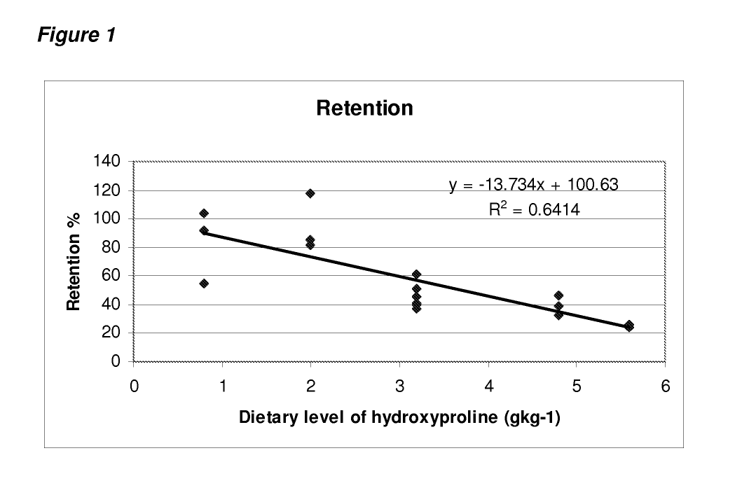

FIG. 1 demonstrates that the retention of Hydroxyproline is statistically correlated to the dietary level. Low dietary levels (0.8 g per kg diet) give retention of close to 100% in the fish while higher dietary levels (>4.5 g per kg diet) gives a lower level of retention of 20-30%. This in turn indicates that Hydroxyproline has some biological impact. The deleterious effect is observed at dietary levels of less 3-4 g per kg diet.

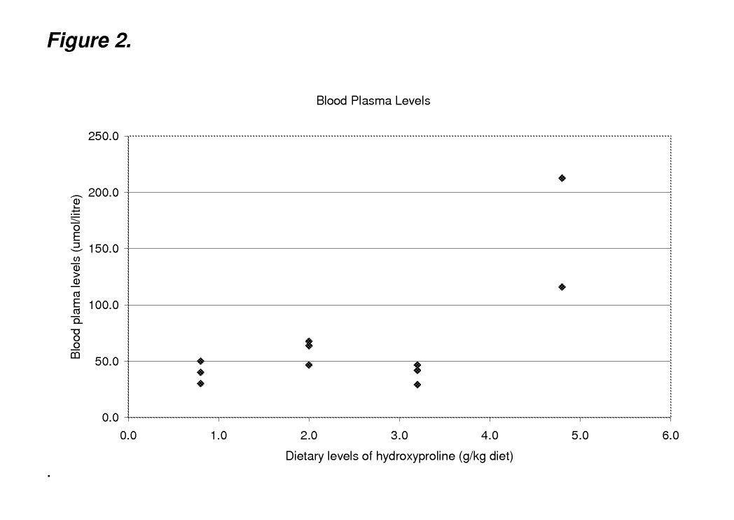

FIG. 2 demonstrates the blood plasma levels of Hydroxyproline in trout fed for 90 days with diets varying in dietary Hydroxyproline. The y axis shows levels of Hydroxyproline in plasma measured in micromoles per 100 ml (μM/100 ml) and the x-axis shows the dietary level of component in g per kg diet (g/kg diet). At low levels (0.8 g per kg) the plasma levels are much lower than previously known (normal values) and indicate that dietary supplement is too low. The results also indicate that Hydroxyproline is easily retrieved from the plasma and incorporated in the tissues. Dietary levels >4 g per kg give very high plasma levels (the highest described in the literature) and indicate that there is a surplus.

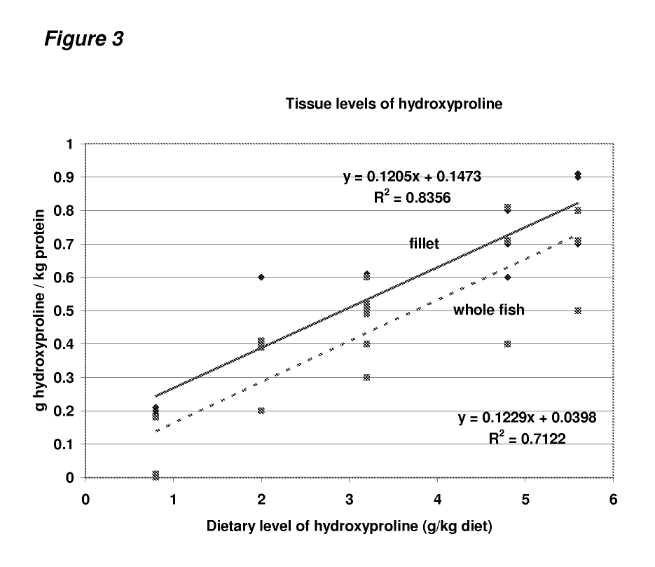

FIG. 3 demonstrates the levels of Hydroxyproline in tissue (filet and whole fish, respectively) from trout fed for 90 days with diets varying in dietary hydroxyproline. The data shows the same tendencies as that for plasma levels and leads to the same conclusions.

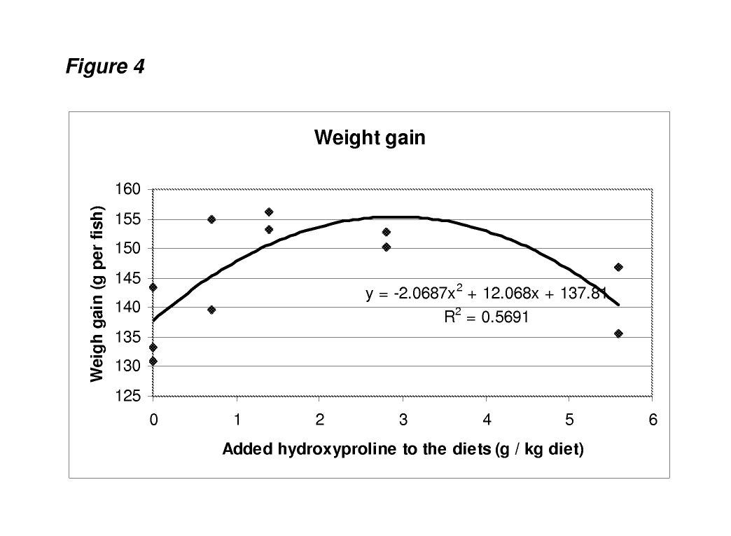

FIG. 4 demonstrates the weight gain in salmon fed different levels of dietary hydroxyproline. The figure demonstrates that dietary inclusion of hydroxyproline leads to a growth promoting effect on final weight and weight gain.

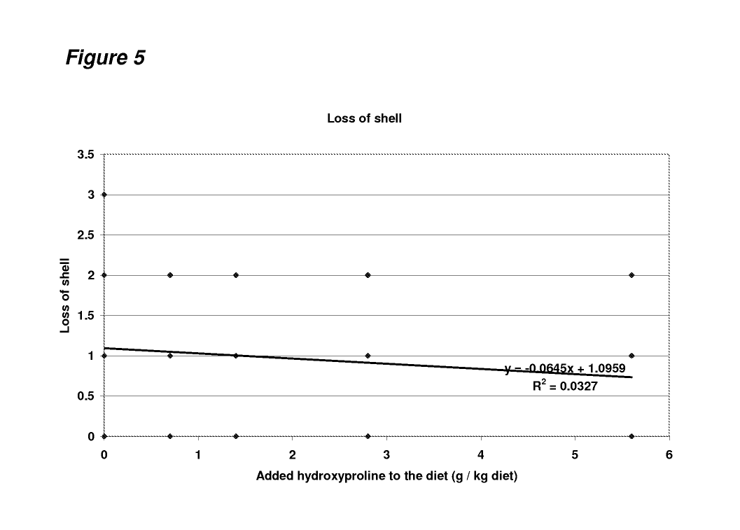

FIG. 5 demonstrates the effect of levels of dietary hydroxyproline on the loss of fish scales (shell loss) from the surface of the fish. (0=no loss of scales observed, 5=serious scale loss, n=110 fish). Increasing amounts of hydroxyproline leads to lower levels of fish scale loss.

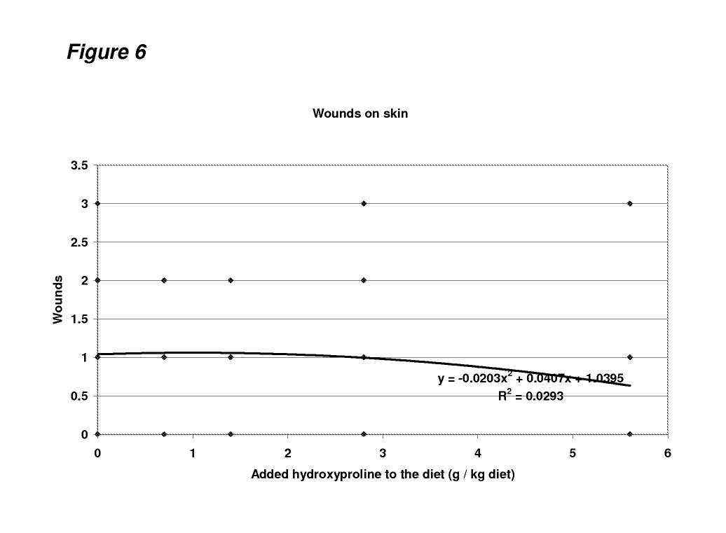

FIG. 6 demonstrates the effect of degree of dietary hydroxyproline on wounding on fish skin in fish fed different levels of dietary hydroxyproline. (0=no wounds observed, 5=serious wounds, n=110 fish). Increasing amounts of dietary hydroxyproline leads to lesser degree of wounding.

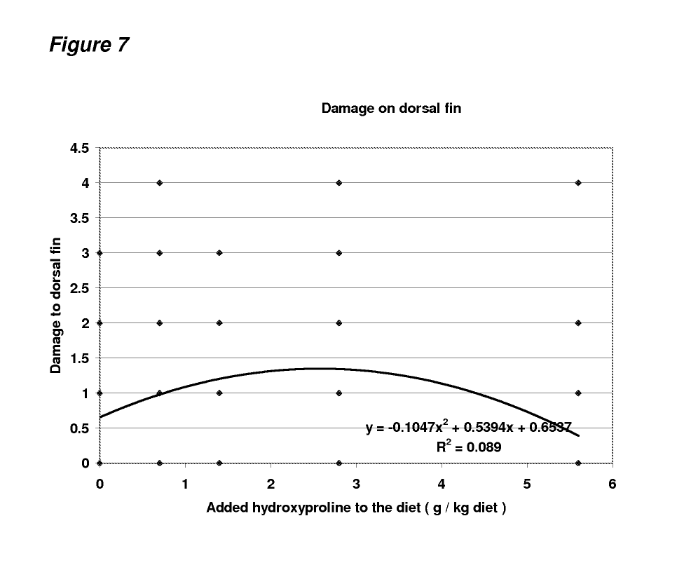

FIG. 7 demonstrates the effect of dietary hydroxyproline on degree of damage to the dorsal fin. (0=no damage observed, 5=serious fin damage, n=110 fish). Increasing amounts of dietary hydroxyproline leads to lesser degree of damage to the dorsal fin.

### DEFINITIONS

Hydroxyproline is an amino acid produced by hydroxylation of the amino acid Proline Hydroxyproline differs from Proline by the presence of a hydroxyl (OH) group attached to the C (gamma) atom. Other Hydroxyprolines also exist in nature, notably 2,3-cis 3,4-trans-diHydroxyproline, as well as L- and D-isomers. The term hydroxyproline as used herein refers to all the above mentioned forms.

The term oligopeptide as used herein encompasses dipeptides.

The term Gingivitis as used herein refers to the inflammation of the gums around the teeth.

The term enteritis as used herein refers to the inflammation and wounds of the small intestine.

The term osteoporosis as used herein refers to a condition that features loss of the normal bone density and elasticity leading to fragile bones.

The term psoriasis as used herein refers to a condition which is believed to be an immune-mediated disease affecting the skin and joints. It commonly causes red scaly plaques to appear on the skin.

A wound is defined herein as a type of physical trauma wherein the skin is torn, cut or punctured (an open wound), or where blunt force trauma causes a contusion (a closed wound). A wound is also defined herein as a condition where the connective tissue is weak and vulnerable due to lack of hydroxyproline and thus the protective layer is disrupted.

The term amino acids is defined herein as Any synthetic or naturally occurring amino carboxylic acid, including any amino acid occurring in peptides and polypeptides including proteins and enzymes synthesized in vivo.

The term Bioactive agent is defined herein as any agent, drug, compound, composition of matter or mixture which provides a beneficial pharmacological effect that can be demonstrated in-vivo or in vitro. This includes beneficial pharmacological effects which can be demonstrated in an individual on a diet comprising an edible food, a food supplement, such as a composition of vitamins, a nutrient, or a nutriceutical comprising the bioactive agent. Also, the beneficial pharmacological effect can be observed in an individual being administered a medicament (drug), a combination of medicaments, a vaccine, or other beneficial agents comprising the bioactive agent.

Bioactive agents comprising a survival enhancing activity: Survival enhancement is an important parameter when e.g. treating an individual for a disease, or when rearing aquatic species or raising fish in industrial fish farms. Bioactive agents comprising a survival enhancing effect are able to improve the survival. Thus such a bioactive agent is also called a survival enhancing agent.

Feed or food: Used herein these terms include feed or food additives, feed or food supplements, feed or food premixes.

Polypeptide: the term “polypeptide” as used herein covers proteins, peptides and polypeptides, wherein said proteins, peptides or polypeptides may or may not have been post-translationally modified. Post-translational modification may for example be phosphorylation, methylation, and/or glucosylation.

%: if nothing else has been stated all the %-values in the present document is weight-by-weight %.

Moisture: in one embodiment the moisture content of the feed composition is the water content of the feed composition.

### DETAILED DESCRIPTION

Hydroxyproline

Hydroxyproline is present in natural protein sources at varying levels. Plant protein has normally very low levels of Hydroxyproline—normally less than 0.1% of protein. Fish meal contains about 1% of protein. The highest levels of Hydroxyproline are found in fish bone meal and in feather meal (about 5.5% of protein). Bone meals from animals have even higher levels of Hydroxyproline about 14-15% of protein.

The feed in this invention include products that have a Hydroxyproline content higher than 15% of protein. Conventional feed supplements generally have very low digestibility, lower than 50%. The

Hydroxyproline—Forms

Hydroxyproline is an amino acid produced by hydroxylation of the amino acid Proline Hydroxyproline differs from Proline by the presence of a hydroxyl (OH) group attached to the C (gamma) atom. Other Hydroxyprolines also exist in nature, notably 2,3-cis 3,4-trans-diHydroxyproline, as well as L- and D-isomers. The term hydroxyproline as used herein refers to all the above mentioned forms.

The present invention may comprise free hydroxyproline, or hydroxyprolyl residues such as those part of an oligopeptide.

The present invention comprises different formulations of hydroxyproline. Examples of such formulations are encapsulated e.g., micro-encapsulated and slow-release pellets With regard to the requirement levels, please be aware that the digestibility of the ingredients high in the component (feather meal and bone meal) have very low digestibility, presumably lower than 50%. A crystalline component will probably have a digestibility of close to 100%.

The invention concerns a composition comprising Hydroxyproline in the range of 0.1% (w/w) to 100% (w/w) of protein or a composition comprising a derivative of Hydroxyproline in the range of 0.1% (w/w) to 100% (w/w) of protein. For pharmaceutical applications pure Hydroxyproline could be applied.

The invention relates to all forms of hydroxyproline. The natural form of Hydroxyproline is present in blood plasma. Ionic forms and other derivatives of Hydroxyproline might be transformed to the pure form during digestion and/or intestinal absorption. The invention further relates to derivatives including a chelated Hydroxyproline compound (e.g. for slow release) as well as novel and inventive formulations of e.g. acylated derivatives of Hydroxyproline. An example is “biodegradable microsphere” comprising Hydroxyproline. The composition may further comprise a carrier and/or essential components in fish feed composition and/or secondary components of fish feed composition.

Fish Food

The present invention relates in one embodiment to a fish feed composition comprising Hydroxyproline. The fish feed composition can be any fish feed composition including a fish feed composition comprising one or more of the ingredients described herein below.

The composition can be used for stimulation of growth in fish. The natural fish prey for carnivorous fish such as salmonoids has with respect to the major components, the following typical composition on dry weight basis: 10-90 wt % of protein, 20-50 wt % of lipids, 5-50 wt % of ashes and nitrogen free extracts. The variation in composition of the natural prey for carnivorous fish is relatively large. This is i.e. caused by the fact that in smaller prey fish the volume of the stomach constitutes a relatively larger proportion of the total weight as compared to that in larger prey fish. Additionally, seasonal fluctuations will affect the composition of the fish prey which e.g. implies that the lipid content is at the highest level in some seasons and at the lowest level in other seasons.

Dietary nutrients are essential for the construction of living tissues. They also are a source of stored energy for fish digestion, absorption, growth, reproduction and the other life processes. The nutritional value of a dietary ingredient is in part dependant on its ability to supply energy. Physiological fuel values are used to calculate and balance available energy values in prepared diets. They typically average 4, 4, and 9 kcal/g for protein, carbohydrate and lipid, respectively.

To create an optimum diet, the ratio of protein to energy must be determined separately for each fish species. Excess energy relative to protein content in the diet may result in high lipid deposition. Because fish feed to meet their energy requirements, diets with excessive energy levels may result in decreased feed intake and reduced weight gain. Similarly, a diet with inadequate energy content can result in reduced weight gain because the fish cannot eat enough feed to satisfy their energy requirements for growth. Properly formulated prepared feeds have a well-balanced energy to protein ratio.

Fish nutrition has advanced dramatically in recent years with development of balanced commercial diets that promote optimal fish growth and health. Furthermore, the development of new species-specific diet formulations supports the fish farming industry.

Commercially available dry feed compositions for carnivorous fish have a content of nutrients which differs significantly from that of the natural prey fish. Thus, such feed compositions commonly have a relatively high content of carbohydrates which are incorporated to act as a binding/structure forming agent in the pellets/granules of the feed. The carbohydrate sources are typically starch-containing cereals such as wheat or cereal starches.

Primary Ingredients of the Fish Food

Prepared or artificial fish diets may be either complete or supplemental. Complete fish diets supply all the ingredients (protein, carbohydrates, fats, vitamins, and minerals) necessary for the optimal growth and health of the fish. Most fish farmers use complete diets, those containing all the required protein (18-50%), lipid (5-45%), carbohydrate (5-25%), ash (<15%), phosphorus (<5%), water (<12%), and trace amounts of vitamins, and minerals. When fish are reared in high density indoor systems or confined in cages and cannot forage freely on natural feeds, they must be provided a complete diet.

In contrast, supplemental (incomplete, partial) diets are intended only to help support the natural food (insects, algae, small fish) normally available to the fish. Supplemental diets do not contain a full complement of vitamins or minerals, but are used to help fortify the naturally available diet with extra protein, carbohydrate and/or lipid.

Protein

Because protein is the most expensive part of fish feed, it is important to accurately determine the protein requirements for each species and size of cultured fish. Proteins are formed by linkages of individual amino acids. 10 of the amino acids are essential meaning that they cannot be synthesized by fish. The 10 essential amino acids that must be supplied by the diet are: methionine, arginine, threonine, tryptophan, histidine, isoleucine, lysine, leucine, valine and phenylalanine. Of these, lysine and methionine are often the first limiting amino acids. Fish feeds prepared with plant (soybean meal) protein typically are low in methionine; therefore, extra methionine must be added to soybean-meal based diets in order to promote optimal growth and health.

Protein levels in aquaculture feeds generally average 18-20% for marine shrimp, 28-32% for catfish, 32-38% for tilapia, 38-42% for hybrid striped bass. Protein requirements usually are lower for herbivorous fish (plant eating) and omnivorous fish (plant-animal eaters) than they are for carnivorous (flesh-eating) fish, and are higher for fish reared in high density (recirculating aquaculture) than low density (pond aquaculture) systems.

Protein requirements generally are higher for smaller fish. As fish grow larger, their protein requirements usually decrease. Protein requirements also vary with rearing environment, water temperature and water quality, as well as the genetic composition and feeding rates of the fish. Protein is used for fish growth if adequate levels of fats and carbohydrates are present in the diet. If not, protein may be used for energy and life support rather than growth.

The protein source may be a liquid or dry non-milk protein source, such as stick water (also referred to as size), fish meal, blood meal, meat meal, bone meal, liquid or dry yeast or proteins derived from a vegetable source including as examples soy beans, peas, maize or cereals. Evidently, a protein source of marine origin has an amino acid composition which is nutritionally appropriate for growing fish, i.e. such a protein contains essential amino acids in the appropriate amounts. This may not be the case with other protein sources, and it may therefore, when non-marine proteins are used in accordance with the invention, be required to supplement the feed composition with appropriate amounts of certain essential amino acids with respect to which the selected protein is deficient, including as examples tryptophan and methionine. A currently preferred protein source is a marine protein source such as fish meal or fish material having a water content in excess of 10 wt % including the semi-manufactured press cake material from fish meal production and stick water also from the processing of fish raw material for fish meal production. In certain fish feed compositions e.g. the so-called “weaning” feed compositions, it is preferred to select fish protein sources of a high quality e.g. having a low content of soluble nitrogen components, a low content of biogenic amines, a low content of carbohydrate, a low content of lipids or containing lipids having a low degree of rancidity. Furthermore, the digestibility of the protein should be high such as at least 90%.

Lipids

Lipids (fats) are high-energy nutrients that can be utilized to partially spare (substitute for) protein in aquaculture feeds. Lipids supply about twice the energy as proteins and carbohydrates. Lipids typically comprise about 15%-50% of fish diets, supply essential fatty acids (EFA) and serve as transporters for fat-soluble vitamins.

Simple lipids include fatty acids and triacylglycerols. Fish typically require fatty acids of the omega 3 and 6 (n-3 and n-6) families. Fatty acids can be: a) saturated fatty acids (SFA, no double bonds), b) polyunsaturated fatty acids (PUFA, >2 double bonds), or c) highly unsaturated fatty acids (HUFA; >4 double bonds). Marine fish oils are naturally high (>30%) in omega 3 HUFA, and are excellent sources of lipids for the manufacture of fish diets.

Marine fish typically require n-3 HUFA for optimal growth and health, usually in quantities ranging from 0.5-2.0% of dry diet. The two major EFA of this group are eicosapentaenoic acid (EPA: 20:5n-3) and docosahexaenoic acid (DHA:22:6n-3). Freshwater fish do not require the long chain HUFA, but often require an 18 carbon n-3 fatty acid, linolenic acid (18:3-n-3), in quantities ranging from 0.5 to 1.5% of dry diet. This fatty acid cannot be produced by freshwater fish and must be supplied in the diet. Many freshwater fish can take this fatty acid, and through enzyme systems elongate (add carbon atoms) to the hydrocarbon chain, and then further desaturate (add double bonds) to this longer hydrocarbon chain. Through these enzyme systems, freshwater fish can manufacture the longer chain n-3 HUFA, EPA and DHA, which are necessary for other metabolic functions and as cellular membrane components. Marine fish typically do not possess these elongation and desaturation enzyme systems, and require long chain n-3 HUFA in their diets. Other fish species, such as tilapia, require fatty acids of the n-6 family, while still others, such as carp or eels, require a combination of n-3 and n-6 fatty acids.

The lipid can be any vegetable lipid and/or marine lipid which may either be solid or liquid at room temperature. One presently preferred lipid is a fish oil, including an at least partially hydrogenated fish oil. However, the lipid may also be selected from other sources such as oils and fats from other animal sources and vegetable oils and fats.

Carbohydrates

Carbohydrates (starches and sugars) are the most economical and inexpensive sources of energy for fish diets. Although not essential, carbohydrates are included in aquaculture diets to reduce feed costs and for their binding activity during feed manufacturing. Dietary starches are useful in the extrusion manufacture of floating feeds. Cooking starch during the extrusion process makes it more biologically available to fish.

In fish, carbohydrates are stored as glycogen that can be mobilized to satisfy energy demands. They are a major energy source for mammals, but are not used efficiently by fish. For example, mammals can extract about 4 kcal of energy from 1 gram of carbohydrate, whereas fish can only extract about 1.6 kcal from the same amount of carbohydrate. Up to about 20% of dietary carbohydrates can be used by fish.

Vitamins

Vitamins are organic compounds necessary in the diet for normal fish growth and health. They often are not synthesized by fish, and must be supplied in the diet. The two groups of vitamins are water-soluble and fat-soluble. Water-soluble vitamins include: the B vitamins, choline, inositol, folic acid, pantothenic acid, biotin and ascorbic acid (vitamin C). The fat-soluble vitamins include A vitamins, retinols; the D vitamins, cholecaciferols; E vitamins, the tocopherols (antioxidants); and K vitamins such as menadione. Deficiency of each vitamin has certain specific symptoms, but reduced growth is the most common symptom of any vitamin deficiency.

A useful source of vitamins is yeast cell mass either in the form a dry yeast or as a liquid yeast cell suspension, i.e. yeast cream.

Minerals

Minerals are inorganic elements necessary in the diet for normal body functions. They can be divided into two groups (macro-minerals and micro-minerals) based on the quantity required in the diet and the amount present in fish. Common macro-minerals are sodium, chloride, potassium and phosphorous. These minerals regulate osmotic balance and aid in bone formation and integrity. Micro-minerals (trace minerals) are required in small amounts as components in enzyme and hormone systems. Common trace minerals are copper, chromium, iodine, zinc and selenium. Fish can absorb many minerals directly from the water through their gills and skin, allowing them to compensate to some extent for mineral deficiencies in their diet.

A useful source of mineral micronutrients is yeast cell mass either in the form a dry yeast or as a liquid yeast cell suspension, i.e. yeast cream.

Secondary Ingredients of the Fish Food

The feed composition may comprise an emulsifying agent (emulsifier), such as a fatty acid, a mono- di- or triglyceride, an organic ester of mono-glycerides, a polyglycerol ester of fatty acids, a propylene glycol ester, a sorbitan ester of fatty acids, a polyoxyethylene sorbitan ester, a sodium stearoyl lactylate, a calcium stearoyl lactylate, a sucrose ester of fatty acids, a sucro-glyceride, a lecithin or a mixture of such emulsifiers. Particularly preferred emulsifying agents are water-insoluble. In specific embodiments the emulsifier is derived from a marine source.

The amount of emulsifying agent is typically in the range of 0.1 to 5 wt %, calculated on the amount of lipid such as in the range of 0.5 to 2 wt %. Based on the finished feed product, the amount of emulsifying agent is typically in the range of 0.05 to 1 wt %. It may be preferred to select an emulsifying agent which is solid at room temperature and having a relatively high melting point such as in the range of 50-60° C. It has been found that by incorporating such an emulsifying agent, the finished feed mixture particles when cooled to ambient temperature set relatively rapidly to provide solid, coherent feed particles which essentially do not disintegrate when administered to the fish breeding environments.

In useful specific embodiments of the invention, the feed composition contains a hydrocolloid such as a non-protein hydrocolloid, preferably a polysaccharide of vegetable or microbial origin. In this context, useful hydrocolloids include a gum such as e.g. guar gum, xanthan gum, gum arabic, LBG, gellan gum, a pectin, a carrageenan, an alginate, a starch including starch derivatives and a water soluble cellulose derivative such as CMC.

In accordance with the invention, the feed composition may comprise further ingredients such as water soluble and water-insoluble vitamins, mineral components, flavouring agents or antioxidants such as e.g. ascorbic acid, ascorbyl palmitate, ascorbyl stearate, BHA (butylated hydroxyanisole), BHT (butylated hydroxytoluene), propyl gallate and tocopherols or preserving agents.

In accordance with the invention, the feed composition can also be supplemented with pharmaceutically active substances. Such active substances typically include antibiotics, vaccines and substances which have a stimulatory effect on the immune system of the growing fish.

Feed Types

The present invention relates in one embodiment to a fish feed composition comprising Hydroxyproline. The fish feed composition can be any type of feed including the ones mentioned herein below.

Commercial fish diets are manufactured as either extruded (floating or buoyant) or pressure-pelleted (sinking) feeds. Both floating and sinking feed can produce satisfactory growth, but some fish species prefer floating, others sinking. Shrimp, for example, will not accept a floating feed, but most fish species can be trained to accept a floating pellet.

Extruded feeds are more expensive due to the higher manufacturing costs. Usually, it is advantageous to feed a floating (extruded) feed, because the farmer can directly observe the feeding intensity of his fish and adjust feeding rates accordingly. Determining whether feeding rates are too low or too high is important in maximizing fish growth and feed use efficiency.

Feed is available in a variety of sizes ranging from fine crumbles for small fish to large (½ inch or larger) pellets. The pellet size should be approximately 20-30% of the size of the fish species mouth gape. Feeding too small a pellet results in inefficient feeding because more energy is used in finding and eating more pellets. Conversely, pellets that are too large will depress feeding and, in the extreme, cause choking.

The composition is suitable for use as a fish feed composition and using the range of 0.7-5.6 gram of Hydroxyproline per kg fish feed composition. Aqua feed should contain 0.5 to 15 g Hydroxyproline per kg diet. The fish feed composition can be dried e.g. freeze-dried, frozen, algae wafers, floating or sinking flake fish feed composition, floating or sinking stick fish feed composition, pellet fish feed composition or slowly dissolving fish feed composition. The fish feed composition can also be used for feeding of a fish population to be used as “live feed”.

Fish Feed Composition Ingredients

In one embodiment the present invention relates to a fish feed composition comprising High quality fish meal e.g. in the range from 30 to 60%, such as from 30-31%, for example 31-32%, such as from 32-33%, for example 33-34%, such as from 34-35%, for example 35-36%, such as from 36-37%, for example 37-38%, such as from 38-39%, for example 39-40%, such as from 40-41%, for example 41-42%, such as from 42-43%, for example 43-44%, such as from 44-45%, for example 45-46%, such as from 46-47%, for example 47-48%, such as from 48-49%, for example 49-50%, such as from 50-51%, for example 51-52%, such as from 52-53%, for example 53-54%, such as from 54-55%, for example 55-56%, such as from 56-57%, for example 57-58%, such as from 58-59%, for example 59-60%.

In another embodiment the present invention relates to a fish feed composition comprising krill meal e.g. in the range from 0-10%, such as from 0-0.25%, for example from 0.25%-0.5%, such as from 0.5-0.75%, for example from 0.75%-1.0%, such as from 1.0-1.25%, for example from 1.25%-1.5%, such as from 1.5-1.75%, for example from 1.75%-2.0%, such as from 2.0-2.25%, for example from 2.25%-2.5%, such as from 2.5-2.75%, for example from 2.75%-3.0%, such as from 3.0-3.25%, for example from 3.25%-3.5%, such as from 3.5-3.75%, for example from 3.75%-4.0%, such as from 4.0-4.25%, for example from 4.25%-4.5%, such as from 4.5-4.75%, for example from 4.75%-5.0%, such as from 5.0-5.25%, for example from 5.25%-5.5%, such as from 5.5-5.75%, for example from 5.75%-6.0%, such as from 6.0-6.25%, for example from 6.25%-6.5%, such as from 6.5-6.75%, for example from 6.75%-7.0%, such as from 7.0-7.25%, for example from 7.25%-7.5%, such as from 7.5-7.75%, for example from 7.75%-8.0%, such as from 8.0-8.25%, for example from 8.25%-8.5%, such as from 8.5-8.75%, for example from 8.75%-9.0%, such as from 9.0-9.25%, for example from 9.25%-9.5%, such as from 9.5-9.75%, for example from 9.75%-10.0%.

In another embodiment the present invention relates to a fish feed composition comprising corn starch e.g. in the range from 0-10%, such as from 0-0.25%, for example from 0.25%-0.5%, such as from 0.5-0.75%, for example from 0.75%-1.0%, such as from 1.0-1.25%, for example from 1.25%-1.5%, such as from 1.5-1.75%, for example from 1.75%-2.0%, such as from 2.0-2.25%, for example from 2.25%-2.5%, such as from 2.5-2.75%, for example from 2.75%-3.0%, such as from 3.0-3.25%, for example from 3.25%-3.5%, such as from 3.5-3.75%, for example from 3.75%-4.0%, such as from 4.0-4.25%, for example from 4.25%-4.5%, such as from 4.5-4.75%, for example from 4.75%-5.0%, such as from 5.0-5.25%, for example from 5.25%-5.5%, such as from 5.5-5.75%, for example from 5.75%-6.0%, such as from 6.0-6.25%, for example from 6.25%-6.5%, such as from 6.5-6.75%, for example from 6.75%-7.0%, such as from 7.0-7.25%, for example from 7.25%-7.5%, such as from 7.5-7.75%, for example from 7.75%-8.0%, such as from 8.0-8.25%, for example from 8.25%-8.5%, such as from 8.5-8.75%, for example from 8.75%-9.0%, such as from 9.0-9.25%, for example from 9.25%-9.5%, such as from 9.5-9.75%, for example from 9.75%-10.0%.

In another embodiment the present invention relates to a fish feed composition comprising algae meal e.g. in the range from 0-10%, such as from 0-0.25%, for example from 0.25%-0.5%, such as from 0.5-0.75%, for example from 0.75%-1.0%, such as from 1.0-1.25%, for example from 1.25%-1.5%, such as from 1.5-1.75%, for example from 1.75%-2.0%, such as from 2.0-2.25%, for example from 2.25%-2.5%, such as from 2.5-2.75%, for example from 2.75%-3.0%, such as from 3.0-3.25%, for example from 3.25%-3.5%, such as from 3.5-3.75%, for example from 3.75%-4.0%, such as from 4.0-4.25%, for example from 4.25%-4.5%, such as from 4.5-4.75%, for example from 4.75%-5.0%, %, such as from 5.0-5.25%, for example from 5.25%-5.5%, such as from 5.5-5.75%, for example from 5.75%-6.0%, such as from 6.0-6.25%, for example from 6.25%-6.5%, such as from 6.5-6.75%, for example from 6.75%-7.0%, such as from 7.0-7.25%, for example from 7.25%-7.5%, such as from 7.5-7.75%, for example from 7.75%-8.0%, such as from 8.0-8.25%, for example from 8.25%-8.5%, such as from 8.5-8.75%, for example from 8.75%-9.0%, such as from 9.0-9.25%, for example from 9.25%-9.5%, such as from 9.5-9.75%, for example from 9.75%-10.0%.

In another embodiment the present invention relates to a fish feed composition comprising mineral mix e.g. in the range from 0-5%, such as from 0-0.1%, for example from 0.1-0.2%, such as from 0.2-0.3%, for example from 0.3-0.4%, such as from 0.4-0.5%, for example from 0.5-0.6%, such as from 0.6-0.7%, for example from 0.7-0.8%, such as from 0.8-0.9%, for example from 0.9-1.0%, such as from 1.0-1.1%, for example from 1.1-1.2%, such as from 1.2-1.3%, for example from 1.3-1.4%, such as from 1.4-1.5%, for example from 1.5-1.6%, such as from 1.6-1.7%, for example from 1.7-1.8%, such as from 1.8-1.9%, for example from 1.9-2.0%, such as from 2.0-2.2%, for example from 2.2-2.4%, such as from 2.4-2.6%, for example from 2.6-2.8%, such as from 2.8-3.0%, for example from 3.0-3.2%, such as from 3.2-3.4%, for example from 3.4-3.6%, such as from 3.6-3.8%, for example from 3.8-4.0%, such as from 4.0-4.2%, for example from 4.2-4.4%, such as from 4.4-4.6%, for example from 4.6-4.8%, such as from 4.8-5.0%.

In another embodiment the present invention relates to a fish feed composition comprising a vitamin mix e.g. in the range from 0-10%, such as from 0-0.25%, for example from 0.25%-0.5%, such as from 0.5-0.75%, for example from 0.75%-1.0%, such as from 1.0-1.25%, for example from 1.25%-1.5%, such as from 1.5-1.75%, for example from 1.75%-2.0%, such as from 2.0-2.25%, for example from 2.25%-2.5%, such as from 2.5-2.75%, for example from 2.75%-3.0%, such as from 3.0-3.25%, for example from 3.25%-3.5%, such as from 3.5-3.75%, for example from 3.75%-4.0%, such as from 4.0-4.25%, for example from 4.25%-4.5%, such as from 4.5-4.75%, for example from 4.75%-5.0%, %, such as from 5.0-5.25%, for example from 5.25%-5.5%, such as from 5.5-5.75%, for example from 5.75%-6.0%, such as from 6.0-6.25%, for example from 6.25%-6.5%, such as from 6.5-6.75%, for example from 6.75%-7.0%, such as from 7.0-7.25%, for example from 7.25%-7.5%, such as from 7.5-7.75%, for example from 7.75%-8.0%, such as from 8.0-8.25%, for example from 8.25%-8.5%, such as from 8.5-8.75%, for example from 8.75%-9.0%, such as from 9.0-9.25%, for example from 9.25%-9.5%, such as from 9.5-9.75%, for example from 9.75%-10.0%.

In another embodiment the present invention relates to a fish feed composition comprising betafin (betain) e.g. in the range from 0-10%, such as from 0-0.25%, for example from 0.25%-0.5%, such as from 0.5-0.75%, for example from 0.75%-1.0%, such as from 1.0-1.25%, for example from 1.25%-1.5%, such as from 1.5-1.75%, for example from 1.75%-2.0%, such as from 2.0-2.25%, for example from 2.25%-2.5%, such as from 2.5-2.75%, for example from 2.75%-3.0%, such as from 3.0-3.25%, for example from 3.25%-3.5%, such as from 3.5-3.75%, for example from 3.75%-4.0%, such as from 4.0-4.25%, for example from 4.25%-4.5%, such as from 4.5-4.75%, for example from 4.75%-5.0%, %, such as from 5.0-5.25%, for example from 5.25%-5.5%, such as from 5.5-5.75%, for example from 5.75%-6.0%, such as from 6.0-6.25%, for example from 6.25%-6.5%, such as from 6.5-6.75%, for example from 6.75%-7.0%, such as from 7.0-7.25%, for example from 7.25%-7.5%, such as from 7.5-7.75%, for example from 7.75%-8.0%, such as from 8.0-8.25%, for example from 8.25%-8.5%, such as from 8.5-8.75%, for example from 8.75%-9.0%, such as from 9.0-9.25%, for example from 9.25%-9.5%, such as from 9.5-9.75%, for example from 9.75%-10.0%.

In another embodiment the present invention relates to a fish feed composition comprising raw wheat e.g. in the range from 0-10%, such as from 0-0.25%, for example from 0.25%-0.5%, such as from 0.5-0.75%, for example from 0.75%-1.0%, such as from 1.0-1.25%, for example from 1.25%-1.5%, such as from 1.5-1.75%, for example from 1.75%-2.0%, such as from 2.0-2.25%, for example from 2.25%-2.5%, such as from 2.5-2.75%, for example from 2.75%-3.0%, such as from 3.0-3.25%, for example from 3.25%-3.5%, such as from 3.5-3.75%, for example from 3.75%-4.0%, such as from 4.0-4.25%, for example from 4.25%-4.5%, such as from 4.5-4.75%, for example from 4.75%-5.0%, %, such as from 5.0-5.25%, for example from 5.25%-5.5%, such as from 5.5-5.75%, for example from 5.75%-6.0%, such as from 6.0-6.25%, for example from 6.25%-6.5%, such as from 6.5-6.75%, for example from 6.75%-7.0%, such as from 7.0-7.25%, for example from 7.25%-7.5%, such as from 7.5-7.75%, for example from 7.75%-8.0%, such as from 8.0-8.25%, for example from 8.25%-8.5%, such as from 8.5-8.75%, for example from 8.75%-9.0%, such as from 9.0-9.25%, for example from 9.25%-9.5%, such as from 9.5-9.75%, for example from 9.75%-10.0%.

In another embodiment the present invention relates to a fish feed composition comprising dicalsiumphosphate e.g. in the range from 0-10%, such as from 0-0.25%, for example from 0.25%-0.5%, such as from 0.5-0.75%, for example from 0.75%-1.0%, such as from 1.0-1.25%, for example from 1.25%-1.5%, such as from 1.5-1.75%, for example from 1.75%-2.0%, such as from 2.0-2.25%, for example from 2.25%-2.5%, such as from 2.5-2.75%, for example from 2.75%-3.0%, such as from 3.0-3.25%, for example from 3.25%-3.5%, such as from 3.5-3.75%, for example from 3.75%-4.0%, such as from 4.0-4.25%, for example from 4.25%-4.5%, such as from 4.5-4.75%, for example from 4.75%-5.0%, %, such as from 5.0-5.25%, for example from 5.25%-5.5%, such as from 5.5-5.75%, for example from 5.75%-6.0%, such as from 6.0-6.25%, for example from 6.25%-6.5%, such as from 6.5-6.75%, for example from 6.75%-7.0%, such as from 7.0-7.25%, for example from 7.25%-7.5%, such as from 7.5-7.75%, for example from 7.75%-8.0%, such as from 8.0-8.25%, for example from 8.25%-8.5%, such as from 8.5-8.75%, for example from 8.75%-9.0%, such as from 9.0-9.25%, for example from 9.25%-9.5%, such as from 9.5-9.75%, for example from 9.75%-10.0%.

In another embodiment the present invention relates to a fish feed composition comprising Inositol e.g. in the range from 0-1%, such as from 0-0.01%, for example from 0.01-0.02%, such as from 0.02-0.03%, for example from 0.03-0.04%, such as from 0.04-0.05%, for example from 0.05-0.06%, such as from 0.06-0.07%, for example from 0.07-0.08%, such as from 0.08-0.09%, for example from 0.09-0.10%, such as from 0.10-0.11%, for example from 0.11-0.12%, such as from 0.12-0.13%, for example from 0.13-0.14%, such as from 0.14-0.15%, for example from 0.15-0.16%, such as from 0.16-0.17%, for example from 0.17-0.18%, such as from 0.18-0.19%, for example from 0.19-0.20%, such as from 0.2-0.3%, for example from 0.3-0.4%, such as from 0.4-0.5%, for example from 0.5-0.6%, such as from 0.6-0.7%, for example from 0.7-0.8%, such as from 0.8-0.9%, for example from 0.9-1.0%.

In another embodiment the present invention relates to a fish feed composition comprising yeast extract e.g. in the range from 0-10%, such as from 0-0.25%, for example from 0.25%-0.5%, such as from 0.5-0.75%, for example from 0.75%-1.0%, such as from 1.0-1.25%, for example from 1.25%-1.5%, such as from 1.5-1.75%, for example from 1.75%-2.0%, such as from 2.0-2.25%, for example from 2.25%-2.5%, such as from 2.5-2.75%, for example from 2.75%-3.0%, such as from 3.0-3.25%, for example from 3.25%-3.5%, such as from 3.5-3.75%, for example from 3.75%-4.0%, such as from 4.0-4.25%, for example from 4.25%-4.5%, such as from 4.5-4.75%, for example from 4.75%-5.0%, %, such as from 5.0-5.25%, for example from 5.25%-5.5%, such as from 5.5-5.75%, for example from 5.75%-6.0%, such as from 6.0-6.25%, for example from 6.25%-6.5%, such as from 6.5-6.75%, for example from 6.75%-7.0%, such as from 7.0-7.25%, for example from 7.25%-7.5%, such as from 7.5-7.75%, for example from 7.75%-8.0%, such as from 8.0-8.25%, for example from 8.25%-8.5%, such as from 8.5-8.75%, for example from 8.75%-9.0%, such as from 9.0-9.25%, for example from 9.25%-9.5%, such as from 9.5-9.75%, for example from 9.75%-10.0%.

In another embodiment the present invention relates to a fish feed composition comprising cholesterol e.g. in the range from 0-10%, such as from 0-0.25%, for example from 0.25%-0.5%, such as from 0.5-0.75%, for example from 0.75%-1.0%, such as from 1.0-1.25%, for example from 1.25%-1.5%, such as from 1.5-1.75%, for example from 1.75%-2.0%, such as from 2.0-2.25%, for example from 2.25%-2.5%, such as from 2.5-2.75%, for example from 2.75%-3.0%, such as from 3.0-3.25%, for example from 3.25%-3.5%, such as from 3.5-3.75%, for example from 3.75%-4.0%, such as from 4.0-4.25%, for example from 4.25%-4.5%, such as from 4.5-4.75%, for example from 4.75%-5.0%, %, such as from 5.0-5.25%, for example from 5.25%-5.5%, such as from 5.5-5.75%, for example from 5.75%-6.0%, such as from 6.0-6.25%, for example from 6.25%-6.5%, such as from 6.5-6.75%, for example from 6.75%-7.0%, such as from 7.0-7.25%, for example from 7.25%-7.5%, such as from 7.5-7.75%, for example from 7.75%-8.0%, such as from 8.0-8.25%, for example from 8.25%-8.5%, such as from 8.5-8.75%, for example from 8.75%-9.0%, such as from 9.0-9.25%, for example from 9.25%-9.5%, such as from 9.5-9.75%, for example from 9.75%-10.0%.

In another embodiment the present invention relates to a fish feed composition comprising vitamin c e.g. in the range from 0-2%, such as from 0-0.01%, for example from 0.01-0.02%, such as from 0.02-0.03%, for example from 0.03-0.04%, such as from 0.04-0.05%, for example from 0.05-0.06%, such as from 0.06-0.07%, for example from 0.07-0.08%, such as from 0.08-0.09%, for example from 0.09-0.10%, such as from 0.10-0.11%, for example from 0.11-0.12%, such as from 0.12-0.13%, for example from 0.13-0.14%, such as from 0.14-0.15%, for example from 0.15-0.16%, such as from 0.16-0.17%, for example from 0.17-0.18%, such as from 0.18-0.19%, for example from 0.19-0.20%, such as from 0.20-0.21%, for example from 0.21-0.22%, such as from 0.22-0.23%, for example from 0.23-0.24%, such as from 0.24-0.25%, for example from 0.25-0.26%, such as from 0.26-0.27%, for example from 0.27-0.28%, such as from 0.28-0.29%, for example from 0.29-0.30%, such as from 0.30-0.32%, for example from 0.32-0.34%, such as from 0.34-0.36%, for example from 0.36-0.38%, such as from 0.38-0.40%, for example from 0.40-0.5%, such as from 0.5-0.6%, for example from 0.6-0.7%, such as from 0.7-0.8%, for example from 0.8-0.9%, such as from 0.9-1.0%, for example from 1.0-1.1%, such as from 1.1-1.2%, for example from 1.2-1.3%, such as from 1.3-1.4%, for example from 1.4-1.5%, such as from 1.5-1.6%, for example from 1.6-1.7%, such as from 1.7-1.8%, for example from 1.8-1.9%, such as from 1.9-2.0%.

In another embodiment the present invention relates to a fish feed composition comprising Soya Lecitin e.g. in the range from 0-10%, such as from 0-0.25%, for example from 0.25%-0.5%, such as from 0.5-0.75%, for example from 0.75%-1.0%, such as from 1.0-1.25%, for example from 1.25%-1.5%, such as from 1.5-1.75%, for example from 1.75%-2.0%, such as from 2.0-2.25%, for example from 2.25%-2.5%, such as from 2.5-2.75%, for example from 2.75%-3.0%, such as from 3.0-3.25%, for example from 3.25%-3.5%, such as from 3.5-3.75%, for example from 3.75%-4.0%, such as from 4.0-4.25%, for example from 4.25%-4.5%, such as from 4.5-4.75%, for example from 4.75%-5.0%, %, such as from 5.0-5.25%, for example from 5.25%-5.5%, such as from 5.5-5.75%, for example from 5.75%-6.0%, such as from 6.0-6.25%, for example from 6.25%-6.5%, such as from 6.5-6.75%, for example from 6.75%-7.0%, such as from 7.0-7.25%, for example from 7.25%-7.5%, such as from 7.5-7.75%, for example from 7.75%-8.0%, such as from 8.0-8.25%, for example from 8.25%-8.5%, such as from 8.5-8.75%, for example from 8.75%-9.0%, such as from 9.0-9.25%, for example from 9.25%-9.5%, such as from 9.5-9.75%, for example from 9.75%-10.0%.

In another embodiment the present invention relates to a fish feed composition comprising fish oil e.g. in the range from 0-10%, such as from 0-0.25%, for example from 0.25%-0.5%, such as from 0.5-0.75%, for example from 0.75%-1.0%, such as from 1.0-1.25%, for example from 1.25%-1.5%, such as from 1.5-1.75%, for example from 1.75%-2.0%, such as from 2.0-2.25%, for example from 2.25%-2.5%, such as from 2.5-2.75%, for example from 2.75%-3.0%, such as from 3.0-3.25%, for example from 3.25%-3.5%, such as from 3.5-3.75%, for example from 3.75%-4.0%, such as from 4.0-4.25%, for example from 4.25%-4.5%, such as from 4.5-4.75%, for example from 4.75%-5.0%, %, such as from 5.0-5.25%, for example from 5.25%-5.5%, such as from 5.5-5.75%, for example from 5.75%-6.0%, such as from 6.0-6.25%, for example from 6.25%-6.5%, such as from 6.5-6.75%, for example from 6.75%-7.0%, such as from 7.0-7.25%, for example from 7.25%-7.5%, such as from 7.5-7.75%, for example from 7.75%-8.0%, such as from 8.0-8.25%, for example from 8.25%-8.5%, such as from 8.5-8.75%, for example from 8.75%-9.0%, such as from 9.0-9.25%, for example from 9.25%-9.5%, such as from 9.5-9.75%, for example from 9.75%-10.0%.

In another embodiment the present invention relates to a fish feed composition comprising stickwater e.g in the range from 0-20%, such as from 0-0.25%, for example from 0.25%-0.5%, such as from 0.5-0.75%, for example from 0.75%-1.0%, such as from 1.0-1.25%, for example from 1.25%-1.5%, such as from 1.5-1.75%, for example from 1.75%-2.0%, such as from 2.0-2.25%, for example from 2.25%-2.5%, such as from 2.5-2.75%, for example from 2.75%-3.0%, such as from 3.0-3.25%, for example from 3.25%-3.5%, such as from 3.5-3.75%, for example from 3.75%-4.0%, such as from 4.0-4.25%, for example from 4.25%-4.5%, such as from 4.5-4.75%, for example from 4.75%-5.0%, %, such as from 5.0-5.25%, for example from 5.25%-5.5%, such as from 5.5-5.75%, for example from 5.75%-6.0%, such as from 6.0-6.25%, for example from 6.25%-6.5%, such as from 6.5-6.75%, for example from 6.75%-7.0%, such as from 7.0-7.25%, for example from 7.25%-7.5%, such as from 7.5-7.75%, for example from 7.75%-8.0%, such as from 8.0-8.25%, for example from 8.25%-8.5%, such as from 8.5-8.75%, for example from 8.75%-9.0%, such as from 9.0-9.25%, for example from 9.25%-9.5%, such as from 9.5-9.75%, for example from 9.75%-10.0%, such as from 10-10.25%, for example from 10.25%-10.5%, such as from 10.5-10.75%, for example from 10.75%-11.0%, such as from 11.0-11.25%, for example from 11.25%-11.5%, such as from 11.5-11.75%, for example from 11.75%-12.0%, such as from 12.0-12.25%, for example from 12.25%-12.5%, such as from 12.5-12.75%, for example from 12.75%-13.0%, such as from 13.0-13.25%, for example from 13.25%-13.5%, such as from 13.5-13.75%, for example from 13.75%-14.0%, such as from 14.0-14.25%, for example from 14.25%-14.5%, such as from 14.5-14.75%, for example from 14.75%-15.0%, %, such as from 15.0-15.25%, for example from 15.25%-15.5%, such as from 15.5-15.75%, for example from 15.75%-16.0%, such as from 16.0-16.25%, for example from 16.25%-16.5%, such as from 16.5-16.75%, for example from 16.75%-17.0%, such as from 17.0-17.25%, for example from 17.25%-17.5%, such as from 17.5-17.75%, for example from 17.75%-18.0%, such as from 18.0-18.25%, for example from 18.25%-18.5%, such as from 18.5-18.75%, for example from 18.75%-19.0%, such as from 19.0-19.25%, for example from 19.25%-19.5%, such as from 19.5-19.75%, for example from 19.75%-20.0%.

In another embodiment the present invention relates to a fish feed composition comprising Astaxanthin e.g. in the range from 0-1%, such as from 0-0.01%, for example from 0.01-0.02%, such as from 0.02-0.03%, for example from 0.03-0.04%, such as from 0.04-0.05%, for example from 0.05-0.06%, such as from 0.06-0.07%, for example from 0.07-0.08%, such as from 0.08-0.09%, for example from 0.09-0.10%, such as from 0.10-0.11%, for example from 0.11-0.12%, such as from 0.12-0.13%, for example from 0.13-0.14%, such as from 0.14-0.15%, for example from 0.15-0.16%, such as from 0.16-0.17%, for example from 0.17-0.18%, such as from 0.18-0.19%, for example from 0.19-0.20%, such as from 0.2-0.3%, for example from 0.3-0.4%, such as from 0.4-0.5%, for example from 0.5-0.6%, such as from 0.6-0.7%, for example from 0.7-0.8%, such as from 0.8-0.9%, for example from 0.9-1.0%.

In another embodiment the present invention relates to a fish feed composition comprising fish hydrolysate e.g. in the range from 0-10%, such as from 0-0.25%, for example from 0.25%-0.5%, such as from 0.5-0.75%, for example from 0.75%-1.0%, such as from 1.0-1.25%, for example from 1.25%-1.5%, such as from 1.5-1.75%, for example from 1.75%-2.0%, such as from 2.0-2.25%, for example from 2.25%-2.5%, such as from 2.5-2.75%, for example from 2.75%-3.0%, such as from 3.0-3.25%, for example from 3.25%-3.5%, such as from 3.5-3.75%, for example from 3.75%-4.0%, such as from 4.0-4.25%, for example from 4.25%-4.5%, such as from 4.5-4.75%, for example from 4.75%-5.0%, %, such as from 5.0-5.25%, for example from 5.25%-5.5%, such as from 5.5-5.75%, for example from 5.75%-6.0%, such as from 6.0-6.25%, for example from 6.25%-6.5%, such as from 6.5-6.75%, for example from 6.75%-7.0%, such as from 7.0-7.25%, for example from 7.25%-7.5%, such as from 7.5-7.75%, for example from 7.75%-8.0%, such as from 8.0-8.25%, for example from 8.25%-8.5%, such as from 8.5-8.75%, for example from 8.75%-9.0%, such as from 9.0-9.25%, for example from 9.25%-9.5%, such as from 9.5-9.75%, for example from 9.75%-10.0%.

In another embodiment the present invention relates to a fish feed composition comprising gelatin e.g. in the range from 0-20%, such as from 0-0.25%, for example from 0.25%-0.5%, such as from 0.5-0.75%, for example from 0.75%-1.0%, such as from 1.0-1.25%, for example from 1.25%-1.5%, such as from 1.5-1.75%, for example from 1.75%-2.0%, such as from 2.0-2.25%, for example from 2.25%-2.5%, such as from 2.5-2.75%, for example from 2.75%-3.0%, such as from 3.0-3.25%, for example from 3.25%-3.5%, such as from 3.5-3.75%, for example from 3.75%-4.0%, such as from 4.0-4.25%, for example from 4.25%-4.5%, such as from 4.5-4.75%, for example from 4.75%-5.0%, %, such as from 5.0-5.25%, for example from 5.25%-5.5%, such as from 5.5-5.75%, for example from 5.75%-6.0%, such as from 6.0-6.25%, for example from 6.25%-6.5%, such as from 6.5-6.75%, for example from 6.75%-7.0%, such as from 7.0-7.25%, for example from 7.25%-7.5%, such as from 7.5-7.75%, for example from 7.75%-8.0%, such as from 8.0-8.25%, for example from 8.25%-8.5%, such as from 8.5-8.75%, for example from 8.75%-9.0%, such as from 9.0-9.25%, for example from 9.25%-9.5%, such as from 9.5-9.75%, for example from 9.75%-10.0%, such as from 10-10.25%, for example from 10.25%-10.5%, such as from 10.5-10.75%, for example from 10.75%-11.0%, such as from 11.0-11.25%, for example from 11.25%-11.5%, such as from 11.5-11.75%, for example from 11.75%-12.0%, such as from 12.0-12.25%, for example from 12.25%-12.5%, such as from 12.5-12.75%, for example from 12.75%-13.0%, such as from 13.0-13.25%, for example from 13.25%-13.5%, such as from 13.5-13.75%, for example from 13.75%-14.0%, such as from 14.0-14.25%, for example from 14.25%-14.5%, such as from 14.5-14.75%, for example from 14.75%-15.0%, %, such as from 15.0-15.25%, for example from 15.25%-15.5%, such as from 15.5-15.75%, for example from 15.75%-16.0%, such as from 16.0-16.25%, for example from 16.25%-16.5%, such as from 16.5-16.75%, for example from 16.75%-17.0%, such as from 17.0-17.25%, for example from 17.25%-17.5%, such as from 17.5-17.75%, for example from 17.75%-18.0%, such as from 18.0-18.25%, for example from 18.25%-18.5%, such as from 18.5-18.75%, for example from 18.75%-19.0%, such as from 19.0-19.25%, for example from 19.25%-19.5%, such as from 19.5-19.75%, for example from 19.75%-20.0%.

In another embodiment the present invention relates to a fish feed composition comprising an immune stimulant e.g. in the range from 0-10%, such as from 0-0.25%, for example from 0.25%-0.5%, such as from 0.5-0.75%, for example from 0.75%-1.0%, such as from 1.0-1.25%, for example from 1.25%-1.5%, such as from 1.5-1.75%, for example from 1.75%-2.0%, such as from 2.0-2.25%, for example from 2.25%-2.5%, such as from 2.5-2.75%, for example from 2.75%-3.0%, such as from 3.0-3.25%, for example from 3.25%-3.5%, such as from 3.5-3.75%, for example from 3.75%-4.0%, such as from 4.0-4.25%, for example from 4.25%-4.5%, such as from 4.5-4.75%, for example from 4.75%-5.0%, %, such as from 5.0-5.25%, for example from 5.25%-5.5%, such as from 5.5-5.75%, for example from 5.75%-6.0%, such as from 6.0-6.25%, for example from 6.25%-6.5%, such as from 6.5-6.75%, for example from 6.75%-7.0%, such as from 7.0-7.25%, for example from 7.25%-7.5%, such as from 7.5-7.75%, for example from 7.75%-8.0%, such as from 8.0-8.25%, for example from 8.25%-8.5%, such as from 8.5-8.75%, for example from 8.75%-9.0%, such as from 9.0-9.25%, for example from 9.25%-9.5%, such as from 9.5-9.75%, for example from 9.75%-10.0%.

In another embodiment the fish feed composition comprises from 40 to 80% of protein, such as from 40-41%, for example 41-42%, such as from 42-43%, for example 43-44%, such as from 44-45%, for example 45-46%, such as from 46-47%, for example 47-48%, such as from 48-49%, for example 49-50%, such as from 50-51%, for example 51-52%, such as from 52-53%, for example 53-54%, such as from 54-55%, for example 55-56%, such as from 56-57%, for example 57-58%, such as from 58-59%, for example 59-60%, such as from 60-61%, for example 61-62%, such as from 62-63%, for example 63-64%, such as from 64-65%, for example 65-66%, such as from 66-67%, for example 67-68%, such as from 68-69%, for example 69-70%, such as from 70-71%, for example 71-72%, such as from 72-73%, for example 73-74%, such as from 74-75%, for example 75-76%, such as from 76-77%, for example 77-78%, such as from 78-79%, for example 79-80%.

In another embodiment the fish feed composition comprises from 0-20% lipid, such as from 0-0.25%, for example from 0.25%-0.5%, such as from 0.5-0.75%, for example from 0.75%-1.0%, such as from 1.0-1.25%, for example from 1.25%-1.5%, such as from 1.5-1.75%, for example from 1.75%-2.0%, such as from 2.0-2.25%, for example from 2.25%-2.5%, such as from 2.5-2.75%, for example from 2.75%-3.0%, such as from 3.0-3.25%, for example from 3.25%-3.5%, such as from 3.5-3.75%, for example from 3.75%-4.0%, such as from 4.0-4.25%, for example from 4.25%-4.5%, such as from 4.5-4.75%, for example from 4.75%-5.0%, %, such as from 5.0-5.25%, for example from 5.25%-5.5%, such as from 5.5-5.75%, for example from 5.75%-6.0%, such as from 6.0-6.25%, for example from 6.25%-6.5%, such as from 6.5-6.75%, for example from 6.75%-7.0%, such as from 7.0-7.25%, for example from 7.25%-7.5%, such as from 7.5-7.75%, for example from 7.75%-8.0%, such as from 8.0-8.25%, for example from 8.25%-8.5%, such as from 8.5-8.75%, for example from 8.75%-9.0%, such as from 9.0-9.25%, for example from 9.25%-9.5%, such as from 9.5-9.75%, for example from 9.75%-10.0%, such as from 10-10.25%, for example from 10.25%-10.5%, such as from 10.5-10.75%, for example from 10.75%-11.0%, such as from 11.0-11.25%, for example from 11.25%-11.5%, such as from 11.5-11.75%, for example from 11.75%-12.0%, such as from 12.0-12.25%, for example from 12.25%-12.5%, such as from 12.5-12.75%, for example from 12.75%-13.0%, such as from 13.0-13.25%, for example from 13.25%-13.5%, such as from 13.5-13.75%, for example from 13.75%-14.0%, such as from 14.0-14.25%, for example from 14.25%-14.5%, such as from 14.5-14.75%, for example from 14.75%-15.0%, %, such as from 15.0-15.25%, for example from 15.25%-15.5%, such as from 15.5-15.75%, for example from 15.75%-16.0%, such as from 16.0-16.25%, for example from 16.25%-16.5%, such as from 16.5-16.75%, for example from 16.75%-17.0%, such as from 17.0-17.25%, for example from 17.25%-17.5%, such as from 17.5-17.75%, for example from 17.75%-18.0%, such as from 18.0-18.25%, for example from 18.25%-18.5%, such as from 18.5-18.75%, for example from 18.75%-19.0%, such as from 19.0-19.25%, for example from 19.25%-19.5%, such as from 19.5-19.75%, for example from 19.75%-20.0%.

In another embodiment the fish feed composition comprises from 1-20% of ash, such as from 0-0.25%, for example from 0.25%-0.5%, such as from 0.5-0.75%, for example from 0.75%-1.0%, such as from 1.0-1.25%, for example from 1.25%-1.5%, such as from 1.5-1.75%, for example from 1.75%-2.0%, such as from 2.0-2.25%, for example from 2.25%-2.5%, such as from 2.5-2.75%, for example from 2.75%-3.0%, such as from 3.0-3.25%, for example from 3.25%-3.5%, such as from 3.5-3.75%, for example from 3.75%-4.0%, such as from 4.0-4.25%, for example from 4.25%-4.5%, such as from 4.5-4.75%, for example from 4.75%-5.0%, %, such as from 5.0-5.25%, for example from 5.25%-5.5%, such as from 5.5-5.75%, for example from 5.75%-6.0%, such as from 6.0-6.25%, for example from 6.25%-6.5%, such as from 6.5-6.75%, for example from 6.75%-7.0%, such as from 7.0-7.25%, for example from 7.25%-7.5%, such as from 7.5-7.75%, for example from 7.75%-8.0%, such as from 8.0-8.25%, for example from 8.25%-8.5%, such as from 8.5-8.75%, for example from 8.75%-9.0%, such as from 9.0-9.25%, for example from 9.25%-9.5%, such as from 9.5-9.75%, for example from 9.75%-10.0%, such as from 10-10.25%, for example from 10.25%-10.5%, such as from 10.5-10.75%, for example from 10.75%-11.0%, such as from 11.0-11.25%, for example from 11.25%-11.5%, such as from 11.5-11.75%, for example from 11.75%-12.0%, such as from 12.0-12.25%, for example from 12.25%-12.5%, such as from 12.5-12.75%, for example from 12.75%-13.0%, such as from 13.0-13.25%, for example from 13.25%-13.5%, such as from 13.5-13.75%, for example from 13.75%-14.0%, such as from 14.0-14.25%, for example from 14.25%-14.5%, such as from 14.5-14.75%, for example from 14.75%-15.0%, %, such as from 15.0-15.25%, for example from 15.25%-15.5%, such as from 15.5-15.75%, for example from 15.75%-16.0%, such as from 16.0-16.25%, for example from 16.25%-16.5%, such as from 16.5-16.75%, for example from 16.75%-17.0%, such as from 17.0-17.25%, for example from 17.25%-17.5%, such as from 17.5-17.75%, for example from 17.75%-18.0%, such as from 18.0-18.25%, for example from 18.25%-18.5%, such as from 18.5-18.75%, for example from 18.75%-19.0%, such as from 19.0-19.25%, for example from 19.25%-19.5%, such as from 19.5-19.75%, for example from 19.75%-20.0%.

In another embodiment the fish feed composition comprises from 1-20% of moisture, such as from 0-0.25%, for example from 0.25%-0.5%, such as from 0.5-0.75%, for example from 0.75%-1.0%, such as from 1.0-1.25%, for example from 1.25%-1.5%, such as from 1.5-1.75%, for example from 1.75%-2.0%, such as from 2.0-2.25%, for example from 2.25%-2.5%, such as from 2.5-2.75%, for example from 2.75%-3.0%, such as from 3.0-3.25%, for example from 3.25%-3.5%, such as from 3.5-3.75%, for example from 3.75%-4.0%, such as from 4.0-4.25%, for example from 4.25%-4.5%, such as from 4.5-4.75%, for example from 4.75%-5.0%, %, such as from 5.0-5.25%, for example from 5.25%-5.5%, such as from 5.5-5.75%, for example from 5.75%-6.0%, such as from 6.0-6.25%, for example from 6.25%-6.5%, such as from 6.5-6.75%, for example from 6.75%-7.0%, such as from 7.0-7.25%, for example from 7.25%-7.5%, such as from 7.5-7.75%, for example from 7.75%-8.0%, such as from 8.0-8.25%, for example from 8.25%-8.5%, such as from 8.5-8.75%, for example from 8.75%-9.0%, such as from 9.0-9.25%, for example from 9.25%-9.5%, such as from 9.5-9.75%, for example from 9.75%-10.0%, such as from 10-10.25%, for example from 10.25%-10.5%, such as from 10.5-10.75%, for example from 10.75%-11.0%, such as from 11.0-11.25%, for example from 11.25%-11.5%, such as from 11.5-11.75%, for example from 11.75%-12.0%, such as from 12.0-12.25%, for example from 12.25%-12.5%, such as from 12.5-12.75%, for example from 12.75%-13.0%, such as from 13.0-13.25%, for example from 13.25%-13.5%, such as from 13.5-13.75%, for example from 13.75%-14.0%, such as from 14.0-14.25%, for example from 14.25%-14.5%, such as from 14.5-14.75%, for example from 14.75%-15.0%, %, such as from 15.0-15.25%, for example from 15.25%-15.5%, such as from 15.5-15.75%, for example from 15.75%-16.0%, such as from 16.0-16.25%, for example from 16.25%-16.5%, such as from 16.5-16.75%, for example from 16.75%-17.0%, such as from 17.0-17.25%, for example from 17.25%-17.5%, such as from 17.5-17.75%, for example from 17.75%-18.0%, such as from 18.0-18.25%, for example from 18.25%-18.5%, such as from 18.5-18.75%, for example from 18.75%-19.0%, such as from 19.0-19.25%, for example from 19.25%-19.5%, such as from 19.5-19.75%, for example from 19.75%-20.0%.

In another embodiment the fish feed composition comprises from 1-20% of carbohydrate, such as from 0-0.25%, for example from 0.25%-0.5%, such as from 0.5-0.75%, for example from 0.75%-1.0%, such as from 1.0-1.25%, for example from 1.25%-1.5%, such as from 1.5-1.75%, for example from 1.75%-2.0%, such as from 2.0-2.25%, for example from 2.25%-2.5%, such as from 2.5-2.75%, for example from 2.75%-3.0%, such as from 3.0-3.25%, for example from 3.25%-3.5%, such as from 3.5-3.75%, for example from 3.75%-4.0%, such as from 4.0-4.25%, for example from 4.25%-4.5%, such as from 4.5-4.75%, for example from 4.75%-5.0%, %, such as from 5.0-5.25%, for example from 5.25%-5.5%, such as from 5.5-5.75%, for example from 5.75%-6.0%, such as from 6.0-6.25%, for example from 6.25%-6.5%, such as from 6.5-6.75%, for example from 6.75%-7.0%, such as from 7.0-7.25%, for example from 7.25%-7.5%, such as from 7.5-7.75%, for example from 7.75%-8.0%, such as from 8.0-8.25%, for example from 8.25%-8.5%, such as from 8.5-8.75%, for example from 8.75%-9.0%, such as from 9.0-9.25%, for example from 9.25%-9.5%, such as from 9.5-9.75%, for example from 9.75%-10.0%, such as from 10-10.25%, for example from 10.25%-10.5%, such as from 10.5-10.75%, for example from 10.75%-11.0%, such as from 11.0-11.25%, for example from 11.25%-11.5%, such as from 11.5-11.75%, for example from 11.75%-12.0%, such as from 12.0-12.25%, for example from 12.25%-12.5%, such as from 12.5-12.75%, for example from 12.75%-13.0%, such as from 13.0-13.25%, for example from 13.25%-13.5%, such as from 13.5-13.75%, for example from 13.75%-14.0%, such as from 14.0-14.25%, for example from 14.25%-14.5%, such as from 14.5-14.75%, for example from 14.75%-15.0%, %, such as from 15.0-15.25%, for example from 15.25%-15.5%, such as from 15.5-15.75%, for example from 15.75%-16.0%, such as from 16.0-16.25%, for example from 16.25%-16.5%, such as from 16.5-16.75%, for example from 16.75%-17.0%, such as from 17.0-17.25%, for example from 17.25%-17.5%, such as from 17.5-17.75%, for example from 17.75%-18.0%, such as from 18.0-18.25%, for example from 18.25%-18.5%, such as from 18.5-18.75%, for example from 18.75%-19.0%, such as from 19.0-19.25%, for example from 19.25%-19.5%, such as from 19.5-19.75%, for example from 19.75%-20.0%.

In one embodiment the present invention relates to a fish feed composition with an Alanine content (amino acid g/16 g N) in the range from 0-20 g/16 g N, such as from 0-0.1 g/16 g N, for example 0.1-0.2 g/16 g N, such as from 0.2-0.3 g/16 g N, for example 0.3-0.4 g/16 g N, such as from 0.4-0.5 g/16 g N, for example 0.5-0.6 g/16 g N, such as from 0.6-0.7 g/16 g N, for example 0.7-0.8 g/16 g N, such as from 0.8-0.9 g/16 g N, for example 0.9-1.0 g/16 g N, such as from 1.0-1.1 g/16 g N, for example 1.1-1.2 g/16 g N, such as from 1.2-1.3 g/16 g N, for example 1.3-1.4 g/16 g N, such as from 1.4-1.5 g/16 g N, for example 1.5-1.6 g/16 g N, such as from 1.6-1.7 g/16 g N, for example 1.7-1.8 g/16 g N, such as from 1.8-1.9 g/16 g N, for example 1.9-2.0 g/16 g N, such as from 2.0-2.1 g/16 g N, for example 2.1-2.2 g/16 g N, such as from 2.2-2.3 g/16 g N, for example 2.3-2.4 g/16 g N, such as from 2.4-2.5 g/16 g N, for example 2.5-2.6 g/16 g N, such as from 2.6-2.7 g/16 g N, for example 2.7-2.8 g/16 g N, such as from 2.8-2.9 g/16 g N, for example 2.9-3.0 g/16 g N, such as from 3.0-3.1 g/16 g N, for example 3.1-3.2 g/16 g N, such as from 3.2-3.3 g/16 g N, for example 3.3-3.4 g/16 g N, such as from 3.4-3.5 g/16 g N, for example 3.5-3.6 g/16 g N, such as from 3.6-3.7 g/16 g N, for example 3.7-3.8 g/16 g N, such as from 3.8-3.9 g/16 g N, for example 3.9-4.0 g/16 g N, such as from 4.0-4.1 g/16 g N, for example 4.1-4.2 g/16 g N, such as from 4.2-4.3 g/16 g N, for example 4.3-4.4 g/16 g N, such as from 4.4-4.5 g/16 g N, for example 4.5-4.6 g/16 g N, such as from 4.6-4.7 g/16 g N, for example 4.7-4.8 g/16 g N, such as from 4.8-4.9 g/16 g N, for example 4.9-5.0 g/16 g N, such as from 5.0-5.1 g/16 g N, for example 5.1-5.2 g/16 g N, such as from 5.2-5.3 g/16 g N, for example 5.3-5.4 g/16 g N, such as from 5.4-5.5 g/16 g N, for example 5.5-5.6 g/16 g N, such as from 5.6-5.7 g/16 g N, for example 5.7-5.8 g/16 g N, such as from 5.8-5.9 g/16 g N, for example 5.9-6.0 g/16 g N, such as from 6.0-6.1 g/16 g N, for example 6.1-6.2 g/16 g N, such as from 6.2-6.3 g/16 g N, for example 6.3-6.4 g/16 g N, such as from 6.4-6.5 g/16 g N, for example 6.5-6.6 g/16 g N, such as from 6.6-6.7 g/16 g N, for example 6.7-6.8 g/16 g N, such as from 6.8-6.9 g/16 g N, for example 6.9-7.0 g/16 g N, such as from 7.0-7.1 g/16 g N, for example 7.1-7.2 g/16 g N, such as from 7.2-7.3 g/16 g N, for example 7.3-7.4 g/16 g N, such as from 7.4-7.5 g/16 g N, for example 7.5-7.6 g/16 g N, such as from 7.6-7.7 g/16 g N, for example 7.7-7.8 g/16 g N, such as from 7.8-7.9 g/16 g N, for example 7.9-8.0 g/16 g N, such as from 8.0-8.1 g/16 g N, for example 8.1-8.2 g/16 g N, such as from 8.2-8.3 g/16 g N, for example 8.3-8.4 g/16 g N, such as from 8.4-8.5 g/16 g N, for example 8.5-8.6 g/16 g N, such as from 8.6-8.7 g/16 g N, for example 8.7-8.8 g/16 g N, such as from 8.8-8.9 g/16 g N, for example 8.9-9.0 g/16 g N, such as from 9.0-9.1 g/16 g N, for example 9.1-9.2 g/16 g N, such as from 9.2-9.3 g/16 g N, for example 9.3-9.4 g/16 g N, such as from 9.4-9.5 g/16 g N, for example 9.5-9.6 g/16 g N, such as from 9.6-9.7 g/16 g N, for example 9.7-9.8 g/16 g N, such as from 9.8-9.9 g/16 g N, for example 9.9-10.0 g/16 g N, such as from 10.0-10.1 g/16 g N, for example 10.1-10.2 g/16 g N, such as from 10.2-10.3 g/16 g N, for example 10.3-10.4 g/16 g N, such as from 10.4-10.5 g/16 g N, for example 10.5-10.6 g/16 g N, such as from 10.6-10.7 g/16 g N, for example 10.7-10.8 g/16 g N, such as from 10.8-10.9 g/16 g N, for example 10.9-11.0 g/16 g N, such as from 11.0-11.1 g/16 g N, for example 11.1-11.2 g/16 g N, such as from 11.2-11.3 g/16 g N, for example 11.3-11.4 g/16 g N, such as from 11.4-11.5 g/16 g N, for example 11.5-11.6 g/16 g N, such as from 11.6-11.7 g/16 g N, for example 11.7-11.8 g/16 g N, such as from 11.8-11.9 g/16 g N, for example 11.9-12.0 g/16 g N, such as from 12.0-12.1 g/16 g N, for example 12.1-12.2 g/16 g N, such as from 12.2-12.3 g/16 g N, for example 12.3-12.4 g/16 g N, such as from 12.4-12.5 g/16 g N, for example 12.5-12.6 g/16 g N, such as from 12.6-12.7 g/16 g N, for example 12.7-12.8 g/16 g N, such as from 12.8-12.9 g/16 g N, for example 12.9-13.0 g/16 g N, such as from 13.0-13.1 g/16 g N, for example 13.1-13.2 g/16 g N, such as from 13.2-13.3 g/16 g N, for example 13.3-13.4 g/16 g N, such as from 13.4-13.5 g/16 g N, for example 13.5-13.6 g/16 g N, such as from 13.6-13.7 g/16 g N, for example 13.7-13.8 g/16 g N, such as from 13.8-13.9 g/16 g N, for example 13.9-14.0 g/16 g N, such as from 14.0-14.1 g/16 g N, for example 14.1-14.2 g/16 g N, such as from 14.2-14.3 g/16 g N, for example 14.3-14.4 g/16 g N, such as from 14.4-14.5 g/16 g N, for example 14.5-14.6 g/16 g N, such as from 14.6-14.7 g/16 g N, for example 14.7-14.8 g/16 g N, such as from 14.8-14.9 g/16 g N, for example 14.9-15.0 g/16 g N, such as from 15-16 g/16 g N, for example 16-17 g/16 g N, such as from 17-18 g/16 g N, for example 18-19 g/16 g N, such as from 19-20 g/16 g N.

In one embodiment the present invention relates to a fish feed composition with an Arginine content (amino acid g/16 g N) in the range from 0-20 g/16 g N, such as from 0-0.1 g/16 g N, for example 0.1-0.2 g/16 g N, such as from 0.2-0.3 g/16 g N, for example 0.3-0.4 g/16 g N, such as from 0.4-0.5 g/16 g N, for example 0.5-0.6 g/16 g N, such as from 0.6-0.7 g/16 g N, for example 0.7-0.8 g/16 g N, such as from 0.8-0.9 g/16 g N, for example 0.9-1.0 g/16 g N, such as from 1.0-1.1 g/16 g N, for example 1.1-1.2 g/16 g N, such as from 1.2-1.3 g/16 g N, for example 1.3-1.4 g/16 g N, such as from 1.4-1.5 g/16 g N, for example 1.5-1.6 g/16 g N, such as from 1.6-1.7 g/16 g N, for example 1.7-1.8 g/16 g N, such as from 1.8-1.9 g/16 g N, for example 1.9-2.0 g/16 g N, such as from 2.0-2.1 g/16 g N, for example 2.1-2.2 g/16 g N, such as from 2.2-2.3 g/16 g N, for example 2.3-2.4 g/16 g N, such as from 2.4-2.5 g/16 g N, for example 2.5-2.6 g/16 g N, such as from 2.6-2.7 g/16 g N, for example 2.7-2.8 g/16 g N, such as from 2.8-2.9 g/16 g N, for example 2.9-3.0 g/16 g N, such as from 3.0-3.1 g/16 g N, for example 3.1-3.2 g/16 g N, such as from 3.2-3.3 g/16 g N, for example 3.3-3.4 g/16 g N, such as from 3.4-3.5 g/16 g N, for example 3.5-3.6 g/16 g N, such as from 3.6-3.7 g/16 g N, for example 3.7-3.8 g/16 g N, such as from 3.8-3.9 g/16 g N, for example 3.9-4.0 g/16 g N, such as from 4.0-4.1 g/16 g N, for example 4.1-4.2 g/16 g N, such as from 4.2-4.3 g/16 g N, for example 4.3-4.4 g/16 g N, such as from 4.4-4.5 g/16 g N, for example 4.5-4.6 g/16 g N, such as from 4.6-4.7 g/16 g N, for example 4.7-4.8 g/16 g N, such as from 4.8-4.9 g/16 g N, for example 4.9-5.0 g/16 g N, such as from 5.0-5.1 g/16 g N, for example 5.1-5.2 g/16 g N, such as from 5.2-5.3 g/16 g N, for example 5.3-5.4 g/16 g N, such as from 5.4-5.5 g/16 g N, for example 5.5-5.6 g/16 g N, such as from 5.6-5.7 g/16 g N, for example 5.7-5.8 g/16 g N, such as from 5.8-5.9 g/16 g N, for example 5.9-6.0 g/16 g N, such as from 6.0-6.1 g/16 g N, for example 6.1-6.2 g/16 g N, such as from 6.2-6.3 g/16 g N, for example 6.3-6.4 g/16 g N, such as from 6.4-6.5 g/16 g N, for example 6.5-6.6 g/16 g N, such as from 6.6-6.7 g/16 g N, for example 6.7-6.8 g/16 g N, such as from 6.8-6.9 g/16 g N, for example 6.9-7.0 g/16 g N, such as from 7.0-7.1 g/16 g N, for example 7.1-7.2 g/16 g N, such as from 7.2-7.3 g/16 g N, for example 7.3-7.4 g/16 g N, such as from 7.4-7.5 g/16 g N, for example 7.5-7.6 g/16 g N, such as from 7.6-7.7 g/16 g N, for example 7.7-7.8 g/16 g N, such as from 7.8-7.9 g/16 g N, for example 7.9-8.0 g/16 g N, such as from 8.0-8.1 g/16 g N, for example 8.1-8.2 g/16 g N, such as from 8.2-8.3 g/16 g N, for example 8.3-8.4 g/16 g N, such as from 8.4-8.5 g/16 g N, for example 8.5-8.6 g/16 g N, such as from 8.6-8.7 g/16 g N, for example 8.7-8.8 g/16 g N, such as from 8.8-8.9 g/16 g N, for example 8.9-9.0 g/16 g N, such as from 9.0-9.1 g/16 g N, for example 9.1-9.2 g/16 g N, such as from 9.2-9.3 g/16 g N, for example 9.3-9.4 g/16 g N, such as from 9.4-9.5 g/16 g N, for example 9.5-9.6 g/16 g N, such as from 9.6-9.7 g/16 g N, for example 9.7-9.8 g/16 g N, such as from 9.8-9.9 g/16 g N, for example 9.9-10.0 g/16 g N, such as from 10.0-10.1 g/16 g N, for example 10.1-10.2 g/16 g N, such as from 10.2-10.3 g/16 g N, for example 10.3-10.4 g/16 g N, such as from 10.4-10.5 g/16 g N, for example 10.5-10.6 g/16 g N, such as from 10.6-10.7 g/16 g N, for example 10.7-10.8 g/16 g N, such as from 10.8-10.9 g/16 g N, for example 10.9-11.0 g/16 g N, such as from 11.0-11.1 g/16 g N, for example 11.1-11.2 g/16 g N, such as from 11.2-11.3 g/16 g N, for example 11.3-11.4 g/16 g N, such as from 11.4-11.5 g/16 g N, for example 11.5-11.6 g/16 g N, such as from 11.6-11.7 g/16 g N, for example 11.7-11.8 g/16 g N, such as from 11.8-11.9 g/16 g N, for example 11.9-12.0 g/16 g N, such as from 12.0-12.1 g/16 g N, for example 12.1-12.2 g/16 g N, such as from 12.2-12.3 g/16 g N, for example 12.3-12.4 g/16 g N, such as from 12.4-12.5 g/16 g N, for example 12.5-12.6 g/16 g N, such as from 12.6-12.7 g/16 g N, for example 12.7-12.8 g/16 g N, such as from 12.8-12.9 g/16 g N, for example 12.9-13.0 g/16 g N, such as from 13.0-13.1 g/16 g N, for example 13.1-13.2 g/16 g N, such as from 13.2-13.3 g/16 g N, for example 13.3-13.4 g/16 g N, such as from 13.4-13.5 g/16 g N, for example 13.5-13.6 g/16 g N, such as from 13.6-13.7 g/16 g N, for example 13.7-13.8 g/16 g N, such as from 13.8-13.9 g/16 g N, for example 13.9-14.0 g/16 g N, such as from 14.0-14.1 g/16 g N, for example 14.1-14.2 g/16 g N, such as from 14.2-14.3 g/16 g N, for example 14.3-14.4 g/16 g N, such as from 14.4-14.5 g/16 g N, for example 14.5-14.6 g/16 g N, such as from 14.6-14.7 g/16 g N, for example 14.7-14.8 g/16 g N, such as from 14.8-14.9 g/16 g N, for example 14.9-15.0 g/16 g N, such as from 15-16 g/16 g N, for example 16-17 g/16 g N, such as from 17-18 g/16 g N, for example 18-19 g/16 g N, such as from 19-20 g/16 g N.

In one embodiment the present invention relates to a fish feed composition with an Aspartic acid content (amino acid g/16 g N) in the range from 0-20 g/16 g N, such as from 0-0.1 g/16 g N, for example 0.1-0.2 g/16 g N, such as from 0.2-0.3 g/16 g N, for example 0.3-0.4 g/16 g N, such as from 0.4-0.5 g/16 g N, for example 0.5-0.6 g/16 g N, such as from 0.6-0.7 g/16 g N, for example 0.7-0.8 g/16 g N, such as from 0.8-0.9 g/16 g N, for example 0.9-1.0 g/16 g N, such as from 1.0-1.1 g/16 g N, for example 1.1-1.2 g/16 g N, such as from 1.2-1.3 g/16 g N, for example 1.3-1.4 g/16 g N, such as from 1.4-1.5 g/16 g N, for example 1.5-1.6 g/16 g N, such as from 1.6-1.7 g/16 g N, for example 1.7-1.8 g/16 g N, such as from 1.8-1.9 g/16 g N, for example 1.9-2.0 g/16 g N, such as from 2.0-2.1 g/16 g N, for example 2.1-2.2 g/16 g N, such as from 2.2-2.3 g/16 g N, for example 2.3-2.4 g/16 g N, such as from 2.4-2.5 g/16 g N, for example 2.5-2.6 g/16 g N, such as from 2.6-2.7 g/16 g N, for example 2.7-2.8 g/16 g N, such as from 2.8-2.9 g/16 g N, for example 2.9-3.0 g/16 g N, such as from 3.0-3.1 g/16 g N, for example 3.1-3.2 g/16 g N, such as from 3.2-3.3 g/16 g N, for example 3.3-3.4 g/16 g N, such as from 3.4-3.5 g/16 g N, for example 3.5-3.6 g/16 g N, such as from 3.6-3.7 g/16 g N, for example 3.7-3.8 g/16 g N, such as from 3.8-3.9 g/16 g N, for example 3.9-4.0 g/16 g N, such as from 4.0-4.1 g/16 g N, for example 4.1-4.2 g/16 g N, such as from 4.2-4.3 g/16 g N, for example 4.3-4.4 g/16 g N, such as from 4.4-4.5 g/16 g N, for example 4.5-4.6 g/16 g N, such as from 4.6-4.7 g/16 g N, for example 4.7-4.8 g/16 g N, such as from 4.8-4.9 g/16 g N, for example 4.9-5.0 g/16 g N, such as from 5.0-5.1 g/16 g N, for example 5.1-5.2 g/16 g N, such as from 5.2-5.3 g/16 g N, for example 5.3-5.4 g/16 g N, such as from 5.4-5.5 g/16 g N, for example 5.5-5.6 g/16 g N, such as from 5.6-5.7 g/16 g N, for example 5.7-5.8 g/16 g N, such as from 5.8-5.9 g/16 g N, for example 5.9-6.0 g/16 g N, such as from 6.0-6.1 g/16 g N, for example 6.1-6.2 g/16 g N, such as from 6.2-6.3 g/16 g N, for example 6.3-6.4 g/16 g N, such as from 6.4-6.5 g/16 g N, for example 6.5-6.6 g/16 g N, such as from 6.6-6.7 g/16 g N, for example 6.7-6.8 g/16 g N, such as from 6.8-6.9 g/16 g N, for example 6.9-7.0 g/16 g N, such as from 7.0-7.1 g/16 g N, for example 7.1-7.2 g/16 g N, such as from 7.2-7.3 g/16 g N, for example 7.3-7.4 g/16 g N, such as from 7.4-7.5 g/16 g N, for example 7.5-7.6 g/16 g N, such as from 7.6-7.7 g/16 g N, for example 7.7-7.8 g/16 g N, such as from 7.8-7.9 g/16 g N, for example 7.9-8.0 g/16 g N, such as from 8.0-8.1 g/16 g N, for example 8.1-8.2 g/16 g N, such as from 8.2-8.3 g/16 g N, for example 8.3-8.4 g/16 g N, such as from 8.4-8.5 g/16 g N, for example 8.5-8.6 g/16 g N, such as from 8.6-8.7 g/16 g N, for example 8.7-8.8 g/16 g N, such as from 8.8-8.9 g/16 g N, for example 8.9-9.0 g/16 g N, such as from 9.0-9.1 g/16 g N, for example 9.1-9.2 g/16 g N, such as from 9.2-9.3 g/16 g N, for example 9.3-9.4 g/16 g N, such as from 9.4-9.5 g/16 g N, for example 9.5-9.6 g/16 g N, such as from 9.6-9.7 g/16 g N, for example 9.7-9.8 g/16 g N, such as from 9.8-9.9 g/16 g N, for example 9.9-10.0 g/16 g N, such as from 10.0-10.1 g/16 g N, for example 10.1-10.2 g/16 g N, such as from 10.2-10.3 g/16 g N, for example 10.3-10.4 g/16 g N, such as from 10.4-10.5 g/16 g N, for example 10.5-10.6 g/16 g N, such as from 10.6-10.7 g/16 g N, for example 10.7-10.8 g/16 g N, such as from 10.8-10.9 g/16 g N, for example 10.9-11.0 g/16 g N, such as from 11.0-11.1 g/16 g N, for example 11.1-11.2 g/16 g N, such as from 11.2-11.3 g/16 g N, for example 11.3-11.4 g/16 g N, such as from 11.4-11.5 g/16 g N, for example 11.5-11.6 g/16 g N, such as from 11.6-11.7 g/16 g N, for example 11.7-11.8 g/16 g N, such as from 11.8-11.9 g/16 g N, for example 11.9-12.0 g/16 g N, such as from 12.0-12.1 g/16 g N, for example 12.1-12.2 g/16 g N, such as from 12.2-12.3 g/16 g N, for example 12.3-12.4 g/16 g N, such as from 12.4-12.5 g/16 g N, for example 12.5-12.6 g/16 g N, such as from 12.6-12.7 g/16 g N, for example 12.7-12.8 g/16 g N, such as from 12.8-12.9 g/16 g N, for example 12.9-13.0 g/16 g N, such as from 13.0-13.1 g/16 g N, for example 13.1-13.2 g/16 g N, such as from 13.2-13.3 g/16 g N, for example 13.3-13.4 g/16 g N, such as from 13.4-13.5 g/16 g N, for example 13.5-13.6 g/16 g N, such as from 13.6-13.7 g/16 g N, for example 13.7-13.8 g/16 g N, such as from 13.8-13.9 g/16 g N, for example 13.9-14.0 g/16 g N, such as from 14.0-14.1 g/16 g N, for example 14.1-14.2 g/16 g N, such as from 14.2-14.3 g/16 g N, for example 14.3-14.4 g/16 g N, such as from 14.4-14.5 g/16 g N, for example 14.5-14.6 g/16 g N, such as from 14.6-14.7 g/16 g N, for example 14.7-14.8 g/16 g N, such as from 14.8-14.9 g/16 g N, for example 14.9-15.0 g/16 g N, such as from 15-16 g/16 g N, for example 16-17 g/16 g N, such as from 17-18 g/16 g N, for example 18-19 g/16 g N, such as from 19-20 g/16 g N.

In one embodiment the present invention relates to a fish feed composition with a Cystein content (amino acid g/16 g N) in the range from 0-20 g/16 g N, such as from 0-0.1 g/16 g N, for example 0.1-0.2 g/16 g N, such as from 0.2-0.3 g/16 g N, for example 0.3-0.4 g/16 g N, such as from 0.4-0.5 g/16 g N, for example 0.5-0.6 g/16 g N, such as from 0.6-0.7 g/16 g N, for example 0.7-0.8 g/16 g N, such as from 0.8-0.9 g/16 g N, for example 0.9-1.0 g/16 g N, such as from 1.0-1.1 g/16 g N, for example 1.1-1.2 g/16 g N, such as from 1.2-1.3 g/16 g N, for example 1.3-1.4 g/16 g N, such as from 1.4-1.5 g/16 g N, for example 1.5-1.6 g/16 g N, such as from 1.6-1.7 g/16 g N, for example 1.7-1.8 g/16 g N, such as from 1.8-1.9 g/16 g N, for example 1.9-2.0 g/16 g N, such as from 2.0-2.1 g/16 g N, for example 2.1-2.2 g/16 g N, such as from 2.2-2.3 g/16 g N, for example 2.3-2.4 g/16 g N, such as from 2.4-2.5 g/16 g N, for example 2.5-2.6 g/16 g N, such as from 2.6-2.7 g/16 g N, for example 2.7-2.8 g/16 g N, such as from 2.8-2.9 g/16 g N, for example 2.9-3.0 g/16 g N, such as from 3.0-3.1 g/16 g N, for example 3.1-3.2 g/16 g N, such as from 3.2-3.3 g/16 g N, for example 3.3-3.4 g/16 g N, such as from 3.4-3.5 g/16 g N, for example 3.5-3.6 g/16 g N, such as from 3.6-3.7 g/16 g N, for example 3.7-3.8 g/16 g N, such as from 3.8-3.9 g/16 g N, for example 3.9-4.0 g/16 g N, such as from 4.0-4.1 g/16 g N, for example 4.1-4.2 g/16 g N, such as from 4.2-4.3 g/16 g N, for example 4.3-4.4 g/16 g N, such as from 4.4-4.5 g/16 g N, for example 4.5-4.6 g/16 g N, such as from 4.6-4.7 g/16 g N, for example 4.7-4.8 g/16 g N, such as from 4.8-4.9 g/16 g N, for example 4.9-5.0 g/16 g N, such as from 5.0-5.1 g/16 g N, for example 5.1-5.2 g/16 g N, such as from 5.2-5.3 g/16 g N, for example 5.3-5.4 g/16 g N, such as from 5.4-5.5 g/16 g N, for example 5.5-5.6 g/16 g N, such as from 5.6-5.7 g/16 g N, for example 5.7-5.8 g/16 g N, such as from 5.8-5.9 g/16 g N, for example 5.9-6.0 g/16 g N, such as from 6.0-6.1 g/16 g N, for example 6.1-6.2 g/16 g N, such as from 6.2-6.3 g/16 g N, for example 6.3-6.4 g/16 g N, such as from 6.4-6.5 g/16 g N, for example 6.5-6.6 g/16 g N, such as from 6.6-6.7 g/16 g N, for example 6.7-6.8 g/16 g N, such as from 6.8-6.9 g/16 g N, for example 6.9-7.0 g/16 g N, such as from 7.0-7.1 g/16 g N, for example 7.1-7.2 g/16 g N, such as from 7.2-7.3 g/16 g N, for example 7.3-7.4 g/16 g N, such as from 7.4-7.5 g/16 g N, for example 7.5-7.6 g/16 g N, such as from 7.6-7.7 g/16 g N, for example 7.7-7.8 g/16 g N, such as from 7.8-7.9 g/16 g N, for example 7.9-8.0 g/16 g N, such as from 8.0-8.1 g/16 g N, for example 8.1-8.2 g/16 g N, such as from 8.2-8.3 g/16 g N, for example 8.3-8.4 g/16 g N, such as from 8.4-8.5 g/16 g N, for example 8.5-8.6 g/16 g N, such as from 8.6-8.7 g/16 g N, for example 8.7-8.8 g/16 g N, such as from 8.8-8.9 g/16 g N, for example 8.9-9.0 g/16 g N, such as from 9.0-9.1 g/16 g N, for example 9.1-9.2 g/16 g N, such as from 9.2-9.3 g/16 g N, for example 9.3-9.4 g/16 g N, such as from 9.4-9.5 g/16 g N, for example 9.5-9.6 g/16 g N, such as from 9.6-9.7 g/16 g N, for example 9.7-9.8 g/16 g N, such as from 9.8-9.9 g/16 g N, for example 9.9-10.0 g/16 g N, such as from 10.0-10.1 g/16 g N, for example 10.1-10.2 g/16 g N, such as from 10.2-10.3 g/16 g N, for example 10.3-10.4 g/16 g N, such as from 10.4-10.5 g/16 g N, for example 10.5-10.6 g/16 g N, such as from 10.6-10.7 g/16 g N, for example 10.7-10.8 g/16 g N, such as from 10.8-10.9 g/16 g N, for example 10.9-11.0 g/16 g N, such as from 11.0-11.1 g/16 g N, for example 11.1-11.2 g/16 g N, such as from 11.2-11.3 g/16 g N, for example 11.3-11.4 g/16 g N, such as from 11.4-11.5 g/16 g N, for example 11.5-11.6 g/16 g N, such as from 11.6-11.7 g/16 g N, for example 11.7-11.8 g/16 g N, such as from 11.8-11.9 g/16 g N, for example 11.9-12.0 g/16 g N, such as from 12.0-12.1 g/16 g N, for example 12.1-12.2 g/16 g N, such as from 12.2-12.3 g/16 g N, for example 12.3-12.4 g/16 g N, such as from 12.4-12.5 g/16 g N, for example 12.5-12.6 g/16 g N, such as from 12.6-12.7 g/16 g N, for example 12.7-12.8 g/16 g N, such as from 12.8-12.9 g/16 g N, for example 12.9-13.0 g/16 g N, such as from 13.0-13.1 g/16 g N, for example 13.1-13.2 g/16 g N, such as from 13.2-13.3 g/16 g N, for example 13.3-13.4 g/16 g N, such as from 13.4-13.5 g/16 g N, for example 13.5-13.6 g/16 g N, such as from 13.6-13.7 g/16 g N, for example 13.7-13.8 g/16 g N, such as from 13.8-13.9 g/16 g N, for example 13.9-14.0 g/16 g N, such as from 14.0-14.1 g/16 g N, for example 14.1-14.2 g/16 g N, such as from 14.2-14.3 g/16 g N, for example 14.3-14.4 g/16 g N, such as from 14.4-14.5 g/16 g N, for example 14.5-14.6 g/16 g N, such as from 14.6-14.7 g/16 g N, for example 14.7-14.8 g/16 g N, such as from 14.8-14.9 g/16 g N, for example 14.9-15.0 g/16 g N, such as from 15-16 g/16 g N, for example 16-17 g/16 g N, such as from 17-18 g/16 g N, for example 18-19 g/16 g N, such as from 19-20 g/16 g N.

In one embodiment the present invention relates to a fish feed composition with a Glutamic acid content (amino acid g/16 g N) in the range from 0-20 g/16 g N, such as from 0-0.1 g/16 g N, for example 0.1-0.2 g/16 g N, such as from 0.2-0.3 g/16 g N, for example 0.3-0.4 g/16 g N, such as from 0.4-0.5 g/16 g N, for example 0.5-0.6 g/16 g N, such as from 0.6-0.7 g/16 g N, for example 0.7-0.8 g/16 g N, such as from 0.8-0.9 g/16 g N, for example 0.9-1.0 g/16 g N, such as from 1.0-1.1 g/16 g N, for example 1.1-1.2 g/16 g N, such as from 1.2-1.3 g/16 g N, for example 1.3-1.4 g/16 g N, such as from 1.4-1.5 g/16 g N, for example 1.5-1.6 g/16 g N, such as from 1.6-1.7 g/16 g N, for example 1.7-1.8 g/16 g N, such as from 1.8-1.9 g/16 g N, for example 1.9-2.0 g/16 g N, such as from 2.0-2.1 g/16 g N, for example 2.1-2.2 g/16 g N, such as from 2.2-2.3 g/16 g N, for example 2.3-2.4 g/16 g N, such as from 2.4-2.5 g/16 g N, for example 2.5-2.6 g/16 g N, such as from 2.6-2.7 g/16 g N, for example 2.7-2.8 g/16 g N, such as from 2.8-2.9 g/16 g N, for example 2.9-3.0 g/16 g N, such as from 3.0-3.1 g/16 g N, for example 3.1-3.2 g/16 g N, such as from 3.2-3.3 g/16 g N, for example 3.3-3.4 g/16 g N, such as from 3.4-3.5 g/16 g N, for example 3.5-3.6 g/16 g N, such as from 3.6-3.7 g/16 g N, for example 3.7-3.8 g/16 g N, such as from 3.8-3.9 g/16 g N, for example 3.9-4.0 g/16 g N, such as from 4.0-4.1 g/16 g N, for example 4.1-4.2 g/16 g N, such as from 4.2-4.3 g/16 g N, for example 4.3-4.4 g/16 g N, such as from 4.4-4.5 g/16 g N, for example 4.5-4.6 g/16 g N, such as from 4.6-4.7 g/16 g N, for example 4.7-4.8 g/16 g N, such as from 4.8-4.9 g/16 g N, for example 4.9-5.0 g/16 g N, such as from 5.0-5.1 g/16 g N, for example 5.1-5.2 g/16 g N, such as from 5.2-5.3 g/16 g N, for example 5.3-5.4 g/16 g N, such as from 5.4-5.5 g/16 g N, for example 5.5-5.6 g/16 g N, such as from 5.6-5.7 g/16 g N, for example 5.7-5.8 g/16 g N, such as from 5.8-5.9 g/16 g N, for example 5.9-6.0 g/16 g N, such as from 6.0-6.1 g/16 g N, for example 6.1-6.2 g/16 g N, such as from 6.2-6.3 g/16 g N, for example 6.3-6.4 g/16 g N, such as from 6.4-6.5 g/16 g N, for example 6.5-6.6 g/16 g N, such as from 6.6-6.7 g/16 g N, for example 6.7-6.8 g/16 g N, such as from 6.8-6.9 g/16 g N, for example 6.9-7.0 g/16 g N, such as from 7.0-7.1 g/16 g N, for example 7.1-7.2 g/16 g N, such as from 7.2-7.3 g/16 g N, for example 7.3-7.4 g/16 g N, such as from 7.4-7.5 g/16 g N, for example 7.5-7.6 g/16 g N, such as from 7.6-7.7 g/16 g N, for example 7.7-7.8 g/16 g N, such as from 7.8-7.9 g/16 g N, for example 7.9-8.0 g/16 g N, such as from 8.0-8.1 g/16 g N, for example 8.1-8.2 g/16 g N, such as from 8.2-8.3 g/16 g N, for example 8.3-8.4 g/16 g N, such as from 8.4-8.5 g/16 g N, for example 8.5-8.6 g/16 g N, such as from 8.6-8.7 g/16 g N, for example 8.7-8.8 g/16 g N, such as from 8.8-8.9 g/16 g N, for example 8.9-9.0 g/16 g N, such as from 9.0-9.1 g/16 g N, for example 9.1-9.2 g/16 g N, such as from 9.2-9.3 g/16 g N, for example 9.3-9.4 g/16 g N, such as from 9.4-9.5 g/16 g N, for example 9.5-9.6 g/16 g N, such as from 9.6-9.7 g/16 g N, for example 9.7-9.8 g/16 g N, such as from 9.8-9.9 g/16 g N, for example 9.9-10.0 g/16 g N, such as from 10.0-10.1 g/16 g N, for example 10.1-10.2 g/16 g N, such as from 10.2-10.3 g/16 g N, for example 10.3-10.4 g/16 g N, such as from 10.4-10.5 g/16 g N, for example 10.5-10.6 g/16 g N, such as from 10.6-10.7 g/16 g N, for example 10.7-10.8 g/16 g N, such as from 10.8-10.9 g/16 g N, for example 10.9-11.0 g/16 g N, such as from 11.0-11.1 g/16 g N, for example 11.1-11.2 g/16 g N, such as from 11.2-11.3 g/16 g N, for example 11.3-11.4 g/16 g N, such as from 11.4-11.5 g/16 g N, for example 11.5-11.6 g/16 g N, such as from 11.6-11.7 g/16 g N, for example 11.7-11.8 g/16 g N, such as from 11.8-11.9 g/16 g N, for example 11.9-12.0 g/16 g N, such as from 12.0-12.1 g/16 g N, for example 12.1-12.2 g/16 g N, such as from 12.2-12.3 g/16 g N, for example 12.3-12.4 g/16 g N, such as from 12.4-12.5 g/16 g N, for example 12.5-12.6 g/16 g N, such as from 12.6-12.7 g/16 g N, for example 12.7-12.8 g/16 g N, such as from 12.8-12.9 g/16 g N, for example 12.9-13.0 g/16 g N, such as from 13.0-13.1 g/16 g N, for example 13.1-13.2 g/16 g N, such as from 13.2-13.3 g/16 g N, for example 13.3-13.4 g/16 g N, such as from 13.4-13.5 g/16 g N, for example 13.5-13.6 g/16 g N, such as from 13.6-13.7 g/16 g N, for example 13.7-13.8 g/16 g N, such as from 13.8-13.9 g/16 g N, for example 13.9-14.0 g/16 g N, such as from 14.0-14.1 g/16 g N, for example 14.1-14.2 g/16 g N, such as from 14.2-14.3 g/16 g N, for example 14.3-14.4 g/16 g N, such as from 14.4-14.5 g/16 g N, for example 14.5-14.6 g/16 g N, such as from 14.6-14.7 g/16 g N, for example 14.7-14.8 g/16 g N, such as from 14.8-14.9 g/16 g N, for example 14.9-15.0 g/16 g N, such as from 15-16 g/16 g N, for example 16-17 g/16 g N, such as from 17-18 g/16 g N, for example 18-19 g/16 g N, such as from 19-20 g/16 g N.

In one embodiment the present invention relates to a fish feed composition with a Glycine content (amino acid g/16 g N) in the range from 0-20 g/16 g N, such as from 0-0.1 g/16 g N, for example 0.1-0.2 g/16 g N, such as from 0.2-0.3 g/16 g N, for example 0.3-0.4 g/16 g N, such as from 0.4-0.5 g/16 g N, for example 0.5-0.6 g/16 g N, such as from 0.6-0.7 g/16 g N, for example 0.7-0.8 g/16 g N, such as from 0.8-0.9 g/16 g N, for example 0.9-1.0 g/16 g N, such as from 1.0-1.1 g/16 g N, for example 1.1-1.2 g/16 g N, such as from 1.2-1.3 g/16 g N, for example 1.3-1.4 g/16 g N, such as from 1.4-1.5 g/16 g N, for example 1.5-1.6 g/16 g N, such as from 1.6-1.7 g/16 g N, for example 1.7-1.8 g/16 g N, such as from 1.8-1.9 g/16 g N, for example 1.9-2.0 g/16 g N, such as from 2.0-2.1 g/16 g N, for example 2.1-2.2 g/16 g N, such as from 2.2-2.3 g/16 g N, for example 2.3-2.4 g/16 g N, such as from 2.4-2.5 g/16 g N, for example 2.5-2.6 g/16 g N, such as from 2.6-2.7 g/16 g N, for example 2.7-2.8 g/16 g N, such as from 2.8-2.9 g/16 g N, for example 2.9-3.0 g/16 g N, such as from 3.0-3.1 g/16 g N, for example 3.1-3.2 g/16 g N, such as from 3.2-3.3 g/16 g N, for example 3.3-3.4 g/16 g N, such as from 3.4-3.5 g/16 g N, for example 3.5-3.6 g/16 g N, such as from 3.6-3.7 g/16 g N, for example 3.7-3.8 g/16 g N, such as from 3.8-3.9 g/16 g N, for example 3.9-4.0 g/16 g N, such as from 4.0-4.1 g/16 g N, for example 4.1-4.2 g/16 g N, such as from 4.2-4.3 g/16 g N, for example 4.3-4.4 g/16 g N, such as from 4.4-4.5 g/16 g N, for example 4.5-4.6 g/16 g N, such as from 4.6-4.7 g/16 g N, for example 4.7-4.8 g/16 g N, such as from 4.8-4.9 g/16 g N, for example 4.9-5.0 g/16 g N, such as from 5.0-5.1 g/16 g N, for example 5.1-5.2 g/16 g N, such as from 5.2-5.3 g/16 g N, for example 5.3-5.4 g/16 g N, such as from 5.4-5.5 g/16 g N, for example 5.5-5.6 g/16 g N, such as from 5.6-5.7 g/16 g N, for example 5.7-5.8 g/16 g N, such as from 5.8-5.9 g/16 g N, for example 5.9-6.0 g/16 g N, such as from 6.0-6.1 g/16 g N, for example 6.1-6.2 g/16 g N, such as from 6.2-6.3 g/16 g N, for example 6.3-6.4 g/16 g N, such as from 6.4-6.5 g/16 g N, for example 6.5-6.6 g/16 g N, such as from 6.6-6.7 g/16 g N, for example 6.7-6.8 g/16 g N, such as from 6.8-6.9 g/16 g N, for example 6.9-7.0 g/16 g N, such as from 7.0-7.1 g/16 g N, for example 7.1-7.2 g/16 g N, such as from 7.2-7.3 g/16 g N, for example 7.3-7.4 g/16 g N, such as from 7.4-7.5 g/16 g N, for example 7.5-7.6 g/16 g N, such as from 7.6-7.7 g/16 g N, for example 7.7-7.8 g/16 g N, such as from 7.8-7.9 g/16 g N, for example 7.9-8.0 g/16 g N, such as from 8.0-8.1 g/16 g N, for example 8.1-8.2 g/16 g N, such as from 8.2-8.3 g/16 g N, for example 8.3-8.4 g/16 g N, such as from 8.4-8.5 g/16 g N, for example 8.5-8.6 g/16 g N, such as from 8.6-8.7 g/16 g N, for example 8.7-8.8 g/16 g N, such as from 8.8-8.9 g/16 g N, for example 8.9-9.0 g/16 g N, such as from 9.0-9.1 g/16 g N, for example 9.1-9.2 g/16 g N, such as from 9.2-9.3 g/16 g N, for example 9.3-9.4 g/16 g N, such as from 9.4-9.5 g/16 g N, for example 9.5-9.6 g/16 g N, such as from 9.6-9.7 g/16 g N, for example 9.7-9.8 g/16 g N, such as from 9.8-9.9 g/16 g N, for example 9.9-10.0 g/16 g N, such as from 10.0-10.1 g/16 g N, for example 10.1-10.2 g/16 g N, such as from 10.2-10.3 g/16 g N, for example 10.3-10.4 g/16 g N, such as from 10.4-10.5 g/16 g N, for example 10.5-10.6 g/16 g N, such as from 10.6-10.7 g/16 g N, for example 10.7-10.8 g/16 g N, such as from 10.8-10.9 g/16 g N, for example 10.9-11.0 g/16 g N, such as from 11.0-11.1 g/16 g N, for example 11.1-11.2 g/16 g N, such as from 11.2-11.3 g/16 g N, for example 11.3-11.4 g/16 g N, such as from 11.4-11.5 g/16 g N, for example 11.5-11.6 g/16 g N, such as from 11.6-11.7 g/16 g N, for example 11.7-11.8 g/16 g N, such as from 11.8-11.9 g/16 g N, for example 11.9-12.0 g/16 g N, such as from 12.0-12.1 g/16 g N, for example 12.1-12.2 g/16 g N, such as from 12.2-12.3 g/16 g N, for example 12.3-12.4 g/16 g N, such as from 12.4-12.5 g/16 g N, for example 12.5-12.6 g/16 g N, such as from 12.6-12.7 g/16 g N, for example 12.7-12.8 g/16 g N, such as from 12.8-12.9 g/16 g N, for example 12.9-13.0 g/16 g N, such as from 13.0-13.1 g/16 g N, for example 13.1-13.2 g/16 g N, such as from 13.2-13.3 g/16 g N, for example 13.3-13.4 g/16 g N, such as from 13.4-13.5 g/16 g N, for example 13.5-13.6 g/16 g N, such as from 13.6-13.7 g/16 g N, for example 13.7-13.8 g/16 g N, such as from 13.8-13.9 g/16 g N, for example 13.9-14.0 g/16 g N, such as from 14.0-14.1 g/16 g N, for example 14.1-14.2 g/16 g N, such as from 14.2-14.3 g/16 g N, for example 14.3-14.4 g/16 g N, such as from 14.4-14.5 g/16 g N, for example 14.5-14.6 g/16 g N, such as from 14.6-14.7 g/16 g N, for example 14.7-14.8 g/16 g N, such as from 14.8-14.9 g/16 g N, for example 14.9-15.0 g/16 g N, such as from 15-16 g/16 g N, for example 16-17 g/16 g N, such as from 17-18 g/16 g N, for example 18-19 g/16 g N, such as from 19-20 g/16 g N.

In one embodiment the present invention relates to a fish feed composition with a Histidine content (amino acid g/16 g N) in the range from 0-20 g/16 g N, such as from 0-0.1 g/16 g N, for example 0.1-0.2 g/16 g N, such as from 0.2-0.3 g/16 g N, for example 0.3-0.4 g/16 g N, such as from 0.4-0.5 g/16 g N, for example 0.5-0.6 g/16 g N, such as from 0.6-0.7 g/16 g N, for example 0.7-0.8 g/16 g N, such as from 0.8-0.9 g/16 g N, for example 0.9-1.0 g/16 g N, such as from 1.0-1.1 g/16 g N, for example 1.1-1.2 g/16 g N, such as from 1.2-1.3 g/16 g N, for example 1.3-1.4 g/16 g N, such as from 1.4-1.5 g/16 g N, for example 1.5-1.6 g/16 g N, such as from 1.6-1.7 g/16 g N, for example 1.7-1.8 g/16 g N, such as from 1.8-1.9 g/16 g N, for example 1.9-2.0 g/16 g N, such as from 2.0-2.1 g/16 g N, for example 2.1-2.2 g/16 g N, such as from 2.2-2.3 g/16 g N, for example 2.3-2.4 g/16 g N, such as from 2.4-2.5 g/16 g N, for example 2.5-2.6 g/16 g N, such as from 2.6-2.7 g/16 g N, for example 2.7-2.8 g/16 g N, such as from 2.8-2.9 g/16 g N, for example 2.9-3.0 g/16 g N, such as from 3.0-3.1 g/16 g N, for example 3.1-3.2 g/16 g N, such as from 3.2-3.3 g/16 g N, for example 3.3-3.4 g/16 g N, such as from 3.4-3.5 g/16 g N, for example 3.5-3.6 g/16 g N, such as from 3.6-3.7 g/16 g N, for example 3.7-3.8 g/16 g N, such as from 3.8-3.9 g/16 g N, for example 3.9-4.0 g/16 g N, such as from 4.0-4.1 g/16 g N, for example 4.1-4.2 g/16 g N, such as from 4.2-4.3 g/16 g N, for example 4.3-4.4 g/16 g N, such as from 4.4-4.5 g/16 g N, for example 4.5-4.6 g/16 g N, such as from 4.6-4.7 g/16 g N, for example 4.7-4.8 g/16 g N, such as from 4.8-4.9 g/16 g N, for example 4.9-5.0 g/16 g N, such as from 5.0-5.1 g/16 g N, for example 5.1-5.2 g/16 g N, such as from 5.2-5.3 g/16 g N, for example 5.3-5.4 g/16 g N, such as from 5.4-5.5 g/16 g N, for example 5.5-5.6 g/16 g N, such as from 5.6-5.7 g/16 g N, for example 5.7-5.8 g/16 g N, such as from 5.8-5.9 g/16 g N, for example 5.9-6.0 g/16 g N, such as from 6.0-6.1 g/16 g N, for example 6.1-6.2 g/16 g N, such as from 6.2-6.3 g/16 g N, for example 6.3-6.4 g/16 g N, such as from 6.4-6.5 g/16 g N, for example 6.5-6.6 g/16 g N, such as from 6.6-6.7 g/16 g N, for example 6.7-6.8 g/16 g N, such as from 6.8-6.9 g/16 g N, for example 6.9-7.0 g/16 g N, such as from 7.0-7.1 g/16 g N, for example 7.1-7.2 g/16 g N, such as from 7.2-7.3 g/16 g N, for example 7.3-7.4 g/16 g N, such as from 7.4-7.5 g/16 g N, for example 7.5-7.6 g/16 g N, such as from 7.6-7.7 g/16 g N, for example 7.7-7.8 g/16 g N, such as from 7.8-7.9 g/16 g N, for example 7.9-8.0 g/16 g N, such as from 8.0-8.1 g/16 g N, for example 8.1-8.2 g/16 g N, such as from 8.2-8.3 g/16 g N, for example 8.3-8.4 g/16 g N, such as from 8.4-8.5 g/16 g N, for example 8.5-8.6 g/16 g N, such as from 8.6-8.7 g/16 g N, for example 8.7-8.8 g/16 g N, such as from 8.8-8.9 g/16 g N, for example 8.9-9.0 g/16 g N, such as from 9.0-9.1 g/16 g N, for example 9.1-9.2 g/16 g N, such as from 9.2-9.3 g/16 g N, for example 9.3-9.4 g/16 g N, such as from 9.4-9.5 g/16 g N, for example 9.5-9.6 g/16 g N, such as from 9.6-9.7 g/16 g N, for example 9.7-9.8 g/16 g N, such as from 9.8-9.9 g/16 g N, for example 9.9-10.0 g/16 g N, such as from 10.0-10.1 g/16 g N, for example 10.1-10.2 g/16 g N, such as from 10.2-10.3 g/16 g N, for example 10.3-10.4 g/16 g N, such as from 10.4-10.5 g/16 g N, for example 10.5-10.6 g/16 g N, such as from 10.6-10.7 g/16 g N, for example 10.7-10.8 g/16 g N, such as from 10.8-10.9 g/16 g N, for example 10.9-11.0 g/16 g N, such as from 11.0-11.1 g/16 g N, for example 11.1-11.2 g/16 g N, such as from 11.2-11.3 g/16 g N, for example 11.3-11.4 g/16 g N, such as from 11.4-11.5 g/16 g N, for example 11.5-11.6 g/16 g N, such as from 11.6-11.7 g/16 g N, for example 11.7-11.8 g/16 g N, such as from 11.8-11.9 g/16 g N, for example 11.9-12.0 g/16 g N, such as from 12.0-12.1 g/16 g N, for example 12.1-12.2 g/16 g N, such as from 12.2-12.3 g/16 g N, for example 12.3-12.4 g/16 g N, such as from 12.4-12.5 g/16 g N, for example 12.5-12.6 g/16 g N, such as from 12.6-12.7 g/16 g N, for example 12.7-12.8 g/16 g N, such as from 12.8-12.9 g/16 g N, for example 12.9-13.0 g/16 g N, such as from 13.0-13.1 g/16 g N, for example 13.1-13.2 g/16 g N, such as from 13.2-13.3 g/16 g N, for example 13.3-13.4 g/16 g N, such as from 13.4-13.5 g/16 g N, for example 13.5-13.6 g/16 g N, such as from 13.6-13.7 g/16 g N, for example 13.7-13.8 g/16 g N, such as from 13.8-13.9 g/16 g N, for example 13.9-14.0 g/16 g N, such as from 14.0-14.1 g/16 g N, for example 14.1-14.2 g/16 g N, such as from 14.2-14.3 g/16 g N, for example 14.3-14.4 g/16 g N, such as from 14.4-14.5 g/16 g N, for example 14.5-14.6 g/16 g N, such as from 14.6-14.7 g/16 g N, for example 14.7-14.8 g/16 g N, such as from 14.8-14.9 g/16 g N, for example 14.9-15.0 g/16 g N, such as from 15-16 g/16 g N, for example 16-17 g/16 g N, such as from 17-18 g/16 g N, for example 18-19 g/16 g N, such as from 19-20 g/16 g N.

In one embodiment the present invention relates to a fish feed composition with an Isoleucine content (amino acid g/16 g N) in the range from 0-20 g/16 g N, such as from 0-0.1 g/16 g N, for example 0.1-0.2 g/16 g N, such as from 0.2-0.3 g/16 g N, for example 0.3-0.4 g/16 g N, such as from 0.4-0.5 g/16 g N, for example 0.5-0.6 g/16 g N, such as from 0.6-0.7 g/16 g N, for example 0.7-0.8 g/16 g N, such as from 0.8-0.9 g/16 g N, for example 0.9-1.0 g/16 g N, such as from 1.0-1.1 g/16 g N, for example 1.1-1.2 g/16 g N, such as from 1.2-1.3 g/16 g N, for example 1.3-1.4 g/16 g N, such as from 1.4-1.5 g/16 g N, for example 1.5-1.6 g/16 g N, such as from 1.6-1.7 g/16 g N, for example 1.7-1.8 g/16 g N, such as from 1.8-1.9 g/16 g N, for example 1.9-2.0 g/16 g N, such as from 2.0-2.1 g/16 g N, for example 2.1-2.2 g/16 g N, such as from 2.2-2.3 g/16 g N, for example 2.3-2.4 g/16 g N, such as from 2.4-2.5 g/16 g N, for example 2.5-2.6 g/16 g N, such as from 2.6-2.7 g/16 g N, for example 2.7-2.8 g/16 g N, such as from 2.8-2.9 g/16 g N, for example 2.9-3.0 g/16 g N, such as from 3.0-3.1 g/16 g N, for example 3.1-3.2 g/16 g N, such as from 3.2-3.3 g/16 g N, for example 3.3-3.4 g/16 g N, such as from 3.4-3.5 g/16 g N, for example 3.5-3.6 g/16 g N, such as from 3.6-3.7 g/16 g N, for example 3.7-3.8 g/16 g N, such as from 3.8-3.9 g/16 g N, for example 3.9-4.0 g/16 g N, such as from 4.0-4.1 g/16 g N, for example 4.1-4.2 g/16 g N, such as from 4.2-4.3 g/16 g N, for example 4.3-4.4 g/16 g N, such as from 4.4-4.5 g/16 g N, for example 4.5-4.6 g/16 g N, such as from 4.6-4.7 g/16 g N, for example 4.7-4.8 g/16 g N, such as from 4.8-4.9 g/16 g N, for example 4.9-5.0 g/16 g N, such as from 5.0-5.1 g/16 g N, for example 5.1-5.2 g/16 g N, such as from 5.2-5.3 g/16 g N, for example 5.3-5.4 g/16 g N, such as from 5.4-5.5 g/16 g N, for example 5.5-5.6 g/16 g N, such as from 5.6-5.7 g/16 g N, for example 5.7-5.8 g/16 g N, such as from 5.8-5.9 g/16 g N, for example 5.9-6.0 g/16 g N, such as from 6.0-6.1 g/16 g N, for example 6.1-6.2 g/16 g N, such as from 6.2-6.3 g/16 g N, for example 6.3-6.4 g/16 g N, such as from 6.4-6.5 g/16 g N, for example 6.5-6.6 g/16 g N, such as from 6.6-6.7 g/16 g N, for example 6.7-6.8 g/16 g N, such as from 6.8-6.9 g/16 g N, for example 6.9-7.0 g/16 g N, such as from 7.0-7.1 g/16 g N, for example 7.1-7.2 g/16 g N, such as from 7.2-7.3 g/16 g N, for example 7.3-7.4 g/16 g N, such as from 7.4-7.5 g/16 g N, for example 7.5-7.6 g/16 g N, such as from 7.6-7.7 g/16 g N, for example 7.7-7.8 g/16 g N, such as from 7.8-7.9 g/16 g N, for example 7.9-8.0 g/16 g N, such as from 8.0-8.1 g/16 g N, for example 8.1-8.2 g/16 g N, such as from 8.2-8.3 g/16 g N, for example 8.3-8.4 g/16 g N, such as from 8.4-8.5 g/16 g N, for example 8.5-8.6 g/16 g N, such as from 8.6-8.7 g/16 g N, for example 8.7-8.8 g/16 g N, such as from 8.8-8.9 g/16 g N, for example 8.9-9.0 g/16 g N, such as from 9.0-9.1 g/16 g N, for example 9.1-9.2 g/16 g N, such as from 9.2-9.3 g/16 g N, for example 9.3-9.4 g/16 g N, such as from 9.4-9.5 g/16 g N, for example 9.5-9.6 g/16 g N, such as from 9.6-9.7 g/16 g N, for example 9.7-9.8 g/16 g N, such as from 9.8-9.9 g/16 g N, for example 9.9-10.0 g/16 g N, such as from 10.0-10.1 g/16 g N, for example 10.1-10.2 g/16 g N, such as from 10.2-10.3 g/16 g N, for example 10.3-10.4 g/16 g N, such as from 10.4-10.5 g/16 g N, for example 10.5-10.6 g/16 g N, such as from 10.6-10.7 g/16 g N, for example 10.7-10.8 g/16 g N, such as from 10.8-10.9 g/16 g N, for example 10.9-11.0 g/16 g N, such as from 11.0-11.1 g/16 g N, for example 11.1-11.2 g/16 g N, such as from 11.2-11.3 g/16 g N, for example 11.3-11.4 g/16 g N, such as from 11.4-11.5 g/16 g N, for example 11.5-11.6 g/16 g N, such as from 11.6-11.7 g/16 g N, for example 11.7-11.8 g/16 g N, such as from 11.8-11.9 g/16 g N, for example 11.9-12.0 g/16 g N, such as from 12.0-12.1 g/16 g N, for example 12.1-12.2 g/16 g N, such as from 12.2-12.3 g/16 g N, for example 12.3-12.4 g/16 g N, such as from 12.4-12.5 g/16 g N, for example 12.5-12.6 g/16 g N, such as from 12.6-12.7 g/16 g N, for example 12.7-12.8 g/16 g N, such as from 12.8-12.9 g/16 g N, for example 12.9-13.0 g/16 g N, such as from 13.0-13.1 g/16 g N, for example 13.1-13.2 g/16 g N, such as from 13.2-13.3 g/16 g N, for example 13.3-13.4 g/16 g N, such as from 13.4-13.5 g/16 g N, for example 13.5-13.6 g/16 g N, such as from 13.6-13.7 g/16 g N, for example 13.7-13.8 g/16 g N, such as from 13.8-13.9 g/16 g N, for example 13.9-14.0 g/16 g N, such as from 14.0-14.1 g/16 g N, for example 14.1-14.2 g/16 g N, such as from 14.2-14.3 g/16 g N, for example 14.3-14.4 g/16 g N, such as from 14.4-14.5 g/16 g N, for example 14.5-14.6 g/16 g N, such as from 14.6-14.7 g/16 g N, for example 14.7-14.8 g/16 g N, such as from 14.8-14.9 g/16 g N, for example 14.9-15.0 g/16 g N, such as from 15-16 g/16 g N, for example 16-17 g/16 g N, such as from 17-18 g/16 g N, for example 18-19 g/16 g N, such as from 19-20 g/16 g N.

In one embodiment the present invention relates to a fish feed composition with a Leucine content (amino acid g/16 g N) in the range from 0-20 g/16 g N, such as from 0-0.1 g/16 g N, for example 0.1-0.2 g/16 g N, such as from 0.2-0.3 g/16 g N, for example 0.3-0.4 g/16 g N, such as from 0.4-0.5 g/16 g N, for example 0.5-0.6 g/16 g N, such as from 0.6-0.7 g/16 g N, for example 0.7-0.8 g/16 g N, such as from 0.8-0.9 g/16 g N, for example 0.9-1.0 g/16 g N, such as from 1.0-1.1 g/16 g N, for example 1.1-1.2 g/16 g N, such as from 1.2-1.3 g/16 g N, for example 1.3-1.4 g/16 g N, such as from 1.4-1.5 g/16 g N, for example 1.5-1.6 g/16 g N, such as from 1.6-1.7 g/16 g N, for example 1.7-1.8 g/16 g N, such as from 1.8-1.9 g/16 g N, for example 1.9-2.0 g/16 g N, such as from 2.0-2.1 g/16 g N, for example 2.1-2.2 g/16 g N, such as from 2.2-2.3 g/16 g N, for example 2.3-2.4 g/16 g N, such as from 2.4-2.5 g/16 g N, for example 2.5-2.6 g/16 g N, such as from 2.6-2.7 g/16 g N, for example 2.7-2.8 g/16 g N, such as from 2.8-2.9 g/16 g N, for example 2.9-3.0 g/16 g N, such as from 3.0-3.1 g/16 g N, for example 3.1-3.2 g/16 g N, such as from 3.2-3.3 g/16 g N, for example 3.3-3.4 g/16 g N, such as from 3.4-3.5 g/16 g N, for example 3.5-3.6 g/16 g N, such as from 3.6-3.7 g/16 g N, for example 3.7-3.8 g/16 g N, such as from 3.8-3.9 g/16 g N, for example 3.9-4.0 g/16 g N, such as from 4.0-4.1 g/16 g N, for example 4.1-4.2 g/16 g N, such as from 4.2-4.3 g/16 g N, for example 4.3-4.4 g/16 g N, such as from 4.4-4.5 g/16 g N, for example 4.5-4.6 g/16 g N, such as from 4.6-4.7 g/16 g N, for example 4.7-4.8 g/16 g N, such as from 4.8-4.9 g/16 g N, for example 4.9-5.0 g/16 g N, such as from 5.0-5.1 g/16 g N, for example 5.1-5.2 g/16 g N, such as from 5.2-5.3 g/16 g N, for example 5.3-5.4 g/16 g N, such as from 5.4-5.5 g/16 g N, for example 5.5-5.6 g/16 g N, such as from 5.6-5.7 g/16 g N, for example 5.7-5.8 g/16 g N, such as from 5.8-5.9 g/16 g N, for example 5.9-6.0 g/16 g N, such as from 6.0-6.1 g/16 g N, for example 6.1-6.2 g/16 g N, such as from 6.2-6.3 g/16 g N, for example 6.3-6.4 g/16 g N, such as from 6.4-6.5 g/16 g N, for example 6.5-6.6 g/16 g N, such as from 6.6-6.7 g/16 g N, for example 6.7-6.8 g/16 g N, such as from 6.8-6.9 g/16 g N, for example 6.9-7.0 g/16 g N, such as from 7.0-7.1 g/16 g N, for example 7.1-7.2 g/16 g N, such as from 7.2-7.3 g/16 g N, for example 7.3-7.4 g/16 g N, such as from 7.4-7.5 g/16 g N, for example 7.5-7.6 g/16 g N, such as from 7.6-7.7 g/16 g N, for example 7.7-7.8 g/16 g N, such as from 7.8-7.9 g/16 g N, for example 7.9-8.0 g/16 g N, such as from 8.0-8.1 g/16 g N, for example 8.1-8.2 g/16 g N, such as from 8.2-8.3 g/16 g N, for example 8.3-8.4 g/16 g N, such as from 8.4-8.5 g/16 g N, for example 8.5-8.6 g/16 g N, such as from 8.6-8.7 g/16 g N, for example 8.7-8.8 g/16 g N, such as from 8.8-8.9 g/16 g N, for example 8.9-9.0 g/16 g N, such as from 9.0-9.1 g/16 g N, for example 9.1-9.2 g/16 g N, such as from 9.2-9.3 g/16 g N, for example 9.3-9.4 g/16 g N, such as from 9.4-9.5 g/16 g N, for example 9.5-9.6 g/16 g N, such as from 9.6-9.7 g/16 g N, for example 9.7-9.8 g/16 g N, such as from 9.8-9.9 g/16 g N, for example 9.9-10.0 g/16 g N, such as from 10.0-10.1 g/16 g N, for example 10.1-10.2 g/16 g N, such as from 10.2-10.3 g/16 g N, for example 10.3-10.4 g/16 g N, such as from 10.4-10.5 g/16 g N, for example 10.5-10.6 g/16 g N, such as from 10.6-10.7 g/16 g N, for example 10.7-10.8 g/16 g N, such as from 10.8-10.9 g/16 g N, for example 10.9-11.0 g/16 g N, such as from 11.0-11.1 g/16 g N, for example 11.1-11.2 g/16 g N, such as from 11.2-11.3 g/16 g N, for example 11.3-11.4 g/16 g N, such as from 11.4-11.5 g/16 g N, for example 11.5-11.6 g/16 g N, such as from 11.6-11.7 g/16 g N, for example 11.7-11.8 g/16 g N, such as from 11.8-11.9 g/16 g N, for example 11.9-12.0 g/16 g N, such as from 12.0-12.1 g/16 g N, for example 12.1-12.2 g/16 g N, such as from 12.2-12.3 g/16 g N, for example 12.3-12.4 g/16 g N, such as from 12.4-12.5 g/16 g N, for example 12.5-12.6 g/16 g N, such as from 12.6-12.7 g/16 g N, for example 12.7-12.8 g/16 g N, such as from 12.8-12.9 g/16 g N, for example 12.9-13.0 g/16 g N, such as from 13.0-13.1 g/16 g N, for example 13.1-13.2 g/16 g N, such as from 13.2-13.3 g/16 g N, for example 13.3-13.4 g/16 g N, such as from 13.4-13.5 g/16 g N, for example 13.5-13.6 g/16 g N, such as from 13.6-13.7 g/16 g N, for example 13.7-13.8 g/16 g N, such as from 13.8-13.9 g/16 g N, for example 13.9-14.0 g/16 g N, such as from 14.0-14.1 g/16 g N, for example 14.1-14.2 g/16 g N, such as from 14.2-14.3 g/16 g N, for example 14.3-14.4 g/16 g N, such as from 14.4-14.5 g/16 g N, for example 14.5-14.6 g/16 g N, such as from 14.6-14.7 g/16 g N, for example 14.7-14.8 g/16 g N, such as from 14.8-14.9 g/16 g N, for example 14.9-15.0 g/16 g N, such as from 15-16 g/16 g N, for example 16-17 g/16 g N, such as from 17-18 g/16 g N, for example 18-19 g/16 g N, such as from 19-20 g/16 g N.

In one embodiment the present invention relates to a fish feed composition with a Lysine content (amino acid g/16 g N) in the range from 0-20 g/16 g N, such as from 0-0.1 g/16 g N, for example 0.1-0.2 g/16 g N, such as from 0.2-0.3 g/16 g N, for example 0.3-0.4 g/16 g N, such as from 0.4-0.5 g/16 g N, for example 0.5-0.6 g/16 g N, such as from 0.6-0.7 g/16 g N, for example 0.7-0.8 g/16 g N, such as from 0.8-0.9 g/16 g N, for example 0.9-1.0 g/16 g N, such as from 1.0-1.1 g/16 g N, for example 1.1-1.2 g/16 g N, such as from 1.2-1.3 g/16 g N, for example 1.3-1.4 g/16 g N, such as from 1.4-1.5 g/16 g N, for example 1.5-1.6 g/16 g N, such as from 1.6-1.7 g/16 g N, for example 1.7-1.8 g/16 g N, such as from 1.8-1.9 g/16 g N, for example 1.9-2.0 g/16 g N, such as from 2.0-2.1 g/16 g N, for example 2.1-2.2 g/16 g N, such as from 2.2-2.3 g/16 g N, for example 2.3-2.4 g/16 g N, such as from 2.4-2.5 g/16 g N, for example 2.5-2.6 g/16 g N, such as from 2.6-2.7 g/16 g N, for example 2.7-2.8 g/16 g N, such as from 2.8-2.9 g/16 g N, for example 2.9-3.0 g/16 g N, such as from 3.0-3.1 g/16 g N, for example 3.1-3.2 g/16 g N, such as from 3.2-3.3 g/16 g N, for example 3.3-3.4 g/16 g N, such as from 3.4-3.5 g/16 g N, for example 3.5-3.6 g/16 g N, such as from 3.6-3.7 g/16 g N, for example 3.7-3.8 g/16 g N, such as from 3.8-3.9 g/16 g N, for example 3.9-4.0 g/16 g N, such as from 4.0-4.1 g/16 g N, for example 4.1-4.2 g/16 g N, such as from 4.2-4.3 g/16 g N, for example 4.3-4.4 g/16 g N, such as from 4.4-4.5 g/16 g N, for example 4.5-4.6 g/16 g N, such as from 4.6-4.7 g/16 g N, for example 4.7-4.8 g/16 g N, such as from 4.8-4.9 g/16 g N, for example 4.9-5.0 g/16 g N, such as from 5.0-5.1 g/16 g N, for example 5.1-5.2 g/16 g N, such as from 5.2-5.3 g/16 g N, for example 5.3-5.4 g/16 g N, such as from 5.4-5.5 g/16 g N, for example 5.5-5.6 g/16 g N, such as from 5.6-5.7 g/16 g N, for example 5.7-5.8 g/16 g N, such as from 5.8-5.9 g/16 g N, for example 5.9-6.0 g/16 g N, such as from 6.0-6.1 g/16 g N, for example 6.1-6.2 g/16 g N, such as from 6.2-6.3 g/16 g N, for example 6.3-6.4 g/16 g N, such as from 6.4-6.5 g/16 g N, for example 6.5-6.6 g/16 g N, such as from 6.6-6.7 g/16 g N, for example 6.7-6.8 g/16 g N, such as from 6.8-6.9 g/16 g N, for example 6.9-7.0 g/16 g N, such as from 7.0-7.1 g/16 g N, for example 7.1-7.2 g/16 g N, such as from 7.2-7.3 g/16 g N, for example 7.3-7.4 g/16 g N, such as from 7.4-7.5 g/16 g N, for example 7.5-7.6 g/16 g N, such as from 7.6-7.7 g/16 g N, for example 7.7-7.8 g/16 g N, such as from 7.8-7.9 g/16 g N, for example 7.9-8.0 g/16 g N, such as from 8.0-8.1 g/16 g N, for example 8.1-8.2 g/16 g N, such as from 8.2-8.3 g/16 g N, for example 8.3-8.4 g/16 g N, such as from 8.4-8.5 g/16 g N, for example 8.5-8.6 g/16 g N, such as from 8.6-8.7 g/16 g N, for example 8.7-8.8 g/16 g N, such as from 8.8-8.9 g/16 g N, for example 8.9-9.0 g/16 g N, such as from 9.0-9.1 g/16 g N, for example 9.1-9.2 g/16 g N, such as from 9.2-9.3 g/16 g N, for example 9.3-9.4 g/16 g N, such as from 9.4-9.5 g/16 g N, for example 9.5-9.6 g/16 g N, such as from 9.6-9.7 g/16 g N, for example 9.7-9.8 g/16 g N, such as from 9.8-9.9 g/16 g N, for example 9.9-10.0 g/16 g N, such as from 10.0-10.1 g/16 g N, for example 10.1-10.2 g/16 g N, such as from 10.2-10.3 g/16 g N, for example 10.3-10.4 g/16 g N, such as from 10.4-10.5 g/16 g N, for example 10.5-10.6 g/16 g N, such as from 10.6-10.7 g/16 g N, for example 10.7-10.8 g/16 g N, such as from 10.8-10.9 g/16 g N, for example 10.9-11.0 g/16 g N, such as from 11.0-11.1 g/16 g N, for example 11.1-11.2 g/16 g N, such as from 11.2-11.3 g/16 g N, for example 11.3-11.4 g/16 g N, such as from 11.4-11.5 g/16 g N, for example 11.5-11.6 g/16 g N, such as from 11.6-11.7 g/16 g N, for example 11.7-11.8 g/16 g N, such as from 11.8-11.9 g/16 g N, for example 11.9-12.0 g/16 g N, such as from 12.0-12.1 g/16 g N, for example 12.1-12.2 g/16 g N, such as from 12.2-12.3 g/16 g N, for example 12.3-12.4 g/16 g N, such as from 12.4-12.5 g/16 g N, for example 12.5-12.6 g/16 g N, such as from 12.6-12.7 g/16 g N, for example 12.7-12.8 g/16 g N, such as from 12.8-12.9 g/16 g N, for example 12.9-13.0 g/16 g N, such as from 13.0-13.1 g/16 g N, for example 13.1-13.2 g/16 g N, such as from 13.2-13.3 g/16 g N, for example 13.3-13.4 g/16 g N, such as from 13.4-13.5 g/16 g N, for example 13.5-13.6 g/16 g N, such as from 13.6-13.7 g/16 g N, for example 13.7-13.8 g/16 g N, such as from 13.8-13.9 g/16 g N, for example 13.9-14.0 g/16 g N, such as from 14.0-14.1 g/16 g N, for example 14.1-14.2 g/16 g N, such as from 14.2-14.3 g/16 g N, for example 14.3-14.4 g/16 g N, such as from 14.4-14.5 g/16 g N, for example 14.5-14.6 g/16 g N, such as from 14.6-14.7 g/16 g N, for example 14.7-14.8 g/16 g N, such as from 14.8-14.9 g/16 g N, for example 14.9-15.0 g/16 g N, such as from 15-16 g/16 g N, for example 16-17 g/16 g N, such as from 17-18 g/16 g N, for example 18-19 g/16 g N, such as from 19-20 g/16 g N.

In one embodiment the present invention relates to a fish feed composition with a Methionine content (amino acid g/16 g N) in the range from 0-20 g/16 g N, such as from 0-0.1 g/16 g N, for example 0.1-0.2 g/16 g N, such as from 0.2-0.3 g/16 g N, for example 0.3-0.4 g/16 g N, such as from 0.4-0.5 g/16 g N, for example 0.5-0.6 g/16 g N, such as from 0.6-0.7 g/16 g N, for example 0.7-0.8 g/16 g N, such as from 0.8-0.9 g/16 g N, for example 0.9-1.0 g/16 g N, such as from 1.0-1.1 g/16 g N, for example 1.1-1.2 g/16 g N, such as from 1.2-1.3 g/16 g N, for example 1.3-1.4 g/16 g N, such as from 1.4-1.5 g/16 g N, for example 1.5-1.6 g/16 g N, such as from 1.6-1.7 g/16 g N, for example 1.7-1.8 g/16 g N, such as from 1.8-1.9 g/16 g N, for example 1.9-2.0 g/16 g N, such as from 2.0-2.1 g/16 g N, for example 2.1-2.2 g/16 g N, such as from 2.2-2.3 g/16 g N, for example 2.3-2.4 g/16 g N, such as from 2.4-2.5 g/16 g N, for example 2.5-2.6 g/16 g N, such as from 2.6-2.7 g/16 g N, for example 2.7-2.8 g/16 g N, such as from 2.8-2.9 g/16 g N, for example 2.9-3.0 g/16 g N, such as from 3.0-3.1 g/16 g N, for example 3.1-3.2 g/16 g N, such as from 3.2-3.3 g/16 g N, for example 3.3-3.4 g/16 g N, such as from 3.4-3.5 g/16 g N, for example 3.5-3.6 g/16 g N, such as from 3.6-3.7 g/16 g N, for example 3.7-3.8 g/16 g N, such as from 3.8-3.9 g/16 g N, for example 3.9-4.0 g/16 g N, such as from 4.0-4.1 g/16 g N, for example 4.1-4.2 g/16 g N, such as from 4.2-4.3 g/16 g N, for example 4.3-4.4 g/16 g N, such as from 4.4-4.5 g/16 g N, for example 4.5-4.6 g/16 g N, such as from 4.6-4.7 g/16 g N, for example 4.7-4.8 g/16 g N, such as from 4.8-4.9 g/16 g N, for example 4.9-5.0 g/16 g N, such as from 5.0-5.1 g/16 g N, for example 5.1-5.2 g/16 g N, such as from 5.2-5.3 g/16 g N, for example 5.3-5.4 g/16 g N, such as from 5.4-5.5 g/16 g N, for example 5.5-5.6 g/16 g N, such as from 5.6-5.7 g/16 g N, for example 5.7-5.8 g/16 g N, such as from 5.8-5.9 g/16 g N, for example 5.9-6.0 g/16 g N, such as from 6.0-6.1 g/16 g N, for example 6.1-6.2 g/16 g N, such as from 6.2-6.3 g/16 g N, for example 6.3-6.4 g/16 g N, such as from 6.4-6.5 g/16 g N, for example 6.5-6.6 g/16 g N, such as from 6.6-6.7 g/16 g N, for example 6.7-6.8 g/16 g N, such as from 6.8-6.9 g/16 g N, for example 6.9-7.0 g/16 g N, such as from 7.0-7.1 g/16 g N, for example 7.1-7.2 g/16 g N, such as from 7.2-7.3 g/16 g N, for example 7.3-7.4 g/16 g N, such as from 7.4-7.5 g/16 g N, for example 7.5-7.6 g/16 g N, such as from 7.6-7.7 g/16 g N, for example 7.7-7.8 g/16 g N, such as from 7.8-7.9 g/16 g N, for example 7.9-8.0 g/16 g N, such as from 8.0-8.1 g/16 g N, for example 8.1-8.2 g/16 g N, such as from 8.2-8.3 g/16 g N, for example 8.3-8.4 g/16 g N, such as from 8.4-8.5 g/16 g N, for example 8.5-8.6 g/16 g N, such as from 8.6-8.7 g/16 g N, for example 8.7-8.8 g/16 g N, such as from 8.8-8.9 g/16 g N, for example 8.9-9.0 g/16 g N, such as from 9.0-9.1 g/16 g N, for example 9.1-9.2 g/16 g N, such as from 9.2-9.3 g/16 g N, for example 9.3-9.4 g/16 g N, such as from 9.4-9.5 g/16 g N, for example 9.5-9.6 g/16 g N, such as from 9.6-9.7 g/16 g N, for example 9.7-9.8 g/16 g N, such as from 9.8-9.9 g/16 g N, for example 9.9-10.0 g/16 g N, such as from 10.0-10.1 g/16 g N, for example 10.1-10.2 g/16 g N, such as from 10.2-10.3 g/16 g N, for example 10.3-10.4 g/16 g N, such as from 10.4-10.5 g/16 g N, for example 10.5-10.6 g/16 g N, such as from 10.6-10.7 g/16 g N, for example 10.7-10.8 g/16 g N, such as from 10.8-10.9 g/16 g N, for example 10.9-11.0 g/16 g N, such as from 11.0-11.1 g/16 g N, for example 11.1-11.2 g/16 g N, such as from 11.2-11.3 g/16 g N, for example 11.3-11.4 g/16 g N, such as from 11.4-11.5 g/16 g N, for example 11.5-11.6 g/16 g N, such as from 11.6-11.7 g/16 g N, for example 11.7-11.8 g/16 g N, such as from 11.8-11.9 g/16 g N, for example 11.9-12.0 g/16 g N, such as from 12.0-12.1 g/16 g N, for example 12.1-12.2 g/16 g N, such as from 12.2-12.3 g/16 g N, for example 12.3-12.4 g/16 g N, such as from 12.4-12.5 g/16 g N, for example 12.5-12.6 g/16 g N, such as from 12.6-12.7 g/16 g N, for example 12.7-12.8 g/16 g N, such as from 12.8-12.9 g/16 g N, for example 12.9-13.0 g/16 g N, such as from 13.0-13.1 g/16 g N, for example 13.1-13.2 g/16 g N, such as from 13.2-13.3 g/16 g N, for example 13.3-13.4 g/16 g N, such as from 13.4-13.5 g/16 g N, for example 13.5-13.6 g/16 g N, such as from 13.6-13.7 g/16 g N, for example 13.7-13.8 g/16 g N, such as from 13.8-13.9 g/16 g N, for example 13.9-14.0 g/16 g N, such as from 14.0-14.1 g/16 g N, for example 14.1-14.2 g/16 g N, such as from 14.2-14.3 g/16 g N, for example 14.3-14.4 g/16 g N, such as from 14.4-14.5 g/16 g N, for example 14.5-14.6 g/16 g N, such as from 14.6-14.7 g/16 g N, for example 14.7-14.8 g/16 g N, such as from 14.8-14.9 g/16 g N, for example 14.9-15.0 g/16 g N, such as from 15-16 g/16 g N, for example 16-17 g/16 g N, such as from 17-18 g/16 g N, for example 18-19 g/16 g N, such as from 19-20 g/16 g N.

In one embodiment the present invention relates to a fish feed composition with a Phenylalanine content (amino acid g/16 g N) in the range from 0-20 g/16 g N, such as from 0-0.1 g/16 g N, for example 0.1-0.2 g/16 g N, such as from 0.2-0.3 g/16 g N, for example 0.3-0.4 g/16 g N, such as from 0.4-0.5 g/16 g N, for example 0.5-0.6 g/16 g N, such as from 0.6-0.7 g/16 g N, for example 0.7-0.8 g/16 g N, such as from 0.8-0.9 g/16 g N, for example 0.9-1.0 g/16 g N, such as from 1.0-1.1 g/16 g N, for example 1.1-1.2 g/16 g N, such as from 1.2-1.3 g/16 g N, for example 1.3-1.4 g/16 g N, such as from 1.4-1.5 g/16 g N, for example 1.5-1.6 g/16 g N, such as from 1.6-1.7 g/16 g N, for example 1.7-1.8 g/16 g N, such as from 1.8-1.9 g/16 g N, for example 1.9-2.0 g/16 g N, such as from 2.0-2.1 g/16 g N, for example 2.1-2.2 g/16 g N, such as from 2.2-2.3 g/16 g N, for example 2.3-2.4 g/16 g N, such as from 2.4-2.5 g/16 g N, for example 2.5-2.6 g/16 g N, such as from 2.6-2.7 g/16 g N, for example 2.7-2.8 g/16 g N, such as from 2.8-2.9 g/16 g N, for example 2.9-3.0 g/16 g N, such as from 3.0-3.1 g/16 g N, for example 3.1-3.2 g/16 g N, such as from 3.2-3.3 g/16 g N, for example 3.3-3.4 g/16 g N, such as from 3.4-3.5 g/16 g N, for example 3.5-3.6 g/16 g N, such as from 3.6-3.7 g/16 g N, for example 3.7-3.8 g/16 g N, such as from 3.8-3.9 g/16 g N, for example 3.9-4.0 g/16 g N, such as from 4.0-4.1 g/16 g N, for example 4.1-4.2 g/16 g N, such as from 4.2-4.3 g/16 g N, for example 4.3-4.4 g/16 g N, such as from 4.4-4.5 g/16 g N, for example 4.5-4.6 g/16 g N, such as from 4.6-4.7 g/16 g N, for example 4.7-4.8 g/16 g N, such as from 4.8-4.9 g/16 g N, for example 4.9-5.0 g/16 g N, such as from 5.0-5.1 g/16 g N, for example 5.1-5.2 g/16 g N, such as from 5.2-5.3 g/16 g N, for example 5.3-5.4 g/16 g N, such as from 5.4-5.5 g/16 g N, for example 5.5-5.6 g/16 g N, such as from 5.6-5.7 g/16 g N, for example 5.7-5.8 g/16 g N, such as from 5.8-5.9 g/16 g N, for example 5.9-6.0 g/16 g N, such as from 6.0-6.1 g/16 g N, for example 6.1-6.2 g/16 g N, such as from 6.2-6.3 g/16 g N, for example 6.3-6.4 g/16 g N, such as from 6.4-6.5 g/16 g N, for example 6.5-6.6 g/16 g N, such as from 6.6-6.7 g/16 g N, for example 6.7-6.8 g/16 g N, such as from 6.8-6.9 g/16 g N, for example 6.9-7.0 g/16 g N, such as from 7.0-7.1 g/16 g N, for example 7.1-7.2 g/16 g N, such as from 7.2-7.3 g/16 g N, for example 7.3-7.4 g/16 g N, such as from 7.4-7.5 g/16 g N, for example 7.5-7.6 g/16 g N, such as from 7.6-7.7 g/16 g N, for example 7.7-7.8 g/16 g N, such as from 7.8-7.9 g/16 g N, for example 7.9-8.0 g/16 g N, such as from 8.0-8.1 g/16 g N, for example 8.1-8.2 g/16 g N, such as from 8.2-8.3 g/16 g N, for example 8.3-8.4 g/16 g N, such as from 8.4-8.5 g/16 g N, for example 8.5-8.6 g/16 g N, such as from 8.6-8.7 g/16 g N, for example 8.7-8.8 g/16 g N, such as from 8.8-8.9 g/16 g N, for example 8.9-9.0 g/16 g N, such as from 9.0-9.1 g/16 g N, for example 9.1-9.2 g/16 g N, such as from 9.2-9.3 g/16 g N, for example 9.3-9.4 g/16 g N, such as from 9.4-9.5 g/16 g N, for example 9.5-9.6 g/16 g N, such as from 9.6-9.7 g/16 g N, for example 9.7-9.8 g/16 g N, such as from 9.8-9.9 g/16 g N, for example 9.9-10.0 g/16 g N, such as from 10.0-10.1 g/16 g N, for example 10.1-10.2 g/16 g N, such as from 10.2-10.3 g/16 g N, for example 10.3-10.4 g/16 g N, such as from 10.4-10.5 g/16 g N, for example 10.5-10.6 g/16 g N, such as from 10.6-10.7 g/16 g N, for example 10.7-10.8 g/16 g N, such as from 10.8-10.9 g/16 g N, for example 10.9-11.0 g/16 g N, such as from 11.0-11.1 g/16 g N, for example 11.1-11.2 g/16 g N, such as from 11.2-11.3 g/16 g N, for example 11.3-11.4 g/16 g N, such as from 11.4-11.5 g/16 g N, for example 11.5-11.6 g/16 g N, such as from 11.6-11.7 g/16 g N, for example 11.7-11.8 g/16 g N, such as from 11.8-11.9 g/16 g N, for example 11.9-12.0 g/16 g N, such as from 12.0-12.1 g/16 g N, for example 12.1-12.2 g/16 g N, such as from 12.2-12.3 g/16 g N, for example 12.3-12.4 g/16 g N, such as from 12.4-12.5 g/16 g N, for example 12.5-12.6 g/16 g N, such as from 12.6-12.7 g/16 g N, for example 12.7-12.8 g/16 g N, such as from 12.8-12.9 g/16 g N, for example 12.9-13.0 g/16 g N, such as from 13.0-13.1 g/16 g N, for example 13.1-13.2 g/16 g N, such as from 13.2-13.3 g/16 g N, for example 13.3-13.4 g/16 g N, such as from 13.4-13.5 g/16 g N, for example 13.5-13.6 g/16 g N, such as from 13.6-13.7 g/16 g N, for example 13.7-13.8 g/16 g N, such as from 13.8-13.9 g/16 g N, for example 13.9-14.0 g/16 g N, such as from 14.0-14.1 g/16 g N, for example 14.1-14.2 g/16 g N, such as from 14.2-14.3 g/16 g N, for example 14.3-14.4 g/16 g N, such as from 14.4-14.5 g/16 g N, for example 14.5-14.6 g/16 g N, such as from 14.6-14.7 g/16 g N, for example 14.7-14.8 g/16 g N, such as from 14.8-14.9 g/16 g N, for example 14.9-15.0 g/16 g N, such as from 15-16 g/16 g N, for example 16-17 g/16 g N, such as from 17-18 g/16 g N, for example 18-19 g/16 g N, such as from 19-20 g/16 g N.

In one embodiment the present invention relates to a fish feed composition with a Proline content (amino acid g/16 g N) in the range from 0-20 g/16 g N, such as from 0-0.1 g/16 g N, for example 0.1-0.2 g/16 g N, such as from 0.2-0.3 g/16 g N, for example 0.3-0.4 g/16 g N, such as from 0.4-0.5 g/16 g N, for example 0.5-0.6 g/16 g N, such as from 0.6-0.7 g/16 g N, for example 0.7-0.8 g/16 g N, such as from 0.8-0.9 g/16 g N, for example 0.9-1.0 g/16 g N, such as from 1.0-1.1 g/16 g N, for example 1.1-1.2 g/16 g N, such as from 1.2-1.3 g/16 g N, for example 1.3-1.4 g/16 g N, such as from 1.4-1.5 g/16 g N, for example 1.5-1.6 g/16 g N, such as from 1.6-1.7 g/16 g N, for example 1.7-1.8 g/16 g N, such as from 1.8-1.9 g/16 g N, for example 1.9-2.0 g/16 g N, such as from 2.0-2.1 g/16 g N, for example 2.1-2.2 g/16 g N, such as from 2.2-2.3 g/16 g N, for example 2.3-2.4 g/16 g N, such as from 2.4-2.5 g/16 g N, for example 2.5-2.6 g/16 g N, such as from 2.6-2.7 g/16 g N, for example 2.7-2.8 g/16 g N, such as from 2.8-2.9 g/16 g N, for example 2.9-3.0 g/16 g N, such as from 3.0-3.1 g/16 g N, for example 3.1-3.2 g/16 g N, such as from 3.2-3.3 g/16 g N, for example 3.3-3.4 g/16 g N, such as from 3.4-3.5 g/16 g N, for example 3.5-3.6 g/16 g N, such as from 3.6-3.7 g/16 g N, for example 3.7-3.8 g/16 g N, such as from 3.8-3.9 g/16 g N, for example 3.9-4.0 g/16 g N, such as from 4.0-4.1 g/16 g N, for example 4.1-4.2 g/16 g N, such as from 4.2-4.3 g/16 g N, for example 4.3-4.4 g/16 g N, such as from 4.4-4.5 g/16 g N, for example 4.5-4.6 g/16 g N, such as from 4.6-4.7 g/16 g N, for example 4.7-4.8 g/16 g N, such as from 4.8-4.9 g/16 g N, for example 4.9-5.0 g/16 g N, such as from 5.0-5.1 g/16 g N, for example 5.1-5.2 g/16 g N, such as from 5.2-5.3 g/16 g N, for example 5.3-5.4 g/16 g N, such as from 5.4-5.5 g/16 g N, for example 5.5-5.6 g/16 g N, such as from 5.6-5.7 g/16 g N, for example 5.7-5.8 g/16 g N, such as from 5.8-5.9 g/16 g N, for example 5.9-6.0 g/16 g N, such as from 6.0-6.1 g/16 g N, for example 6.1-6.2 g/16 g N, such as from 6.2-6.3 g/16 g N, for example 6.3-6.4 g/16 g N, such as from 6.4-6.5 g/16 g N, for example 6.5-6.6 g/16 g N, such as from 6.6-6.7 g/16 g N, for example 6.7-6.8 g/16 g N, such as from 6.8-6.9 g/16 g N, for example 6.9-7.0 g/16 g N, such as from 7.0-7.1 g/16 g N, for example 7.1-7.2 g/16 g N, such as from 7.2-7.3 g/16 g N, for example 7.3-7.4 g/16 g N, such as from 7.4-7.5 g/16 g N, for example 7.5-7.6 g/16 g N, such as from 7.6-7.7 g/16 g N, for example 7.7-7.8 g/16 g N, such as from 7.8-7.9 g/16 g N, for example 7.9-8.0 g/16 g N, such as from 8.0-8.1 g/16 g N, for example 8.1-8.2 g/16 g N, such as from 8.2-8.3 g/16 g N, for example 8.3-8.4 g/16 g N, such as from 8.4-8.5 g/16 g N, for example 8.5-8.6 g/16 g N, such as from 8.6-8.7 g/16 g N, for example 8.7-8.8 g/16 g N, such as from 8.8-8.9 g/16 g N, for example 8.9-9.0 g/16 g N, such as from 9.0-9.1 g/16 g N, for example 9.1-9.2 g/16 g N, such as from 9.2-9.3 g/16 g N, for example 9.3-9.4 g/16 g N, such as from 9.4-9.5 g/16 g N, for example 9.5-9.6 g/16 g N, such as from 9.6-9.7 g/16 g N, for example 9.7-9.8 g/16 g N, such as from 9.8-9.9 g/16 g N, for example 9.9-10.0 g/16 g N, such as from 10.0-10.1 g/16 g N, for example 10.1-10.2 g/16 g N, such as from 10.2-10.3 g/16 g N, for example 10.3-10.4 g/16 g N, such as from 10.4-10.5 g/16 g N, for example 10.5-10.6 g/16 g N, such as from 10.6-10.7 g/16 g N, for example 10.7-10.8 g/16 g N, such as from 10.8-10.9 g/16 g N, for example 10.9-11.0 g/16 g N, such as from 11.0-11.1 g/16 g N, for example 11.1-11.2 g/16 g N, such as from 11.2-11.3 g/16 g N, for example 11.3-11.4 g/16 g N, such as from 11.4-11.5 g/16 g N, for example 11.5-11.6 g/16 g N, such as from 11.6-11.7 g/16 g N, for example 11.7-11.8 g/16 g N, such as from 11.8-11.9 g/16 g N, for example 11.9-12.0 g/16 g N, such as from 12.0-12.1 g/16 g N, for example 12.1-12.2 g/16 g N, such as from 12.2-12.3 g/16 g N, for example 12.3-12.4 g/16 g N, such as from 12.4-12.5 g/16 g N, for example 12.5-12.6 g/16 g N, such as from 12.6-12.7 g/16 g N, for example 12.7-12.8 g/16 g N, such as from 12.8-12.9 g/16 g N, for example 12.9-13.0 g/16 g N, such as from 13.0-13.1 g/16 g N, for example 13.1-13.2 g/16 g N, such as from 13.2-13.3 g/16 g N, for example 13.3-13.4 g/16 g N, such as from 13.4-13.5 g/16 g N, for example 13.5-13.6 g/16 g N, such as from 13.6-13.7 g/16 g N, for example 13.7-13.8 g/16 g N, such as from 13.8-13.9 g/16 g N, for example 13.9-14.0 g/16 g N, such as from 14.0-14.1 g/16 g N, for example 14.1-14.2 g/16 g N, such as from 14.2-14.3 g/16 g N, for example 14.3-14.4 g/16 g N, such as from 14.4-14.5 g/16 g N, for example 14.5-14.6 g/16 g N, such as from 14.6-14.7 g/16 g N, for example 14.7-14.8 g/16 g N, such as from 14.8-14.9 g/16 g N, for example 14.9-15.0 g/16 g N, such as from 15-16 g/16 g N, for example 16-17 g/16 g N, such as from 17-18 g/16 g N, for example 18-19 g/16 g N, such as from 19-20 g/16 g N.

In one embodiment the present invention relates to a fish feed composition with a Serine content (amino acid g/16 g N) in the range from 0-20 g/16 g N, such as from 0-0.1 g/16 g N, for example 0.1-0.2 g/16 g N, such as from 0.2-0.3 g/16 g N, for example 0.3-0.4 g/16 g N, such as from 0.4-0.5 g/16 g N, for example 0.5-0.6 g/16 g N, such as from 0.6-0.7 g/16 g N, for example 0.7-0.8 g/16 g N, such as from 0.8-0.9 g/16 g N, for example 0.9-1.0 g/16 g N, such as from 1.0-1.1 g/16 g N, for example 1.1-1.2 g/16 g N, such as from 1.2-1.3 g/16 g N, for example 1.3-1.4 g/16 g N, such as from 1.4-1.5 g/16 g N, for example 1.5-1.6 g/16 g N, such as from 1.6-1.7 g/16 g N, for example 1.7-1.8 g/16 g N, such as from 1.8-1.9 g/16 g N, for example 1.9-2.0 g/16 g N, such as from 2.0-2.1 g/16 g N, for example 2.1-2.2 g/16 g N, such as from 2.2-2.3 g/16 g N, for example 2.3-2.4 g/16 g N, such as from 2.4-2.5 g/16 g N, for example 2.5-2.6 g/16 g N, such as from 2.6-2.7 g/16 g N, for example 2.7-2.8 g/16 g N, such as from 2.8-2.9 g/16 g N, for example 2.9-3.0 g/16 g N, such as from 3.0-3.1 g/16 g N, for example 3.1-3.2 g/16 g N, such as from 3.2-3.3 g/16 g N, for example 3.3-3.4 g/16 g N, such as from 3.4-3.5 g/16 g N, for example 3.5-3.6 g/16 g N, such as from 3.6-3.7 g/16 g N, for example 3.7-3.8 g/16 g N, such as from 3.8-3.9 g/16 g N, for example 3.9-4.0 g/16 g N, such as from 4.0-4.1 g/16 g N, for example 4.1-4.2 g/16 g N, such as from 4.2-4.3 g/16 g N, for example 4.3-4.4 g/16 g N, such as from 4.4-4.5 g/16 g N, for example 4.5-4.6 g/16 g N, such as from 4.6-4.7 g/16 g N, for example 4.7-4.8 g/16 g N, such as from 4.8-4.9 g/16 g N, for example 4.9-5.0 g/16 g N, such as from 5.0-5.1 g/16 g N, for example 5.1-5.2 g/16 g N, such as from 5.2-5.3 g/16 g N, for example 5.3-5.4 g/16 g N, such as from 5.4-5.5 g/16 g N, for example 5.5-5.6 g/16 g N, such as from 5.6-5.7 g/16 g N, for example 5.7-5.8 g/16 g N, such as from 5.8-5.9 g/16 g N, for example 5.9-6.0 g/16 g N, such as from 6.0-6.1 g/16 g N, for example 6.1-6.2 g/16 g N, such as from 6.2-6.3 g/16 g N, for example 6.3-6.4 g/16 g N, such as from 6.4-6.5 g/16 g N, for example 6.5-6.6 g/16 g N, such as from 6.6-6.7 g/16 g N, for example 6.7-6.8 g/16 g N, such as from 6.8-6.9 g/16 g N, for example 6.9-7.0 g/16 g N, such as from 7.0-7.1 g/16 g N, for example 7.1-7.2 g/16 g N, such as from 7.2-7.3 g/16 g N, for example 7.3-7.4 g/16 g N, such as from 7.4-7.5 g/16 g N, for example 7.5-7.6 g/16 g N, such as from 7.6-7.7 g/16 g N, for example 7.7-7.8 g/16 g N, such as from 7.8-7.9 g/16 g N, for example 7.9-8.0 g/16 g N, such as from 8.0-8.1 g/16 g N, for example 8.1-8.2 g/16 g N, such as from 8.2-8.3 g/16 g N, for example 8.3-8.4 g/16 g N, such as from 8.4-8.5 g/16 g N, for example 8.5-8.6 g/16 g N, such as from 8.6-8.7 g/16 g N, for example 8.7-8.8 g/16 g N, such as from 8.8-8.9 g/16 g N, for example 8.9-9.0 g/16 g N, such as from 9.0-9.1 g/16 g N, for example 9.1-9.2 g/16 g N, such as from 9.2-9.3 g/16 g N, for example 9.3-9.4 g/16 g N, such as from 9.4-9.5 g/16 g N, for example 9.5-9.6 g/16 g N, such as from 9.6-9.7 g/16 g N, for example 9.7-9.8 g/16 g N, such as from 9.8-9.9 g/16 g N, for example 9.9-10.0 g/16 g N, such as from 10.0-10.1 g/16 g N, for example 10.1-10.2 g/16 g N, such as from 10.2-10.3 g/16 g N, for example 10.3-10.4 g/16 g N, such as from 10.4-10.5 g/16 g N, for example 10.5-10.6 g/16 g N, such as from 10.6-10.7 g/16 g N, for example 10.7-10.8 g/16 g N, such as from 10.8-10.9 g/16 g N, for example 10.9-11.0 g/16 g N, such as from 11.0-11.1 g/16 g N, for example 11.1-11.2 g/16 g N, such as from 11.2-11.3 g/16 g N, for example 11.3-11.4 g/16 g N, such as from 11.4-11.5 g/16 g N, for example 11.5-11.6 g/16 g N, such as from 11.6-11.7 g/16 g N, for example 11.7-11.8 g/16 g N, such as from 11.8-11.9 g/16 g N, for example 11.9-12.0 g/16 g N, such as from 12.0-12.1 g/16 g N, for example 12.1-12.2 g/16 g N, such as from 12.2-12.3 g/16 g N, for example 12.3-12.4 g/16 g N, such as from 12.4-12.5 g/16 g N, for example 12.5-12.6 g/16 g N, such as from 12.6-12.7 g/16 g N, for example 12.7-12.8 g/16 g N, such as from 12.8-12.9 g/16 g N, for example 12.9-13.0 g/16 g N, such as from 13.0-13.1 g/16 g N, for example 13.1-13.2 g/16 g N, such as from 13.2-13.3 g/16 g N, for example 13.3-13.4 g/16 g N, such as from 13.4-13.5 g/16 g N, for example 13.5-13.6 g/16 g N, such as from 13.6-13.7 g/16 g N, for example 13.7-13.8 g/16 g N, such as from 13.8-13.9 g/16 g N, for example 13.9-14.0 g/16 g N, such as from 14.0-14.1 g/16 g N, for example 14.1-14.2 g/16 g N, such as from 14.2-14.3 g/16 g N, for example 14.3-14.4 g/16 g N, such as from 14.4-14.5 g/16 g N, for example 14.5-14.6 g/16 g N, such as from 14.6-14.7 g/16 g N, for example 14.7-14.8 g/16 g N, such as from 14.8-14.9 g/16 g N, for example 14.9-15.0 g/16 g N, such as from 15-16 g/16 g N, for example 16-17 g/16 g N, such as from 17-18 g/16 g N, for example 18-19 g/16 g N, such as from 19-20 g/16 g N.

In one embodiment the present invention relates to a fish feed composition with a Threonine content (amino acid g/16 g N) in the range from 0-20 g/16 g N, such as from 0-0.1 g/16 g N, for example 0.1-0.2 g/16 g N, such as from 0.2-0.3 g/16 g N, for example 0.3-0.4 g/16 g N, such as from 0.4-0.5 g/16 g N, for example 0.5-0.6 g/16 g N, such as from 0.6-0.7 g/16 g N, for example 0.7-0.8 g/16 g N, such as from 0.8-0.9 g/16 g N, for example 0.9-1.0 g/16 g N, such as from 1.0-1.1 g/16 g N, for example 1.1-1.2 g/16 g N, such as from 1.2-1.3 g/16 g N, for example 1.3-1.4 g/16 g N, such as from 1.4-1.5 g/16 g N, for example 1.5-1.6 g/16 g N, such as from 1.6-1.7 g/16 g N, for example 1.7-1.8 g/16 g N, such as from 1.8-1.9 g/16 g N, for example 1.9-2.0 g/16 g N, such as from 2.0-2.1 g/16 g N, for example 2.1-2.2 g/16 g N, such as from 2.2-2.3 g/16 g N, for example 2.3-2.4 g/16 g N, such as from 2.4-2.5 g/16 g N, for example 2.5-2.6 g/16 g N, such as from 2.6-2.7 g/16 g N, for example 2.7-2.8 g/16 g N, such as from 2.8-2.9 g/16 g N, for example 2.9-3.0 g/16 g N, such as from 3.0-3.1 g/16 g N, for example 3.1-3.2 g/16 g N, such as from 3.2-3.3 g/16 g N, for example 3.3-3.4 g/16 g N, such as from 3.4-3.5 g/16 g N, for example 3.5-3.6 g/16 g N, such as from 3.6-3.7 g/16 g N, for example 3.7-3.8 g/16 g N, such as from 3.8-3.9 g/16 g N, for example 3.9-4.0 g/16 g N, such as from 4.0-4.1 g/16 g N, for example 4.1-4.2 g/16 g N, such as from 4.2-4.3 g/16 g N, for example 4.3-4.4 g/16 g N, such as from 4.4-4.5 g/16 g N, for example 4.5-4.6 g/16 g N, such as from 4.6-4.7 g/16 g N, for example 4.7-4.8 g/16 g N, such as from 4.8-4.9 g/16 g N, for example 4.9-5.0 g/16 g N, such as from 5.0-5.1 g/16 g N, for example 5.1-5.2 g/16 g N, such as from 5.2-5.3 g/16 g N, for example 5.3-5.4 g/16 g N, such as from 5.4-5.5 g/16 g N, for example 5.5-5.6 g/16 g N, such as from 5.6-5.7 g/16 g N, for example 5.7-5.8 g/16 g N, such as from 5.8-5.9 g/16 g N, for example 5.9-6.0 g/16 g N, such as from 6.0-6.1 g/16 g N, for example 6.1-6.2 g/16 g N, such as from 6.2-6.3 g/16 g N, for example 6.3-6.4 g/16 g N, such as from 6.4-6.5 g/16 g N, for example 6.5-6.6 g/16 g N, such as from 6.6-6.7 g/16 g N, for example 6.7-6.8 g/16 g N, such as from 6.8-6.9 g/16 g N, for example 6.9-7.0 g/16 g N, such as from 7.0-7.1 g/16 g N, for example 7.1-7.2 g/16 g N, such as from 7.2-7.3 g/16 g N, for example 7.3-7.4 g/16 g N, such as from 7.4-7.5 g/16 g N, for example 7.5-7.6 g/16 g N, such as from 7.6-7.7 g/16 g N, for example 7.7-7.8 g/16 g N, such as from 7.8-7.9 g/16 g N, for example 7.9-8.0 g/16 g N, such as from 8.0-8.1 g/16 g N, for example 8.1-8.2 g/16 g N, such as from 8.2-8.3 g/16 g N, for example 8.3-8.4 g/16 g N, such as from 8.4-8.5 g/16 g N, for example 8.5-8.6 g/16 g N, such as from 8.6-8.7 g/16 g N, for example 8.7-8.8 g/16 g N, such as from 8.8-8.9 g/16 g N, for example 8.9-9.0 g/16 g N, such as from 9.0-9.1 g/16 g N, for example 9.1-9.2 g/16 g N, such as from 9.2-9.3 g/16 g N, for example 9.3-9.4 g/16 g N, such as from 9.4-9.5 g/16 g N, for example 9.5-9.6 g/16 g N, such as from 9.6-9.7 g/16 g N, for example 9.7-9.8 g/16 g N, such as from 9.8-9.9 g/16 g N, for example 9.9-10.0 g/16 g N, such as from 10.0-10.1 g/16 g N, for example 10.1-10.2 g/16 g N, such as from 10.2-10.3 g/16 g N, for example 10.3-10.4 g/16 g N, such as from 10.4-10.5 g/16 g N, for example 10.5-10.6 g/16 g N, such as from 10.6-10.7 g/16 g N, for example 10.7-10.8 g/16 g N, such as from 10.8-10.9 g/16 g N, for example 10.9-11.0 g/16 g N, such as from 11.0-11.1 g/16 g N, for example 11.1-11.2 g/16 g N, such as from 11.2-11.3 g/16 g N, for example 11.3-11.4 g/16 g N, such as from 11.4-11.5 g/16 g N, for example 11.5-11.6 g/16 g N, such as from 11.6-11.7 g/16 g N, for example 11.7-11.8 g/16 g N, such as from 11.8-11.9 g/16 g N, for example 11.9-12.0 g/16 g N, such as from 12.0-12.1 g/16 g N, for example 12.1-12.2 g/16 g N, such as from 12.2-12.3 g/16 g N, for example 12.3-12.4 g/16 g N, such as from 12.4-12.5 g/16 g N, for example 12.5-12.6 g/16 g N, such as from 12.6-12.7 g/16 g N, for example 12.7-12.8 g/16 g N, such as from 12.8-12.9 g/16 g N, for example 12.9-13.0 g/16 g N, such as from 13.0-13.1 g/16 g N, for example 13.1-13.2 g/16 g N, such as from 13.2-13.3 g/16 g N, for example 13.3-13.4 g/16 g N, such as from 13.4-13.5 g/16 g N, for example 13.5-13.6 g/16 g N, such as from 13.6-13.7 g/16 g N, for example 13.7-13.8 g/16 g N, such as from 13.8-13.9 g/16 g N, for example 13.9-14.0 g/16 g N, such as from 14.0-14.1 g/16 g N, for example 14.1-14.2 g/16 g N, such as from 14.2-14.3 g/16 g N, for example 14.3-14.4 g/16 g N, such as from 14.4-14.5 g/16 g N, for example 14.5-14.6 g/16 g N, such as from 14.6-14.7 g/16 g N, for example 14.7-14.8 g/16 g N, such as from 14.8-14.9 g/16 g N, for example 14.9-15.0 g/16 g N, such as from 15-16 g/16 g N, for example 16-17 g/16 g N, such as from 17-18 g/16 g N, for example 18-19 g/16 g N, such as from 19-20 g/16 g N.

In one embodiment the present invention relates to a fish feed composition with a

Tryptophan content (amino acid g/16 g N) in the range from 0-20 g/16 g N, such as from 0-0.1 g/16 g N, for example 0.1-0.2 g/16 g N, such as from 0.2-0.3 g/16 g N, for example 0.3-0.4 g/16 g N, such as from 0.4-0.5 g/16 g N, for example 0.5-0.6 g/16 g N, such as from 0.6-0.7 g/16 g N, for example 0.7-0.8 g/16 g N, such as from 0.8-0.9 g/16 g N, for example 0.9-1.0 g/16 g N, such as from 1.0-1.1 g/16 g N, for example 1.1-1.2 g/16 g N, such as from 1.2-1.3 g/16 g N, for example 1.3-1.4 g/16 g N, such as from 1.4-1.5 g/16 g N, for example 1.5-1.6 g/16 g N, such as from 1.6-1.7 g/16 g N, for example 1.7-1.8 g/16 g N, such as from 1.8-1.9 g/16 g N, for example 1.9-2.0 g/16 g N, such as from 2.0-2.1 g/16 g N, for example 2.1-2.2 g/16 g N, such as from 2.2-2.3 g/16 g N, for example 2.3-2.4 g/16 g N, such as from 2.4-2.5 g/16 g N, for example 2.5-2.6 g/16 g N, such as from 2.6-2.7 g/16 g N, for example 2.7-2.8 g/16 g N, such as from 2.8-2.9 g/16 g N, for example 2.9-3.0 g/16 g N, such as from 3.0-3.1 g/16 g N, for example 3.1-3.2 g/16 g N, such as from 3.2-3.3 g/16 g N, for example 3.3-3.4 g/16 g N, such as from 3.4-3.5 g/16 g N, for example 3.5-3.6 g/16 g N, such as from 3.6-3.7 g/16 g N, for example 3.7-3.8 g/16 g N, such as from 3.8-3.9 g/16 g N, for example 3.9-4.0 g/16 g N, such as from 4.0-4.1 g/16 g N, for example 4.1-4.2 g/16 g N, such as from 4.2-4.3 g/16 g N, for example 4.3-4.4 g/16 g N, such as from 4.4-4.5 g/16 g N, for example 4.5-4.6 g/16 g N, such as from 4.6-4.7 g/16 g N, for example 4.7-4.8 g/16 g N, such as from 4.8-4.9 g/16 g N, for example 4.9-5.0 g/16 g N, such as from 5.0-5.1 g/16 g N, for example 5.1-5.2 g/16 g N, such as from 5.2-5.3 g/16 g N, for example 5.3-5.4 g/16 g N, such as from 5.4-5.5 g/16 g N, for example 5.5-5.6 g/16 g N, such as from 5.6-5.7 g/16 g N, for example 5.7-5.8 g/16 g N, such as from 5.8-5.9 g/16 g N, for example 5.9-6.0 g/16 g N, such as from 6.0-6.1 g/16 g N, for example 6.1-6.2 g/16 g N, such as from 6.2-6.3 g/16 g N, for example 6.3-6.4 g/16 g N, such as from 6.4-6.5 g/16 g N, for example 6.5-6.6 g/16 g N, such as from 6.6-6.7 g/16 g N, for example 6.7-6.8 g/16 g N, such as from 6.8-6.9 g/16 g N, for example 6.9-7.0 g/16 g N, such as from 7.0-7.1 g/16 g N, for example 7.1-7.2 g/16 g N, such as from 7.2-7.3 g/16 g N, for example 7.3-7.4 g/16 g N, such as from 7.4-7.5 g/16 g N, for example 7.5-7.6 g/16 g N, such as from 7.6-7.7 g/16 g N, for example 7.7-7.8 g/16 g N, such as from 7.8-7.9 g/16 g N, for example 7.9-8.0 g/16 g N, such as from 8.0-8.1 g/16 g N, for example 8.1-8.2 g/16 g N, such as from 8.2-8.3 g/16 g N, for example 8.3-8.4 g/16 g N, such as from 8.4-8.5 g/16 g N, for example 8.5-8.6 g/16 g N, such as from 8.6-8.7 g/16 g N, for example 8.7-8.8 g/16 g N, such as from 8.8-8.9 g/16 g N, for example 8.9-9.0 g/16 g N, such as from 9.0-9.1 g/16 g N, for example 9.1-9.2 g/16 g N, such as from 9.2-9.3 g/16 g N, for example 9.3-9.4 g/16 g N, such as from 9.4-9.5 g/16 g N, for example 9.5-9.6 g/16 g N, such as from 9.6-9.7 g/16 g N, for example 9.7-9.8 g/16 g N, such as from 9.8-9.9 g/16 g N, for example 9.9-10.0 g/16 g N, such as from 10.0-10.1 g/16 g N, for example 10.1-10.2 g/16 g N, such as from 10.2-10.3 g/16 g N, for example 10.3-10.4 g/16 g N, such as from 10.4-10.5 g/16 g N, for example 10.5-10.6 g/16 g N, such as from 10.6-10.7 g/16 g N, for example 10.7-10.8 g/16 g N, such as from 10.8-10.9 g/16 g N, for example 10.9-11.0 g/16 g N, such as from 11.0-11.1 g/16 g N, for example 11.1-11.2 g/16 g N, such as from 11.2-11.3 g/16 g N, for example 11.3-11.4 g/16 g N, such as from 11.4-11.5 g/16 g N, for example 11.5-11.6 g/16 g N, such as from 11.6-11.7 g/16 g N, for example 11.7-11.8 g/16 g N, such as from 11.8-11.9 g/16 g N, for example 11.9-12.0 g/16 g N, such as from 12.0-12.1 g/16 g N, for example 12.1-12.2 g/16 g N, such as from 12.2-12.3 g/16 g N, for example 12.3-12.4 g/16 g N, such as from 12.4-12.5 g/16 g N, for example 12.5-12.6 g/16 g N, such as from 12.6-12.7 g/16 g N, for example 12.7-12.8 g/16 g N, such as from 12.8-12.9 g/16 g N, for example 12.9-13.0 g/16 g N, such as from 13.0-13.1 g/16 g N, for example 13.1-13.2 g/16 g N, such as from 13.2-13.3 g/16 g N, for example 13.3-13.4 g/16 g N, such as from 13.4-13.5 g/16 g N, for example 13.5-13.6 g/16 g N, such as from 13.6-13.7 g/16 g N, for example 13.7-13.8 g/16 g N, such as from 13.8-13.9 g/16 g N, for example 13.9-14.0 g/16 g N, such as from 14.0-14.1 g/16 g N, for example 14.1-14.2 g/16 g N, such as from 14.2-14.3 g/16 g N, for example 14.3-14.4 g/16 g N, such as from 14.4-14.5 g/16 g N, for example 14.5-14.6 g/16 g N, such as from 14.6-14.7 g/16 g N, for example 14.7-14.8 g/16 g N, such as from 14.8-14.9 g/16 g N, for example 14.9-15.0 g/16 g N, such as from 15-16 g/16 g N, for example 16-17 g/16 g N, such as from 17-18 g/16 g N, for example 18-19 g/16 g N, such as from 19-20 g/16 g N.

In one embodiment the present invention relates to a fish feed composition with a Tyrosine content (amino acid g/16 g N) in the range from 0-20 g/16 g N, such as from 0-0.1 g/16 g N, for example 0.1-0.2 g/16 g N, such as from 0.2-0.3 g/16 g N, for example 0.3-0.4 g/16 g N, such as from 0.4-0.5 g/16 g N, for example 0.5-0.6 g/16 g N, such as from 0.6-0.7 g/16 g N, for example 0.7-0.8 g/16 g N, such as from 0.8-0.9 g/16 g N, for example 0.9-1.0 g/16 g N, such as from 1.0-1.1 g/16 g N, for example 1.1-1.2 g/16 g N, such as from 1.2-1.3 g/16 g N, for example 1.3-1.4 g/16 g N, such as from 1.4-1.5 g/16 g N, for example 1.5-1.6 g/16 g N, such as from 1.6-1.7 g/16 g N, for example 1.7-1.8 g/16 g N, such as from 1.8-1.9 g/16 g N, for example 1.9-2.0 g/16 g N, such as from 2.0-2.1 g/16 g N, for example 2.1-2.2 g/16 g N, such as from 2.2-2.3 g/16 g N, for example 2.3-2.4 g/16 g N, such as from 2.4-2.5 g/16 g N, for example 2.5-2.6 g/16 g N, such as from 2.6-2.7 g/16 g N, for example 2.7-2.8 g/16 g N, such as from 2.8-2.9 g/16 g N, for example 2.9-3.0 g/16 g N, such as from 3.0-3.1 g/16 g N, for example 3.1-3.2 g/16 g N, such as from 3.2-3.3 g/16 g N, for example 3.3-3.4 g/16 g N, such as from 3.4-3.5 g/16 g N, for example 3.5-3.6 g/16 g N, such as from 3.6-3.7 g/16 g N, for example 3.7-3.8 g/16 g N, such as from 3.8-3.9 g/16 g N, for example 3.9-4.0 g/16 g N, such as from 4.0-4.1 g/16 g N, for example 4.1-4.2 g/16 g N, such as from 4.2-4.3 g/16 g N, for example 4.3-4.4 g/16 g N, such as from 4.4-4.5 g/16 g N, for example 4.5-4.6 g/16 g N, such as from 4.6-4.7 g/16 g N, for example 4.7-4.8 g/16 g N, such as from 4.8-4.9 g/16 g N, for example 4.9-5.0 g/16 g N, such as from 5.0-5.1 g/16 g N, for example 5.1-5.2 g/16 g N, such as from 5.2-5.3 g/16 g N, for example 5.3-5.4 g/16 g N, such as from 5.4-5.5 g/16 g N, for example 5.5-5.6 g/16 g N, such as from 5.6-5.7 g/16 g N, for example 5.7-5.8 g/16 g N, such as from 5.8-5.9 g/16 g N, for example 5.9-6.0 g/16 g N, such as from 6.0-6.1 g/16 g N, for example 6.1-6.2 g/16 g N, such as from 6.2-6.3 g/16 g N, for example 6.3-6.4 g/16 g N, such as from 6.4-6.5 g/16 g N, for example 6.5-6.6 g/16 g N, such as from 6.6-6.7 g/16 g N, for example 6.7-6.8 g/16 g N, such as from 6.8-6.9 g/16 g N, for example 6.9-7.0 g/16 g N, such as from 7.0-7.1 g/16 g N, for example 7.1-7.2 g/16 g N, such as from 7.2-7.3 g/16 g N, for example 7.3-7.4 g/16 g N, such as from 7.4-7.5 g/16 g N, for example 7.5-7.6 g/16 g N, such as from 7.6-7.7 g/16 g N, for example 7.7-7.8 g/16 g N, such as from 7.8-7.9 g/16 g N, for example 7.9-8.0 g/16 g N, such as from 8.0-8.1 g/16 g N, for example 8.1-8.2 g/16 g N, such as from 8.2-8.3 g/16 g N, for example 8.3-8.4 g/16 g N, such as from 8.4-8.5 g/16 g N, for example 8.5-8.6 g/16 g N, such as from 8.6-8.7 g/16 g N, for example 8.7-8.8 g/16 g N, such as from 8.8-8.9 g/16 g N, for example 8.9-9.0 g/16 g N, such as from 9.0-9.1 g/16 g N, for example 9.1-9.2 g/16 g N, such as from 9.2-9.3 g/16 g N, for example 9.3-9.4 g/16 g N, such as from 9.4-9.5 g/16 g N, for example 9.5-9.6 g/16 g N, such as from 9.6-9.7 g/16 g N, for example 9.7-9.8 g/16 g N, such as from 9.8-9.9 g/16 g N, for example 9.9-10.0 g/16 g N, such as from 10.0-10.1 g/16 g N, for example 10.1-10.2 g/16 g N, such as from 10.2-10.3 g/16 g N, for example 10.3-10.4 g/16 g N, such as from 10.4-10.5 g/16 g N, for example 10.5-10.6 g/16 g N, such as from 10.6-10.7 g/16 g N, for example 10.7-10.8 g/16 g N, such as from 10.8-10.9 g/16 g N, for example 10.9-11.0 g/16 g N, such as from 11.0-11.1 g/16 g N, for example 11.1-11.2 g/16 g N, such as from 11.2-11.3 g/16 g N, for example 11.3-11.4 g/16 g N, such as from 11.4-11.5 g/16 g N, for example 11.5-11.6 g/16 g N, such as from 11.6-11.7 g/16 g N, for example 11.7-11.8 g/16 g N, such as from 11.8-11.9 g/16 g N, for example 11.9-12.0 g/16 g N, such as from 12.0-12.1 g/16 g N, for example 12.1-12.2 g/16 g N, such as from 12.2-12.3 g/16 g N, for example 12.3-12.4 g/16 g N, such as from 12.4-12.5 g/16 g N, for example 12.5-12.6 g/16 g N, such as from 12.6-12.7 g/16 g N, for example 12.7-12.8 g/16 g N, such as from 12.8-12.9 g/16 g N, for example 12.9-13.0 g/16 g N, such as from 13.0-13.1 g/16 g N, for example 13.1-13.2 g/16 g N, such as from 13.2-13.3 g/16 g N, for example 13.3-13.4 g/16 g N, such as from 13.4-13.5 g/16 g N, for example 13.5-13.6 g/16 g N, such as from 13.6-13.7 g/16 g N, for example 13.7-13.8 g/16 g N, such as from 13.8-13.9 g/16 g N, for example 13.9-14.0 g/16 g N, such as from 14.0-14.1 g/16 g N, for example 14.1-14.2 g/16 g N, such as from 14.2-14.3 g/16 g N, for example 14.3-14.4 g/16 g N, such as from 14.4-14.5 g/16 g N, for example 14.5-14.6 g/16 g N, such as from 14.6-14.7 g/16 g N, for example 14.7-14.8 g/16 g N, such as from 14.8-14.9 g/16 g N, for example 14.9-15.0 g/16 g N, such as from 15-16 g/16 g N, for example 16-17 g/16 g N, such as from 17-18 g/16 g N, for example 18-19 g/16 g N, such as from 19-20 g/16 g N.

In one embodiment the present invention relates to a fish feed composition with a Taurine content (amino acid g/16 g N) in the range from 0-20 g/16 g N, such as from 0-0.1 g/16 g N, for example 0.1-0.2 g/16 g N, such as from 0.2-0.3 g/16 g N, for example 0.3-0.4 g/16 g N, such as from 0.4-0.5 g/16 g N, for example 0.5-0.6 g/16 g N, such as from 0.6-0.7 g/16 g N, for example 0.7-0.8 g/16 g N, such as from 0.8-0.9 g/16 g N, for example 0.9-1.0 g/16 g N, such as from 1.0-1.1 g/16 g N, for example 1.1-1.2 g/16 g N, such as from 1.2-1.3 g/16 g N, for example 1.3-1.4 g/16 g N, such as from 1.4-1.5 g/16 g N, for example 1.5-1.6 g/16 g N, such as from 1.6-1.7 g/16 g N, for example 1.7-1.8 g/16 g N, such as from 1.8-1.9 g/16 g N, for example 1.9-2.0 g/16 g N, such as from 2.0-2.1 g/16 g N, for example 2.1-2.2 g/16 g N, such as from 2.2-2.3 g/16 g N, for example 2.3-2.4 g/16 g N, such as from 2.4-2.5 g/16 g N, for example 2.5-2.6 g/16 g N, such as from 2.6-2.7 g/16 g N, for example 2.7-2.8 g/16 g N, such as from 2.8-2.9 g/16 g N, for example 2.9-3.0 g/16 g N, such as from 3.0-3.1 g/16 g N, for example 3.1-3.2 g/16 g N, such as from 3.2-3.3 g/16 g N, for example 3.3-3.4 g/16 g N, such as from 3.4-3.5 g/16 g N, for example 3.5-3.6 g/16 g N, such as from 3.6-3.7 g/16 g N, for example 3.7-3.8 g/16 g N, such as from 3.8-3.9 g/16 g N, for example 3.9-4.0 g/16 g N, such as from 4.0-4.1 g/16 g N, for example 4.1-4.2 g/16 g N, such as from 4.2-4.3 g/16 g N, for example 4.3-4.4 g/16 g N, such as from 4.4-4.5 g/16 g N, for example 4.5-4.6 g/16 g N, such as from 4.6-4.7 g/16 g N, for example 4.7-4.8 g/16 g N, such as from 4.8-4.9 g/16 g N, for example 4.9-5.0 g/16 g N, such as from 5.0-5.1 g/16 g N, for example 5.1-5.2 g/16 g N, such as from 5.2-5.3 g/16 g N, for example 5.3-5.4 g/16 g N, such as from 5.4-5.5 g/16 g N, for example 5.5-5.6 g/16 g N, such as from 5.6-5.7 g/16 g N, for example 5.7-5.8 g/16 g N, such as from 5.8-5.9 g/16 g N, for example 5.9-6.0 g/16 g N, such as from 6.0-6.1 g/16 g N, for example 6.1-6.2 g/16 g N, such as from 6.2-6.3 g/16 g N, for example 6.3-6.4 g/16 g N, such as from 6.4-6.5 g/16 g N, for example 6.5-6.6 g/16 g N, such as from 6.6-6.7 g/16 g N, for example 6.7-6.8 g/16 g N, such as from 6.8-6.9 g/16 g N, for example 6.9-7.0 g/16 g N, such as from 7.0-7.1 g/16 g N, for example 7.1-7.2 g/16 g N, such as from 7.2-7.3 g/16 g N, for example 7.3-7.4 g/16 g N, such as from 7.4-7.5 g/16 g N, for example 7.5-7.6 g/16 g N, such as from 7.6-7.7 g/16 g N, for example 7.7-7.8 g/16 g N, such as from 7.8-7.9 g/16 g N, for example 7.9-8.0 g/16 g N, such as from 8.0-8.1 g/16 g N, for example 8.1-8.2 g/16 g N, such as from 8.2-8.3 g/16 g N, for example 8.3-8.4 g/16 g N, such as from 8.4-8.5 g/16 g N, for example 8.5-8.6 g/16 g N, such as from 8.6-8.7 g/16 g N, for example 8.7-8.8 g/16 g N, such as from 8.8-8.9 g/16 g N, for example 8.9-9.0 g/16 g N, such as from 9.0-9.1 g/16 g N, for example 9.1-9.2 g/16 g N, such as from 9.2-9.3 g/16 g N, for example 9.3-9.4 g/16 g N, such as from 9.4-9.5 g/16 g N, for example 9.5-9.6 g/16 g N, such as from 9.6-9.7 g/16 g N, for example 9.7-9.8 g/16 g N, such as from 9.8-9.9 g/16 g N, for example 9.9-10.0 g/16 g N, such as from 10.0-10.1 g/16 g N, for example 10.1-10.2 g/16 g N, such as from 10.2-10.3 g/16 g N, for example 10.3-10.4 g/16 g N, such as from 10.4-10.5 g/16 g N, for example 10.5-10.6 g/16 g N, such as from 10.6-10.7 g/16 g N, for example 10.7-10.8 g/16 g N, such as from 10.8-10.9 g/16 g N, for example 10.9-11.0 g/16 g N, such as from 11.0-11.1 g/16 g N, for example 11.1-11.2 g/16 g N, such as from 11.2-11.3 g/16 g N, for example 11.3-11.4 g/16 g N, such as from 11.4-11.5 g/16 g N, for example 11.5-11.6 g/16 g N, such as from 11.6-11.7 g/16 g N, for example 11.7-11.8 g/16 g N, such as from 11.8-11.9 g/16 g N, for example 11.9-12.0 g/16 g N, such as from 12.0-12.1 g/16 g N, for example 12.1-12.2 g/16 g N, such as from 12.2-12.3 g/16 g N, for example 12.3-12.4 g/16 g N, such as from 12.4-12.5 g/16 g N, for example 12.5-12.6 g/16 g N, such as from 12.6-12.7 g/16 g N, for example 12.7-12.8 g/16 g N, such as from 12.8-12.9 g/16 g N, for example 12.9-13.0 g/16 g N, such as from 13.0-13.1 g/16 g N, for example 13.1-13.2 g/16 g N, such as from 13.2-13.3 g/16 g N, for example 13.3-13.4 g/16 g N, such as from 13.4-13.5 g/16 g N, for example 13.5-13.6 g/16 g N, such as from 13.6-13.7 g/16 g N, for example 13.7-13.8 g/16 g N, such as from 13.8-13.9 g/16 g N, for example 13.9-14.0 g/16 g N, such as from 14.0-14.1 g/16 g N, for example 14.1-14.2 g/16 g N, such as from 14.2-14.3 g/16 g N, for example 14.3-14.4 g/16 g N, such as from 14.4-14.5 g/16 g N, for example 14.5-14.6 g/16 g N, such as from 14.6-14.7 g/16 g N, for example 14.7-14.8 g/16 g N, such as from 14.8-14.9 g/16 g N, for example 14.9-15.0 g/16 g N, such as from 15-16 g/16 g N, for example 16-17 g/16 g N, such as from 17-18 g/16 g N, for example 18-19 g/16 g N, such as from 19-20 g/16 g N.

In one embodiment the present invention relates to a fish feed composition with a Valine content (amino acid g/16 g N) in the range from 0-20 g/16 g N, such as from 0-0.1 g/16 g N, for example 0.1-0.2 g/16 g N, such as from 0.2-0.3 g/16 g N, for example 0.3-0.4 g/16 g N, such as from 0.4-0.5 g/16 g N, for example 0.5-0.6 g/16 g N, such as from 0.6-0.7 g/16 g N, for example 0.7-0.8 g/16 g N, such as from 0.8-0.9 g/16 g N, for example 0.9-1.0 g/16 g N, such as from 1.0-1.1 g/16 g N, for example 1.1-1.2 g/16 g N, such as from 1.2-1.3 g/16 g N, for example 1.3-1.4 g/16 g N, such as from 1.4-1.5 g/16 g N, for example 1.5-1.6 g/16 g N, such as from 1.6-1.7 g/16 g N, for example 1.7-1.8 g/16 g N, such as from 1.8-1.9 g/16 g N, for example 1.9-2.0 g/16 g N, such as from 2.0-2.1 g/16 g N, for example 2.1-2.2 g/16 g N, such as from 2.2-2.3 g/16 g N, for example 2.3-2.4 g/16 g N, such as from 2.4-2.5 g/16 g N, for example 2.5-2.6 g/16 g N, such as from 2.6-2.7 g/16 g N, for example 2.7-2.8 g/16 g N, such as from 2.8-2.9 g/16 g N, for example 2.9-3.0 g/16 g N, such as from 3.0-3.1 g/16 g N, for example 3.1-3.2 g/16 g N, such as from 3.2-3.3 g/16 g N, for example 3.3-3.4 g/16 g N, such as from 3.4-3.5 g/16 g N, for example 3.5-3.6 g/16 g N, such as from 3.6-3.7 g/16 g N, for example 3.7-3.8 g/16 g N, such as from 3.8-3.9 g/16 g N, for example 3.9-4.0 g/16 g N, such as from 4.0-4.1 g/16 g N, for example 4.1-4.2 g/16 g N, such as from 4.2-4.3 g/16 g N, for example 4.3-4.4 g/16 g N, such as from 4.4-4.5 g/16 g N, for example 4.5-4.6 g/16 g N, such as from 4.6-4.7 g/16 g N, for example 4.7-4.8 g/16 g N, such as from 4.8-4.9 g/16 g N, for example 4.9-5.0 g/16 g N, such as from 5.0-5.1 g/16 g N, for example 5.1-5.2 g/16 g N, such as from 5.2-5.3 g/16 g N, for example 5.3-5.4 g/16 g N, such as from 5.4-5.5 g/16 g N, for example 5.5-5.6 g/16 g N, such as from 5.6-5.7 g/16 g N, for example 5.7-5.8 g/16 g N, such as from 5.8-5.9 g/16 g N, for example 5.9-6.0 g/16 g N, such as from 6.0-6.1 g/16 g N, for example 6.1-6.2 g/16 g N, such as from 6.2-6.3 g/16 g N, for example 6.3-6.4 g/16 g N, such as from 6.4-6.5 g/16 g N, for example 6.5-6.6 g/16 g N, such as from 6.6-6.7 g/16 g N, for example 6.7-6.8 g/16 g N, such as from 6.8-6.9 g/16 g N, for example 6.9-7.0 g/16 g N, such as from 7.0-7.1 g/16 g N, for example 7.1-7.2 g/16 g N, such as from 7.2-7.3 g/16 g N, for example 7.3-7.4 g/16 g N, such as from 7.4-7.5 g/16 g N, for example 7.5-7.6 g/16 g N, such as from 7.6-7.7 g/16 g N, for example 7.7-7.8 g/16 g N, such as from 7.8-7.9 g/16 g N, for example 7.9-8.0 g/16 g N, such as from 8.0-8.1 g/16 g N, for example 8.1-8.2 g/16 g N, such as from 8.2-8.3 g/16 g N, for example 8.3-8.4 g/16 g N, such as from 8.4-8.5 g/16 g N, for example 8.5-8.6 g/16 g N, such as from 8.6-8.7 g/16 g N, for example 8.7-8.8 g/16 g N, such as from 8.8-8.9 g/16 g N, for example 8.9-9.0 g/16 g N, such as from 9.0-9.1 g/16 g N, for example 9.1-9.2 g/16 g N, such as from 9.2-9.3 g/16 g N, for example 9.3-9.4 g/16 g N, such as from 9.4-9.5 g/16 g N, for example 9.5-9.6 g/16 g N, such as from 9.6-9.7 g/16 g N, for example 9.7-9.8 g/16 g N, such as from 9.8-9.9 g/16 g N, for example 9.9-10.0 g/16 g N, such as from 10.0-10.1 g/16 g N, for example 10.1-10.2 g/16 g N, such as from 10.2-10.3 g/16 g N, for example 10.3-10.4 g/16 g N, such as from 10.4-10.5 g/16 g N, for example 10.5-10.6 g/16 g N, such as from 10.6-10.7 g/16 g N, for example 10.7-10.8 g/16 g N, such as from 10.8-10.9 g/16 g N, for example 10.9-11.0 g/16 g N, such as from 11.0-11.1 g/16 g N, for example 11.1-11.2 g/16 g N, such as from 11.2-11.3 g/16 g N, for example 11.3-11.4 g/16 g N, such as from 11.4-11.5 g/16 g N, for example 11.5-11.6 g/16 g N, such as from 11.6-11.7 g/16 g N, for example 11.7-11.8 g/16 g N, such as from 11.8-11.9 g/16 g N, for example 11.9-12.0 g/16 g N, such as from 12.0-12.1 g/16 g N, for example 12.1-12.2 g/16 g N, such as from 12.2-12.3 g/16 g N, for example 12.3-12.4 g/16 g N, such as from 12.4-12.5 g/16 g N, for example 12.5-12.6 g/16 g N, such as from 12.6-12.7 g/16 g N, for example 12.7-12.8 g/16 g N, such as from 12.8-12.9 g/16 g N, for example 12.9-13.0 g/16 g N, such as from 13.0-13.1 g/16 g N, for example 13.1-13.2 g/16 g N, such as from 13.2-13.3 g/16 g N, for example 13.3-13.4 g/16 g N, such as from 13.4-13.5 g/16 g N, for example 13.5-13.6 g/16 g N, such as from 13.6-13.7 g/16 g N, for example 13.7-13.8 g/16 g N, such as from 13.8-13.9 g/16 g N, for example 13.9-14.0 g/16 g N, such as from 14.0-14.1 g/16 g N, for example 14.1-14.2 g/16 g N, such as from 14.2-14.3 g/16 g N, for example 14.3-14.4 g/16 g N, such as from 14.4-14.5 g/16 g N, for example 14.5-14.6 g/16 g N, such as from 14.6-14.7 g/16 g N, for example 14.7-14.8 g/16 g N, such as from 14.8-14.9 g/16 g N, for example 14.9-15.0 g/16 g N, such as from 15-16 g/16 g N, for example 16-17 g/16 g N, such as from 17-18 g/16 g N, for example 18-19 g/16 g N, such as from 19-20 g/16 g N.

In one embodiment the present invention relates to a fish feed composition with a 14:0 content (g/100 g lipid) in the range from 0-20 g/100 g lipid, such as from 0-0.1 g/100 g lipid, for example 0.1-0.2 g/100 g lipid, such as from 0.2-0.3 g/100 g lipid, for example 0.3-0.4 g/100 g lipid, such as from 0.4-0.5 g/100 g lipid, for example 0.5-0.6 g/100 g lipid, such as from 0.6-0.7 g/100 g lipid, for example 0.7-0.8 g/100 g lipid, such as from 0.8-0.9 g/100 g lipid, for example 0.9-1.0 g/100 g lipid, such as from 1.0-1.1 g/100 g lipid, for example 1.1-1.2 g/100 g lipid, such as from 1.2-1.3 g/100 g lipid, for example 1.3-1.4 g/100 g lipid, such as from 1.4-1.5 g/100 g lipid, for example 1.5-1.6 g/100 g lipid, such as from 1.6-1.7 g/100 g lipid, for example 1.7-1.8 g/100 g lipid, such as from 1.8-1.9 g/100 g lipid, for example 1.9-2.0 g/100 g lipid, such as from 2.0-2.1 g/100 g lipid, for example 2.1-2.2 g/100 g lipid, such as from 2.2-2.3 g/100 g lipid, for example 2.3-2.4 g/100 g lipid, such as from 2.4-2.5 g/100 g lipid, for example 2.5-2.6 g/100 g lipid, such as from 2.6-2.7 g/100 g lipid, for example 2.7-2.8 g/100 g lipid, such as from 2.8-2.9 g/100 g lipid, for example 2.9-3.0 g/100 g lipid, such as from 3.0-3.1 g/100 g lipid, for example 3.1-3.2 g/100 g lipid, such as from 3.2-3.3 g/100 g lipid, for example 3.3-3.4 g/100 g lipid, such as from 3.4-3.5 g/100 g lipid, for example 3.5-3.6 g/100 g lipid, such as from 3.6-3.7 g/100 g lipid, for example 3.7-3.8 g/100 g lipid, such as from 3.8-3.9 g/100 g lipid, for example 3.9-4.0 g/100 g lipid, such as from 4.0-4.1 g/100 g lipid, for example 4.1-4.2 g/100 g lipid, such as from 4.2-4.3 g/100 g lipid, for example 4.3-4.4 g/100 g lipid, such as from 4.4-4.5 g/100 g lipid, for example 4.5-4.6 g/100 g lipid, such as from 4.6-4.7 g/100 g lipid, for example 4.7-4.8 g/100 g lipid, such as from 4.8-4.9 g/100 g lipid, for example 4.9-5.0 g/100 g lipid, such as from 5.0-5.1 g/100 g lipid, for example 5.1-5.2 g/100 g lipid, such as from 5.2-5.3 g/100 g lipid, for example 5.3-5.4 g/100 g lipid, such as from 5.4-5.5 g/100 g lipid, for example 5.5-5.6 g/100 g lipid, such as from 5.6-5.7 g/100 g lipid, for example 5.7-5.8 g/100 g lipid, such as from 5.8-5.9 g/100 g lipid, for example 5.9-6.0 g/100 g lipid, such as from 6.0-6.1 g/100 g lipid, for example 6.1-6.2 g/100 g lipid, such as from 6.2-6.3 g/100 g lipid, for example 6.3-6.4 g/100 g lipid, such as from 6.4-6.5 g/100 g lipid, for example 6.5-6.6 g/100 g lipid, such as from 6.6-6.7 g/100 g lipid, for example 6.7-6.8 g/100 g lipid, such as from 6.8-6.9 g/100 g lipid, for example 6.9-7.0 g/100 g lipid, such as from 7.0-7.1 g/100 g lipid, for example 7.1-7.2 g/100 g lipid, such as from 7.2-7.3 g/100 g lipid, for example 7.3-7.4 g/100 g lipid, such as from 7.4-7.5 g/100 g lipid, for example 7.5-7.6 g/100 g lipid, such as from 7.6-7.7 g/100 g lipid, for example 7.7-7.8 g/100 g lipid, such as from 7.8-7.9 g/100 g lipid, for example 7.9-8.0 g/100 g lipid, such as from 8.0-8.1 g/100 g lipid, for example 8.1-8.2 g/100 g lipid, such as from 8.2-8.3 g/100 g lipid, for example 8.3-8.4 g/100 g lipid, such as from 8.4-8.5 g/100 g lipid, for example 8.5-8.6 g/100 g lipid, such as from 8.6-8.7 g/100 g lipid, for example 8.7-8.8 g/100 g lipid, such as from 8.8-8.9 g/100 g lipid, for example 8.9-9.0 g/100 g lipid, such as from 9.0-9.1 g/100 g lipid, for example 9.1-9.2 g/100 g lipid, such as from 9.2-9.3 g/100 g lipid, for example 9.3-9.4 g/100 g lipid, such as from 9.4-9.5 g/100 g lipid, for example 9.5-9.6 g/100 g lipid, such as from 9.6-9.7 g/100 g lipid, for example 9.7-9.8 g/100 g lipid, such as from 9.8-9.9 g/100 g lipid, for example 9.9-10.0 g/100 g lipid, such as from 10.0-10.1 g/100 g lipid, for example 10.1-10.2 g/100 g lipid, such as from 10.2-10.3 g/100 g lipid, for example 10.3-10.4 g/100 g lipid, such as from 10.4-10.5 g/100 g lipid, for example 10.5-10.6 g/100 g lipid, such as from 10.6-10.7 g/100 g lipid, for example 10.7-10.8 g/100 g lipid, such as from 10.8-10.9 g/100 g lipid, for example 10.9-11.0 g/100 g lipid, such as from 11.0-11.1 g/100 g lipid, for example 11.1-11.2 g/100 g lipid, such as from 11.2-11.3 g/100 g lipid, for example 11.3-11.4 g/100 g lipid, such as from 11.4-11.5 g/100 g lipid, for example 11.5-11.6 g/100 g lipid, such as from 11.6-11.7 g/100 g lipid, for example 11.7-11.8 g/100 g lipid, such as from 11.8-11.9 g/100 g lipid, for example 11.9-12.0 g/100 g lipid, such as from 12.0-12.1 g/100 g lipid, for example 12.1-12.2 g/100 g lipid, such as from 12.2-12.3 g/100 g lipid, for example 12.3-12.4 g/100 g lipid, such as from 12.4-12.5 g/100 g lipid, for example 12.5-12.6 g/100 g lipid, such as from 12.6-12.7 g/100 g lipid, for example 12.7-12.8 g/100 g lipid, such as from 12.8-12.9 g/100 g lipid, for example 12.9-13.0 g/100 g lipid, such as from 13.0-13.1 g/100 g lipid, for example 13.1-13.2 g/100 g lipid, such as from 13.2-13.3 g/100 g lipid, for example 13.3-13.4 g/100 g lipid, such as from 13.4-13.5 g/100 g lipid, for example 13.5-13.6 g/100 g lipid, such as from 13.6-13.7 g/100 g lipid, for example 13.7-13.8 g/100 g lipid, such as from 13.8-13.9 g/100 g lipid, for example 13.9-14.0 g/100 g lipid, such as from 14.0-14.1 g/100 g lipid, for example 14.1-14.2 g/100 g lipid, such as from 14.2-14.3 g/100 g lipid, for example 14.3-14.4 g/100 g lipid, such as from 14.4-14.5 g/100 g lipid, for example 14.5-14.6 g/100 g lipid, such as from 14.6-14.7 g/100 g lipid, for example 14.7-14.8 g/100 g lipid, such as from 14.8-14.9 g/100 g lipid, for example 14.9-15.0 g/100 g lipid, such as from 15-16 g/100 g lipid, for example 16-17 g/100 g lipid, such as from 17-18 g/100 g lipid, for example 18-19 g/100 g lipid, such as from 19-20 g/100 g lipid.

In one embodiment the present invention relates to a fish feed composition with a 16:0 content (g/100 g lipid) in the range from 0-20 g/100 g lipid, such as from 0-0.1 g/100 g lipid, for example 0.1-0.2 g/100 g lipid, such as from 0.2-0.3 g/100 g lipid, for example 0.3-0.4 g/100 g lipid, such as from 0.4-0.5 g/100 g lipid, for example 0.5-0.6 g/100 g lipid, such as from 0.6-0.7 g/100 g lipid, for example 0.7-0.8 g/100 g lipid, such as from 0.8-0.9 g/100 g lipid, for example 0.9-1.0 g/100 g lipid, such as from 1.0-1.1 g/100 g lipid, for example 1.1-1.2 g/100 g lipid, such as from 1.2-1.3 g/100 g lipid, for example 1.3-1.4 g/100 g lipid, such as from 1.4-1.5 g/100 g lipid, for example 1.5-1.6 g/100 g lipid, such as from 1.6-1.7 g/100 g lipid, for example 1.7-1.8 g/100 g lipid, such as from 1.8-1.9 g/100 g lipid, for example 1.9-2.0 g/100 g lipid, such as from 2.0-2.1 g/100 g lipid, for example 2.1-2.2 g/100 g lipid, such as from 2.2-2.3 g/100 g lipid, for example 2.3-2.4 g/100 g lipid, such as from 2.4-2.5 g/100 g lipid, for example 2.5-2.6 g/100 g lipid, such as from 2.6-2.7 g/100 g lipid, for example 2.7-2.8 g/100 g lipid, such as from 2.8-2.9 g/100 g lipid, for example 2.9-3.0 g/100 g lipid, such as from 3.0-3.1 g/100 g lipid, for example 3.1-3.2 g/100 g lipid, such as from 3.2-3.3 g/100 g lipid, for example 3.3-3.4 g/100 g lipid, such as from 3.4-3.5 g/100 g lipid, for example 3.5-3.6 g/100 g lipid, such as from 3.6-3.7 g/100 g lipid, for example 3.7-3.8 g/100 g lipid, such as from 3.8-3.9 g/100 g lipid, for example 3.9-4.0 g/100 g lipid, such as from 4.0-4.1 g/100 g lipid, for example 4.1-4.2 g/100 g lipid, such as from 4.2-4.3 g/100 g lipid, for example 4.3-4.4 g/100 g lipid, such as from 4.4-4.5 g/100 g lipid, for example 4.5-4.6 g/100 g lipid, such as from 4.6-4.7 g/100 g lipid, for example 4.7-4.8 g/100 g lipid, such as from 4.8-4.9 g/100 g lipid, for example 4.9-5.0 g/100 g lipid, such as from 5.0-5.1 g/100 g lipid, for example 5.1-5.2 g/100 g lipid, such as from 5.2-5.3 g/100 g lipid, for example 5.3-5.4 g/100 g lipid, such as from 5.4-5.5 g/100 g lipid, for example 5.5-5.6 g/100 g lipid, such as from 5.6-5.7 g/100 g lipid, for example 5.7-5.8 g/100 g lipid, such as from 5.8-5.9 g/100 g lipid, for example 5.9-6.0 g/100 g lipid, such as from 6.0-6.1 g/100 g lipid, for example 6.1-6.2 g/100 g lipid, such as from 6.2-6.3 g/100 g lipid, for example 6.3-6.4 g/100 g lipid, such as from 6.4-6.5 g/100 g lipid, for example 6.5-6.6 g/100 g lipid, such as from 6.6-6.7 g/100 g lipid, for example 6.7-6.8 g/100 g lipid, such as from 6.8-6.9 g/100 g lipid, for example 6.9-7.0 g/100 g lipid, such as from 7.0-7.1 g/100 g lipid, for example 7.1-7.2 g/100 g lipid, such as from 7.2-7.3 g/100 g lipid, for example 7.3-7.4 g/100 g lipid, such as from 7.4-7.5 g/100 g lipid, for example 7.5-7.6 g/100 g lipid, such as from 7.6-7.7 g/100 g lipid, for example 7.7-7.8 g/100 g lipid, such as from 7.8-7.9 g/100 g lipid, for example 7.9-8.0 g/100 g lipid, such as from 8.0-8.1 g/100 g lipid, for example 8.1-8.2 g/100 g lipid, such as from 8.2-8.3 g/100 g lipid, for example 8.3-8.4 g/100 g lipid, such as from 8.4-8.5 g/100 g lipid, for example 8.5-8.6 g/100 g lipid, such as from 8.6-8.7 g/100 g lipid, for example 8.7-8.8 g/100 g lipid, such as from 8.8-8.9 g/100 g lipid, for example 8.9-9.0 g/100 g lipid, such as from 9.0-9.1 g/100 g lipid, for example 9.1-9.2 g/100 g lipid, such as from 9.2-9.3 g/100 g lipid, for example 9.3-9.4 g/100 g lipid, such as from 9.4-9.5 g/100 g lipid, for example 9.5-9.6 g/100 g lipid, such as from 9.6-9.7 g/100 g lipid, for example 9.7-9.8 g/100 g lipid, such as from 9.8-9.9 g/100 g lipid, for example 9.9-10.0 g/100 g lipid, such as from 10.0-10.1 g/100 g lipid, for example 10.1-10.2 g/100 g lipid, such as from 10.2-10.3 g/100 g lipid, for example 10.3-10.4 g/100 g lipid, such as from 10.4-10.5 g/100 g lipid, for example 10.5-10.6 g/100 g lipid, such as from 10.6-10.7 g/100 g lipid, for example 10.7-10.8 g/100 g lipid, such as from 10.8-10.9 g/100 g lipid, for example 10.9-11.0 g/100 g lipid, such as from 11.0-11.1 g/100 g lipid, for example 11.1-11.2 g/100 g lipid, such as from 11.2-11.3 g/100 g lipid, for example 11.3-11.4 g/100 g lipid, such as from 11.4-11.5 g/100 g lipid, for example 11.5-11.6 g/100 g lipid, such as from 11.6-11.7 g/100 g lipid, for example 11.7-11.8 g/100 g lipid, such as from 11.8-11.9 g/100 g lipid, for example 11.9-12.0 g/100 g lipid, such as from 12.0-12.1 g/100 g lipid, for example 12.1-12.2 g/100 g lipid, such as from 12.2-12.3 g/100 g lipid, for example 12.3-12.4 g/100 g lipid, such as from 12.4-12.5 g/100 g lipid, for example 12.5-12.6 g/100 g lipid, such as from 12.6-12.7 g/100 g lipid, for example 12.7-12.8 g/100 g lipid, such as from 12.8-12.9 g/100 g lipid, for example 12.9-13.0 g/100 g lipid, such as from 13.0-13.1 g/100 g lipid, for example 13.1-13.2 g/100 g lipid, such as from 13.2-13.3 g/100 g lipid, for example 13.3-13.4 g/100 g lipid, such as from 13.4-13.5 g/100 g lipid, for example 13.5-13.6 g/100 g lipid, such as from 13.6-13.7 g/100 g lipid, for example 13.7-13.8 g/100 g lipid, such as from 13.8-13.9 g/100 g lipid, for example 13.9-14.0 g/100 g lipid, such as from 14.0-14.1 g/100 g lipid, for example 14.1-14.2 g/100 g lipid, such as from 14.2-14.3 g/100 g lipid, for example 14.3-14.4 g/100 g lipid, such as from 14.4-14.5 g/100 g lipid, for example 14.5-14.6 g/100 g lipid, such as from 14.6-14.7 g/100 g lipid, for example 14.7-14.8 g/100 g lipid, such as from 14.8-14.9 g/100 g lipid, for example 14.9-15.0 g/100 g lipid, such as from 15-16 g/100 g lipid, for example 16-17 g/100 g lipid, such as from 17-18 g/100 g lipid, for example 18-19 g/100 g lipid, such as from 19-20 g/100 g lipid.

In one embodiment the present invention relates to a fish feed composition with a 18:0 content (g/100 g lipid) in the range from 0-20 g/100 g lipid, such as from 0-0.1 g/100 g lipid, for example 0.1-0.2 g/100 g lipid, such as from 0.2-0.3 g/100 g lipid, for example 0.3-0.4 g/100 g lipid, such as from 0.4-0.5 g/100 g lipid, for example 0.5-0.6 g/100 g lipid, such as from 0.6-0.7 g/100 g lipid, for example 0.7-0.8 g/100 g lipid, such as from 0.8-0.9 g/100 g lipid, for example 0.9-1.0 g/100 g lipid, such as from 1.0-1.1 g/100 g lipid, for example 1.1-1.2 g/100 g lipid, such as from 1.2-1.3 g/100 g lipid, for example 1.3-1.4 g/100 g lipid, such as from 1.4-1.5 g/100 g lipid, for example 1.5-1.6 g/100 g lipid, such as from 1.6-1.7 g/100 g lipid, for example 1.7-1.8 g/100 g lipid, such as from 1.8-1.9 g/100 g lipid, for example 1.9-2.0 g/100 g lipid, such as from 2.0-2.1 g/100 g lipid, for example 2.1-2.2 g/100 g lipid, such as from 2.2-2.3 g/100 g lipid, for example 2.3-2.4 g/100 g lipid, such as from 2.4-2.5 g/100 g lipid, for example 2.5-2.6 g/100 g lipid, such as from 2.6-2.7 g/100 g lipid, for example 2.7-2.8 g/100 g lipid, such as from 2.8-2.9 g/100 g lipid, for example 2.9-3.0 g/100 g lipid, such as from 3.0-3.1 g/100 g lipid, for example 3.1-3.2 g/100 g lipid, such as from 3.2-3.3 g/100 g lipid, for example 3.3-3.4 g/100 g lipid, such as from 3.4-3.5 g/100 g lipid, for example 3.5-3.6 g/100 g lipid, such as from 3.6-3.7 g/100 g lipid, for example 3.7-3.8 g/100 g lipid, such as from 3.8-3.9 g/100 g lipid, for example 3.9-4.0 g/100 g lipid, such as from 4.0-4.1 g/100 g lipid, for example 4.1-4.2 g/100 g lipid, such as from 4.2-4.3 g/100 g lipid, for example 4.3-4.4 g/100 g lipid, such as from 4.4-4.5 g/100 g lipid, for example 4.5-4.6 g/100 g lipid, such as from 4.6-4.7 g/100 g lipid, for example 4.7-4.8 g/100 g lipid, such as from 4.8-4.9 g/100 g lipid, for example 4.9-5.0 g/100 g lipid, such as from 5.0-5.1 g/100 g lipid, for example 5.1-5.2 g/100 g lipid, such as from 5.2-5.3 g/100 g lipid, for example 5.3-5.4 g/100 g lipid, such as from 5.4-5.5 g/100 g lipid, for example 5.5-5.6 g/100 g lipid, such as from 5.6-5.7 g/100 g lipid, for example 5.7-5.8 g/100 g lipid, such as from 5.8-5.9 g/100 g lipid, for example 5.9-6.0 g/100 g lipid, such as from 6.0-6.1 g/100 g lipid, for example 6.1-6.2 g/100 g lipid, such as from 6.2-6.3 g/100 g lipid, for example 6.3-6.4 g/100 g lipid, such as from 6.4-6.5 g/100 g lipid, for example 6.5-6.6 g/100 g lipid, such as from 6.6-6.7 g/100 g lipid, for example 6.7-6.8 g/100 g lipid, such as from 6.8-6.9 g/100 g lipid, for example 6.9-7.0 g/100 g lipid, such as from 7.0-7.1 g/100 g lipid, for example 7.1-7.2 g/100 g lipid, such as from 7.2-7.3 g/100 g lipid, for example 7.3-7.4 g/100 g lipid, such as from 7.4-7.5 g/100 g lipid, for example 7.5-7.6 g/100 g lipid, such as from 7.6-7.7 g/100 g lipid, for example 7.7-7.8 g/100 g lipid, such as from 7.8-7.9 g/100 g lipid, for example 7.9-8.0 g/100 g lipid, such as from 8.0-8.1 g/100 g lipid, for example 8.1-8.2 g/100 g lipid, such as from 8.2-8.3 g/100 g lipid, for example 8.3-8.4 g/100 g lipid, such as from 8.4-8.5 g/100 g lipid, for example 8.5-8.6 g/100 g lipid, such as from 8.6-8.7 g/100 g lipid, for example 8.7-8.8 g/100 g lipid, such as from 8.8-8.9 g/100 g lipid, for example 8.9-9.0 g/100 g lipid, such as from 9.0-9.1 g/100 g lipid, for example 9.1-9.2 g/100 g lipid, such as from 9.2-9.3 g/100 g lipid, for example 9.3-9.4 g/100 g lipid, such as from 9.4-9.5 g/100 g lipid, for example 9.5-9.6 g/100 g lipid, such as from 9.6-9.7 g/100 g lipid, for example 9.7-9.8 g/100 g lipid, such as from 9.8-9.9 g/100 g lipid, for example 9.9-10.0 g/100 g lipid, such as from 10.0-10.1 g/100 g lipid, for example 10.1-10.2 g/100 g lipid, such as from 10.2-10.3 g/100 g lipid, for example 10.3-10.4 g/100 g lipid, such as from 10.4-10.5 g/100 g lipid, for example 10.5-10.6 g/100 g lipid, such as from 10.6-10.7 g/100 g lipid, for example 10.7-10.8 g/100 g lipid, such as from 10.8-10.9 g/100 g lipid, for example 10.9-11.0 g/100 g lipid, such as from 11.0-11.1 g/100 g lipid, for example 11.1-11.2 g/100 g lipid, such as from 11.2-11.3 g/100 g lipid, for example 11.3-11.4 g/100 g lipid, such as from 11.4-11.5 g/100 g lipid, for example 11.5-11.6 g/100 g lipid, such as from 11.6-11.7 g/100 g lipid, for example 11.7-11.8 g/100 g lipid, such as from 11.8-11.9 g/100 g lipid, for example 11.9-12.0 g/100 g lipid, such as from 12.0-12.1 g/100 g lipid, for example 12.1-12.2 g/100 g lipid, such as from 12.2-12.3 g/100 g lipid, for example 12.3-12.4 g/100 g lipid, such as from 12.4-12.5 g/100 g lipid, for example 12.5-12.6 g/100 g lipid, such as from 12.6-12.7 g/100 g lipid, for example 12.7-12.8 g/100 g lipid, such as from 12.8-12.9 g/100 g lipid, for example 12.9-13.0 g/100 g lipid, such as from 13.0-13.1 g/100 g lipid, for example 13.1-13.2 g/100 g lipid, such as from 13.2-13.3 g/100 g lipid, for example 13.3-13.4 g/100 g lipid, such as from 13.4-13.5 g/100 g lipid, for example 13.5-13.6 g/100 g lipid, such as from 13.6-13.7 g/100 g lipid, for example 13.7-13.8 g/100 g lipid, such as from 13.8-13.9 g/100 g lipid, for example 13.9-14.0 g/100 g lipid, such as from 14.0-14.1 g/100 g lipid, for example 14.1-14.2 g/100 g lipid, such as from 14.2-14.3 g/100 g lipid, for example 14.3-14.4 g/100 g lipid, such as from 14.4-14.5 g/100 g lipid, for example 14.5-14.6 g/100 g lipid, such as from 14.6-14.7 g/100 g lipid, for example 14.7-14.8 g/100 g lipid, such as from 14.8-14.9 g/100 g lipid, for example 14.9-15.0 g/100 g lipid, such as from 15-16 g/100 g lipid, for example 16-17 g/100 g lipid, such as from 17-18 g/100 g lipid, for example 18-19 g/100 g lipid, such as from 19-20 g/100 g lipid.

In one embodiment the present invention relates to a fish feed composition with a 20:0 content (g/100 g lipid) in the range from 0-20 g/100 g lipid, such as from 0-0.1 g/100 g lipid, for example 0.1-0.2 g/100 g lipid, such as from 0.2-0.3 g/100 g lipid, for example 0.3-0.4 g/100 g lipid, such as from 0.4-0.5 g/100 g lipid, for example 0.5-0.6 g/100 g lipid, such as from 0.6-0.7 g/100 g lipid, for example 0.7-0.8 g/100 g lipid, such as from 0.8-0.9 g/100 g lipid, for example 0.9-1.0 g/100 g lipid, such as from 1.0-1.1 g/100 g lipid, for example 1.1-1.2 g/100 g lipid, such as from 1.2-1.3 g/100 g lipid, for example 1.3-1.4 g/100 g lipid, such as from 1.4-1.5 g/100 g lipid, for example 1.5-1.6 g/100 g lipid, such as from 1.6-1.7 g/100 g lipid, for example 1.7-1.8 g/100 g lipid, such as from 1.8-1.9 g/100 g lipid, for example 1.9-2.0 g/100 g lipid, such as from 2.0-2.1 g/100 g lipid, for example 2.1-2.2 g/100 g lipid, such as from 2.2-2.3 g/100 g lipid, for example 2.3-2.4 g/100 g lipid, such as from 2.4-2.5 g/100 g lipid, for example 2.5-2.6 g/100 g lipid, such as from 2.6-2.7 g/100 g lipid, for example 2.7-2.8 g/100 g lipid, such as from 2.8-2.9 g/100 g lipid, for example 2.9-3.0 g/100 g lipid, such as from 3.0-3.1 g/100 g lipid, for example 3.1-3.2 g/100 g lipid, such as from 3.2-3.3 g/100 g lipid, for example 3.3-3.4 g/100 g lipid, such as from 3.4-3.5 g/100 g lipid, for example 3.5-3.6 g/100 g lipid, such as from 3.6-3.7 g/100 g lipid, for example 3.7-3.8 g/100 g lipid, such as from 3.8-3.9 g/100 g lipid, for example 3.9-4.0 g/100 g lipid, such as from 4.0-4.1 g/100 g lipid, for example 4.1-4.2 g/100 g lipid, such as from 4.2-4.3 g/100 g lipid, for example 4.3-4.4 g/100 g lipid, such as from 4.4-4.5 g/100 g lipid, for example 4.5-4.6 g/100 g lipid, such as from 4.6-4.7 g/100 g lipid, for example 4.7-4.8 g/100 g lipid, such as from 4.8-4.9 g/100 g lipid, for example 4.9-5.0 g/100 g lipid, such as from 5.0-5.1 g/100 g lipid, for example 5.1-5.2 g/100 g lipid, such as from 5.2-5.3 g/100 g lipid, for example 5.3-5.4 g/100 g lipid, such as from 5.4-5.5 g/100 g lipid, for example 5.5-5.6 g/100 g lipid, such as from 5.6-5.7 g/100 g lipid, for example 5.7-5.8 g/100 g lipid, such as from 5.8-5.9 g/100 g lipid, for example 5.9-6.0 g/100 g lipid, such as from 6.0-6.1 g/100 g lipid, for example 6.1-6.2 g/100 g lipid, such as from 6.2-6.3 g/100 g lipid, for example 6.3-6.4 g/100 g lipid, such as from 6.4-6.5 g/100 g lipid, for example 6.5-6.6 g/100 g lipid, such as from 6.6-6.7 g/100 g lipid, for example 6.7-6.8 g/100 g lipid, such as from 6.8-6.9 g/100 g lipid, for example 6.9-7.0 g/100 g lipid, such as from 7.0-7.1 g/100 g lipid, for example 7.1-7.2 g/100 g lipid, such as from 7.2-7.3 g/100 g lipid, for example 7.3-7.4 g/100 g lipid, such as from 7.4-7.5 g/100 g lipid, for example 7.5-7.6 g/100 g lipid, such as from 7.6-7.7 g/100 g lipid, for example 7.7-7.8 g/100 g lipid, such as from 7.8-7.9 g/100 g lipid, for example 7.9-8.0 g/100 g lipid, such as from 8.0-8.1 g/100 g lipid, for example 8.1-8.2 g/100 g lipid, such as from 8.2-8.3 g/100 g lipid, for example 8.3-8.4 g/100 g lipid, such as from 8.4-8.5 g/100 g lipid, for example 8.5-8.6 g/100 g lipid, such as from 8.6-8.7 g/100 g lipid, for example 8.7-8.8 g/100 g lipid, such as from 8.8-8.9 g/100 g lipid, for example 8.9-9.0 g/100 g lipid, such as from 9.0-9.1 g/100 g lipid, for example 9.1-9.2 g/100 g lipid, such as from 9.2-9.3 g/100 g lipid, for example 9.3-9.4 g/100 g lipid, such as from 9.4-9.5 g/100 g lipid, for example 9.5-9.6 g/100 g lipid, such as from 9.6-9.7 g/100 g lipid, for example 9.7-9.8 g/100 g lipid, such as from 9.8-9.9 g/100 g lipid, for example 9.9-10.0 g/100 g lipid, such as from 10.0-10.1 g/100 g lipid, for example 10.1-10.2 g/100 g lipid, such as from 10.2-10.3 g/100 g lipid, for example 10.3-10.4 g/100 g lipid, such as from 10.4-10.5 g/100 g lipid, for example 10.5-10.6 g/100 g lipid, such as from 10.6-10.7 g/100 g lipid, for example 10.7-10.8 g/100 g lipid, such as from 10.8-10.9 g/100 g lipid, for example 10.9-11.0 g/100 g lipid, such as from 11.0-11.1 g/100 g lipid, for example 11.1-11.2 g/100 g lipid, such as from 11.2-11.3 g/100 g lipid, for example 11.3-11.4 g/100 g lipid, such as from 11.4-11.5 g/100 g lipid, for example 11.5-11.6 g/100 g lipid, such as from 11.6-11.7 g/100 g lipid, for example 11.7-11.8 g/100 g lipid, such as from 11.8-11.9 g/100 g lipid, for example 11.9-12.0 g/100 g lipid, such as from 12.0-12.1 g/100 g lipid, for example 12.1-12.2 g/100 g lipid, such as from 12.2-12.3 g/100 g lipid, for example 12.3-12.4 g/100 g lipid, such as from 12.4-12.5 g/100 g lipid, for example 12.5-12.6 g/100 g lipid, such as from 12.6-12.7 g/100 g lipid, for example 12.7-12.8 g/100 g lipid, such as from 12.8-12.9 g/100 g lipid, for example 12.9-13.0 g/100 g lipid, such as from 13.0-13.1 g/100 g lipid, for example 13.1-13.2 g/100 g lipid, such as from 13.2-13.3 g/100 g lipid, for example 13.3-13.4 g/100 g lipid, such as from 13.4-13.5 g/100 g lipid, for example 13.5-13.6 g/100 g lipid, such as from 13.6-13.7 g/100 g lipid, for example 13.7-13.8 g/100 g lipid, such as from 13.8-13.9 g/100 g lipid, for example 13.9-14.0 g/100 g lipid, such as from 14.0-14.1 g/100 g lipid, for example 14.1-14.2 g/100 g lipid, such as from 14.2-14.3 g/100 g lipid, for example 14.3-14.4 g/100 g lipid, such as from 14.4-14.5 g/100 g lipid, for example 14.5-14.6 g/100 g lipid, such as from 14.6-14.7 g/100 g lipid, for example 14.7-14.8 g/100 g lipid, such as from 14.8-14.9 g/100 g lipid, for example 14.9-15.0 g/100 g lipid, such as from 15-16 g/100 g lipid, for example 16-17 g/100 g lipid, such as from 17-18 g/100 g lipid, for example 18-19 g/100 g lipid, such as from 19-20 g/100 g lipid.

In one embodiment the present invention relates to a fish feed composition with a 16:1 content (g/100 g lipid) in the range from 0-20 g/100 g lipid, such as from 0-0.1 g/100 g lipid, for example 0.1-0.2 g/100 g lipid, such as from 0.2-0.3 g/100 g lipid, for example 0.3-0.4 g/100 g lipid, such as from 0.4-0.5 g/100 g lipid, for example 0.5-0.6 g/100 g lipid, such as from 0.6-0.7 g/100 g lipid, for example 0.7-0.8 g/100 g lipid, such as from 0.8-0.9 g/100 g lipid, for example 0.9-1.0 g/100 g lipid, such as from 1.0-1.1 g/100 g lipid, for example 1.1-1.2 g/100 g lipid, such as from 1.2-1.3 g/100 g lipid, for example 1.3-1.4 g/100 g lipid, such as from 1.4-1.5 g/100 g lipid, for example 1.5-1.6 g/100 g lipid, such as from 1.6-1.7 g/100 g lipid, for example 1.7-1.8 g/100 g lipid, such as from 1.8-1.9 g/100 g lipid, for example 1.9-2.0 g/100 g lipid, such as from 2.0-2.1 g/100 g lipid, for example 2.1-2.2 g/100 g lipid, such as from 2.2-2.3 g/100 g lipid, for example 2.3-2.4 g/100 g lipid, such as from 2.4-2.5 g/100 g lipid, for example 2.5-2.6 g/100 g lipid, such as from 2.6-2.7 g/100 g lipid, for example 2.7-2.8 g/100 g lipid, such as from 2.8-2.9 g/100 g lipid, for example 2.9-3.0 g/100 g lipid, such as from 3.0-3.1 g/100 g lipid, for example 3.1-3.2 g/100 g lipid, such as from 3.2-3.3 g/100 g lipid, for example 3.3-3.4 g/100 g lipid, such as from 3.4-3.5 g/100 g lipid, for example 3.5-3.6 g/100 g lipid, such as from 3.6-3.7 g/100 g lipid, for example 3.7-3.8 g/100 g lipid, such as from 3.8-3.9 g/100 g lipid, for example 3.9-4.0 g/100 g lipid, such as from 4.0-4.1 g/100 g lipid, for example 4.1-4.2 g/100 g lipid, such as from 4.2-4.3 g/100 g lipid, for example 4.3-4.4 g/100 g lipid, such as from 4.4-4.5 g/100 g lipid, for example 4.5-4.6 g/100 g lipid, such as from 4.6-4.7 g/100 g lipid, for example 4.7-4.8 g/100 g lipid, such as from 4.8-4.9 g/100 g lipid, for example 4.9-5.0 g/100 g lipid, such as from 5.0-5.1 g/100 g lipid, for example 5.1-5.2 g/100 g lipid, such as from 5.2-5.3 g/100 g lipid, for example 5.3-5.4 g/100 g lipid, such as from 5.4-5.5 g/100 g lipid, for example 5.5-5.6 g/100 g lipid, such as from 5.6-5.7 g/100 g lipid, for example 5.7-5.8 g/100 g lipid, such as from 5.8-5.9 g/100 g lipid, for example 5.9-6.0 g/100 g lipid, such as from 6.0-6.1 g/100 g lipid, for example 6.1-6.2 g/100 g lipid, such as from 6.2-6.3 g/100 g lipid, for example 6.3-6.4 g/100 g lipid, such as from 6.4-6.5 g/100 g lipid, for example 6.5-6.6 g/100 g lipid, such as from 6.6-6.7 g/100 g lipid, for example 6.7-6.8 g/100 g lipid, such as from 6.8-6.9 g/100 g lipid, for example 6.9-7.0 g/100 g lipid, such as from 7.0-7.1 g/100 g lipid, for example 7.1-7.2 g/100 g lipid, such as from 7.2-7.3 g/100 g lipid, for example 7.3-7.4 g/100 g lipid, such as from 7.4-7.5 g/100 g lipid, for example 7.5-7.6 g/100 g lipid, such as from 7.6-7.7 g/100 g lipid, for example 7.7-7.8 g/100 g lipid, such as from 7.8-7.9 g/100 g lipid, for example 7.9-8.0 g/100 g lipid, such as from 8.0-8.1 g/100 g lipid, for example 8.1-8.2 g/100 g lipid, such as from 8.2-8.3 g/100 g lipid, for example 8.3-8.4 g/100 g lipid, such as from 8.4-8.5 g/100 g lipid, for example 8.5-8.6 g/100 g lipid, such as from 8.6-8.7 g/100 g lipid, for example 8.7-8.8 g/100 g lipid, such as from 8.8-8.9 g/100 g lipid, for example 8.9-9.0 g/100 g lipid, such as from 9.0-9.1 g/100 g lipid, for example 9.1-9.2 g/100 g lipid, such as from 9.2-9.3 g/100 g lipid, for example 9.3-9.4 g/100 g lipid, such as from 9.4-9.5 g/100 g lipid, for example 9.5-9.6 g/100 g lipid, such as from 9.6-9.7 g/100 g lipid, for example 9.7-9.8 g/100 g lipid, such as from 9.8-9.9 g/100 g lipid, for example 9.9-10.0 g/100 g lipid, such as from 10.0-10.1 g/100 g lipid, for example 10.1-10.2 g/100 g lipid, such as from 10.2-10.3 g/100 g lipid, for example 10.3-10.4 g/100 g lipid, such as from 10.4-10.5 g/100 g lipid, for example 10.5-10.6 g/100 g lipid, such as from 10.6-10.7 g/100 g lipid, for example 10.7-10.8 g/100 g lipid, such as from 10.8-10.9 g/100 g lipid, for example 10.9-11.0 g/100 g lipid, such as from 11.0-11.1 g/100 g lipid, for example 11.1-11.2 g/100 g lipid, such as from 11.2-11.3 g/100 g lipid, for example 11.3-11.4 g/100 g lipid, such as from 11.4-11.5 g/100 g lipid, for example 11.5-11.6 g/100 g lipid, such as from 11.6-11.7 g/100 g lipid, for example 11.7-11.8 g/100 g lipid, such as from 11.8-11.9 g/100 g lipid, for example 11.9-12.0 g/100 g lipid, such as from 12.0-12.1 g/100 g lipid, for example 12.1-12.2 g/100 g lipid, such as from 12.2-12.3 g/100 g lipid, for example 12.3-12.4 g/100 g lipid, such as from 12.4-12.5 g/100 g lipid, for example 12.5-12.6 g/100 g lipid, such as from 12.6-12.7 g/100 g lipid, for example 12.7-12.8 g/100 g lipid, such as from 12.8-12.9 g/100 g lipid, for example 12.9-13.0 g/100 g lipid, such as from 13.0-13.1 g/100 g lipid, for example 13.1-13.2 g/100 g lipid, such as from 13.2-13.3 g/100 g lipid, for example 13.3-13.4 g/100 g lipid, such as from 13.4-13.5 g/100 g lipid, for example 13.5-13.6 g/100 g lipid, such as from 13.6-13.7 g/100 g lipid, for example 13.7-13.8 g/100 g lipid, such as from 13.8-13.9 g/100 g lipid, for example 13.9-14.0 g/100 g lipid, such as from 14.0-14.1 g/100 g lipid, for example 14.1-14.2 g/100 g lipid, such as from 14.2-14.3 g/100 g lipid, for example 14.3-14.4 g/100 g lipid, such as from 14.4-14.5 g/100 g lipid, for example 14.5-14.6 g/100 g lipid, such as from 14.6-14.7 g/100 g lipid, for example 14.7-14.8 g/100 g lipid, such as from 14.8-14.9 g/100 g lipid, for example 14.9-15.0 g/100 g lipid, such as from 15-16 g/100 g lipid, for example 16-17 g/100 g lipid, such as from 17-18 g/100 g lipid, for example 18-19 g/100 g lipid, such as from 19-20 g/100 g lipid.

In one embodiment the present invention relates to a fish feed composition with a 18:1 content (g/100 g lipid) in the range from 0-20 g/100 g lipid, such as from 0-0.1 g/100 g lipid, for example 0.1-0.2 g/100 g lipid, such as from 0.2-0.3 g/100 g lipid, for example 0.3-0.4 g/100 g lipid, such as from 0.4-0.5 g/100 g lipid, for example 0.5-0.6 g/100 g lipid, such as from 0.6-0.7 g/100 g lipid, for example 0.7-0.8 g/100 g lipid, such as from 0.8-0.9 g/100 g lipid, for example 0.9-1.0 g/100 g lipid, such as from 1.0-1.1 g/100 g lipid, for example 1.1-1.2 g/100 g lipid, such as from 1.2-1.3 g/100 g lipid, for example 1.3-1.4 g/100 g lipid, such as from 1.4-1.5 g/100 g lipid, for example 1.5-1.6 g/100 g lipid, such as from 1.6-1.7 g/100 g lipid, for example 1.7-1.8 g/100 g lipid, such as from 1.8-1.9 g/100 g lipid, for example 1.9-2.0 g/100 g lipid, such as from 2.0-2.1 g/100 g lipid, for example 2.1-2.2 g/100 g lipid, such as from 2.2-2.3 g/100 g lipid, for example 2.3-2.4 g/100 g lipid, such as from 2.4-2.5 g/100 g lipid, for example 2.5-2.6 g/100 g lipid, such as from 2.6-2.7 g/100 g lipid, for example 2.7-2.8 g/100 g lipid, such as from 2.8-2.9 g/100 g lipid, for example 2.9-3.0 g/100 g lipid, such as from 3.0-3.1 g/100 g lipid, for example 3.1-3.2 g/100 g lipid, such as from 3.2-3.3 g/100 g lipid, for example 3.3-3.4 g/100 g lipid, such as from 3.4-3.5 g/100 g lipid, for example 3.5-3.6 g/100 g lipid, such as from 3.6-3.7 g/100 g lipid, for example 3.7-3.8 g/100 g lipid, such as from 3.8-3.9 g/100 g lipid, for example 3.9-4.0 g/100 g lipid, such as from 4.0-4.1 g/100 g lipid, for example 4.1-4.2 g/100 g lipid, such as from 4.2-4.3 g/100 g lipid, for example 4.3-4.4 g/100 g lipid, such as from 4.4-4.5 g/100 g lipid, for example 4.5-4.6 g/100 g lipid, such as from 4.6-4.7 g/100 g lipid, for example 4.7-4.8 g/100 g lipid, such as from 4.8-4.9 g/100 g lipid, for example 4.9-5.0 g/100 g lipid, such as from 5.0-5.1 g/100 g lipid, for example 5.1-5.2 g/100 g lipid, such as from 5.2-5.3 g/100 g lipid, for example 5.3-5.4 g/100 g lipid, such as from 5.4-5.5 g/100 g lipid, for example 5.5-5.6 g/100 g lipid, such as from 5.6-5.7 g/100 g lipid, for example 5.7-5.8 g/100 g lipid, such as from 5.8-5.9 g/100 g lipid, for example 5.9-6.0 g/100 g lipid, such as from 6.0-6.1 g/100 g lipid, for example 6.1-6.2 g/100 g lipid, such as from 6.2-6.3 g/100 g lipid, for example 6.3-6.4 g/100 g lipid, such as from 6.4-6.5 g/100 g lipid, for example 6.5-6.6 g/100 g lipid, such as from 6.6-6.7 g/100 g lipid, for example 6.7-6.8 g/100 g lipid, such as from 6.8-6.9 g/100 g lipid, for example 6.9-7.0 g/100 g lipid, such as from 7.0-7.1 g/100 g lipid, for example 7.1-7.2 g/100 g lipid, such as from 7.2-7.3 g/100 g lipid, for example 7.3-7.4 g/100 g lipid, such as from 7.4-7.5 g/100 g lipid, for example 7.5-7.6 g/100 g lipid, such as from 7.6-7.7 g/100 g lipid, for example 7.7-7.8 g/100 g lipid, such as from 7.8-7.9 g/100 g lipid, for example 7.9-8.0 g/100 g lipid, such as from 8.0-8.1 g/100 g lipid, for example 8.1-8.2 g/100 g lipid, such as from 8.2-8.3 g/100 g lipid, for example 8.3-8.4 g/100 g lipid, such as from 8.4-8.5 g/100 g lipid, for example 8.5-8.6 g/100 g lipid, such as from 8.6-8.7 g/100 g lipid, for example 8.7-8.8 g/100 g lipid, such as from 8.8-8.9 g/100 g lipid, for example 8.9-9.0 g/100 g lipid, such as from 9.0-9.1 g/100 g lipid, for example 9.1-9.2 g/100 g lipid, such as from 9.2-9.3 g/100 g lipid, for example 9.3-9.4 g/100 g lipid, such as from 9.4-9.5 g/100 g lipid, for example 9.5-9.6 g/100 g lipid, such as from 9.6-9.7 g/100 g lipid, for example 9.7-9.8 g/100 g lipid, such as from 9.8-9.9 g/100 g lipid, for example 9.9-10.0 g/100 g lipid, such as from 10.0-10.1 g/100 g lipid, for example 10.1-10.2 g/100 g lipid, such as from 10.2-10.3 g/100 g lipid, for example 10.3-10.4 g/100 g lipid, such as from 10.4-10.5 g/100 g lipid, for example 10.5-10.6 g/100 g lipid, such as from 10.6-10.7 g/100 g lipid, for example 10.7-10.8 g/100 g lipid, such as from 10.8-10.9 g/100 g lipid, for example 10.9-11.0 g/100 g lipid, such as from 11.0-11.1 g/100 g lipid, for example 11.1-11.2 g/100 g lipid, such as from 11.2-11.3 g/100 g lipid, for example 11.3-11.4 g/100 g lipid, such as from 11.4-11.5 g/100 g lipid, for example 11.5-11.6 g/100 g lipid, such as from 11.6-11.7 g/100 g lipid, for example 11.7-11.8 g/100 g lipid, such as from 11.8-11.9 g/100 g lipid, for example 11.9-12.0 g/100 g lipid, such as from 12.0-12.1 g/100 g lipid, for example 12.1-12.2 g/100 g lipid, such as from 12.2-12.3 g/100 g lipid, for example 12.3-12.4 g/100 g lipid, such as from 12.4-12.5 g/100 g lipid, for example 12.5-12.6 g/100 g lipid, such as from 12.6-12.7 g/100 g lipid, for example 12.7-12.8 g/100 g lipid, such as from 12.8-12.9 g/100 g lipid, for example 12.9-13.0 g/100 g lipid, such as from 13.0-13.1 g/100 g lipid, for example 13.1-13.2 g/100 g lipid, such as from 13.2-13.3 g/100 g lipid, for example 13.3-13.4 g/100 g lipid, such as from 13.4-13.5 g/100 g lipid, for example 13.5-13.6 g/100 g lipid, such as from 13.6-13.7 g/100 g lipid, for example 13.7-13.8 g/100 g lipid, such as from 13.8-13.9 g/100 g lipid, for example 13.9-14.0 g/100 g lipid, such as from 14.0-14.1 g/100 g lipid, for example 14.1-14.2 g/100 g lipid, such as from 14.2-14.3 g/100 g lipid, for example 14.3-14.4 g/100 g lipid, such as from 14.4-14.5 g/100 g lipid, for example 14.5-14.6 g/100 g lipid, such as from 14.6-14.7 g/100 g lipid, for example 14.7-14.8 g/100 g lipid, such as from 14.8-14.9 g/100 g lipid, for example 14.9-15.0 g/100 g lipid, such as from 15-16 g/100 g lipid, for example 16-17 g/100 g lipid, such as from 17-18 g/100 g lipid, for example 18-19 g/100 g lipid, such as from 19-20 g/100 g lipid.

In one embodiment the present invention relates to a fish feed composition with a 20:1 content (g/100 g lipid) in the range from 0-20 g/100 g lipid, such as from 0-0.1 g/100 g lipid, for example 0.1-0.2 g/100 g lipid, such as from 0.2-0.3 g/100 g lipid, for example 0.3-0.4 g/100 g lipid, such as from 0.4-0.5 g/100 g lipid, for example 0.5-0.6 g/100 g lipid, such as from 0.6-0.7 g/100 g lipid, for example 0.7-0.8 g/100 g lipid, such as from 0.8-0.9 g/100 g lipid, for example 0.9-1.0 g/100 g lipid, such as from 1.0-1.1 g/100 g lipid, for example 1.1-1.2 g/100 g lipid, such as from 1.2-1.3 g/100 g lipid, for example 1.3-1.4 g/100 g lipid, such as from 1.4-1.5 g/100 g lipid, for example 1.5-1.6 g/100 g lipid, such as from 1.6-1.7 g/100 g lipid, for example 1.7-1.8 g/100 g lipid, such as from 1.8-1.9 g/100 g lipid, for example 1.9-2.0 g/100 g lipid, such as from 2.0-2.1 g/100 g lipid, for example 2.1-2.2 g/100 g lipid, such as from 2.2-2.3 g/100 g lipid, for example 2.3-2.4 g/100 g lipid, such as from 2.4-2.5 g/100 g lipid, for example 2.5-2.6 g/100 g lipid, such as from 2.6-2.7 g/100 g lipid, for example 2.7-2.8 g/100 g lipid, such as from 2.8-2.9 g/100 g lipid, for example 2.9-3.0 g/100 g lipid, such as from 3.0-3.1 g/100 g lipid, for example 3.1-3.2 g/100 g lipid, such as from 3.2-3.3 g/100 g lipid, for example 3.3-3.4 g/100 g lipid, such as from 3.4-3.5 g/100 g lipid, for example 3.5-3.6 g/100 g lipid, such as from 3.6-3.7 g/100 g lipid, for example 3.7-3.8 g/100 g lipid, such as from 3.8-3.9 g/100 g lipid, for example 3.9-4.0 g/100 g lipid, such as from 4.0-4.1 g/100 g lipid, for example 4.1-4.2 g/100 g lipid, such as from 4.2-4.3 g/100 g lipid, for example 4.3-4.4 g/100 g lipid, such as from 4.4-4.5 g/100 g lipid, for example 4.5-4.6 g/100 g lipid, such as from 4.6-4.7 g/100 g lipid, for example 4.7-4.8 g/100 g lipid, such as from 4.8-4.9 g/100 g lipid, for example 4.9-5.0 g/100 g lipid, such as from 5.0-5.1 g/100 g lipid, for example 5.1-5.2 g/100 g lipid, such as from 5.2-5.3 g/100 g lipid, for example 5.3-5.4 g/100 g lipid, such as from 5.4-5.5 g/100 g lipid, for example 5.5-5.6 g/100 g lipid, such as from 5.6-5.7 g/100 g lipid, for example 5.7-5.8 g/100 g lipid, such as from 5.8-5.9 g/100 g lipid, for example 5.9-6.0 g/100 g lipid, such as from 6.0-6.1 g/100 g lipid, for example 6.1-6.2 g/100 g lipid, such as from 6.2-6.3 g/100 g lipid, for example 6.3-6.4 g/100 g lipid, such as from 6.4-6.5 g/100 g lipid, for example 6.5-6.6 g/100 g lipid, such as from 6.6-6.7 g/100 g lipid, for example 6.7-6.8 g/100 g lipid, such as from 6.8-6.9 g/100 g lipid, for example 6.9-7.0 g/100 g lipid, such as from 7.0-7.1 g/100 g lipid, for example 7.1-7.2 g/100 g lipid, such as from 7.2-7.3 g/100 g lipid, for example 7.3-7.4 g/100 g lipid, such as from 7.4-7.5 g/100 g lipid, for example 7.5-7.6 g/100 g lipid, such as from 7.6-7.7 g/100 g lipid, for example 7.7-7.8 g/100 g lipid, such as from 7.8-7.9 g/100 g lipid, for example 7.9-8.0 g/100 g lipid, such as from 8.0-8.1 g/100 g lipid, for example 8.1-8.2 g/100 g lipid, such as from 8.2-8.3 g/100 g lipid, for example 8.3-8.4 g/100 g lipid, such as from 8.4-8.5 g/100 g lipid, for example 8.5-8.6 g/100 g lipid, such as from 8.6-8.7 g/100 g lipid, for example 8.7-8.8 g/100 g lipid, such as from 8.8-8.9 g/100 g lipid, for example 8.9-9.0 g/100 g lipid, such as from 9.0-9.1 g/100 g lipid, for example 9.1-9.2 g/100 g lipid, such as from 9.2-9.3 g/100 g lipid, for example 9.3-9.4 g/100 g lipid, such as from 9.4-9.5 g/100 g lipid, for example 9.5-9.6 g/100 g lipid, such as from 9.6-9.7 g/100 g lipid, for example 9.7-9.8 g/100 g lipid, such as from 9.8-9.9 g/100 g lipid, for example 9.9-10.0 g/100 g lipid, such as from 10.0-10.1 g/100 g lipid, for example 10.1-10.2 g/100 g lipid, such as from 10.2-10.3 g/100 g lipid, for example 10.3-10.4 g/100 g lipid, such as from 10.4-10.5 g/100 g lipid, for example 10.5-10.6 g/100 g lipid, such as from 10.6-10.7 g/100 g lipid, for example 10.7-10.8 g/100 g lipid, such as from 10.8-10.9 g/100 g lipid, for example 10.9-11.0 g/100 g lipid, such as from 11.0-11.1 g/100 g lipid, for example 11.1-11.2 g/100 g lipid, such as from 11.2-11.3 g/100 g lipid, for example 11.3-11.4 g/100 g lipid, such as from 11.4-11.5 g/100 g lipid, for example 11.5-11.6 g/100 g lipid, such as from 11.6-11.7 g/100 g lipid, for example 11.7-11.8 g/100 g lipid, such as from 11.8-11.9 g/100 g lipid, for example 11.9-12.0 g/100 g lipid, such as from 12.0-12.1 g/100 g lipid, for example 12.1-12.2 g/100 g lipid, such as from 12.2-12.3 g/100 g lipid, for example 12.3-12.4 g/100 g lipid, such as from 12.4-12.5 g/100 g lipid, for example 12.5-12.6 g/100 g lipid, such as from 12.6-12.7 g/100 g lipid, for example 12.7-12.8 g/100 g lipid, such as from 12.8-12.9 g/100 g lipid, for example 12.9-13.0 g/100 g lipid, such as from 13.0-13.1 g/100 g lipid, for example 13.1-13.2 g/100 g lipid, such as from 13.2-13.3 g/100 g lipid, for example 13.3-13.4 g/100 g lipid, such as from 13.4-13.5 g/100 g lipid, for example 13.5-13.6 g/100 g lipid, such as from 13.6-13.7 g/100 g lipid, for example 13.7-13.8 g/100 g lipid, such as from 13.8-13.9 g/100 g lipid, for example 13.9-14.0 g/100 g lipid, such as from 14.0-14.1 g/100 g lipid, for example 14.1-14.2 g/100 g lipid, such as from 14.2-14.3 g/100 g lipid, for example 14.3-14.4 g/100 g lipid, such as from 14.4-14.5 g/100 g lipid, for example 14.5-14.6 g/100 g lipid, such as from 14.6-14.7 g/100 g lipid, for example 14.7-14.8 g/100 g lipid, such as from 14.8-14.9 g/100 g lipid, for example 14.9-15.0 g/100 g lipid, such as from 15-16 g/100 g lipid, for example 16-17 g/100 g lipid, such as from 17-18 g/100 g lipid, for example 18-19 g/100 g lipid, such as from 19-20 g/100 g lipid.

In one embodiment the present invention relates to a fish feed composition with a 22:1 content (g/100 g lipid) in the range from 0-20 g/100 g lipid, such as from 0-0.1 g/100 g lipid, for example 0.1-0.2 g/100 g lipid, such as from 0.2-0.3 g/100 g lipid, for example 0.3-0.4 g/100 g lipid, such as from 0.4-0.5 g/100 g lipid, for example 0.5-0.6 g/100 g lipid, such as from 0.6-0.7 g/100 g lipid, for example 0.7-0.8 g/100 g lipid, such as from 0.8-0.9 g/100 g lipid, for example 0.9-1.0 g/100 g lipid, such as from 1.0-1.1 g/100 g lipid, for example 1.1-1.2 g/100 g lipid, such as from 1.2-1.3 g/100 g lipid, for example 1.3-1.4 g/100 g lipid, such as from 1.4-1.5 g/100 g lipid, for example 1.5-1.6 g/100 g lipid, such as from 1.6-1.7 g/100 g lipid, for example 1.7-1.8 g/100 g lipid, such as from 1.8-1.9 g/100 g lipid, for example 1.9-2.0 g/100 g lipid, such as from 2.0-2.1 g/100 g lipid, for example 2.1-2.2 g/100 g lipid, such as from 2.2-2.3 g/100 g lipid, for example 2.3-2.4 g/100 g lipid, such as from 2.4-2.5 g/100 g lipid, for example 2.5-2.6 g/100 g lipid, such as from 2.6-2.7 g/100 g lipid, for example 2.7-2.8 g/100 g lipid, such as from 2.8-2.9 g/100 g lipid, for example 2.9-3.0 g/100 g lipid, such as from 3.0-3.1 g/100 g lipid, for example 3.1-3.2 g/100 g lipid, such as from 3.2-3.3 g/100 g lipid, for example 3.3-3.4 g/100 g lipid, such as from 3.4-3.5 g/100 g lipid, for example 3.5-3.6 g/100 g lipid, such as from 3.6-3.7 g/100 g lipid, for example 3.7-3.8 g/100 g lipid, such as from 3.8-3.9 g/100 g lipid, for example 3.9-4.0 g/100 g lipid, such as from 4.0-4.1 g/100 g lipid, for example 4.1-4.2 g/100 g lipid, such as from 4.2-4.3 g/100 g lipid, for example 4.3-4.4 g/100 g lipid, such as from 4.4-4.5 g/100 g lipid, for example 4.5-4.6 g/100 g lipid, such as from 4.6-4.7 g/100 g lipid, for example 4.7-4.8 g/100 g lipid, such as from 4.8-4.9 g/100 g lipid, for example 4.9-5.0 g/100 g lipid, such as from 5.0-5.1 g/100 g lipid, for example 5.1-5.2 g/100 g lipid, such as from 5.2-5.3 g/100 g lipid, for example 5.3-5.4 g/100 g lipid, such as from 5.4-5.5 g/100 g lipid, for example 5.5-5.6 g/100 g lipid, such as from 5.6-5.7 g/100 g lipid, for example 5.7-5.8 g/100 g lipid, such as from 5.8-5.9 g/100 g lipid, for example 5.9-6.0 g/100 g lipid, such as from 6.0-6.1 g/100 g lipid, for example 6.1-6.2 g/100 g lipid, such as from 6.2-6.3 g/100 g lipid, for example 6.3-6.4 g/100 g lipid, such as from 6.4-6.5 g/100 g lipid, for example 6.5-6.6 g/100 g lipid, such as from 6.6-6.7 g/100 g lipid, for example 6.7-6.8 g/100 g lipid, such as from 6.8-6.9 g/100 g lipid, for example 6.9-7.0 g/100 g lipid, such as from 7.0-7.1 g/100 g lipid, for example 7.1-7.2 g/100 g lipid, such as from 7.2-7.3 g/100 g lipid, for example 7.3-7.4 g/100 g lipid, such as from 7.4-7.5 g/100 g lipid, for example 7.5-7.6 g/100 g lipid, such as from 7.6-7.7 g/100 g lipid, for example 7.7-7.8 g/100 g lipid, such as from 7.8-7.9 g/100 g lipid, for example 7.9-8.0 g/100 g lipid, such as from 8.0-8.1 g/100 g lipid, for example 8.1-8.2 g/100 g lipid, such as from 8.2-8.3 g/100 g lipid, for example 8.3-8.4 g/100 g lipid, such as from 8.4-8.5 g/100 g lipid, for example 8.5-8.6 g/100 g lipid, such as from 8.6-8.7 g/100 g lipid, for example 8.7-8.8 g/100 g lipid, such as from 8.8-8.9 g/100 g lipid, for example 8.9-9.0 g/100 g lipid, such as from 9.0-9.1 g/100 g lipid, for example 9.1-9.2 g/100 g lipid, such as from 9.2-9.3 g/100 g lipid, for example 9.3-9.4 g/100 g lipid, such as from 9.4-9.5 g/100 g lipid, for example 9.5-9.6 g/100 g lipid, such as from 9.6-9.7 g/100 g lipid, for example 9.7-9.8 g/100 g lipid, such as from 9.8-9.9 g/100 g lipid, for example 9.9-10.0 g/100 g lipid, such as from 10.0-10.1 g/100 g lipid, for example 10.1-10.2 g/100 g lipid, such as from 10.2-10.3 g/100 g lipid, for example 10.3-10.4 g/100 g lipid, such as from 10.4-10.5 g/100 g lipid, for example 10.5-10.6 g/100 g lipid, such as from 10.6-10.7 g/100 g lipid, for example 10.7-10.8 g/100 g lipid, such as from 10.8-10.9 g/100 g lipid, for example 10.9-11.0 g/100 g lipid, such as from 11.0-11.1 g/100 g lipid, for example 11.1-11.2 g/100 g lipid, such as from 11.2-11.3 g/100 g lipid, for example 11.3-11.4 g/100 g lipid, such as from 11.4-11.5 g/100 g lipid, for example 11.5-11.6 g/100 g lipid, such as from 11.6-11.7 g/100 g lipid, for example 11.7-11.8 g/100 g lipid, such as from 11.8-11.9 g/100 g lipid, for example 11.9-12.0 g/100 g lipid, such as from 12.0-12.1 g/100 g lipid, for example 12.1-12.2 g/100 g lipid, such as from 12.2-12.3 g/100 g lipid, for example 12.3-12.4 g/100 g lipid, such as from 12.4-12.5 g/100 g lipid, for example 12.5-12.6 g/100 g lipid, such as from 12.6-12.7 g/100 g lipid, for example 12.7-12.8 g/100 g lipid, such as from 12.8-12.9 g/100 g lipid, for example 12.9-13.0 g/100 g lipid, such as from 13.0-13.1 g/100 g lipid, for example 13.1-13.2 g/100 g lipid, such as from 13.2-13.3 g/100 g lipid, for example 13.3-13.4 g/100 g lipid, such as from 13.4-13.5 g/100 g lipid, for example 13.5-13.6 g/100 g lipid, such as from 13.6-13.7 g/100 g lipid, for example 13.7-13.8 g/100 g lipid, such as from 13.8-13.9 g/100 g lipid, for example 13.9-14.0 g/100 g lipid, such as from 14.0-14.1 g/100 g lipid, for example 14.1-14.2 g/100 g lipid, such as from 14.2-14.3 g/100 g lipid, for example 14.3-14.4 g/100 g lipid, such as from 14.4-14.5 g/100 g lipid, for example 14.5-14.6 g/100 g lipid, such as from 14.6-14.7 g/100 g lipid, for example 14.7-14.8 g/100 g lipid, such as from 14.8-14.9 g/100 g lipid, for example 14.9-15.0 g/100 g lipid, such as from 15-16 g/100 g lipid, for example 16-17 g/100 g lipid, such as from 17-18 g/100 g lipid, for example 18-19 g/100 g lipid, such as from 19-20 g/100 g lipid.

In one embodiment the present invention relates to a fish feed composition with a 24:1 n-9 content (g/100 g lipid) in the range from 0-20 g/100 g lipid, such as from 0-0.1 g/100 g lipid, for example 0.1-0.2 g/100 g lipid, such as from 0.2-0.3 g/100 g lipid, for example 0.3-0.4 g/100 g lipid, such as from 0.4-0.5 g/100 g lipid, for example 0.5-0.6 g/100 g lipid, such as from 0.6-0.7 g/100 g lipid, for example 0.7-0.8 g/100 g lipid, such as from 0.8-0.9 g/100 g lipid, for example 0.9-1.0 g/100 g lipid, such as from 1.0-1.1 g/100 g lipid, for example 1.1-1.2 g/100 g lipid, such as from 1.2-1.3 g/100 g lipid, for example 1.3-1.4 g/100 g lipid, such as from 1.4-1.5 g/100 g lipid, for example 1.5-1.6 g/100 g lipid, such as from 1.6-1.7 g/100 g lipid, for example 1.7-1.8 g/100 g lipid, such as from 1.8-1.9 g/100 g lipid, for example 1.9-2.0 g/100 g lipid, such as from 2.0-2.1 g/100 g lipid, for example 2.1-2.2 g/100 g lipid, such as from 2.2-2.3 g/100 g lipid, for example 2.3-2.4 g/100 g lipid, such as from 2.4-2.5 g/100 g lipid, for example 2.5-2.6 g/100 g lipid, such as from 2.6-2.7 g/100 g lipid, for example 2.7-2.8 g/100 g lipid, such as from 2.8-2.9 g/100 g lipid, for example 2.9-3.0 g/100 g lipid, such as from 3.0-3.1 g/100 g lipid, for example 3.1-3.2 g/100 g lipid, such as from 3.2-3.3 g/100 g lipid, for example 3.3-3.4 g/100 g lipid, such as from 3.4-3.5 g/100 g lipid, for example 3.5-3.6 g/100 g lipid, such as from 3.6-3.7 g/100 g lipid, for example 3.7-3.8 g/100 g lipid, such as from 3.8-3.9 g/100 g lipid, for example 3.9-4.0 g/100 g lipid, such as from 4.0-4.1 g/100 g lipid, for example 4.1-4.2 g/100 g lipid, such as from 4.2-4.3 g/100 g lipid, for example 4.3-4.4 g/100 g lipid, such as from 4.4-4.5 g/100 g lipid, for example 4.5-4.6 g/100 g lipid, such as from 4.6-4.7 g/100 g lipid, for example 4.7-4.8 g/100 g lipid, such as from 4.8-4.9 g/100 g lipid, for example 4.9-5.0 g/100 g lipid, such as from 5.0-5.1 g/100 g lipid, for example 5.1-5.2 g/100 g lipid, such as from 5.2-5.3 g/100 g lipid, for example 5.3-5.4 g/100 g lipid, such as from 5.4-5.5 g/100 g lipid, for example 5.5-5.6 g/100 g lipid, such as from 5.6-5.7 g/100 g lipid, for example 5.7-5.8 g/100 g lipid, such as from 5.8-5.9 g/100 g lipid, for example 5.9-6.0 g/100 g lipid, such as from 6.0-6.1 g/100 g lipid, for example 6.1-6.2 g/100 g lipid, such as from 6.2-6.3 g/100 g lipid, for example 6.3-6.4 g/100 g lipid, such as from 6.4-6.5 g/100 g lipid, for example 6.5-6.6 g/100 g lipid, such as from 6.6-6.7 g/100 g lipid, for example 6.7-6.8 g/100 g lipid, such as from 6.8-6.9 g/100 g lipid, for example 6.9-7.0 g/100 g lipid, such as from 7.0-7.1 g/100 g lipid, for example 7.1-7.2 g/100 g lipid, such as from 7.2-7.3 g/100 g lipid, for example 7.3-7.4 g/100 g lipid, such as from 7.4-7.5 g/100 g lipid, for example 7.5-7.6 g/100 g lipid, such as from 7.6-7.7 g/100 g lipid, for example 7.7-7.8 g/100 g lipid, such as from 7.8-7.9 g/100 g lipid, for example 7.9-8.0 g/100 g lipid, such as from 8.0-8.1 g/100 g lipid, for example 8.1-8.2 g/100 g lipid, such as from 8.2-8.3 g/100 g lipid, for example 8.3-8.4 g/100 g lipid, such as from 8.4-8.5 g/100 g lipid, for example 8.5-8.6 g/100 g lipid, such as from 8.6-8.7 g/100 g lipid, for example 8.7-8.8 g/100 g lipid, such as from 8.8-8.9 g/100 g lipid, for example 8.9-9.0 g/100 g lipid, such as from 9.0-9.1 g/100 g lipid, for example 9.1-9.2 g/100 g lipid, such as from 9.2-9.3 g/100 g lipid, for example 9.3-9.4 g/100 g lipid, such as from 9.4-9.5 g/100 g lipid, for example 9.5-9.6 g/100 g lipid, such as from 9.6-9.7 g/100 g lipid, for example 9.7-9.8 g/100 g lipid, such as from 9.8-9.9 g/100 g lipid, for example 9.9-10.0 g/100 g lipid, such as from 10.0-10.1 g/100 g lipid, for example 10.1-10.2 g/100 g lipid, such as from 10.2-10.3 g/100 g lipid, for example 10.3-10.4 g/100 g lipid, such as from 10.4-10.5 g/100 g lipid, for example 10.5-10.6 g/100 g lipid, such as from 10.6-10.7 g/100 g lipid, for example 10.7-10.8 g/100 g lipid, such as from 10.8-10.9 g/100 g lipid, for example 10.9-11.0 g/100 g lipid, such as from 11.0-11.1 g/100 g lipid, for example 11.1-11.2 g/100 g lipid, such as from 11.2-11.3 g/100 g lipid, for example 11.3-11.4 g/100 g lipid, such as from 11.4-11.5 g/100 g lipid, for example 11.5-11.6 g/100 g lipid, such as from 11.6-11.7 g/100 g lipid, for example 11.7-11.8 g/100 g lipid, such as from 11.8-11.9 g/100 g lipid, for example 11.9-12.0 g/100 g lipid, such as from 12.0-12.1 g/100 g lipid, for example 12.1-12.2 g/100 g lipid, such as from 12.2-12.3 g/100 g lipid, for example 12.3-12.4 g/100 g lipid, such as from 12.4-12.5 g/100 g lipid, for example 12.5-12.6 g/100 g lipid, such as from 12.6-12.7 g/100 g lipid, for example 12.7-12.8 g/100 g lipid, such as from 12.8-12.9 g/100 g lipid, for example 12.9-13.0 g/100 g lipid, such as from 13.0-13.1 g/100 g lipid, for example 13.1-13.2 g/100 g lipid, such as from 13.2-13.3 g/100 g lipid, for example 13.3-13.4 g/100 g lipid, such as from 13.4-13.5 g/100 g lipid, for example 13.5-13.6 g/100 g lipid, such as from 13.6-13.7 g/100 g lipid, for example 13.7-13.8 g/100 g lipid, such as from 13.8-13.9 g/100 g lipid, for example 13.9-14.0 g/100 g lipid, such as from 14.0-14.1 g/100 g lipid, for example 14.1-14.2 g/100 g lipid, such as from 14.2-14.3 g/100 g lipid, for example 14.3-14.4 g/100 g lipid, such as from 14.4-14.5 g/100 g lipid, for example 14.5-14.6 g/100 g lipid, such as from 14.6-14.7 g/100 g lipid, for example 14.7-14.8 g/100 g lipid, such as from 14.8-14.9 g/100 g lipid, for example 14.9-15.0 g/100 g lipid, such as from 15-16 g/100 g lipid, for example 16-17 g/100 g lipid, such as from 17-18 g/100 g lipid, for example 18-19 g/100 g lipid, such as from 19-20 g/100 g lipid.

In one embodiment the present invention relates to a fish feed composition with a 16:2 n-4 content (g/100 g lipid) in the range from 0-20 g/100 g lipid, such as from 0-0.1 g/100 g lipid, for example 0.1-0.2 g/100 g lipid, such as from 0.2-0.3 g/100 g lipid, for example 0.3-0.4 g/100 g lipid, such as from 0.4-0.5 g/100 g lipid, for example 0.5-0.6 g/100 g lipid, such as from 0.6-0.7 g/100 g lipid, for example 0.7-0.8 g/100 g lipid, such as from 0.8-0.9 g/100 g lipid, for example 0.9-1.0 g/100 g lipid, such as from 1.0-1.1 g/100 g lipid, for example 1.1-1.2 g/100 g lipid, such as from 1.2-1.3 g/100 g lipid, for example 1.3-1.4 g/100 g lipid, such as from 1.4-1.5 g/100 g lipid, for example 1.5-1.6 g/100 g lipid, such as from 1.6-1.7 g/100 g lipid, for example 1.7-1.8 g/100 g lipid, such as from 1.8-1.9 g/100 g lipid, for example 1.9-2.0 g/100 g lipid, such as from 2.0-2.1 g/100 g lipid, for example 2.1-2.2 g/100 g lipid, such as from 2.2-2.3 g/100 g lipid, for example 2.3-2.4 g/100 g lipid, such as from 2.4-2.5 g/100 g lipid, for example 2.5-2.6 g/100 g lipid, such as from 2.6-2.7 g/100 g lipid, for example 2.7-2.8 g/100 g lipid, such as from 2.8-2.9 g/100 g lipid, for example 2.9-3.0 g/100 g lipid, such as from 3.0-3.1 g/100 g lipid, for example 3.1-3.2 g/100 g lipid, such as from 3.2-3.3 g/100 g lipid, for example 3.3-3.4 g/100 g lipid, such as from 3.4-3.5 g/100 g lipid, for example 3.5-3.6 g/100 g lipid, such as from 3.6-3.7 g/100 g lipid, for example 3.7-3.8 g/100 g lipid, such as from 3.8-3.9 g/100 g lipid, for example 3.9-4.0 g/100 g lipid, such as from 4.0-4.1 g/100 g lipid, for example 4.1-4.2 g/100 g lipid, such as from 4.2-4.3 g/100 g lipid, for example 4.3-4.4 g/100 g lipid, such as from 4.4-4.5 g/100 g lipid, for example 4.5-4.6 g/100 g lipid, such as from 4.6-4.7 g/100 g lipid, for example 4.7-4.8 g/100 g lipid, such as from 4.8-4.9 g/100 g lipid, for example 4.9-5.0 g/100 g lipid, such as from 5.0-5.1 g/100 g lipid, for example 5.1-5.2 g/100 g lipid, such as from 5.2-5.3 g/100 g lipid, for example 5.3-5.4 g/100 g lipid, such as from 5.4-5.5 g/100 g lipid, for example 5.5-5.6 g/100 g lipid, such as from 5.6-5.7 g/100 g lipid, for example 5.7-5.8 g/100 g lipid, such as from 5.8-5.9 g/100 g lipid, for example 5.9-6.0 g/100 g lipid, such as from 6.0-6.1 g/100 g lipid, for example 6.1-6.2 g/100 g lipid, such as from 6.2-6.3 g/100 g lipid, for example 6.3-6.4 g/100 g lipid, such as from 6.4-6.5 g/100 g lipid, for example 6.5-6.6 g/100 g lipid, such as from 6.6-6.7 g/100 g lipid, for example 6.7-6.8 g/100 g lipid, such as from 6.8-6.9 g/100 g lipid, for example 6.9-7.0 g/100 g lipid, such as from 7.0-7.1 g/100 g lipid, for example 7.1-7.2 g/100 g lipid, such as from 7.2-7.3 g/100 g lipid, for example 7.3-7.4 g/100 g lipid, such as from 7.4-7.5 g/100 g lipid, for example 7.5-7.6 g/100 g lipid, such as from 7.6-7.7 g/100 g lipid, for example 7.7-7.8 g/100 g lipid, such as from 7.8-7.9 g/100 g lipid, for example 7.9-8.0 g/100 g lipid, such as from 8.0-8.1 g/100 g lipid, for example 8.1-8.2 g/100 g lipid, such as from 8.2-8.3 g/100 g lipid, for example 8.3-8.4 g/100 g lipid, such as from 8.4-8.5 g/100 g lipid, for example 8.5-8.6 g/100 g lipid, such as from 8.6-8.7 g/100 g lipid, for example 8.7-8.8 g/100 g lipid, such as from 8.8-8.9 g/100 g lipid, for example 8.9-9.0 g/100 g lipid, such as from 9.0-9.1 g/100 g lipid, for example 9.1-9.2 g/100 g lipid, such as from 9.2-9.3 g/100 g lipid, for example 9.3-9.4 g/100 g lipid, such as from 9.4-9.5 g/100 g lipid, for example 9.5-9.6 g/100 g lipid, such as from 9.6-9.7 g/100 g lipid, for example 9.7-9.8 g/100 g lipid, such as from 9.8-9.9 g/100 g lipid, for example 9.9-10.0 g/100 g lipid, such as from 10.0-10.1 g/100 g lipid, for example 10.1-10.2 g/100 g lipid, such as from 10.2-10.3 g/100 g lipid, for example 10.3-10.4 g/100 g lipid, such as from 10.4-10.5 g/100 g lipid, for example 10.5-10.6 g/100 g lipid, such as from 10.6-10.7 g/100 g lipid, for example 10.7-10.8 g/100 g lipid, such as from 10.8-10.9 g/100 g lipid, for example 10.9-11.0 g/100 g lipid, such as from 11.0-11.1 g/100 g lipid, for example 11.1-11.2 g/100 g lipid, such as from 11.2-11.3 g/100 g lipid, for example 11.3-11.4 g/100 g lipid, such as from 11.4-11.5 g/100 g lipid, for example 11.5-11.6 g/100 g lipid, such as from 11.6-11.7 g/100 g lipid, for example 11.7-11.8 g/100 g lipid, such as from 11.8-11.9 g/100 g lipid, for example 11.9-12.0 g/100 g lipid, such as from 12.0-12.1 g/100 g lipid, for example 12.1-12.2 g/100 g lipid, such as from 12.2-12.3 g/100 g lipid, for example 12.3-12.4 g/100 g lipid, such as from 12.4-12.5 g/100 g lipid, for example 12.5-12.6 g/100 g lipid, such as from 12.6-12.7 g/100 g lipid, for example 12.7-12.8 g/100 g lipid, such as from 12.8-12.9 g/100 g lipid, for example 12.9-13.0 g/100 g lipid, such as from 13.0-13.1 g/100 g lipid, for example 13.1-13.2 g/100 g lipid, such as from 13.2-13.3 g/100 g lipid, for example 13.3-13.4 g/100 g lipid, such as from 13.4-13.5 g/100 g lipid, for example 13.5-13.6 g/100 g lipid, such as from 13.6-13.7 g/100 g lipid, for example 13.7-13.8 g/100 g lipid, such as from 13.8-13.9 g/100 g lipid, for example 13.9-14.0 g/100 g lipid, such as from 14.0-14.1 g/100 g lipid, for example 14.1-14.2 g/100 g lipid, such as from 14.2-14.3 g/100 g lipid, for example 14.3-14.4 g/100 g lipid, such as from 14.4-14.5 g/100 g lipid, for example 14.5-14.6 g/100 g lipid, such as from 14.6-14.7 g/100 g lipid, for example 14.7-14.8 g/100 g lipid, such as from 14.8-14.9 g/100 g lipid, for example 14.9-15.0 g/100 g lipid, such as from 15-16 g/100 g lipid, for example 16-17 g/100 g lipid, such as from 17-18 g/100 g lipid, for example 18-19 g/100 g lipid, such as from 19-20 g/100 g lipid.

In one embodiment the present invention relates to a fish feed composition with a 16:3 n-4 content (g/100 g lipid) in the range from 0-20 g/100 g lipid, such as from 0-0.1 g/100 g lipid, for example 0.1-0.2 g/100 g lipid, such as from 0.2-0.3 g/100 g lipid, for example 0.3-0.4 g/100 g lipid, such as from 0.4-0.5 g/100 g lipid, for example 0.5-0.6 g/100 g lipid, such as from 0.6-0.7 g/100 g lipid, for example 0.7-0.8 g/100 g lipid, such as from 0.8-0.9 g/100 g lipid, for example 0.9-1.0 g/100 g lipid, such as from 1.0-1.1 g/100 g lipid, for example 1.1-1.2 g/100 g lipid, such as from 1.2-1.3 g/100 g lipid, for example 1.3-1.4 g/100 g lipid, such as from 1.4-1.5 g/100 g lipid, for example 1.5-1.6 g/100 g lipid, such as from 1.6-1.7 g/100 g lipid, for example 1.7-1.8 g/100 g lipid, such as from 1.8-1.9 g/100 g lipid, for example 1.9-2.0 g/100 g lipid, such as from 2.0-2.1 g/100 g lipid, for example 2.1-2.2 g/100 g lipid, such as from 2.2-2.3 g/100 g lipid, for example 2.3-2.4 g/100 g lipid, such as from 2.4-2.5 g/100 g lipid, for example 2.5-2.6 g/100 g lipid, such as from 2.6-2.7 g/100 g lipid, for example 2.7-2.8 g/100 g lipid, such as from 2.8-2.9 g/100 g lipid, for example 2.9-3.0 g/100 g lipid, such as from 3.0-3.1 g/100 g lipid, for example 3.1-3.2 g/100 g lipid, such as from 3.2-3.3 g/100 g lipid, for example 3.3-3.4 g/100 g lipid, such as from 3.4-3.5 g/100 g lipid, for example 3.5-3.6 g/100 g lipid, such as from 3.6-3.7 g/100 g lipid, for example 3.7-3.8 g/100 g lipid, such as from 3.8-3.9 g/100 g lipid, for example 3.9-4.0 g/100 g lipid, such as from 4.0-4.1 g/100 g lipid, for example 4.1-4.2 g/100 g lipid, such as from 4.2-4.3 g/100 g lipid, for example 4.3-4.4 g/100 g lipid, such as from 4.4-4.5 g/100 g lipid, for example 4.5-4.6 g/100 g lipid, such as from 4.6-4.7 g/100 g lipid, for example 4.7-4.8 g/100 g lipid, such as from 4.8-4.9 g/100 g lipid, for example 4.9-5.0 g/100 g lipid, such as from 5.0-5.1 g/100 g lipid, for example 5.1-5.2 g/100 g lipid, such as from 5.2-5.3 g/100 g lipid, for example 5.3-5.4 g/100 g lipid, such as from 5.4-5.5 g/100 g lipid, for example 5.5-5.6 g/100 g lipid, such as from 5.6-5.7 g/100 g lipid, for example 5.7-5.8 g/100 g lipid, such as from 5.8-5.9 g/100 g lipid, for example 5.9-6.0 g/100 g lipid, such as from 6.0-6.1 g/100 g lipid, for example 6.1-6.2 g/100 g lipid, such as from 6.2-6.3 g/100 g lipid, for example 6.3-6.4 g/100 g lipid, such as from 6.4-6.5 g/100 g lipid, for example 6.5-6.6 g/100 g lipid, such as from 6.6-6.7 g/100 g lipid, for example 6.7-6.8 g/100 g lipid, such as from 6.8-6.9 g/100 g lipid, for example 6.9-7.0 g/100 g lipid, such as from 7.0-7.1 g/100 g lipid, for example 7.1-7.2 g/100 g lipid, such as from 7.2-7.3 g/100 g lipid, for example 7.3-7.4 g/100 g lipid, such as from 7.4-7.5 g/100 g lipid, for example 7.5-7.6 g/100 g lipid, such as from 7.6-7.7 g/100 g lipid, for example 7.7-7.8 g/100 g lipid, such as from 7.8-7.9 g/100 g lipid, for example 7.9-8.0 g/100 g lipid, such as from 8.0-8.1 g/100 g lipid, for example 8.1-8.2 g/100 g lipid, such as from 8.2-8.3 g/100 g lipid, for example 8.3-8.4 g/100 g lipid, such as from 8.4-8.5 g/100 g lipid, for example 8.5-8.6 g/100 g lipid, such as from 8.6-8.7 g/100 g lipid, for example 8.7-8.8 g/100 g lipid, such as from 8.8-8.9 g/100 g lipid, for example 8.9-9.0 g/100 g lipid, such as from 9.0-9.1 g/100 g lipid, for example 9.1-9.2 g/100 g lipid, such as from 9.2-9.3 g/100 g lipid, for example 9.3-9.4 g/100 g lipid, such as from 9.4-9.5 g/100 g lipid, for example 9.5-9.6 g/100 g lipid, such as from 9.6-9.7 g/100 g lipid, for example 9.7-9.8 g/100 g lipid, such as from 9.8-9.9 g/100 g lipid, for example 9.9-10.0 g/100 g lipid, such as from 10.0-10.1 g/100 g lipid, for example 10.1-10.2 g/100 g lipid, such as from 10.2-10.3 g/100 g lipid, for example 10.3-10.4 g/100 g lipid, such as from 10.4-10.5 g/100 g lipid, for example 10.5-10.6 g/100 g lipid, such as from 10.6-10.7 g/100 g lipid, for example 10.7-10.8 g/100 g lipid, such as from 10.8-10.9 g/100 g lipid, for example 10.9-11.0 g/100 g lipid, such as from 11.0-11.1 g/100 g lipid, for example 11.1-11.2 g/100 g lipid, such as from 11.2-11.3 g/100 g lipid, for example 11.3-11.4 g/100 g lipid, such as from 11.4-11.5 g/100 g lipid, for example 11.5-11.6 g/100 g lipid, such as from 11.6-11.7 g/100 g lipid, for example 11.7-11.8 g/100 g lipid, such as from 11.8-11.9 g/100 g lipid, for example 11.9-12.0 g/100 g lipid, such as from 12.0-12.1 g/100 g lipid, for example 12.1-12.2 g/100 g lipid, such as from 12.2-12.3 g/100 g lipid, for example 12.3-12.4 g/100 g lipid, such as from 12.4-12.5 g/100 g lipid, for example 12.5-12.6 g/100 g lipid, such as from 12.6-12.7 g/100 g lipid, for example 12.7-12.8 g/100 g lipid, such as from 12.8-12.9 g/100 g lipid, for example 12.9-13.0 g/100 g lipid, such as from 13.0-13.1 g/100 g lipid, for example 13.1-13.2 g/100 g lipid, such as from 13.2-13.3 g/100 g lipid, for example 13.3-13.4 g/100 g lipid, such as from 13.4-13.5 g/100 g lipid, for example 13.5-13.6 g/100 g lipid, such as from 13.6-13.7 g/100 g lipid, for example 13.7-13.8 g/100 g lipid, such as from 13.8-13.9 g/100 g lipid, for example 13.9-14.0 g/100 g lipid, such as from 14.0-14.1 g/100 g lipid, for example 14.1-14.2 g/100 g lipid, such as from 14.2-14.3 g/100 g lipid, for example 14.3-14.4 g/100 g lipid, such as from 14.4-14.5 g/100 g lipid, for example 14.5-14.6 g/100 g lipid, such as from 14.6-14.7 g/100 g lipid, for example 14.7-14.8 g/100 g lipid, such as from 14.8-14.9 g/100 g lipid, for example 14.9-15.0 g/100 g lipid, such as from 15-16 g/100 g lipid, for example 16-17 g/100 g lipid, such as from 17-18 g/100 g lipid, for example 18-19 g/100 g lipid, such as from 19-20 g/100 g lipid.

In one embodiment the present invention relates to a fish feed composition with a 16:4 n-1 content (g/100 g lipid) in the range from 0-20 g/100 g lipid, such as from 0-0.1 g/100 g lipid, for example 0.1-0.2 g/100 g lipid, such as from 0.2-0.3 g/100 g lipid, for example 0.3-0.4 g/100 g lipid, such as from 0.4-0.5 g/100 g lipid, for example 0.5-0.6 g/100 g lipid, such as from 0.6-0.7 g/100 g lipid, for example 0.7-0.8 g/100 g lipid, such as from 0.8-0.9 g/100 g lipid, for example 0.9-1.0 g/100 g lipid, such as from 1.0-1.1 g/100 g lipid, for example 1.1-1.2 g/100 g lipid, such as from 1.2-1.3 g/100 g lipid, for example 1.3-1.4 g/100 g lipid, such as from 1.4-1.5 g/100 g lipid, for example 1.5-1.6 g/100 g lipid, such as from 1.6-1.7 g/100 g lipid, for example 1.7-1.8 g/100 g lipid, such as from 1.8-1.9 g/100 g lipid, for example 1.9-2.0 g/100 g lipid, such as from 2.0-2.1 g/100 g lipid, for example 2.1-2.2 g/100 g lipid, such as from 2.2-2.3 g/100 g lipid, for example 2.3-2.4 g/100 g lipid, such as from 2.4-2.5 g/100 g lipid, for example 2.5-2.6 g/100 g lipid, such as from 2.6-2.7 g/100 g lipid, for example 2.7-2.8 g/100 g lipid, such as from 2.8-2.9 g/100 g lipid, for example 2.9-3.0 g/100 g lipid, such as from 3.0-3.1 g/100 g lipid, for example 3.1-3.2 g/100 g lipid, such as from 3.2-3.3 g/100 g lipid, for example 3.3-3.4 g/100 g lipid, such as from 3.4-3.5 g/100 g lipid, for example 3.5-3.6 g/100 g lipid, such as from 3.6-3.7 g/100 g lipid, for example 3.7-3.8 g/100 g lipid, such as from 3.8-3.9 g/100 g lipid, for example 3.9-4.0 g/100 g lipid, such as from 4.0-4.1 g/100 g lipid, for example 4.1-4.2 g/100 g lipid, such as from 4.2-4.3 g/100 g lipid, for example 4.3-4.4 g/100 g lipid, such as from 4.4-4.5 g/100 g lipid, for example 4.5-4.6 g/100 g lipid, such as from 4.6-4.7 g/100 g lipid, for example 4.7-4.8 g/100 g lipid, such as from 4.8-4.9 g/100 g lipid, for example 4.9-5.0 g/100 g lipid, such as from 5.0-5.1 g/100 g lipid, for example 5.1-5.2 g/100 g lipid, such as from 5.2-5.3 g/100 g lipid, for example 5.3-5.4 g/100 g lipid, such as from 5.4-5.5 g/100 g lipid, for example 5.5-5.6 g/100 g lipid, such as from 5.6-5.7 g/100 g lipid, for example 5.7-5.8 g/100 g lipid, such as from 5.8-5.9 g/100 g lipid, for example 5.9-6.0 g/100 g lipid, such as from 6.0-6.1 g/100 g lipid, for example 6.1-6.2 g/100 g lipid, such as from 6.2-6.3 g/100 g lipid, for example 6.3-6.4 g/100 g lipid, such as from 6.4-6.5 g/100 g lipid, for example 6.5-6.6 g/100 g lipid, such as from 6.6-6.7 g/100 g lipid, for example 6.7-6.8 g/100 g lipid, such as from 6.8-6.9 g/100 g lipid, for example 6.9-7.0 g/100 g lipid, such as from 7.0-7.1 g/100 g lipid, for example 7.1-7.2 g/100 g lipid, such as from 7.2-7.3 g/100 g lipid, for example 7.3-7.4 g/100 g lipid, such as from 7.4-7.5 g/100 g lipid, for example 7.5-7.6 g/100 g lipid, such as from 7.6-7.7 g/100 g lipid, for example 7.7-7.8 g/100 g lipid, such as from 7.8-7.9 g/100 g lipid, for example 7.9-8.0 g/100 g lipid, such as from 8.0-8.1 g/100 g lipid, for example 8.1-8.2 g/100 g lipid, such as from 8.2-8.3 g/100 g lipid, for example 8.3-8.4 g/100 g lipid, such as from 8.4-8.5 g/100 g lipid, for example 8.5-8.6 g/100 g lipid, such as from 8.6-8.7 g/100 g lipid, for example 8.7-8.8 g/100 g lipid, such as from 8.8-8.9 g/100 g lipid, for example 8.9-9.0 g/100 g lipid, such as from 9.0-9.1 g/100 g lipid, for example 9.1-9.2 g/100 g lipid, such as from 9.2-9.3 g/100 g lipid, for example 9.3-9.4 g/100 g lipid, such as from 9.4-9.5 g/100 g lipid, for example 9.5-9.6 g/100 g lipid, such as from 9.6-9.7 g/100 g lipid, for example 9.7-9.8 g/100 g lipid, such as from 9.8-9.9 g/100 g lipid, for example 9.9-10.0 g/100 g lipid, such as from 10.0-10.1 g/100 g lipid, for example 10.1-10.2 g/100 g lipid, such as from 10.2-10.3 g/100 g lipid, for example 10.3-10.4 g/100 g lipid, such as from 10.4-10.5 g/100 g lipid, for example 10.5-10.6 g/100 g lipid, such as from 10.6-10.7 g/100 g lipid, for example 10.7-10.8 g/100 g lipid, such as from 10.8-10.9 g/100 g lipid, for example 10.9-11.0 g/100 g lipid, such as from 11.0-11.1 g/100 g lipid, for example 11.1-11.2 g/100 g lipid, such as from 11.2-11.3 g/100 g lipid, for example 11.3-11.4 g/100 g lipid, such as from 11.4-11.5 g/100 g lipid, for example 11.5-11.6 g/100 g lipid, such as from 11.6-11.7 g/100 g lipid, for example 11.7-11.8 g/100 g lipid, such as from 11.8-11.9 g/100 g lipid, for example 11.9-12.0 g/100 g lipid, such as from 12.0-12.1 g/100 g lipid, for example 12.1-12.2 g/100 g lipid, such as from 12.2-12.3 g/100 g lipid, for example 12.3-12.4 g/100 g lipid, such as from 12.4-12.5 g/100 g lipid, for example 12.5-12.6 g/100 g lipid, such as from 12.6-12.7 g/100 g lipid, for example 12.7-12.8 g/100 g lipid, such as from 12.8-12.9 g/100 g lipid, for example 12.9-13.0 g/100 g lipid, such as from 13.0-13.1 g/100 g lipid, for example 13.1-13.2 g/100 g lipid, such as from 13.2-13.3 g/100 g lipid, for example 13.3-13.4 g/100 g lipid, such as from 13.4-13.5 g/100 g lipid, for example 13.5-13.6 g/100 g lipid, such as from 13.6-13.7 g/100 g lipid, for example 13.7-13.8 g/100 g lipid, such as from 13.8-13.9 g/100 g lipid, for example 13.9-14.0 g/100 g lipid, such as from 14.0-14.1 g/100 g lipid, for example 14.1-14.2 g/100 g lipid, such as from 14.2-14.3 g/100 g lipid, for example 14.3-14.4 g/100 g lipid, such as from 14.4-14.5 g/100 g lipid, for example 14.5-14.6 g/100 g lipid, such as from 14.6-14.7 g/100 g lipid, for example 14.7-14.8 g/100 g lipid, such as from 14.8-14.9 g/100 g lipid, for example 14.9-15.0 g/100 g lipid, such as from 15-16 g/100 g lipid, for example 16-17 g/100 g lipid, such as from 17-18 g/100 g lipid, for example 18-19 g/100 g lipid, such as from 19-20 g/100 g lipid.

In one embodiment the present invention relates to a fish feed composition with a 18:2 n-6 content (g/100 g lipid) in the range from 0-20 g/100 g lipid, such as from 0-0.1 g/100 g lipid, for example 0.1-0.2 g/100 g lipid, such as from 0.2-0.3 g/100 g lipid, for example 0.3-0.4 g/100 g lipid, such as from 0.4-0.5 g/100 g lipid, for example 0.5-0.6 g/100 g lipid, such as from 0.6-0.7 g/100 g lipid, for example 0.7-0.8 g/100 g lipid, such as from 0.8-0.9 g/100 g lipid, for example 0.9-1.0 g/100 g lipid, such as from 1.0-1.1 g/100 g lipid, for example 1.1-1.2 g/100 g lipid, such as from 1.2-1.3 g/100 g lipid, for example 1.3-1.4 g/100 g lipid, such as from 1.4-1.5 g/100 g lipid, for example 1.5-1.6 g/100 g lipid, such as from 1.6-1.7 g/100 g lipid, for example 1.7-1.8 g/100 g lipid, such as from 1.8-1.9 g/100 g lipid, for example 1.9-2.0 g/100 g lipid, such as from 2.0-2.1 g/100 g lipid, for example 2.1-2.2 g/100 g lipid, such as from 2.2-2.3 g/100 g lipid, for example 2.3-2.4 g/100 g lipid, such as from 2.4-2.5 g/100 g lipid, for example 2.5-2.6 g/100 g lipid, such as from 2.6-2.7 g/100 g lipid, for example 2.7-2.8 g/100 g lipid, such as from 2.8-2.9 g/100 g lipid, for example 2.9-3.0 g/100 g lipid, such as from 3.0-3.1 g/100 g lipid, for example 3.1-3.2 g/100 g lipid, such as from 3.2-3.3 g/100 g lipid, for example 3.3-3.4 g/100 g lipid, such as from 3.4-3.5 g/100 g lipid, for example 3.5-3.6 g/100 g lipid, such as from 3.6-3.7 g/100 g lipid, for example 3.7-3.8 g/100 g lipid, such as from 3.8-3.9 g/100 g lipid, for example 3.9-4.0 g/100 g lipid, such as from 4.0-4.1 g/100 g lipid, for example 4.1-4.2 g/100 g lipid, such as from 4.2-4.3 g/100 g lipid, for example 4.3-4.4 g/100 g lipid, such as from 4.4-4.5 g/100 g lipid, for example 4.5-4.6 g/100 g lipid, such as from 4.6-4.7 g/100 g lipid, for example 4.7-4.8 g/100 g lipid, such as from 4.8-4.9 g/100 g lipid, for example 4.9-5.0 g/100 g lipid, such as from 5.0-5.1 g/100 g lipid, for example 5.1-5.2 g/100 g lipid, such as from 5.2-5.3 g/100 g lipid, for example 5.3-5.4 g/100 g lipid, such as from 5.4-5.5 g/100 g lipid, for example 5.5-5.6 g/100 g lipid, such as from 5.6-5.7 g/100 g lipid, for example 5.7-5.8 g/100 g lipid, such as from 5.8-5.9 g/100 g lipid, for example 5.9-6.0 g/100 g lipid, such as from 6.0-6.1 g/100 g lipid, for example 6.1-6.2 g/100 g lipid, such as from 6.2-6.3 g/100 g lipid, for example 6.3-6.4 g/100 g lipid, such as from 6.4-6.5 g/100 g lipid, for example 6.5-6.6 g/100 g lipid, such as from 6.6-6.7 g/100 g lipid, for example 6.7-6.8 g/100 g lipid, such as from 6.8-6.9 g/100 g lipid, for example 6.9-7.0 g/100 g lipid, such as from 7.0-7.1 g/100 g lipid, for example 7.1-7.2 g/100 g lipid, such as from 7.2-7.3 g/100 g lipid, for example 7.3-7.4 g/100 g lipid, such as from 7.4-7.5 g/100 g lipid, for example 7.5-7.6 g/100 g lipid, such as from 7.6-7.7 g/100 g lipid, for example 7.7-7.8 g/100 g lipid, such as from 7.8-7.9 g/100 g lipid, for example 7.9-8.0 g/100 g lipid, such as from 8.0-8.1 g/100 g lipid, for example 8.1-8.2 g/100 g lipid, such as from 8.2-8.3 g/100 g lipid, for example 8.3-8.4 g/100 g lipid, such as from 8.4-8.5 g/100 g lipid, for example 8.5-8.6 g/100 g lipid, such as from 8.6-8.7 g/100 g lipid, for example 8.7-8.8 g/100 g lipid, such as from 8.8-8.9 g/100 g lipid, for example 8.9-9.0 g/100 g lipid, such as from 9.0-9.1 g/100 g lipid, for example 9.1-9.2 g/100 g lipid, such as from 9.2-9.3 g/100 g lipid, for example 9.3-9.4 g/100 g lipid, such as from 9.4-9.5 g/100 g lipid, for example 9.5-9.6 g/100 g lipid, such as from 9.6-9.7 g/100 g lipid, for example 9.7-9.8 g/100 g lipid, such as from 9.8-9.9 g/100 g lipid, for example 9.9-10.0 g/100 g lipid, such as from 10.0-10.1 g/100 g lipid, for example 10.1-10.2 g/100 g lipid, such as from 10.2-10.3 g/100 g lipid, for example 10.3-10.4 g/100 g lipid, such as from 10.4-10.5 g/100 g lipid, for example 10.5-10.6 g/100 g lipid, such as from 10.6-10.7 g/100 g lipid, for example 10.7-10.8 g/100 g lipid, such as from 10.8-10.9 g/100 g lipid, for example 10.9-11.0 g/100 g lipid, such as from 11.0-11.1 g/100 g lipid, for example 11.1-11.2 g/100 g lipid, such as from 11.2-11.3 g/100 g lipid, for example 11.3-11.4 g/100 g lipid, such as from 11.4-11.5 g/100 g lipid, for example 11.5-11.6 g/100 g lipid, such as from 11.6-11.7 g/100 g lipid, for example 11.7-11.8 g/100 g lipid, such as from 11.8-11.9 g/100 g lipid, for example 11.9-12.0 g/100 g lipid, such as from 12.0-12.1 g/100 g lipid, for example 12.1-12.2 g/100 g lipid, such as from 12.2-12.3 g/100 g lipid, for example 12.3-12.4 g/100 g lipid, such as from 12.4-12.5 g/100 g lipid, for example 12.5-12.6 g/100 g lipid, such as from 12.6-12.7 g/100 g lipid, for example 12.7-12.8 g/100 g lipid, such as from 12.8-12.9 g/100 g lipid, for example 12.9-13.0 g/100 g lipid, such as from 13.0-13.1 g/100 g lipid, for example 13.1-13.2 g/100 g lipid, such as from 13.2-13.3 g/100 g lipid, for example 13.3-13.4 g/100 g lipid, such as from 13.4-13.5 g/100 g lipid, for example 13.5-13.6 g/100 g lipid, such as from 13.6-13.7 g/100 g lipid, for example 13.7-13.8 g/100 g lipid, such as from 13.8-13.9 g/100 g lipid, for example 13.9-14.0 g/100 g lipid, such as from 14.0-14.1 g/100 g lipid, for example 14.1-14.2 g/100 g lipid, such as from 14.2-14.3 g/100 g lipid, for example 14.3-14.4 g/100 g lipid, such as from 14.4-14.5 g/100 g lipid, for example 14.5-14.6 g/100 g lipid, such as from 14.6-14.7 g/100 g lipid, for example 14.7-14.8 g/100 g lipid, such as from 14.8-14.9 g/100 g lipid, for example 14.9-15.0 g/100 g lipid, such as from 15-16 g/100 g lipid, for example 16-17 g/100 g lipid, such as from 17-18 g/100 g lipid, for example 18-19 g/100 g lipid, such as from 19-20 g/100 g lipid.

In one embodiment the present invention relates to a fish feed composition with a 18:3 n-6 content (g/100 g lipid) in the range from 0-20 g/100 g lipid, such as from 0-0.1 g/100 g lipid, for example 0.1-0.2 g/100 g lipid, such as from 0.2-0.3 g/100 g lipid, for example 0.3-0.4 g/100 g lipid, such as from 0.4-0.5 g/100 g lipid, for example 0.5-0.6 g/100 g lipid, such as from 0.6-0.7 g/100 g lipid, for example 0.7-0.8 g/100 g lipid, such as from 0.8-0.9 g/100 g lipid, for example 0.9-1.0 g/100 g lipid, such as from 1.0-1.1 g/100 g lipid, for example 1.1-1.2 g/100 g lipid, such as from 1.2-1.3 g/100 g lipid, for example 1.3-1.4 g/100 g lipid, such as from 1.4-1.5 g/100 g lipid, for example 1.5-1.6 g/100 g lipid, such as from 1.6-1.7 g/100 g lipid, for example 1.7-1.8 g/100 g lipid, such as from 1.8-1.9 g/100 g lipid, for example 1.9-2.0 g/100 g lipid, such as from 2.0-2.1 g/100 g lipid, for example 2.1-2.2 g/100 g lipid, such as from 2.2-2.3 g/100 g lipid, for example 2.3-2.4 g/100 g lipid, such as from 2.4-2.5 g/100 g lipid, for example 2.5-2.6 g/100 g lipid, such as from 2.6-2.7 g/100 g lipid, for example 2.7-2.8 g/100 g lipid, such as from 2.8-2.9 g/100 g lipid, for example 2.9-3.0 g/100 g lipid, such as from 3.0-3.1 g/100 g lipid, for example 3.1-3.2 g/100 g lipid, such as from 3.2-3.3 g/100 g lipid, for example 3.3-3.4 g/100 g lipid, such as from 3.4-3.5 g/100 g lipid, for example 3.5-3.6 g/100 g lipid, such as from 3.6-3.7 g/100 g lipid, for example 3.7-3.8 g/100 g lipid, such as from 3.8-3.9 g/100 g lipid, for example 3.9-4.0 g/100 g lipid, such as from 4.0-4.1 g/100 g lipid, for example 4.1-4.2 g/100 g lipid, such as from 4.2-4.3 g/100 g lipid, for example 4.3-4.4 g/100 g lipid, such as from 4.4-4.5 g/100 g lipid, for example 4.5-4.6 g/100 g lipid, such as from 4.6-4.7 g/100 g lipid, for example 4.7-4.8 g/100 g lipid, such as from 4.8-4.9 g/100 g lipid, for example 4.9-5.0 g/100 g lipid, such as from 5.0-5.1 g/100 g lipid, for example 5.1-5.2 g/100 g lipid, such as from 5.2-5.3 g/100 g lipid, for example 5.3-5.4 g/100 g lipid, such as from 5.4-5.5 g/100 g lipid, for example 5.5-5.6 g/100 g lipid, such as from 5.6-5.7 g/100 g lipid, for example 5.7-5.8 g/100 g lipid, such as from 5.8-5.9 g/100 g lipid, for example 5.9-6.0 g/100 g lipid, such as from 6.0-6.1 g/100 g lipid, for example 6.1-6.2 g/100 g lipid, such as from 6.2-6.3 g/100 g lipid, for example 6.3-6.4 g/100 g lipid, such as from 6.4-6.5 g/100 g lipid, for example 6.5-6.6 g/100 g lipid, such as from 6.6-6.7 g/100 g lipid, for example 6.7-6.8 g/100 g lipid, such as from 6.8-6.9 g/100 g lipid, for example 6.9-7.0 g/100 g lipid, such as from 7.0-7.1 g/100 g lipid, for example 7.1-7.2 g/100 g lipid, such as from 7.2-7.3 g/100 g lipid, for example 7.3-7.4 g/100 g lipid, such as from 7.4-7.5 g/100 g lipid, for example 7.5-7.6 g/100 g lipid, such as from 7.6-7.7 g/100 g lipid, for example 7.7-7.8 g/100 g lipid, such as from 7.8-7.9 g/100 g lipid, for example 7.9-8.0 g/100 g lipid, such as from 8.0-8.1 g/100 g lipid, for example 8.1-8.2 g/100 g lipid, such as from 8.2-8.3 g/100 g lipid, for example 8.3-8.4 g/100 g lipid, such as from 8.4-8.5 g/100 g lipid, for example 8.5-8.6 g/100 g lipid, such as from 8.6-8.7 g/100 g lipid, for example 8.7-8.8 g/100 g lipid, such as from 8.8-8.9 g/100 g lipid, for example 8.9-9.0 g/100 g lipid, such as from 9.0-9.1 g/100 g lipid, for example 9.1-9.2 g/100 g lipid, such as from 9.2-9.3 g/100 g lipid, for example 9.3-9.4 g/100 g lipid, such as from 9.4-9.5 g/100 g lipid, for example 9.5-9.6 g/100 g lipid, such as from 9.6-9.7 g/100 g lipid, for example 9.7-9.8 g/100 g lipid, such as from 9.8-9.9 g/100 g lipid, for example 9.9-10.0 g/100 g lipid, such as from 10.0-10.1 g/100 g lipid, for example 10.1-10.2 g/100 g lipid, such as from 10.2-10.3 g/100 g lipid, for example 10.3-10.4 g/100 g lipid, such as from 10.4-10.5 g/100 g lipid, for example 10.5-10.6 g/100 g lipid, such as from 10.6-10.7 g/100 g lipid, for example 10.7-10.8 g/100 g lipid, such as from 10.8-10.9 g/100 g lipid, for example 10.9-11.0 g/100 g lipid, such as from 11.0-11.1 g/100 g lipid, for example 11.1-11.2 g/100 g lipid, such as from 11.2-11.3 g/100 g lipid, for example 11.3-11.4 g/100 g lipid, such as from 11.4-11.5 g/100 g lipid, for example 11.5-11.6 g/100 g lipid, such as from 11.6-11.7 g/100 g lipid, for example 11.7-11.8 g/100 g lipid, such as from 11.8-11.9 g/100 g lipid, for example 11.9-12.0 g/100 g lipid, such as from 12.0-12.1 g/100 g lipid, for example 12.1-12.2 g/100 g lipid, such as from 12.2-12.3 g/100 g lipid, for example 12.3-12.4 g/100 g lipid, such as from 12.4-12.5 g/100 g lipid, for example 12.5-12.6 g/100 g lipid, such as from 12.6-12.7 g/100 g lipid, for example 12.7-12.8 g/100 g lipid, such as from 12.8-12.9 g/100 g lipid, for example 12.9-13.0 g/100 g lipid, such as from 13.0-13.1 g/100 g lipid, for example 13.1-13.2 g/100 g lipid, such as from 13.2-13.3 g/100 g lipid, for example 13.3-13.4 g/100 g lipid, such as from 13.4-13.5 g/100 g lipid, for example 13.5-13.6 g/100 g lipid, such as from 13.6-13.7 g/100 g lipid, for example 13.7-13.8 g/100 g lipid, such as from 13.8-13.9 g/100 g lipid, for example 13.9-14.0 g/100 g lipid, such as from 14.0-14.1 g/100 g lipid, for example 14.1-14.2 g/100 g lipid, such as from 14.2-14.3 g/100 g lipid, for example 14.3-14.4 g/100 g lipid, such as from 14.4-14.5 g/100 g lipid, for example 14.5-14.6 g/100 g lipid, such as from 14.6-14.7 g/100 g lipid, for example 14.7-14.8 g/100 g lipid, such as from 14.8-14.9 g/100 g lipid, for example 14.9-15.0 g/100 g lipid, such as from 15-16 g/100 g lipid, for example 16-17 g/100 g lipid, such as from 17-18 g/100 g lipid, for example 18-19 g/100 g lipid, such as from 19-20 g/100 g lipid.

In one embodiment the present invention relates to a fish feed composition with a 20:2 n-6 content (g/100 g lipid) in the range from 0-20 g/100 g lipid, such as from 0-0.1 g/100 g lipid, for example 0.1-0.2 g/100 g lipid, such as from 0.2-0.3 g/100 g lipid, for example 0.3-0.4 g/100 g lipid, such as from 0.4-0.5 g/100 g lipid, for example 0.5-0.6 g/100 g lipid, such as from 0.6-0.7 g/100 g lipid, for example 0.7-0.8 g/100 g lipid, such as from 0.8-0.9 g/100 g lipid, for example 0.9-1.0 g/100 g lipid, such as from 1.0-1.1 g/100 g lipid, for example 1.1-1.2 g/100 g lipid, such as from 1.2-1.3 g/100 g lipid, for example 1.3-1.4 g/100 g lipid, such as from 1.4-1.5 g/100 g lipid, for example 1.5-1.6 g/100 g lipid, such as from 1.6-1.7 g/100 g lipid, for example 1.7-1.8 g/100 g lipid, such as from 1.8-1.9 g/100 g lipid, for example 1.9-2.0 g/100 g lipid, such as from 2.0-2.1 g/100 g lipid, for example 2.1-2.2 g/100 g lipid, such as from 2.2-2.3 g/100 g lipid, for example 2.3-2.4 g/100 g lipid, such as from 2.4-2.5 g/100 g lipid, for example 2.5-2.6 g/100 g lipid, such as from 2.6-2.7 g/100 g lipid, for example 2.7-2.8 g/100 g lipid, such as from 2.8-2.9 g/100 g lipid, for example 2.9-3.0 g/100 g lipid, such as from 3.0-3.1 g/100 g lipid, for example 3.1-3.2 g/100 g lipid, such as from 3.2-3.3 g/100 g lipid, for example 3.3-3.4 g/100 g lipid, such as from 3.4-3.5 g/100 g lipid, for example 3.5-3.6 g/100 g lipid, such as from 3.6-3.7 g/100 g lipid, for example 3.7-3.8 g/100 g lipid, such as from 3.8-3.9 g/100 g lipid, for example 3.9-4.0 g/100 g lipid, such as from 4.0-4.1 g/100 g lipid, for example 4.1-4.2 g/100 g lipid, such as from 4.2-4.3 g/100 g lipid, for example 4.3-4.4 g/100 g lipid, such as from 4.4-4.5 g/100 g lipid, for example 4.5-4.6 g/100 g lipid, such as from 4.6-4.7 g/100 g lipid, for example 4.7-4.8 g/100 g lipid, such as from 4.8-4.9 g/100 g lipid, for example 4.9-5.0 g/100 g lipid, such as from 5.0-5.1 g/100 g lipid, for example 5.1-5.2 g/100 g lipid, such as from 5.2-5.3 g/100 g lipid, for example 5.3-5.4 g/100 g lipid, such as from 5.4-5.5 g/100 g lipid, for example 5.5-5.6 g/100 g lipid, such as from 5.6-5.7 g/100 g lipid, for example 5.7-5.8 g/100 g lipid, such as from 5.8-5.9 g/100 g lipid, for example 5.9-6.0 g/100 g lipid, such as from 6.0-6.1 g/100 g lipid, for example 6.1-6.2 g/100 g lipid, such as from 6.2-6.3 g/100 g lipid, for example 6.3-6.4 g/100 g lipid, such as from 6.4-6.5 g/100 g lipid, for example 6.5-6.6 g/100 g lipid, such as from 6.6-6.7 g/100 g lipid, for example 6.7-6.8 g/100 g lipid, such as from 6.8-6.9 g/100 g lipid, for example 6.9-7.0 g/100 g lipid, such as from 7.0-7.1 g/100 g lipid, for example 7.1-7.2 g/100 g lipid, such as from 7.2-7.3 g/100 g lipid, for example 7.3-7.4 g/100 g lipid, such as from 7.4-7.5 g/100 g lipid, for example 7.5-7.6 g/100 g lipid, such as from 7.6-7.7 g/100 g lipid, for example 7.7-7.8 g/100 g lipid, such as from 7.8-7.9 g/100 g lipid, for example 7.9-8.0 g/100 g lipid, such as from 8.0-8.1 g/100 g lipid, for example 8.1-8.2 g/100 g lipid, such as from 8.2-8.3 g/100 g lipid, for example 8.3-8.4 g/100 g lipid, such as from 8.4-8.5 g/100 g lipid, for example 8.5-8.6 g/100 g lipid, such as from 8.6-8.7 g/100 g lipid, for example 8.7-8.8 g/100 g lipid, such as from 8.8-8.9 g/100 g lipid, for example 8.9-9.0 g/100 g lipid, such as from 9.0-9.1 g/100 g lipid, for example 9.1-9.2 g/100 g lipid, such as from 9.2-9.3 g/100 g lipid, for example 9.3-9.4 g/100 g lipid, such as from 9.4-9.5 g/100 g lipid, for example 9.5-9.6 g/100 g lipid, such as from 9.6-9.7 g/100 g lipid, for example 9.7-9.8 g/100 g lipid, such as from 9.8-9.9 g/100 g lipid, for example 9.9-10.0 g/100 g lipid, such as from 10.0-10.1 g/100 g lipid, for example 10.1-10.2 g/100 g lipid, such as from 10.2-10.3 g/100 g lipid, for example 10.3-10.4 g/100 g lipid, such as from 10.4-10.5 g/100 g lipid, for example 10.5-10.6 g/100 g lipid, such as from 10.6-10.7 g/100 g lipid, for example 10.7-10.8 g/100 g lipid, such as from 10.8-10.9 g/100 g lipid, for example 10.9-11.0 g/100 g lipid, such as from 11.0-11.1 g/100 g lipid, for example 11.1-11.2 g/100 g lipid, such as from 11.2-11.3 g/100 g lipid, for example 11.3-11.4 g/100 g lipid, such as from 11.4-11.5 g/100 g lipid, for example 11.5-11.6 g/100 g lipid, such as from 11.6-11.7 g/100 g lipid, for example 11.7-11.8 g/100 g lipid, such as from 11.8-11.9 g/100 g lipid, for example 11.9-12.0 g/100 g lipid, such as from 12.0-12.1 g/100 g lipid, for example 12.1-12.2 g/100 g lipid, such as from 12.2-12.3 g/100 g lipid, for example 12.3-12.4 g/100 g lipid, such as from 12.4-12.5 g/100 g lipid, for example 12.5-12.6 g/100 g lipid, such as from 12.6-12.7 g/100 g lipid, for example 12.7-12.8 g/100 g lipid, such as from 12.8-12.9 g/100 g lipid, for example 12.9-13.0 g/100 g lipid, such as from 13.0-13.1 g/100 g lipid, for example 13.1-13.2 g/100 g lipid, such as from 13.2-13.3 g/100 g lipid, for example 13.3-13.4 g/100 g lipid, such as from 13.4-13.5 g/100 g lipid, for example 13.5-13.6 g/100 g lipid, such as from 13.6-13.7 g/100 g lipid, for example 13.7-13.8 g/100 g lipid, such as from 13.8-13.9 g/100 g lipid, for example 13.9-14.0 g/100 g lipid, such as from 14.0-14.1 g/100 g lipid, for example 14.1-14.2 g/100 g lipid, such as from 14.2-14.3 g/100 g lipid, for example 14.3-14.4 g/100 g lipid, such as from 14.4-14.5 g/100 g lipid, for example 14.5-14.6 g/100 g lipid, such as from 14.6-14.7 g/100 g lipid, for example 14.7-14.8 g/100 g lipid, such as from 14.8-14.9 g/100 g lipid, for example 14.9-15.0 g/100 g lipid, such as from 15-16 g/100 g lipid, for example 16-17 g/100 g lipid, such as from 17-18 g/100 g lipid, for example 18-19 g/100 g lipid, such as from 19-20 g/100 g lipid.

In one embodiment the present invention relates to a fish feed composition with a 20:3 n-6 content (g/100 g lipid) in the range from 0-20 g/100 g lipid, such as from 0-0.1 g/100 g lipid, for example 0.1-0.2 g/100 g lipid, such as from 0.2-0.3 g/100 g lipid, for example 0.3-0.4 g/100 g lipid, such as from 0.4-0.5 g/100 g lipid, for example 0.5-0.6 g/100 g lipid, such as from 0.6-0.7 g/100 g lipid, for example 0.7-0.8 g/100 g lipid, such as from 0.8-0.9 g/100 g lipid, for example 0.9-1.0 g/100 g lipid, such as from 1.0-1.1 g/100 g lipid, for example 1.1-1.2 g/100 g lipid, such as from 1.2-1.3 g/100 g lipid, for example 1.3-1.4 g/100 g lipid, such as from 1.4-1.5 g/100 g lipid, for example 1.5-1.6 g/100 g lipid, such as from 1.6-1.7 g/100 g lipid, for example 1.7-1.8 g/100 g lipid, such as from 1.8-1.9 g/100 g lipid, for example 1.9-2.0 g/100 g lipid, such as from 2.0-2.1 g/100 g lipid, for example 2.1-2.2 g/100 g lipid, such as from 2.2-2.3 g/100 g lipid, for example 2.3-2.4 g/100 g lipid, such as from 2.4-2.5 g/100 g lipid, for example 2.5-2.6 g/100 g lipid, such as from 2.6-2.7 g/100 g lipid, for example 2.7-2.8 g/100 g lipid, such as from 2.8-2.9 g/100 g lipid, for example 2.9-3.0 g/100 g lipid, such as from 3.0-3.1 g/100 g lipid, for example 3.1-3.2 g/100 g lipid, such as from 3.2-3.3 g/100 g lipid, for example 3.3-3.4 g/100 g lipid, such as from 3.4-3.5 g/100 g lipid, for example 3.5-3.6 g/100 g lipid, such as from 3.6-3.7 g/100 g lipid, for example 3.7-3.8 g/100 g lipid, such as from 3.8-3.9 g/100 g lipid, for example 3.9-4.0 g/100 g lipid, such as from 4.0-4.1 g/100 g lipid, for example 4.1-4.2 g/100 g lipid, such as from 4.2-4.3 g/100 g lipid, for example 4.3-4.4 g/100 g lipid, such as from 4.4-4.5 g/100 g lipid, for example 4.5-4.6 g/100 g lipid, such as from 4.6-4.7 g/100 g lipid, for example 4.7-4.8 g/100 g lipid, such as from 4.8-4.9 g/100 g lipid, for example 4.9-5.0 g/100 g lipid, such as from 5.0-5.1 g/100 g lipid, for example 5.1-5.2 g/100 g lipid, such as from 5.2-5.3 g/100 g lipid, for example 5.3-5.4 g/100 g lipid, such as from 5.4-5.5 g/100 g lipid, for example 5.5-5.6 g/100 g lipid, such as from 5.6-5.7 g/100 g lipid, for example 5.7-5.8 g/100 g lipid, such as from 5.8-5.9 g/100 g lipid, for example 5.9-6.0 g/100 g lipid, such as from 6.0-6.1 g/100 g lipid, for example 6.1-6.2 g/100 g lipid, such as from 6.2-6.3 g/100 g lipid, for example 6.3-6.4 g/100 g lipid, such as from 6.4-6.5 g/100 g lipid, for example 6.5-6.6 g/100 g lipid, such as from 6.6-6.7 g/100 g lipid, for example 6.7-6.8 g/100 g lipid, such as from 6.8-6.9 g/100 g lipid, for example 6.9-7.0 g/100 g lipid, such as from 7.0-7.1 g/100 g lipid, for example 7.1-7.2 g/100 g lipid, such as from 7.2-7.3 g/100 g lipid, for example 7.3-7.4 g/100 g lipid, such as from 7.4-7.5 g/100 g lipid, for example 7.5-7.6 g/100 g lipid, such as from 7.6-7.7 g/100 g lipid, for example 7.7-7.8 g/100 g lipid, such as from 7.8-7.9 g/100 g lipid, for example 7.9-8.0 g/100 g lipid, such as from 8.0-8.1 g/100 g lipid, for example 8.1-8.2 g/100 g lipid, such as from 8.2-8.3 g/100 g lipid, for example 8.3-8.4 g/100 g lipid, such as from 8.4-8.5 g/100 g lipid, for example 8.5-8.6 g/100 g lipid, such as from 8.6-8.7 g/100 g lipid, for example 8.7-8.8 g/100 g lipid, such as from 8.8-8.9 g/100 g lipid, for example 8.9-9.0 g/100 g lipid, such as from 9.0-9.1 g/100 g lipid, for example 9.1-9.2 g/100 g lipid, such as from 9.2-9.3 g/100 g lipid, for example 9.3-9.4 g/100 g lipid, such as from 9.4-9.5 g/100 g lipid, for example 9.5-9.6 g/100 g lipid, such as from 9.6-9.7 g/100 g lipid, for example 9.7-9.8 g/100 g lipid, such as from 9.8-9.9 g/100 g lipid, for example 9.9-10.0 g/100 g lipid, such as from 10.0-10.1 g/100 g lipid, for example 10.1-10.2 g/100 g lipid, such as from 10.2-10.3 g/100 g lipid, for example 10.3-10.4 g/100 g lipid, such as from 10.4-10.5 g/100 g lipid, for example 10.5-10.6 g/100 g lipid, such as from 10.6-10.7 g/100 g lipid, for example 10.7-10.8 g/100 g lipid, such as from 10.8-10.9 g/100 g lipid, for example 10.9-11.0 g/100 g lipid, such as from 11.0-11.1 g/100 g lipid, for example 11.1-11.2 g/100 g lipid, such as from 11.2-11.3 g/100 g lipid, for example 11.3-11.4 g/100 g lipid, such as from 11.4-11.5 g/100 g lipid, for example 11.5-11.6 g/100 g lipid, such as from 11.6-11.7 g/100 g lipid, for example 11.7-11.8 g/100 g lipid, such as from 11.8-11.9 g/100 g lipid, for example 11.9-12.0 g/100 g lipid, such as from 12.0-12.1 g/100 g lipid, for example 12.1-12.2 g/100 g lipid, such as from 12.2-12.3 g/100 g lipid, for example 12.3-12.4 g/100 g lipid, such as from 12.4-12.5 g/100 g lipid, for example 12.5-12.6 g/100 g lipid, such as from 12.6-12.7 g/100 g lipid, for example 12.7-12.8 g/100 g lipid, such as from 12.8-12.9 g/100 g lipid, for example 12.9-13.0 g/100 g lipid, such as from 13.0-13.1 g/100 g lipid, for example 13.1-13.2 g/100 g lipid, such as from 13.2-13.3 g/100 g lipid, for example 13.3-13.4 g/100 g lipid, such as from 13.4-13.5 g/100 g lipid, for example 13.5-13.6 g/100 g lipid, such as from 13.6-13.7 g/100 g lipid, for example 13.7-13.8 g/100 g lipid, such as from 13.8-13.9 g/100 g lipid, for example 13.9-14.0 g/100 g lipid, such as from 14.0-14.1 g/100 g lipid, for example 14.1-14.2 g/100 g lipid, such as from 14.2-14.3 g/100 g lipid, for example 14.3-14.4 g/100 g lipid, such as from 14.4-14.5 g/100 g lipid, for example 14.5-14.6 g/100 g lipid, such as from 14.6-14.7 g/100 g lipid, for example 14.7-14.8 g/100 g lipid, such as from 14.8-14.9 g/100 g lipid, for example 14.9-15.0 g/100 g lipid, such as from 15-16 g/100 g lipid, for example 16-17 g/100 g lipid, such as from 17-18 g/100 g lipid, for example 18-19 g/100 g lipid, such as from 19-20 g/100 g lipid.

In one embodiment the present invention relates to a fish feed composition with a 20:4 n-6 content (g/100 g lipid) in the range from 0-20 g/100 g lipid, such as from 0-0.1 g/100 g lipid, for example 0.1-0.2 g/100 g lipid, such as from 0.2-0.3 g/100 g lipid, for example 0.3-0.4 g/100 g lipid, such as from 0.4-0.5 g/100 g lipid, for example 0.5-0.6 g/100 g lipid, such as from 0.6-0.7 g/100 g lipid, for example 0.7-0.8 g/100 g lipid, such as from 0.8-0.9 g/100 g lipid, for example 0.9-1.0 g/100 g lipid, such as from 1.0-1.1 g/100 g lipid, for example 1.1-1.2 g/100 g lipid, such as from 1.2-1.3 g/100 g lipid, for example 1.3-1.4 g/100 g lipid, such as from 1.4-1.5 g/100 g lipid, for example 1.5-1.6 g/100 g lipid, such as from 1.6-1.7 g/100 g lipid, for example 1.7-1.8 g/100 g lipid, such as from 1.8-1.9 g/100 g lipid, for example 1.9-2.0 g/100 g lipid, such as from 2.0-2.1 g/100 g lipid, for example 2.1-2.2 g/100 g lipid, such as from 2.2-2.3 g/100 g lipid, for example 2.3-2.4 g/100 g lipid, such as from 2.4-2.5 g/100 g lipid, for example 2.5-2.6 g/100 g lipid, such as from 2.6-2.7 g/100 g lipid, for example 2.7-2.8 g/100 g lipid, such as from 2.8-2.9 g/100 g lipid, for example 2.9-3.0 g/100 g lipid, such as from 3.0-3.1 g/100 g lipid, for example 3.1-3.2 g/100 g lipid, such as from 3.2-3.3 g/100 g lipid, for example 3.3-3.4 g/100 g lipid, such as from 3.4-3.5 g/100 g lipid, for example 3.5-3.6 g/100 g lipid, such as from 3.6-3.7 g/100 g lipid, for example 3.7-3.8 g/100 g lipid, such as from 3.8-3.9 g/100 g lipid, for example 3.9-4.0 g/100 g lipid, such as from 4.0-4.1 g/100 g lipid, for example 4.1-4.2 g/100 g lipid, such as from 4.2-4.3 g/100 g lipid, for example 4.3-4.4 g/100 g lipid, such as from 4.4-4.5 g/100 g lipid, for example 4.5-4.6 g/100 g lipid, such as from 4.6-4.7 g/100 g lipid, for example 4.7-4.8 g/100 g lipid, such as from 4.8-4.9 g/100 g lipid, for example 4.9-5.0 g/100 g lipid, such as from 5.0-5.1 g/100 g lipid, for example 5.1-5.2 g/100 g lipid, such as from 5.2-5.3 g/100 g lipid, for example 5.3-5.4 g/100 g lipid, such as from 5.4-5.5 g/100 g lipid, for example 5.5-5.6 g/100 g lipid, such as from 5.6-5.7 g/100 g lipid, for example 5.7-5.8 g/100 g lipid, such as from 5.8-5.9 g/100 g lipid, for example 5.9-6.0 g/100 g lipid, such as from 6.0-6.1 g/100 g lipid, for example 6.1-6.2 g/100 g lipid, such as from 6.2-6.3 g/100 g lipid, for example 6.3-6.4 g/100 g lipid, such as from 6.4-6.5 g/100 g lipid, for example 6.5-6.6 g/100 g lipid, such as from 6.6-6.7 g/100 g lipid, for example 6.7-6.8 g/100 g lipid, such as from 6.8-6.9 g/100 g lipid, for example 6.9-7.0 g/100 g lipid, such as from 7.0-7.1 g/100 g lipid, for example 7.1-7.2 g/100 g lipid, such as from 7.2-7.3 g/100 g lipid, for example 7.3-7.4 g/100 g lipid, such as from 7.4-7.5 g/100 g lipid, for example 7.5-7.6 g/100 g lipid, such as from 7.6-7.7 g/100 g lipid, for example 7.7-7.8 g/100 g lipid, such as from 7.8-7.9 g/100 g lipid, for example 7.9-8.0 g/100 g lipid, such as from 8.0-8.1 g/100 g lipid, for example 8.1-8.2 g/100 g lipid, such as from 8.2-8.3 g/100 g lipid, for example 8.3-8.4 g/100 g lipid, such as from 8.4-8.5 g/100 g lipid, for example 8.5-8.6 g/100 g lipid, such as from 8.6-8.7 g/100 g lipid, for example 8.7-8.8 g/100 g lipid, such as from 8.8-8.9 g/100 g lipid, for example 8.9-9.0 g/100 g lipid, such as from 9.0-9.1 g/100 g lipid, for example 9.1-9.2 g/100 g lipid, such as from 9.2-9.3 g/100 g lipid, for example 9.3-9.4 g/100 g lipid, such as from 9.4-9.5 g/100 g lipid, for example 9.5-9.6 g/100 g lipid, such as from 9.6-9.7 g/100 g lipid, for example 9.7-9.8 g/100 g lipid, such as from 9.8-9.9 g/100 g lipid, for example 9.9-10.0 g/100 g lipid, such as from 10.0-10.1 g/100 g lipid, for example 10.1-10.2 g/100 g lipid, such as from 10.2-10.3 g/100 g lipid, for example 10.3-10.4 g/100 g lipid, such as from 10.4-10.5 g/100 g lipid, for example 10.5-10.6 g/100 g lipid, such as from 10.6-10.7 g/100 g lipid, for example 10.7-10.8 g/100 g lipid, such as from 10.8-10.9 g/100 g lipid, for example 10.9-11.0 g/100 g lipid, such as from 11.0-11.1 g/100 g lipid, for example 11.1-11.2 g/100 g lipid, such as from 11.2-11.3 g/100 g lipid, for example 11.3-11.4 g/100 g lipid, such as from 11.4-11.5 g/100 g lipid, for example 11.5-11.6 g/100 g lipid, such as from 11.6-11.7 g/100 g lipid, for example 11.7-11.8 g/100 g lipid, such as from 11.8-11.9 g/100 g lipid, for example 11.9-12.0 g/100 g lipid, such as from 12.0-12.1 g/100 g lipid, for example 12.1-12.2 g/100 g lipid, such as from 12.2-12.3 g/100 g lipid, for example 12.3-12.4 g/100 g lipid, such as from 12.4-12.5 g/100 g lipid, for example 12.5-12.6 g/100 g lipid, such as from 12.6-12.7 g/100 g lipid, for example 12.7-12.8 g/100 g lipid, such as from 12.8-12.9 g/100 g lipid, for example 12.9-13.0 g/100 g lipid, such as from 13.0-13.1 g/100 g lipid, for example 13.1-13.2 g/100 g lipid, such as from 13.2-13.3 g/100 g lipid, for example 13.3-13.4 g/100 g lipid, such as from 13.4-13.5 g/100 g lipid, for example 13.5-13.6 g/100 g lipid, such as from 13.6-13.7 g/100 g lipid, for example 13.7-13.8 g/100 g lipid, such as from 13.8-13.9 g/100 g lipid, for example 13.9-14.0 g/100 g lipid, such as from 14.0-14.1 g/100 g lipid, for example 14.1-14.2 g/100 g lipid, such as from 14.2-14.3 g/100 g lipid, for example 14.3-14.4 g/100 g lipid, such as from 14.4-14.5 g/100 g lipid, for example 14.5-14.6 g/100 g lipid, such as from 14.6-14.7 g/100 g lipid, for example 14.7-14.8 g/100 g lipid, such as from 14.8-14.9 g/100 g lipid, for example 14.9-15.0 g/100 g lipid, such as from 15-16 g/100 g lipid, for example 16-17 g/100 g lipid, such as from 17-18 g/100 g lipid, for example 18-19 g/100 g lipid, such as from 19-20 g/100 g lipid.

In one embodiment the present invention relates to a fish feed composition with a 22:4 n-6 content (g/100 g lipid) in the range from 0-20 g/100 g lipid, such as from 0-0.1 g/100 g lipid, for example 0.1-0.2 g/100 g lipid, such as from 0.2-0.3 g/100 g lipid, for example 0.3-0.4 g/100 g lipid, such as from 0.4-0.5 g/100 g lipid, for example 0.5-0.6 g/100 g lipid, such as from 0.6-0.7 g/100 g lipid, for example 0.7-0.8 g/100 g lipid, such as from 0.8-0.9 g/100 g lipid, for example 0.9-1.0 g/100 g lipid, such as from 1.0-1.1 g/100 g lipid, for example 1.1-1.2 g/100 g lipid, such as from 1.2-1.3 g/100 g lipid, for example 1.3-1.4 g/100 g lipid, such as from 1.4-1.5 g/100 g lipid, for example 1.5-1.6 g/100 g lipid, such as from 1.6-1.7 g/100 g lipid, for example 1.7-1.8 g/100 g lipid, such as from 1.8-1.9 g/100 g lipid, for example 1.9-2.0 g/100 g lipid, such as from 2.0-2.1 g/100 g lipid, for example 2.1-2.2 g/100 g lipid, such as from 2.2-2.3 g/100 g lipid, for example 2.3-2.4 g/100 g lipid, such as from 2.4-2.5 g/100 g lipid, for example 2.5-2.6 g/100 g lipid, such as from 2.6-2.7 g/100 g lipid, for example 2.7-2.8 g/100 g lipid, such as from 2.8-2.9 g/100 g lipid, for example 2.9-3.0 g/100 g lipid, such as from 3.0-3.1 g/100 g lipid, for example 3.1-3.2 g/100 g lipid, such as from 3.2-3.3 g/100 g lipid, for example 3.3-3.4 g/100 g lipid, such as from 3.4-3.5 g/100 g lipid, for example 3.5-3.6 g/100 g lipid, such as from 3.6-3.7 g/100 g lipid, for example 3.7-3.8 g/100 g lipid, such as from 3.8-3.9 g/100 g lipid, for example 3.9-4.0 g/100 g lipid, such as from 4.0-4.1 g/100 g lipid, for example 4.1-4.2 g/100 g lipid, such as from 4.2-4.3 g/100 g lipid, for example 4.3-4.4 g/100 g lipid, such as from 4.4-4.5 g/100 g lipid, for example 4.5-4.6 g/100 g lipid, such as from 4.6-4.7 g/100 g lipid, for example 4.7-4.8 g/100 g lipid, such as from 4.8-4.9 g/100 g lipid, for example 4.9-5.0 g/100 g lipid, such as from 5.0-5.1 g/100 g lipid, for example 5.1-5.2 g/100 g lipid, such as from 5.2-5.3 g/100 g lipid, for example 5.3-5.4 g/100 g lipid, such as from 5.4-5.5 g/100 g lipid, for example 5.5-5.6 g/100 g lipid, such as from 5.6-5.7 g/100 g lipid, for example 5.7-5.8 g/100 g lipid, such as from 5.8-5.9 g/100 g lipid, for example 5.9-6.0 g/100 g lipid, such as from 6.0-6.1 g/100 g lipid, for example 6.1-6.2 g/100 g lipid, such as from 6.2-6.3 g/100 g lipid, for example 6.3-6.4 g/100 g lipid, such as from 6.4-6.5 g/100 g lipid, for example 6.5-6.6 g/100 g lipid, such as from 6.6-6.7 g/100 g lipid, for example 6.7-6.8 g/100 g lipid, such as from 6.8-6.9 g/100 g lipid, for example 6.9-7.0 g/100 g lipid, such as from 7.0-7.1 g/100 g lipid, for example 7.1-7.2 g/100 g lipid, such as from 7.2-7.3 g/100 g lipid, for example 7.3-7.4 g/100 g lipid, such as from 7.4-7.5 g/100 g lipid, for example 7.5-7.6 g/100 g lipid, such as from 7.6-7.7 g/100 g lipid, for example 7.7-7.8 g/100 g lipid, such as from 7.8-7.9 g/100 g lipid, for example 7.9-8.0 g/100 g lipid, such as from 8.0-8.1 g/100 g lipid, for example 8.1-8.2 g/100 g lipid, such as from 8.2-8.3 g/100 g lipid, for example 8.3-8.4 g/100 g lipid, such as from 8.4-8.5 g/100 g lipid, for example 8.5-8.6 g/100 g lipid, such as from 8.6-8.7 g/100 g lipid, for example 8.7-8.8 g/100 g lipid, such as from 8.8-8.9 g/100 g lipid, for example 8.9-9.0 g/100 g lipid, such as from 9.0-9.1 g/100 g lipid, for example 9.1-9.2 g/100 g lipid, such as from 9.2-9.3 g/100 g lipid, for example 9.3-9.4 g/100 g lipid, such as from 9.4-9.5 g/100 g lipid, for example 9.5-9.6 g/100 g lipid, such as from 9.6-9.7 g/100 g lipid, for example 9.7-9.8 g/100 g lipid, such as from 9.8-9.9 g/100 g lipid, for example 9.9-10.0 g/100 g lipid, such as from 10.0-10.1 g/100 g lipid, for example 10.1-10.2 g/100 g lipid, such as from 10.2-10.3 g/100 g lipid, for example 10.3-10.4 g/100 g lipid, such as from 10.4-10.5 g/100 g lipid, for example 10.5-10.6 g/100 g lipid, such as from 10.6-10.7 g/100 g lipid, for example 10.7-10.8 g/100 g lipid, such as from 10.8-10.9 g/100 g lipid, for example 10.9-11.0 g/100 g lipid, such as from 11.0-11.1 g/100 g lipid, for example 11.1-11.2 g/100 g lipid, such as from 11.2-11.3 g/100 g lipid, for example 11.3-11.4 g/100 g lipid, such as from 11.4-11.5 g/100 g lipid, for example 11.5-11.6 g/100 g lipid, such as from 11.6-11.7 g/100 g lipid, for example 11.7-11.8 g/100 g lipid, such as from 11.8-11.9 g/100 g lipid, for example 11.9-12.0 g/100 g lipid, such as from 12.0-12.1 g/100 g lipid, for example 12.1-12.2 g/100 g lipid, such as from 12.2-12.3 g/100 g lipid, for example 12.3-12.4 g/100 g lipid, such as from 12.4-12.5 g/100 g lipid, for example 12.5-12.6 g/100 g lipid, such as from 12.6-12.7 g/100 g lipid, for example 12.7-12.8 g/100 g lipid, such as from 12.8-12.9 g/100 g lipid, for example 12.9-13.0 g/100 g lipid, such as from 13.0-13.1 g/100 g lipid, for example 13.1-13.2 g/100 g lipid, such as from 13.2-13.3 g/100 g lipid, for example 13.3-13.4 g/100 g lipid, such as from 13.4-13.5 g/100 g lipid, for example 13.5-13.6 g/100 g lipid, such as from 13.6-13.7 g/100 g lipid, for example 13.7-13.8 g/100 g lipid, such as from 13.8-13.9 g/100 g lipid, for example 13.9-14.0 g/100 g lipid, such as from 14.0-14.1 g/100 g lipid, for example 14.1-14.2 g/100 g lipid, such as from 14.2-14.3 g/100 g lipid, for example 14.3-14.4 g/100 g lipid, such as from 14.4-14.5 g/100 g lipid, for example 14.5-14.6 g/100 g lipid, such as from 14.6-14.7 g/100 g lipid, for example 14.7-14.8 g/100 g lipid, such as from 14.8-14.9 g/100 g lipid, for example 14.9-15.0 g/100 g lipid, such as from 15-16 g/100 g lipid, for example 16-17 g/100 g lipid, such as from 17-18 g/100 g lipid, for example 18-19 g/100 g lipid, such as from 19-20 g/100 g lipid.

In one embodiment the present invention relates to a fish feed composition with a 18:3 n-3 content (g/100 g lipid) in the range from 0-20 g/100 g lipid, such as from 0-0.1 g/100 g lipid, for example 0.1-0.2 g/100 g lipid, such as from 0.2-0.3 g/100 g lipid, for example 0.3-0.4 g/100 g lipid, such as from 0.4-0.5 g/100 g lipid, for example 0.5-0.6 g/100 g lipid, such as from 0.6-0.7 g/100 g lipid, for example 0.7-0.8 g/100 g lipid, such as from 0.8-0.9 g/100 g lipid, for example 0.9-1.0 g/100 g lipid, such as from 1.0-1.1 g/100 g lipid, for example 1.1-1.2 g/100 g lipid, such as from 1.2-1.3 g/100 g lipid, for example 1.3-1.4 g/100 g lipid, such as from 1.4-1.5 g/100 g lipid, for example 1.5-1.6 g/100 g lipid, such as from 1.6-1.7 g/100 g lipid, for example 1.7-1.8 g/100 g lipid, such as from 1.8-1.9 g/100 g lipid, for example 1.9-2.0 g/100 g lipid, such as from 2.0-2.1 g/100 g lipid, for example 2.1-2.2 g/100 g lipid, such as from 2.2-2.3 g/100 g lipid, for example 2.3-2.4 g/100 g lipid, such as from 2.4-2.5 g/100 g lipid, for example 2.5-2.6 g/100 g lipid, such as from 2.6-2.7 g/100 g lipid, for example 2.7-2.8 g/100 g lipid, such as from 2.8-2.9 g/100 g lipid, for example 2.9-3.0 g/100 g lipid, such as from 3.0-3.1 g/100 g lipid, for example 3.1-3.2 g/100 g lipid, such as from 3.2-3.3 g/100 g lipid, for example 3.3-3.4 g/100 g lipid, such as from 3.4-3.5 g/100 g lipid, for example 3.5-3.6 g/100 g lipid, such as from 3.6-3.7 g/100 g lipid, for example 3.7-3.8 g/100 g lipid, such as from 3.8-3.9 g/100 g lipid, for example 3.9-4.0 g/100 g lipid, such as from 4.0-4.1 g/100 g lipid, for example 4.1-4.2 g/100 g lipid, such as from 4.2-4.3 g/100 g lipid, for example 4.3-4.4 g/100 g lipid, such as from 4.4-4.5 g/100 g lipid, for example 4.5-4.6 g/100 g lipid, such as from 4.6-4.7 g/100 g lipid, for example 4.7-4.8 g/100 g lipid, such as from 4.8-4.9 g/100 g lipid, for example 4.9-5.0 g/100 g lipid, such as from 5.0-5.1 g/100 g lipid, for example 5.1-5.2 g/100 g lipid, such as from 5.2-5.3 g/100 g lipid, for example 5.3-5.4 g/100 g lipid, such as from 5.4-5.5 g/100 g lipid, for example 5.5-5.6 g/100 g lipid, such as from 5.6-5.7 g/100 g lipid, for example 5.7-5.8 g/100 g lipid, such as from 5.8-5.9 g/100 g lipid, for example 5.9-6.0 g/100 g lipid, such as from 6.0-6.1 g/100 g lipid, for example 6.1-6.2 g/100 g lipid, such as from 6.2-6.3 g/100 g lipid, for example 6.3-6.4 g/100 g lipid, such as from 6.4-6.5 g/100 g lipid, for example 6.5-6.6 g/100 g lipid, such as from 6.6-6.7 g/100 g lipid, for example 6.7-6.8 g/100 g lipid, such as from 6.8-6.9 g/100 g lipid, for example 6.9-7.0 g/100 g lipid, such as from 7.0-7.1 g/100 g lipid, for example 7.1-7.2 g/100 g lipid, such as from 7.2-7.3 g/100 g lipid, for example 7.3-7.4 g/100 g lipid, such as from 7.4-7.5 g/100 g lipid, for example 7.5-7.6 g/100 g lipid, such as from 7.6-7.7 g/100 g lipid, for example 7.7-7.8 g/100 g lipid, such as from 7.8-7.9 g/100 g lipid, for example 7.9-8.0 g/100 g lipid, such as from 8.0-8.1 g/100 g lipid, for example 8.1-8.2 g/100 g lipid, such as from 8.2-8.3 g/100 g lipid, for example 8.3-8.4 g/100 g lipid, such as from 8.4-8.5 g/100 g lipid, for example 8.5-8.6 g/100 g lipid, such as from 8.6-8.7 g/100 g lipid, for example 8.7-8.8 g/100 g lipid, such as from 8.8-8.9 g/100 g lipid, for example 8.9-9.0 g/100 g lipid, such as from 9.0-9.1 g/100 g lipid, for example 9.1-9.2 g/100 g lipid, such as from 9.2-9.3 g/100 g lipid, for example 9.3-9.4 g/100 g lipid, such as from 9.4-9.5 g/100 g lipid, for example 9.5-9.6 g/100 g lipid, such as from 9.6-9.7 g/100 g lipid, for example 9.7-9.8 g/100 g lipid, such as from 9.8-9.9 g/100 g lipid, for example 9.9-10.0 g/100 g lipid, such as from 10.0-10.1 g/100 g lipid, for example 10.1-10.2 g/100 g lipid, such as from 10.2-10.3 g/100 g lipid, for example 10.3-10.4 g/100 g lipid, such as from 10.4-10.5 g/100 g lipid, for example 10.5-10.6 g/100 g lipid, such as from 10.6-10.7 g/100 g lipid, for example 10.7-10.8 g/100 g lipid, such as from 10.8-10.9 g/100 g lipid, for example 10.9-11.0 g/100 g lipid, such as from 11.0-11.1 g/100 g lipid, for example 11.1-11.2 g/100 g lipid, such as from 11.2-11.3 g/100 g lipid, for example 11.3-11.4 g/100 g lipid, such as from 11.4-11.5 g/100 g lipid, for example 11.5-11.6 g/100 g lipid, such as from 11.6-11.7 g/100 g lipid, for example 11.7-11.8 g/100 g lipid, such as from 11.8-11.9 g/100 g lipid, for example 11.9-12.0 g/100 g lipid, such as from 12.0-12.1 g/100 g lipid, for example 12.1-12.2 g/100 g lipid, such as from 12.2-12.3 g/100 g lipid, for example 12.3-12.4 g/100 g lipid, such as from 12.4-12.5 g/100 g lipid, for example 12.5-12.6 g/100 g lipid, such as from 12.6-12.7 g/100 g lipid, for example 12.7-12.8 g/100 g lipid, such as from 12.8-12.9 g/100 g lipid, for example 12.9-13.0 g/100 g lipid, such as from 13.0-13.1 g/100 g lipid, for example 13.1-13.2 g/100 g lipid, such as from 13.2-13.3 g/100 g lipid, for example 13.3-13.4 g/100 g lipid, such as from 13.4-13.5 g/100 g lipid, for example 13.5-13.6 g/100 g lipid, such as from 13.6-13.7 g/100 g lipid, for example 13.7-13.8 g/100 g lipid, such as from 13.8-13.9 g/100 g lipid, for example 13.9-14.0 g/100 g lipid, such as from 14.0-14.1 g/100 g lipid, for example 14.1-14.2 g/100 g lipid, such as from 14.2-14.3 g/100 g lipid, for example 14.3-14.4 g/100 g lipid, such as from 14.4-14.5 g/100 g lipid, for example 14.5-14.6 g/100 g lipid, such as from 14.6-14.7 g/100 g lipid, for example 14.7-14.8 g/100 g lipid, such as from 14.8-14.9 g/100 g lipid, for example 14.9-15.0 g/100 g lipid, such as from 15-16 g/100 g lipid, for example 16-17 g/100 g lipid, such as from 17-18 g/100 g lipid, for example 18-19 g/100 g lipid, such as from 19-20 g/100 g lipid.

In one embodiment the present invention relates to a fish feed composition with a 18:4 n-3 content (g/100 g lipid) in the range from 0-20 g/100 g lipid, such as from 0-0.1 g/100 g lipid, for example 0.1-0.2 g/100 g lipid, such as from 0.2-0.3 g/100 g lipid, for example 0.3-0.4 g/100 g lipid, such as from 0.4-0.5 g/100 g lipid, for example 0.5-0.6 g/100 g lipid, such as from 0.6-0.7 g/100 g lipid, for example 0.7-0.8 g/100 g lipid, such as from 0.8-0.9 g/100 g lipid, for example 0.9-1.0 g/100 g lipid, such as from 1.0-1.1 g/100 g lipid, for example 1.1-1.2 g/100 g lipid, such as from 1.2-1.3 g/100 g lipid, for example 1.3-1.4 g/100 g lipid, such as from 1.4-1.5 g/100 g lipid, for example 1.5-1.6 g/100 g lipid, such as from 1.6-1.7 g/100 g lipid, for example 1.7-1.8 g/100 g lipid, such as from 1.8-1.9 g/100 g lipid, for example 1.9-2.0 g/100 g lipid, such as from 2.0-2.1 g/100 g lipid, for example 2.1-2.2 g/100 g lipid, such as from 2.2-2.3 g/100 g lipid, for example 2.3-2.4 g/100 g lipid, such as from 2.4-2.5 g/100 g lipid, for example 2.5-2.6 g/100 g lipid, such as from 2.6-2.7 g/100 g lipid, for example 2.7-2.8 g/100 g lipid, such as from 2.8-2.9 g/100 g lipid, for example 2.9-3.0 g/100 g lipid, such as from 3.0-3.1 g/100 g lipid, for example 3.1-3.2 g/100 g lipid, such as from 3.2-3.3 g/100 g lipid, for example 3.3-3.4 g/100 g lipid, such as from 3.4-3.5 g/100 g lipid, for example 3.5-3.6 g/100 g lipid, such as from 3.6-3.7 g/100 g lipid, for example 3.7-3.8 g/100 g lipid, such as from 3.8-3.9 g/100 g lipid, for example 3.9-4.0 g/100 g lipid, such as from 4.0-4.1 g/100 g lipid, for example 4.1-4.2 g/100 g lipid, such as from 4.2-4.3 g/100 g lipid, for example 4.3-4.4 g/100 g lipid, such as from 4.4-4.5 g/100 g lipid, for example 4.5-4.6 g/100 g lipid, such as from 4.6-4.7 g/100 g lipid, for example 4.7-4.8 g/100 g lipid, such as from 4.8-4.9 g/100 g lipid, for example 4.9-5.0 g/100 g lipid, such as from 5.0-5.1 g/100 g lipid, for example 5.1-5.2 g/100 g lipid, such as from 5.2-5.3 g/100 g lipid, for example 5.3-5.4 g/100 g lipid, such as from 5.4-5.5 g/100 g lipid, for example 5.5-5.6 g/100 g lipid, such as from 5.6-5.7 g/100 g lipid, for example 5.7-5.8 g/100 g lipid, such as from 5.8-5.9 g/100 g lipid, for example 5.9-6.0 g/100 g lipid, such as from 6.0-6.1 g/100 g lipid, for example 6.1-6.2 g/100 g lipid, such as from 6.2-6.3 g/100 g lipid, for example 6.3-6.4 g/100 g lipid, such as from 6.4-6.5 g/100 g lipid, for example 6.5-6.6 g/100 g lipid, such as from 6.6-6.7 g/100 g lipid, for example 6.7-6.8 g/100 g lipid, such as from 6.8-6.9 g/100 g lipid, for example 6.9-7.0 g/100 g lipid, such as from 7.0-7.1 g/100 g lipid, for example 7.1-7.2 g/100 g lipid, such as from 7.2-7.3 g/100 g lipid, for example 7.3-7.4 g/100 g lipid, such as from 7.4-7.5 g/100 g lipid, for example 7.5-7.6 g/100 g lipid, such as from 7.6-7.7 g/100 g lipid, for example 7.7-7.8 g/100 g lipid, such as from 7.8-7.9 g/100 g lipid, for example 7.9-8.0 g/100 g lipid, such as from 8.0-8.1 g/100 g lipid, for example 8.1-8.2 g/100 g lipid, such as from 8.2-8.3 g/100 g lipid, for example 8.3-8.4 g/100 g lipid, such as from 8.4-8.5 g/100 g lipid, for example 8.5-8.6 g/100 g lipid, such as from 8.6-8.7 g/100 g lipid, for example 8.7-8.8 g/100 g lipid, such as from 8.8-8.9 g/100 g lipid, for example 8.9-9.0 g/100 g lipid, such as from 9.0-9.1 g/100 g lipid, for example 9.1-9.2 g/100 g lipid, such as from 9.2-9.3 g/100 g lipid, for example 9.3-9.4 g/100 g lipid, such as from 9.4-9.5 g/100 g lipid, for example 9.5-9.6 g/100 g lipid, such as from 9.6-9.7 g/100 g lipid, for example 9.7-9.8 g/100 g lipid, such as from 9.8-9.9 g/100 g lipid, for example 9.9-10.0 g/100 g lipid, such as from 10.0-10.1 g/100 g lipid, for example 10.1-10.2 g/100 g lipid, such as from 10.2-10.3 g/100 g lipid, for example 10.3-10.4 g/100 g lipid, such as from 10.4-10.5 g/100 g lipid, for example 10.5-10.6 g/100 g lipid, such as from 10.6-10.7 g/100 g lipid, for example 10.7-10.8 g/100 g lipid, such as from 10.8-10.9 g/100 g lipid, for example 10.9-11.0 g/100 g lipid, such as from 11.0-11.1 g/100 g lipid, for example 11.1-11.2 g/100 g lipid, such as from 11.2-11.3 g/100 g lipid, for example 11.3-11.4 g/100 g lipid, such as from 11.4-11.5 g/100 g lipid, for example 11.5-11.6 g/100 g lipid, such as from 11.6-11.7 g/100 g lipid, for example 11.7-11.8 g/100 g lipid, such as from 11.8-11.9 g/100 g lipid, for example 11.9-12.0 g/100 g lipid, such as from 12.0-12.1 g/100 g lipid, for example 12.1-12.2 g/100 g lipid, such as from 12.2-12.3 g/100 g lipid, for example 12.3-12.4 g/100 g lipid, such as from 12.4-12.5 g/100 g lipid, for example 12.5-12.6 g/100 g lipid, such as from 12.6-12.7 g/100 g lipid, for example 12.7-12.8 g/100 g lipid, such as from 12.8-12.9 g/100 g lipid, for example 12.9-13.0 g/100 g lipid, such as from 13.0-13.1 g/100 g lipid, for example 13.1-13.2 g/100 g lipid, such as from 13.2-13.3 g/100 g lipid, for example 13.3-13.4 g/100 g lipid, such as from 13.4-13.5 g/100 g lipid, for example 13.5-13.6 g/100 g lipid, such as from 13.6-13.7 g/100 g lipid, for example 13.7-13.8 g/100 g lipid, such as from 13.8-13.9 g/100 g lipid, for example 13.9-14.0 g/100 g lipid, such as from 14.0-14.1 g/100 g lipid, for example 14.1-14.2 g/100 g lipid, such as from 14.2-14.3 g/100 g lipid, for example 14.3-14.4 g/100 g lipid, such as from 14.4-14.5 g/100 g lipid, for example 14.5-14.6 g/100 g lipid, such as from 14.6-14.7 g/100 g lipid, for example 14.7-14.8 g/100 g lipid, such as from 14.8-14.9 g/100 g lipid, for example 14.9-15.0 g/100 g lipid, such as from 15-16 g/100 g lipid, for example 16-17 g/100 g lipid, such as from 17-18 g/100 g lipid, for example 18-19 g/100 g lipid, such as from 19-20 g/100 g lipid.

In one embodiment the present invention relates to a fish feed composition with a 20:3 n-3 content (g/100 g lipid) in the range from 0-20 g/100 g lipid, such as from 0-0.1 g/100 g lipid, for example 0.1-0.2 g/100 g lipid, such as from 0.2-0.3 g/100 g lipid, for example 0.3-0.4 g/100 g lipid, such as from 0.4-0.5 g/100 g lipid, for example 0.5-0.6 g/100 g lipid, such as from 0.6-0.7 g/100 g lipid, for example 0.7-0.8 g/100 g lipid, such as from 0.8-0.9 g/100 g lipid, for example 0.9-1.0 g/100 g lipid, such as from 1.0-1.1 g/100 g lipid, for example 1.1-1.2 g/100 g lipid, such as from 1.2-1.3 g/100 g lipid, for example 1.3-1.4 g/100 g lipid, such as from 1.4-1.5 g/100 g lipid, for example 1.5-1.6 g/100 g lipid, such as from 1.6-1.7 g/100 g lipid, for example 1.7-1.8 g/100 g lipid, such as from 1.8-1.9 g/100 g lipid, for example 1.9-2.0 g/100 g lipid, such as from 2.0-2.1 g/100 g lipid, for example 2.1-2.2 g/100 g lipid, such as from 2.2-2.3 g/100 g lipid, for example 2.3-2.4 g/100 g lipid, such as from 2.4-2.5 g/100 g lipid, for example 2.5-2.6 g/100 g lipid, such as from 2.6-2.7 g/100 g lipid, for example 2.7-2.8 g/100 g lipid, such as from 2.8-2.9 g/100 g lipid, for example 2.9-3.0 g/100 g lipid, such as from 3.0-3.1 g/100 g lipid, for example 3.1-3.2 g/100 g lipid, such as from 3.2-3.3 g/100 g lipid, for example 3.3-3.4 g/100 g lipid, such as from 3.4-3.5 g/100 g lipid, for example 3.5-3.6 g/100 g lipid, such as from 3.6-3.7 g/100 g lipid, for example 3.7-3.8 g/100 g lipid, such as from 3.8-3.9 g/100 g lipid, for example 3.9-4.0 g/100 g lipid, such as from 4.0-4.1 g/100 g lipid, for example 4.1-4.2 g/100 g lipid, such as from 4.2-4.3 g/100 g lipid, for example 4.3-4.4 g/100 g lipid, such as from 4.4-4.5 g/100 g lipid, for example 4.5-4.6 g/100 g lipid, such as from 4.6-4.7 g/100 g lipid, for example 4.7-4.8 g/100 g lipid, such as from 4.8-4.9 g/100 g lipid, for example 4.9-5.0 g/100 g lipid, such as from 5.0-5.1 g/100 g lipid, for example 5.1-5.2 g/100 g lipid, such as from 5.2-5.3 g/100 g lipid, for example 5.3-5.4 g/100 g lipid, such as from 5.4-5.5 g/100 g lipid, for example 5.5-5.6 g/100 g lipid, such as from 5.6-5.7 g/100 g lipid, for example 5.7-5.8 g/100 g lipid, such as from 5.8-5.9 g/100 g lipid, for example 5.9-6.0 g/100 g lipid, such as from 6.0-6.1 g/100 g lipid, for example 6.1-6.2 g/100 g lipid, such as from 6.2-6.3 g/100 g lipid, for example 6.3-6.4 g/100 g lipid, such as from 6.4-6.5 g/100 g lipid, for example 6.5-6.6 g/100 g lipid, such as from 6.6-6.7 g/100 g lipid, for example 6.7-6.8 g/100 g lipid, such as from 6.8-6.9 g/100 g lipid, for example 6.9-7.0 g/100 g lipid, such as from 7.0-7.1 g/100 g lipid, for example 7.1-7.2 g/100 g lipid, such as from 7.2-7.3 g/100 g lipid, for example 7.3-7.4 g/100 g lipid, such as from 7.4-7.5 g/100 g lipid, for example 7.5-7.6 g/100 g lipid, such as from 7.6-7.7 g/100 g lipid, for example 7.7-7.8 g/100 g lipid, such as from 7.8-7.9 g/100 g lipid, for example 7.9-8.0 g/100 g lipid, such as from 8.0-8.1 g/100 g lipid, for example 8.1-8.2 g/100 g lipid, such as from 8.2-8.3 g/100 g lipid, for example 8.3-8.4 g/100 g lipid, such as from 8.4-8.5 g/100 g lipid, for example 8.5-8.6 g/100 g lipid, such as from 8.6-8.7 g/100 g lipid, for example 8.7-8.8 g/100 g lipid, such as from 8.8-8.9 g/100 g lipid, for example 8.9-9.0 g/100 g lipid, such as from 9.0-9.1 g/100 g lipid, for example 9.1-9.2 g/100 g lipid, such as from 9.2-9.3 g/100 g lipid, for example 9.3-9.4 g/100 g lipid, such as from 9.4-9.5 g/100 g lipid, for example 9.5-9.6 g/100 g lipid, such as from 9.6-9.7 g/100 g lipid, for example 9.7-9.8 g/100 g lipid, such as from 9.8-9.9 g/100 g lipid, for example 9.9-10.0 g/100 g lipid, such as from 10.0-10.1 g/100 g lipid, for example 10.1-10.2 g/100 g lipid, such as from 10.2-10.3 g/100 g lipid, for example 10.3-10.4 g/100 g lipid, such as from 10.4-10.5 g/100 g lipid, for example 10.5-10.6 g/100 g lipid, such as from 10.6-10.7 g/100 g lipid, for example 10.7-10.8 g/100 g lipid, such as from 10.8-10.9 g/100 g lipid, for example 10.9-11.0 g/100 g lipid, such as from 11.0-11.1 g/100 g lipid, for example 11.1-11.2 g/100 g lipid, such as from 11.2-11.3 g/100 g lipid, for example 11.3-11.4 g/100 g lipid, such as from 11.4-11.5 g/100 g lipid, for example 11.5-11.6 g/100 g lipid, such as from 11.6-11.7 g/100 g lipid, for example 11.7-11.8 g/100 g lipid, such as from 11.8-11.9 g/100 g lipid, for example 11.9-12.0 g/100 g lipid, such as from 12.0-12.1 g/100 g lipid, for example 12.1-12.2 g/100 g lipid, such as from 12.2-12.3 g/100 g lipid, for example 12.3-12.4 g/100 g lipid, such as from 12.4-12.5 g/100 g lipid, for example 12.5-12.6 g/100 g lipid, such as from 12.6-12.7 g/100 g lipid, for example 12.7-12.8 g/100 g lipid, such as from 12.8-12.9 g/100 g lipid, for example 12.9-13.0 g/100 g lipid, such as from 13.0-13.1 g/100 g lipid, for example 13.1-13.2 g/100 g lipid, such as from 13.2-13.3 g/100 g lipid, for example 13.3-13.4 g/100 g lipid, such as from 13.4-13.5 g/100 g lipid, for example 13.5-13.6 g/100 g lipid, such as from 13.6-13.7 g/100 g lipid, for example 13.7-13.8 g/100 g lipid, such as from 13.8-13.9 g/100 g lipid, for example 13.9-14.0 g/100 g lipid, such as from 14.0-14.1 g/100 g lipid, for example 14.1-14.2 g/100 g lipid, such as from 14.2-14.3 g/100 g lipid, for example 14.3-14.4 g/100 g lipid, such as from 14.4-14.5 g/100 g lipid, for example 14.5-14.6 g/100 g lipid, such as from 14.6-14.7 g/100 g lipid, for example 14.7-14.8 g/100 g lipid, such as from 14.8-14.9 g/100 g lipid, for example 14.9-15.0 g/100 g lipid, such as from 15-16 g/100 g lipid, for example 16-17 g/100 g lipid, such as from 17-18 g/100 g lipid, for example 18-19 g/100 g lipid, such as from 19-20 g/100 g lipid.

In one embodiment the present invention relates to a fish feed composition with a 20:4 n-3 content (g/100 g lipid) in the range from 0-20 g/100 g lipid, such as from 0-0.1 g/100 g lipid, for example 0.1-0.2 g/100 g lipid, such as from 0.2-0.3 g/100 g lipid, for example 0.3-0.4 g/100 g lipid, such as from 0.4-0.5 g/100 g lipid, for example 0.5-0.6 g/100 g lipid, such as from 0.6-0.7 g/100 g lipid, for example 0.7-0.8 g/100 g lipid, such as from 0.8-0.9 g/100 g lipid, for example 0.9-1.0 g/100 g lipid, such as from 1.0-1.1 g/100 g lipid, for example 1.1-1.2 g/100 g lipid, such as from 1.2-1.3 g/100 g lipid, for example 1.3-1.4 g/100 g lipid, such as from 1.4-1.5 g/100 g lipid, for example 1.5-1.6 g/100 g lipid, such as from 1.6-1.7 g/100 g lipid, for example 1.7-1.8 g/100 g lipid, such as from 1.8-1.9 g/100 g lipid, for example 1.9-2.0 g/100 g lipid, such as from 2.0-2.1 g/100 g lipid, for example 2.1-2.2 g/100 g lipid, such as from 2.2-2.3 g/100 g lipid, for example 2.3-2.4 g/100 g lipid, such as from 2.4-2.5 g/100 g lipid, for example 2.5-2.6 g/100 g lipid, such as from 2.6-2.7 g/100 g lipid, for example 2.7-2.8 g/100 g lipid, such as from 2.8-2.9 g/100 g lipid, for example 2.9-3.0 g/100 g lipid, such as from 3.0-3.1 g/100 g lipid, for example 3.1-3.2 g/100 g lipid, such as from 3.2-3.3 g/100 g lipid, for example 3.3-3.4 g/100 g lipid, such as from 3.4-3.5 g/100 g lipid, for example 3.5-3.6 g/100 g lipid, such as from 3.6-3.7 g/100 g lipid, for example 3.7-3.8 g/100 g lipid, such as from 3.8-3.9 g/100 g lipid, for example 3.9-4.0 g/100 g lipid, such as from 4.0-4.1 g/100 g lipid, for example 4.1-4.2 g/100 g lipid, such as from 4.2-4.3 g/100 g lipid, for example 4.3-4.4 g/100 g lipid, such as from 4.4-4.5 g/100 g lipid, for example 4.5-4.6 g/100 g lipid, such as from 4.6-4.7 g/100 g lipid, for example 4.7-4.8 g/100 g lipid, such as from 4.8-4.9 g/100 g lipid, for example 4.9-5.0 g/100 g lipid, such as from 5.0-5.1 g/100 g lipid, for example 5.1-5.2 g/100 g lipid, such as from 5.2-5.3 g/100 g lipid, for example 5.3-5.4 g/100 g lipid, such as from 5.4-5.5 g/100 g lipid, for example 5.5-5.6 g/100 g lipid, such as from 5.6-5.7 g/100 g lipid, for example 5.7-5.8 g/100 g lipid, such as from 5.8-5.9 g/100 g lipid, for example 5.9-6.0 g/100 g lipid, such as from 6.0-6.1 g/100 g lipid, for example 6.1-6.2 g/100 g lipid, such as from 6.2-6.3 g/100 g lipid, for example 6.3-6.4 g/100 g lipid, such as from 6.4-6.5 g/100 g lipid, for example 6.5-6.6 g/100 g lipid, such as from 6.6-6.7 g/100 g lipid, for example 6.7-6.8 g/100 g lipid, such as from 6.8-6.9 g/100 g lipid, for example 6.9-7.0 g/100 g lipid, such as from 7.0-7.1 g/100 g lipid, for example 7.1-7.2 g/100 g lipid, such as from 7.2-7.3 g/100 g lipid, for example 7.3-7.4 g/100 g lipid, such as from 7.4-7.5 g/100 g lipid, for example 7.5-7.6 g/100 g lipid, such as from 7.6-7.7 g/100 g lipid, for example 7.7-7.8 g/100 g lipid, such as from 7.8-7.9 g/100 g lipid, for example 7.9-8.0 g/100 g lipid, such as from 8.0-8.1 g/100 g lipid, for example 8.1-8.2 g/100 g lipid, such as from 8.2-8.3 g/100 g lipid, for example 8.3-8.4 g/100 g lipid, such as from 8.4-8.5 g/100 g lipid, for example 8.5-8.6 g/100 g lipid, such as from 8.6-8.7 g/100 g lipid, for example 8.7-8.8 g/100 g lipid, such as from 8.8-8.9 g/100 g lipid, for example 8.9-9.0 g/100 g lipid, such as from 9.0-9.1 g/100 g lipid, for example 9.1-9.2 g/100 g lipid, such as from 9.2-9.3 g/100 g lipid, for example 9.3-9.4 g/100 g lipid, such as from 9.4-9.5 g/100 g lipid, for example 9.5-9.6 g/100 g lipid, such as from 9.6-9.7 g/100 g lipid, for example 9.7-9.8 g/100 g lipid, such as from 9.8-9.9 g/100 g lipid, for example 9.9-10.0 g/100 g lipid, such as from 10.0-10.1 g/100 g lipid, for example 10.1-10.2 g/100 g lipid, such as from 10.2-10.3 g/100 g lipid, for example 10.3-10.4 g/100 g lipid, such as from 10.4-10.5 g/100 g lipid, for example 10.5-10.6 g/100 g lipid, such as from 10.6-10.7 g/100 g lipid, for example 10.7-10.8 g/100 g lipid, such as from 10.8-10.9 g/100 g lipid, for example 10.9-11.0 g/100 g lipid, such as from 11.0-11.1 g/100 g lipid, for example 11.1-11.2 g/100 g lipid, such as from 11.2-11.3 g/100 g lipid, for example 11.3-11.4 g/100 g lipid, such as from 11.4-11.5 g/100 g lipid, for example 11.5-11.6 g/100 g lipid, such as from 11.6-11.7 g/100 g lipid, for example 11.7-11.8 g/100 g lipid, such as from 11.8-11.9 g/100 g lipid, for example 11.9-12.0 g/100 g lipid, such as from 12.0-12.1 g/100 g lipid, for example 12.1-12.2 g/100 g lipid, such as from 12.2-12.3 g/100 g lipid, for example 12.3-12.4 g/100 g lipid, such as from 12.4-12.5 g/100 g lipid, for example 12.5-12.6 g/100 g lipid, such as from 12.6-12.7 g/100 g lipid, for example 12.7-12.8 g/100 g lipid, such as from 12.8-12.9 g/100 g lipid, for example 12.9-13.0 g/100 g lipid, such as from 13.0-13.1 g/100 g lipid, for example 13.1-13.2 g/100 g lipid, such as from 13.2-13.3 g/100 g lipid, for example 13.3-13.4 g/100 g lipid, such as from 13.4-13.5 g/100 g lipid, for example 13.5-13.6 g/100 g lipid, such as from 13.6-13.7 g/100 g lipid, for example 13.7-13.8 g/100 g lipid, such as from 13.8-13.9 g/100 g lipid, for example 13.9-14.0 g/100 g lipid, such as from 14.0-14.1 g/100 g lipid, for example 14.1-14.2 g/100 g lipid, such as from 14.2-14.3 g/100 g lipid, for example 14.3-14.4 g/100 g lipid, such as from 14.4-14.5 g/100 g lipid, for example 14.5-14.6 g/100 g lipid, such as from 14.6-14.7 g/100 g lipid, for example 14.7-14.8 g/100 g lipid, such as from 14.8-14.9 g/100 g lipid, for example 14.9-15.0 g/100 g lipid, such as from 15-16 g/100 g lipid, for example 16-17 g/100 g lipid, such as from 17-18 g/100 g lipid, for example 18-19 g/100 g lipid, such as from 19-20 g/100 g lipid.

In one embodiment the present invention relates to a fish feed composition with a 20:5 n-3 content (g/100 g lipid) in the range from 0-20 g/100 g lipid, such as from 0-0.1 g/100 g lipid, for example 0.1-0.2 g/100 g lipid, such as from 0.2-0.3 g/100 g lipid, for example 0.3-0.4 g/100 g lipid, such as from 0.4-0.5 g/100 g lipid, for example 0.5-0.6 g/100 g lipid, such as from 0.6-0.7 g/100 g lipid, for example 0.7-0.8 g/100 g lipid, such as from 0.8-0.9 g/100 g lipid, for example 0.9-1.0 g/100 g lipid, such as from 1.0-1.1 g/100 g lipid, for example 1.1-1.2 g/100 g lipid, such as from 1.2-1.3 g/100 g lipid, for example 1.3-1.4 g/100 g lipid, such as from 1.4-1.5 g/100 g lipid, for example 1.5-1.6 g/100 g lipid, such as from 1.6-1.7 g/100 g lipid, for example 1.7-1.8 g/100 g lipid, such as from 1.8-1.9 g/100 g lipid, for example 1.9-2.0 g/100 g lipid, such as from 2.0-2.1 g/100 g lipid, for example 2.1-2.2 g/100 g lipid, such as from 2.2-2.3 g/100 g lipid, for example 2.3-2.4 g/100 g lipid, such as from 2.4-2.5 g/100 g lipid, for example 2.5-2.6 g/100 g lipid, such as from 2.6-2.7 g/100 g lipid, for example 2.7-2.8 g/100 g lipid, such as from 2.8-2.9 g/100 g lipid, for example 2.9-3.0 g/100 g lipid, such as from 3.0-3.1 g/100 g lipid, for example 3.1-3.2 g/100 g lipid, such as from 3.2-3.3 g/100 g lipid, for example 3.3-3.4 g/100 g lipid, such as from 3.4-3.5 g/100 g lipid, for example 3.5-3.6 g/100 g lipid, such as from 3.6-3.7 g/100 g lipid, for example 3.7-3.8 g/100 g lipid, such as from 3.8-3.9 g/100 g lipid, for example 3.9-4.0 g/100 g lipid, such as from 4.0-4.1 g/100 g lipid, for example 4.1-4.2 g/100 g lipid, such as from 4.2-4.3 g/100 g lipid, for example 4.3-4.4 g/100 g lipid, such as from 4.4-4.5 g/100 g lipid, for example 4.5-4.6 g/100 g lipid, such as from 4.6-4.7 g/100 g lipid, for example 4.7-4.8 g/100 g lipid, such as from 4.8-4.9 g/100 g lipid, for example 4.9-5.0 g/100 g lipid, such as from 5.0-5.1 g/100 g lipid, for example 5.1-5.2 g/100 g lipid, such as from 5.2-5.3 g/100 g lipid, for example 5.3-5.4 g/100 g lipid, such as from 5.4-5.5 g/100 g lipid, for example 5.5-5.6 g/100 g lipid, such as from 5.6-5.7 g/100 g lipid, for example 5.7-5.8 g/100 g lipid, such as from 5.8-5.9 g/100 g lipid, for example 5.9-6.0 g/100 g lipid, such as from 6.0-6.1 g/100 g lipid, for example 6.1-6.2 g/100 g lipid, such as from 6.2-6.3 g/100 g lipid, for example 6.3-6.4 g/100 g lipid, such as from 6.4-6.5 g/100 g lipid, for example 6.5-6.6 g/100 g lipid, such as from 6.6-6.7 g/100 g lipid, for example 6.7-6.8 g/100 g lipid, such as from 6.8-6.9 g/100 g lipid, for example 6.9-7.0 g/100 g lipid, such as from 7.0-7.1 g/100 g lipid, for example 7.1-7.2 g/100 g lipid, such as from 7.2-7.3 g/100 g lipid, for example 7.3-7.4 g/100 g lipid, such as from 7.4-7.5 g/100 g lipid, for example 7.5-7.6 g/100 g lipid, such as from 7.6-7.7 g/100 g lipid, for example 7.7-7.8 g/100 g lipid, such as from 7.8-7.9 g/100 g lipid, for example 7.9-8.0 g/100 g lipid, such as from 8.0-8.1 g/100 g lipid, for example 8.1-8.2 g/100 g lipid, such as from 8.2-8.3 g/100 g lipid, for example 8.3-8.4 g/100 g lipid, such as from 8.4-8.5 g/100 g lipid, for example 8.5-8.6 g/100 g lipid, such as from 8.6-8.7 g/100 g lipid, for example 8.7-8.8 g/100 g lipid, such as from 8.8-8.9 g/100 g lipid, for example 8.9-9.0 g/100 g lipid, such as from 9.0-9.1 g/100 g lipid, for example 9.1-9.2 g/100 g lipid, such as from 9.2-9.3 g/100 g lipid, for example 9.3-9.4 g/100 g lipid, such as from 9.4-9.5 g/100 g lipid, for example 9.5-9.6 g/100 g lipid, such as from 9.6-9.7 g/100 g lipid, for example 9.7-9.8 g/100 g lipid, such as from 9.8-9.9 g/100 g lipid, for example 9.9-10.0 g/100 g lipid, such as from 10.0-10.1 g/100 g lipid, for example 10.1-10.2 g/100 g lipid, such as from 10.2-10.3 g/100 g lipid, for example 10.3-10.4 g/100 g lipid, such as from 10.4-10.5 g/100 g lipid, for example 10.5-10.6 g/100 g lipid, such as from 10.6-10.7 g/100 g lipid, for example 10.7-10.8 g/100 g lipid, such as from 10.8-10.9 g/100 g lipid, for example 10.9-11.0 g/100 g lipid, such as from 11.0-11.1 g/100 g lipid, for example 11.1-11.2 g/100 g lipid, such as from 11.2-11.3 g/100 g lipid, for example 11.3-11.4 g/100 g lipid, such as from 11.4-11.5 g/100 g lipid, for example 11.5-11.6 g/100 g lipid, such as from 11.6-11.7 g/100 g lipid, for example 11.7-11.8 g/100 g lipid, such as from 11.8-11.9 g/100 g lipid, for example 11.9-12.0 g/100 g lipid, such as from 12.0-12.1 g/100 g lipid, for example 12.1-12.2 g/100 g lipid, such as from 12.2-12.3 g/100 g lipid, for example 12.3-12.4 g/100 g lipid, such as from 12.4-12.5 g/100 g lipid, for example 12.5-12.6 g/100 g lipid, such as from 12.6-12.7 g/100 g lipid, for example 12.7-12.8 g/100 g lipid, such as from 12.8-12.9 g/100 g lipid, for example 12.9-13.0 g/100 g lipid, such as from 13.0-13.1 g/100 g lipid, for example 13.1-13.2 g/100 g lipid, such as from 13.2-13.3 g/100 g lipid, for example 13.3-13.4 g/100 g lipid, such as from 13.4-13.5 g/100 g lipid, for example 13.5-13.6 g/100 g lipid, such as from 13.6-13.7 g/100 g lipid, for example 13.7-13.8 g/100 g lipid, such as from 13.8-13.9 g/100 g lipid, for example 13.9-14.0 g/100 g lipid, such as from 14.0-14.1 g/100 g lipid, for example 14.1-14.2 g/100 g lipid, such as from 14.2-14.3 g/100 g lipid, for example 14.3-14.4 g/100 g lipid, such as from 14.4-14.5 g/100 g lipid, for example 14.5-14.6 g/100 g lipid, such as from 14.6-14.7 g/100 g lipid, for example 14.7-14.8 g/100 g lipid, such as from 14.8-14.9 g/100 g lipid, for example 14.9-15.0 g/100 g lipid, such as from 15-16 g/100 g lipid, for example 16-17 g/100 g lipid, such as from 17-18 g/100 g lipid, for example 18-19 g/100 g lipid, such as from 19-20 g/100 g lipid.

In one embodiment the present invention relates to a fish feed composition with a 21:5 n-3 content (g/100 g lipid) in the range from 0-20 g/100 g lipid, such as from 0-0.1 g/100 g lipid, for example 0.1-0.2 g/100 g lipid, such as from 0.2-0.3 g/100 g lipid, for example 0.3-0.4 g/100 g lipid, such as from 0.4-0.5 g/100 g lipid, for example 0.5-0.6 g/100 g lipid, such as from 0.6-0.7 g/100 g lipid, for example 0.7-0.8 g/100 g lipid, such as from 0.8-0.9 g/100 g lipid, for example 0.9-1.0 g/100 g lipid, such as from 1.0-1.1 g/100 g lipid, for example 1.1-1.2 g/100 g lipid, such as from 1.2-1.3 g/100 g lipid, for example 1.3-1.4 g/100 g lipid, such as from 1.4-1.5 g/100 g lipid, for example 1.5-1.6 g/100 g lipid, such as from 1.6-1.7 g/100 g lipid, for example 1.7-1.8 g/100 g lipid, such as from 1.8-1.9 g/100 g lipid, for example 1.9-2.0 g/100 g lipid, such as from 2.0-2.1 g/100 g lipid, for example 2.1-2.2 g/100 g lipid, such as from 2.2-2.3 g/100 g lipid, for example 2.3-2.4 g/100 g lipid, such as from 2.4-2.5 g/100 g lipid, for example 2.5-2.6 g/100 g lipid, such as from 2.6-2.7 g/100 g lipid, for example 2.7-2.8 g/100 g lipid, such as from 2.8-2.9 g/100 g lipid, for example 2.9-3.0 g/100 g lipid, such as from 3.0-3.1 g/100 g lipid, for example 3.1-3.2 g/100 g lipid, such as from 3.2-3.3 g/100 g lipid, for example 3.3-3.4 g/100 g lipid, such as from 3.4-3.5 g/100 g lipid, for example 3.5-3.6 g/100 g lipid, such as from 3.6-3.7 g/100 g lipid, for example 3.7-3.8 g/100 g lipid, such as from 3.8-3.9 g/100 g lipid, for example 3.9-4.0 g/100 g lipid, such as from 4.0-4.1 g/100 g lipid, for example 4.1-4.2 g/100 g lipid, such as from 4.2-4.3 g/100 g lipid, for example 4.3-4.4 g/100 g lipid, such as from 4.4-4.5 g/100 g lipid, for example 4.5-4.6 g/100 g lipid, such as from 4.6-4.7 g/100 g lipid, for example 4.7-4.8 g/100 g lipid, such as from 4.8-4.9 g/100 g lipid, for example 4.9-5.0 g/100 g lipid, such as from 5.0-5.1 g/100 g lipid, for example 5.1-5.2 g/100 g lipid, such as from 5.2-5.3 g/100 g lipid, for example 5.3-5.4 g/100 g lipid, such as from 5.4-5.5 g/100 g lipid, for example 5.5-5.6 g/100 g lipid, such as from 5.6-5.7 g/100 g lipid, for example 5.7-5.8 g/100 g lipid, such as from 5.8-5.9 g/100 g lipid, for example 5.9-6.0 g/100 g lipid, such as from 6.0-6.1 g/100 g lipid, for example 6.1-6.2 g/100 g lipid, such as from 6.2-6.3 g/100 g lipid, for example 6.3-6.4 g/100 g lipid, such as from 6.4-6.5 g/100 g lipid, for example 6.5-6.6 g/100 g lipid, such as from 6.6-6.7 g/100 g lipid, for example 6.7-6.8 g/100 g lipid, such as from 6.8-6.9 g/100 g lipid, for example 6.9-7.0 g/100 g lipid, such as from 7.0-7.1 g/100 g lipid, for example 7.1-7.2 g/100 g lipid, such as from 7.2-7.3 g/100 g lipid, for example 7.3-7.4 g/100 g lipid, such as from 7.4-7.5 g/100 g lipid, for example 7.5-7.6 g/100 g lipid, such as from 7.6-7.7 g/100 g lipid, for example 7.7-7.8 g/100 g lipid, such as from 7.8-7.9 g/100 g lipid, for example 7.9-8.0 g/100 g lipid, such as from 8.0-8.1 g/100 g lipid, for example 8.1-8.2 g/100 g lipid, such as from 8.2-8.3 g/100 g lipid, for example 8.3-8.4 g/100 g lipid, such as from 8.4-8.5 g/100 g lipid, for example 8.5-8.6 g/100 g lipid, such as from 8.6-8.7 g/100 g lipid, for example 8.7-8.8 g/100 g lipid, such as from 8.8-8.9 g/100 g lipid, for example 8.9-9.0 g/100 g lipid, such as from 9.0-9.1 g/100 g lipid, for example 9.1-9.2 g/100 g lipid, such as from 9.2-9.3 g/100 g lipid, for example 9.3-9.4 g/100 g lipid, such as from 9.4-9.5 g/100 g lipid, for example 9.5-9.6 g/100 g lipid, such as from 9.6-9.7 g/100 g lipid, for example 9.7-9.8 g/100 g lipid, such as from 9.8-9.9 g/100 g lipid, for example 9.9-10.0 g/100 g lipid, such as from 10.0-10.1 g/100 g lipid, for example 10.1-10.2 g/100 g lipid, such as from 10.2-10.3 g/100 g lipid, for example 10.3-10.4 g/100 g lipid, such as from 10.4-10.5 g/100 g lipid, for example 10.5-10.6 g/100 g lipid, such as from 10.6-10.7 g/100 g lipid, for example 10.7-10.8 g/100 g lipid, such as from 10.8-10.9 g/100 g lipid, for example 10.9-11.0 g/100 g lipid, such as from 11.0-11.1 g/100 g lipid, for example 11.1-11.2 g/100 g lipid, such as from 11.2-11.3 g/100 g lipid, for example 11.3-11.4 g/100 g lipid, such as from 11.4-11.5 g/100 g lipid, for example 11.5-11.6 g/100 g lipid, such as from 11.6-11.7 g/100 g lipid, for example 11.7-11.8 g/100 g lipid, such as from 11.8-11.9 g/100 g lipid, for example 11.9-12.0 g/100 g lipid, such as from 12.0-12.1 g/100 g lipid, for example 12.1-12.2 g/100 g lipid, such as from 12.2-12.3 g/100 g lipid, for example 12.3-12.4 g/100 g lipid, such as from 12.4-12.5 g/100 g lipid, for example 12.5-12.6 g/100 g lipid, such as from 12.6-12.7 g/100 g lipid, for example 12.7-12.8 g/100 g lipid, such as from 12.8-12.9 g/100 g lipid, for example 12.9-13.0 g/100 g lipid, such as from 13.0-13.1 g/100 g lipid, for example 13.1-13.2 g/100 g lipid, such as from 13.2-13.3 g/100 g lipid, for example 13.3-13.4 g/100 g lipid, such as from 13.4-13.5 g/100 g lipid, for example 13.5-13.6 g/100 g lipid, such as from 13.6-13.7 g/100 g lipid, for example 13.7-13.8 g/100 g lipid, such as from 13.8-13.9 g/100 g lipid, for example 13.9-14.0 g/100 g lipid, such as from 14.0-14.1 g/100 g lipid, for example 14.1-14.2 g/100 g lipid, such as from 14.2-14.3 g/100 g lipid, for example 14.3-14.4 g/100 g lipid, such as from 14.4-14.5 g/100 g lipid, for example 14.5-14.6 g/100 g lipid, such as from 14.6-14.7 g/100 g lipid, for example 14.7-14.8 g/100 g lipid, such as from 14.8-14.9 g/100 g lipid, for example 14.9-15.0 g/100 g lipid, such as from 15-16 g/100 g lipid, for example 16-17 g/100 g lipid, such as from 17-18 g/100 g lipid, for example 18-19 g/100 g lipid, such as from 19-20 g/100 g lipid.

In one embodiment the present invention relates to a fish feed composition with a 22:5 n-3 content (g/100 g lipid) in the range from 0-20 g/100 g lipid, such as from 0-0.1 g/100 g lipid, for example 0.1-0.2 g/100 g lipid, such as from 0.2-0.3 g/100 g lipid, for example 0.3-0.4 g/100 g lipid, such as from 0.4-0.5 g/100 g lipid, for example 0.5-0.6 g/100 g lipid, such as from 0.6-0.7 g/100 g lipid, for example 0.7-0.8 g/100 g lipid, such as from 0.8-0.9 g/100 g lipid, for example 0.9-1.0 g/100 g lipid, such as from 1.0-1.1 g/100 g lipid, for example 1.1-1.2 g/100 g lipid, such as from 1.2-1.3 g/100 g lipid, for example 1.3-1.4 g/100 g lipid, such as from 1.4-1.5 g/100 g lipid, for example 1.5-1.6 g/100 g lipid, such as from 1.6-1.7 g/100 g lipid, for example 1.7-1.8 g/100 g lipid, such as from 1.8-1.9 g/100 g lipid, for example 1.9-2.0 g/100 g lipid, such as from 2.0-2.1 g/100 g lipid, for example 2.1-2.2 g/100 g lipid, such as from 2.2-2.3 g/100 g lipid, for example 2.3-2.4 g/100 g lipid, such as from 2.4-2.5 g/100 g lipid, for example 2.5-2.6 g/100 g lipid, such as from 2.6-2.7 g/100 g lipid, for example 2.7-2.8 g/100 g lipid, such as from 2.8-2.9 g/100 g lipid, for example 2.9-3.0 g/100 g lipid, such as from 3.0-3.1 g/100 g lipid, for example 3.1-3.2 g/100 g lipid, such as from 3.2-3.3 g/100 g lipid, for example 3.3-3.4 g/100 g lipid, such as from 3.4-3.5 g/100 g lipid, for example 3.5-3.6 g/100 g lipid, such as from 3.6-3.7 g/100 g lipid, for example 3.7-3.8 g/100 g lipid, such as from 3.8-3.9 g/100 g lipid, for example 3.9-4.0 g/100 g lipid, such as from 4.0-4.1 g/100 g lipid, for example 4.1-4.2 g/100 g lipid, such as from 4.2-4.3 g/100 g lipid, for example 4.3-4.4 g/100 g lipid, such as from 4.4-4.5 g/100 g lipid, for example 4.5-4.6 g/100 g lipid, such as from 4.6-4.7 g/100 g lipid, for example 4.7-4.8 g/100 g lipid, such as from 4.8-4.9 g/100 g lipid, for example 4.9-5.0 g/100 g lipid, such as from 5.0-5.1 g/100 g lipid, for example 5.1-5.2 g/100 g lipid, such as from 5.2-5.3 g/100 g lipid, for example 5.3-5.4 g/100 g lipid, such as from 5.4-5.5 g/100 g lipid, for example 5.5-5.6 g/100 g lipid, such as from 5.6-5.7 g/100 g lipid, for example 5.7-5.8 g/100 g lipid, such as from 5.8-5.9 g/100 g lipid, for example 5.9-6.0 g/100 g lipid, such as from 6.0-6.1 g/100 g lipid, for example 6.1-6.2 g/100 g lipid, such as from 6.2-6.3 g/100 g lipid, for example 6.3-6.4 g/100 g lipid, such as from 6.4-6.5 g/100 g lipid, for example 6.5-6.6 g/100 g lipid, such as from 6.6-6.7 g/100 g lipid, for example 6.7-6.8 g/100 g lipid, such as from 6.8-6.9 g/100 g lipid, for example 6.9-7.0 g/100 g lipid, such as from 7.0-7.1 g/100 g lipid, for example 7.1-7.2 g/100 g lipid, such as from 7.2-7.3 g/100 g lipid, for example 7.3-7.4 g/100 g lipid, such as from 7.4-7.5 g/100 g lipid, for example 7.5-7.6 g/100 g lipid, such as from 7.6-7.7 g/100 g lipid, for example 7.7-7.8 g/100 g lipid, such as from 7.8-7.9 g/100 g lipid, for example 7.9-8.0 g/100 g lipid, such as from 8.0-8.1 g/100 g lipid, for example 8.1-8.2 g/100 g lipid, such as from 8.2-8.3 g/100 g lipid, for example 8.3-8.4 g/100 g lipid, such as from 8.4-8.5 g/100 g lipid, for example 8.5-8.6 g/100 g lipid, such as from 8.6-8.7 g/100 g lipid, for example 8.7-8.8 g/100 g lipid, such as from 8.8-8.9 g/100 g lipid, for example 8.9-9.0 g/100 g lipid, such as from 9.0-9.1 g/100 g lipid, for example 9.1-9.2 g/100 g lipid, such as from 9.2-9.3 g/100 g lipid, for example 9.3-9.4 g/100 g lipid, such as from 9.4-9.5 g/100 g lipid, for example 9.5-9.6 g/100 g lipid, such as from 9.6-9.7 g/100 g lipid, for example 9.7-9.8 g/100 g lipid, such as from 9.8-9.9 g/100 g lipid, for example 9.9-10.0 g/100 g lipid, such as from 10.0-10.1 g/100 g lipid, for example 10.1-10.2 g/100 g lipid, such as from 10.2-10.3 g/100 g lipid, for example 10.3-10.4 g/100 g lipid, such as from 10.4-10.5 g/100 g lipid, for example 10.5-10.6 g/100 g lipid, such as from 10.6-10.7 g/100 g lipid, for example 10.7-10.8 g/100 g lipid, such as from 10.8-10.9 g/100 g lipid, for example 10.9-11.0 g/100 g lipid, such as from 11.0-11.1 g/100 g lipid, for example 11.1-11.2 g/100 g lipid, such as from 11.2-11.3 g/100 g lipid, for example 11.3-11.4 g/100 g lipid, such as from 11.4-11.5 g/100 g lipid, for example 11.5-11.6 g/100 g lipid, such as from 11.6-11.7 g/100 g lipid, for example 11.7-11.8 g/100 g lipid, such as from 11.8-11.9 g/100 g lipid, for example 11.9-12.0 g/100 g lipid, such as from 12.0-12.1 g/100 g lipid, for example 12.1-12.2 g/100 g lipid, such as from 12.2-12.3 g/100 g lipid, for example 12.3-12.4 g/100 g lipid, such as from 12.4-12.5 g/100 g lipid, for example 12.5-12.6 g/100 g lipid, such as from 12.6-12.7 g/100 g lipid, for example 12.7-12.8 g/100 g lipid, such as from 12.8-12.9 g/100 g lipid, for example 12.9-13.0 g/100 g lipid, such as from 13.0-13.1 g/100 g lipid, for example 13.1-13.2 g/100 g lipid, such as from 13.2-13.3 g/100 g lipid, for example 13.3-13.4 g/100 g lipid, such as from 13.4-13.5 g/100 g lipid, for example 13.5-13.6 g/100 g lipid, such as from 13.6-13.7 g/100 g lipid, for example 13.7-13.8 g/100 g lipid, such as from 13.8-13.9 g/100 g lipid, for example 13.9-14.0 g/100 g lipid, such as from 14.0-14.1 g/100 g lipid, for example 14.1-14.2 g/100 g lipid, such as from 14.2-14.3 g/100 g lipid, for example 14.3-14.4 g/100 g lipid, such as from 14.4-14.5 g/100 g lipid, for example 14.5-14.6 g/100 g lipid, such as from 14.6-14.7 g/100 g lipid, for example 14.7-14.8 g/100 g lipid, such as from 14.8-14.9 g/100 g lipid, for example 14.9-15.0 g/100 g lipid, such as from 15-16 g/100 g lipid, for example 16-17 g/100 g lipid, such as from 17-18 g/100 g lipid, for example 18-19 g/100 g lipid, such as from 19-20 g/100 g lipid.

In one embodiment the present invention relates to a fish feed composition with a 22:6 n-3 content (g/100 g lipid) in the range from 0-20 g/100 g lipid, such as from 0-0.1 g/100 g lipid, for example 0.1-0.2 g/100 g lipid, such as from 0.2-0.3 g/100 g lipid, for example 0.3-0.4 g/100 g lipid, such as from 0.4-0.5 g/100 g lipid, for example 0.5-0.6 g/100 g lipid, such as from 0.6-0.7 g/100 g lipid, for example 0.7-0.8 g/100 g lipid, such as from 0.8-0.9 g/100 g lipid, for example 0.9-1.0 g/100 g lipid, such as from 1.0-1.1 g/100 g lipid, for example 1.1-1.2 g/100 g lipid, such as from 1.2-1.3 g/100 g lipid, for example 1.3-1.4 g/100 g lipid, such as from 1.4-1.5 g/100 g lipid, for example 1.5-1.6 g/100 g lipid, such as from 1.6-1.7 g/100 g lipid, for example 1.7-1.8 g/100 g lipid, such as from 1.8-1.9 g/100 g lipid, for example 1.9-2.0 g/100 g lipid, such as from 2.0-2.1 g/100 g lipid, for example 2.1-2.2 g/100 g lipid, such as from 2.2-2.3 g/100 g lipid, for example 2.3-2.4 g/100 g lipid, such as from 2.4-2.5 g/100 g lipid, for example 2.5-2.6 g/100 g lipid, such as from 2.6-2.7 g/100 g lipid, for example 2.7-2.8 g/100 g lipid, such as from 2.8-2.9 g/100 g lipid, for example 2.9-3.0 g/100 g lipid, such as from 3.0-3.1 g/100 g lipid, for example 3.1-3.2 g/100 g lipid, such as from 3.2-3.3 g/100 g lipid, for example 3.3-3.4 g/100 g lipid, such as from 3.4-3.5 g/100 g lipid, for example 3.5-3.6 g/100 g lipid, such as from 3.6-3.7 g/100 g lipid, for example 3.7-3.8 g/100 g lipid, such as from 3.8-3.9 g/100 g lipid, for example 3.9-4.0 g/100 g lipid, such as from 4.0-4.1 g/100 g lipid, for example 4.1-4.2 g/100 g lipid, such as from 4.2-4.3 g/100 g lipid, for example 4.3-4.4 g/100 g lipid, such as from 4.4-4.5 g/100 g lipid, for example 4.5-4.6 g/100 g lipid, such as from 4.6-4.7 g/100 g lipid, for example 4.7-4.8 g/100 g lipid, such as from 4.8-4.9 g/100 g lipid, for example 4.9-5.0 g/100 g lipid, such as from 5.0-5.1 g/100 g lipid, for example 5.1-5.2 g/100 g lipid, such as from 5.2-5.3 g/100 g lipid, for example 5.3-5.4 g/100 g lipid, such as from 5.4-5.5 g/100 g lipid, for example 5.5-5.6 g/100 g lipid, such as from 5.6-5.7 g/100 g lipid, for example 5.7-5.8 g/100 g lipid, such as from 5.8-5.9 g/100 g lipid, for example 5.9-6.0 g/100 g lipid, such as from 6.0-6.1 g/100 g lipid, for example 6.1-6.2 g/100 g lipid, such as from 6.2-6.3 g/100 g lipid, for example 6.3-6.4 g/100 g lipid, such as from 6.4-6.5 g/100 g lipid, for example 6.5-6.6 g/100 g lipid, such as from 6.6-6.7 g/100 g lipid, for example 6.7-6.8 g/100 g lipid, such as from 6.8-6.9 g/100 g lipid, for example 6.9-7.0 g/100 g lipid, such as from 7.0-7.1 g/100 g lipid, for example 7.1-7.2 g/100 g lipid, such as from 7.2-7.3 g/100 g lipid, for example 7.3-7.4 g/100 g lipid, such as from 7.4-7.5 g/100 g lipid, for example 7.5-7.6 g/100 g lipid, such as from 7.6-7.7 g/100 g lipid, for example 7.7-7.8 g/100 g lipid, such as from 7.8-7.9 g/100 g lipid, for example 7.9-8.0 g/100 g lipid, such as from 8.0-8.1 g/100 g lipid, for example 8.1-8.2 g/100 g lipid, such as from 8.2-8.3 g/100 g lipid, for example 8.3-8.4 g/100 g lipid, such as from 8.4-8.5 g/100 g lipid, for example 8.5-8.6 g/100 g lipid, such as from 8.6-8.7 g/100 g lipid, for example 8.7-8.8 g/100 g lipid, such as from 8.8-8.9 g/100 g lipid, for example 8.9-9.0 g/100 g lipid, such as from 9.0-9.1 g/100 g lipid, for example 9.1-9.2 g/100 g lipid, such as from 9.2-9.3 g/100 g lipid, for example 9.3-9.4 g/100 g lipid, such as from 9.4-9.5 g/100 g lipid, for example 9.5-9.6 g/100 g lipid, such as from 9.6-9.7 g/100 g lipid, for example 9.7-9.8 g/100 g lipid, such as from 9.8-9.9 g/100 g lipid, for example 9.9-10.0 g/100 g lipid, such as from 10.0-10.1 g/100 g lipid, for example 10.1-10.2 g/100 g lipid, such as from 10.2-10.3 g/100 g lipid, for example 10.3-10.4 g/100 g lipid, such as from 10.4-10.5 g/100 g lipid, for example 10.5-10.6 g/100 g lipid, such as from 10.6-10.7 g/100 g lipid, for example 10.7-10.8 g/100 g lipid, such as from 10.8-10.9 g/100 g lipid, for example 10.9-11.0 g/100 g lipid, such as from 11.0-11.1 g/100 g lipid, for example 11.1-11.2 g/100 g lipid, such as from 11.2-11.3 g/100 g lipid, for example 11.3-11.4 g/100 g lipid, such as from 11.4-11.5 g/100 g lipid, for example 11.5-11.6 g/100 g lipid, such as from 11.6-11.7 g/100 g lipid, for example 11.7-11.8 g/100 g lipid, such as from 11.8-11.9 g/100 g lipid, for example 11.9-12.0 g/100 g lipid, such as from 12.0-12.1 g/100 g lipid, for example 12.1-12.2 g/100 g lipid, such as from 12.2-12.3 g/100 g lipid, for example 12.3-12.4 g/100 g lipid, such as from 12.4-12.5 g/100 g lipid, for example 12.5-12.6 g/100 g lipid, such as from 12.6-12.7 g/100 g lipid, for example 12.7-12.8 g/100 g lipid, such as from 12.8-12.9 g/100 g lipid, for example 12.9-13.0 g/100 g lipid, such as from 13.0-13.1 g/100 g lipid, for example 13.1-13.2 g/100 g lipid, such as from 13.2-13.3 g/100 g lipid, for example 13.3-13.4 g/100 g lipid, such as from 13.4-13.5 g/100 g lipid, for example 13.5-13.6 g/100 g lipid, such as from 13.6-13.7 g/100 g lipid, for example 13.7-13.8 g/100 g lipid, such as from 13.8-13.9 g/100 g lipid, for example 13.9-14.0 g/100 g lipid, such as from 14.0-14.1 g/100 g lipid, for example 14.1-14.2 g/100 g lipid, such as from 14.2-14.3 g/100 g lipid, for example 14.3-14.4 g/100 g lipid, such as from 14.4-14.5 g/100 g lipid, for example 14.5-14.6 g/100 g lipid, such as from 14.6-14.7 g/100 g lipid, for example 14.7-14.8 g/100 g lipid, such as from 14.8-14.9 g/100 g lipid, for example 14.9-15.0 g/100 g lipid, such as from 15-16 g/100 g lipid, for example 16-17 g/100 g lipid, such as from 17-18 g/100 g lipid, for example 18-19 g/100 g lipid, such as from 19-20 g/100 g lipid.

The present invention relates in one embodiment to a fish feed composition with a protein:energy ratio in the range from about 50 mg/kcal to about 200 mg/kcal; for example from about 50 mg/kcal to about 190 mg/kcal, such as from about 50 mg/kcal to about 180 mg/kcal, for example from about 50 mg/kcal to about 170 mg/kcal, such as from about 50 mg/kcal to about 160 mg/kcal, for example from about 50 mg/kcal to about 150 mg/kcal, such as from about 50 mg/kcal to about 140 mg/kcal, for example from about 50 mg/kcal to about 130 mg/kcal, such as from about 50 mg/kcal to about 120 mg/kcal, for example from about 50 mg/kcal to about 110 mg/kcal, such as from about 50 mg/kcal to about 100 mg/kcal, for example from about 50 mg/kcal to about 90 mg/kcal, such as from about 50 mg/kcal to about 80 mg/kcal, for example from about 50 mg/kcal to about 70 mg/kcal, such as from about 50 mg/kcal to about 60 mg/kcal, for example from about 60 mg/kcal to about 200 mg/kcal, such as from about 70 mg/kcal to about 200 mg/kcal, for example from about 80 mg/kcal to about 200 mg/kcal, such as from about 90 mg/kcal to about 200 mg/kcal, for example from about 100 mg/kcal to about 200 mg/kcal, such as from about 110 mg/kcal to about 200 mg/kcal, for example from about 120 mg/kcal to about 200 mg/kcal, such as from about 130 mg/kcal to about 200 mg/kcal, for example from about 140 mg/kcal to about 200 mg/kcal, such as from about 150 mg/kcal to about 200 mg/kcal, for example from about 160 mg/kcal to about 200 mg/kcal, such as from about 170 mg/kcal to about 200 mg/kcal, for example from about 180 mg/kcal to about 200 mg/kcal, such as from about 190 mg/kcal to about 200 mg/kcal, for example from about 50 mg/kcal to about 60 mg/kcal, such as from about 60 mg/kcal to about 70 mg/kcal, for example from about 70 mg/kcal to about 80 mg/kcal, such as from about 80 mg/kcal to about 90 mg/kcal, for example from about 90 mg/kcal to about 100 mg/kcal, such as from about 100 mg/kcal to about 110 mg/kcal, for example from about 110 mg/kcal to about 120 mg/kcal, such as from about 120 mg/kcal to about 130 mg/kcal, for example from about 130 mg/kcal to about 140 mg/kcal, such as from about 140 mg/kcal to about 150 mg/kcal, for example from about 150 mg/kcal to about 160 mg/kcal, such as from about 160 mg/kcal to about 170 mg/kcal, for example from about 170 mg/kcal to about 180 mg/kcal, such as from about 180 mg/kcal to about 190 mg/kcal, for example from about 190 mg/kcal to about 200 mg/kcal.

In one embodiment the present invention relates to a fish feed composition comprising from 500 kcal/kg diet to 5000 kcal/kg diet, such as from 500 to 600 kcal/kg, for example from 600 to 700 kcal/kg, from 700 to 800 kcal/kg, for example from 800 to 900 kcal/kg, from 900 to 1000 kcal/kg, for example from 1000 to 1100 kcal/kg, from 1100 to 1200 kcal/kg, for example from 1200 to 1300 kcal/kg, from 1300 to 1400 kcal/kg, for example from 1400 to 1500 kcal/kg, from 1500 to 1600 kcal/kg, for example from 1600 to 1700 kcal/kg, from 1700 to 1800 kcal/kg, for example from 1800 to 1900 kcal/kg, from 1900 to 2000 kcal/kg, for example from 2000 to 2100 kcal/kg, from 2100 to 2200 kcal/kg, for example from 2200 to 2300 kcal/kg, from 2300 to 2400 kcal/kg, for example from 2400 to 2500 kcal/kg, from 2500 to 2600 kcal/kg, for example from 2600 to 2700 kcal/kg, from 2700 to 2800 kcal/kg, for example from 2800 to 2900 kcal/kg, from 2900 to 3000 kcal/kg, for example from 3000 to 3100 kcal/kg, from 3100 to 3200 kcal/kg, for example from 3200 to 3300 kcal/kg, from 3300 to 3400 kcal/kg, for example from 3400 to 3500 kcal/kg, from 3500 to 3600 kcal/kg, for example from 3600 to 3700 kcal/kg, from 3700 to 3800 kcal/kg, for example from 3800 to 3900 kcal/kg, from 3900 to 4000 kcal/kg, for example from 4000 to 4100 kcal/kg, from 4100 to 4200 kcal/kg, for example from 4200 to 4300 kcal/kg, from 4300 to 4400 kcal/kg, for example from 4400 to 4500 kcal/kg, from 4500 to 4600 kcal/kg, for example from 4600 to 4700 kcal/kg, from 4700 to 4800 kcal/kg, for example from 4800 to 4900 kcal/kg, from 4900 to 5000 kcal/kg.

In an embodiment of the present invention the one or more protein sources comprise between 5 and 95% of the feed, such as between 10 and 90%, such as between 15 and 80%, such as between 20 and 75% such as between 25 and 70%, such as between 30 and 65%, such as between 35 and 60%, such as between 40 and 55%, such as between 45 and 50%, or between 30 and 50%, such as between 35 and 45% such as around 40%, or such as between 50 and 70%, such as between 55 and 65% such as around 60% of the feed composition.

In another embodiment of the present invention the one or more protein sources comprise between 18 and 70% of the feed, such as from 18 to 20%, for example 20 to 22%, such as from 22 to 24%, for example 24 to 26%, such as from 26 to 28%, for example 28 to 30%, such as from 30 to 32%, for example 32 to 34%, such as from 34 to 36%, for example 36 to 38%, such as from 38 to 40%, for example 40 to 42%, such as from 42 to 44%, for example 44 to 46%, such as from 46 to 48%, for example 48 to 50%, such as from 50 to 52%, for example 52 to 54%, such as from 54 to 56%, for example 56 to 58%, such as from 58 to 60%, for example 60 to 62%, such as from 62 to 64%, for example 64 to 66%, such as from 66 to 68%, for example 68 to 70%.

The present invention relates in one embodiment to a fish feed composition wherein the percentage of Arginine of dietary protein is in the range from 0.5 to 20%, such as from 0.5 to 18%, for example from 0.5 to 16%, such as from 0.5 to 14%, for example from 0.5 to 12%, such as from 0.5 to 10%, for example from 0.5 to 8%, such as from 0.5 to 6%, for example from 0.5 to 4%, such as from 0.5 to 2%, for example from 2 to 20%, such as from 4 to 20%, for example from 6 to 20%, such as from 8 to 20%, for example from 10 to 20%, such as from 12 to 20%, for example from 14 to 20%, such as from 16 to 20%, for example from 18 to 20%, such as from 0.5 to 1%, for example from 1 to 1.5%, such as from 1.5 to 2%, for example from 2 to 2.5%, such as from 2 to 3%, for example from 3 to 4%, such as from 4 to 5%, for example from 5 to 6%, such as from 6 to 7%, for example from 7 to 8%, such as from 8 to 9%, for example from 9 to 10%, such as from 10 to 11%, for example from 11 to 12%, such as from 12 to 13%, for example from 13 to 14%, such as from 14 to 15%, for example from 15 to 16%, such as from 16 to 17%, for example from 17 to 18%, such as from 18 to 19%, for example from 19 to 20%, such as from 1.0 to 1.1%, for example from 1.1 to 1.2%, such as from 1.2 to 1.3%, for example from 1.3 to 1.4%, such as from 1.4 to 1.5%, for example from 1.5 to 1.6%, such as from 1.6 to 1.7%, for example from 1.7 to 1.8%, such as from 1.8 to 1.9%, for example from 1.9 to 2.0%, such as from 2.0 to 2.1%, for example from 2.1 to 2.2%, such as from 2.2 to 2.3%, for example from 2.3 to 2.4%, such as from 2.4 to 2.5%, for example from 2.5 to 2.6%, such as from 2.6 to 2.7%, for example from 2.7 to 2.8%, such as from 2.8 to 2.9%, for example from 2.9 to 3.0%, such as from 3.0 to 3.1%, for example from 3.1 to 3.2%, such as from 3.2 to 3.3%, for example from 3.3 to 3.4%, such as from 3.4 to 3.5%, for example from 3.5 to 3.6%, such as from 3.6 to 3.7%, for example from 3.7 to 3.8%, such as from 3.8 to 3.9%, for example from 3.9 to 4.0%, such as from 4.0 to 4.1%, for example from 4.1 to 4.2%, such as from 4.2 to 4.3%, for example from 4.3 to 4.4%, such as from 4.4 to 4.5%, for example from 4.5 to 4.6%, such as from 4.6 to 4.7%, for example from 4.7 to 4.8%, such as from 4.8 to 4.9%, for example from 4.9 to 5.0%, such as from 5.0 to 5.1%, for example from 5.1 to 5.2%, such as from 5.2 to 5.3%, for example from 5.3 to 5.4%, such as from 5.4 to 5.5%, for example from 5.5 to 5.6%, such as from 5.6 to 5.7%, for example from 5.7 to 5.8%, such as from 5.8 to 5.9%, for example from 5.9 to 6.0%, such as from 6.0 to 6.1%, for example from 6.1 to 6.2%, such as from 6.2 to 6.3%, for example from 6.3 to 6.4%, such as from 6.4 to 6.5%, for example from 6.5 to 6.6%, such as from 6.6 to 6.7%, for example from 6.7 to 6.8%, such as from 6.8 to 6.9%, for example from 6.9 to 7.0%, such as from 7.0 to 7.1%, for example from 7.1 to 7.2%, such as from 7.2 to 7.3%, for example from 7.3 to 7.4%, such as from 7.4 to 7.5%, for example from 7.5 to 7.6%, such as from 7.6 to 7.7%, for example from 7.7 to 7.8%, such as from 7.8 to 7.9%, for example from 7.9 to 8.0%.

The present invention relates in one embodiment to a fish feed composition wherein the percentage of Histidine of dietary protein is in the range from 0.5 to 20%, such as from 0.5 to 18%, for example from 0.5 to 16%, such as from 0.5 to 14%, for example from 0.5 to 12%, such as from 0.5 to 10%, for example from 0.5 to 8%, such as from 0.5 to 6%, for example from 0.5 to 4%, such as from 0.5 to 2%, for example from 2 to 20%, such as from 4 to 20%, for example from 6 to 20%, such as from 8 to 20%, for example from 10 to 20%, such as from 12 to 20%, for example from 14 to 20%, such as from 16 to 20%, for example from 18 to 20%, such as from 0.5 to 1%, for example from 1 to 1.5%, such as from 1.5 to 2%, for example from 2 to 2.5%, such as from 2 to 3%, for example from 3 to 4%, such as from 4 to 5%, for example from 5 to 6%, such as from 6 to 7%, for example from 7 to 8%, such as from 8 to 9%, for example from 9 to 10%, such as from 10 to 11%, for example from 11 to 12%, such as from 12 to 13%, for example from 13 to 14%, such as from 14 to 15%, for example from 15 to 16%, such as from 16 to 17%, for example from 17 to 18%, such as from 18 to 19%, for example from 19 to 20%, such as from 1.0 to 1.1%, for example from 1.1 to 1.2%, such as from 1.2 to 1.3%, for example from 1.3 to 1.4%, such as from 1.4 to 1.5%, for example from 1.5 to 1.6%, such as from 1.6 to 1.7%, for example from 1.7 to 1.8%, such as from 1.8 to 1.9%, for example from 1.9 to 2.0%, such as from 2.0 to 2.1%, for example from 2.1 to 2.2%, such as from 2.2 to 2.3%, for example from 2.3 to 2.4%, such as from 2.4 to 2.5%, for example from 2.5 to 2.6%, such as from 2.6 to 2.7%, for example from 2.7 to 2.8%, such as from 2.8 to 2.9%, for example from 2.9 to 3.0%, such as from 3.0 to 3.1%, for example from 3.1 to 3.2%, such as from 3.2 to 3.3%, for example from 3.3 to 3.4%, such as from 3.4 to 3.5%, for example from 3.5 to 3.6%, such as from 3.6 to 3.7%, for example from 3.7 to 3.8%, such as from 3.8 to 3.9%, for example from 3.9 to 4.0%, such as from 4.0 to 4.1%, for example from 4.1 to 4.2%, such as from 4.2 to 4.3%, for example from 4.3 to 4.4%, such as from 4.4 to 4.5%, for example from 4.5 to 4.6%, such as from 4.6 to 4.7%, for example from 4.7 to 4.8%, such as from 4.8 to 4.9%, for example from 4.9 to 5.0%, such as from 5.0 to 5.1%, for example from 5.1 to 5.2%, such as from 5.2 to 5.3%, for example from 5.3 to 5.4%, such as from 5.4 to 5.5%, for example from 5.5 to 5.6%, such as from 5.6 to 5.7%, for example from 5.7 to 5.8%, such as from 5.8 to 5.9%, for example from 5.9 to 6.0%, such as from 6.0 to 6.1%, for example from 6.1 to 6.2%, such as from 6.2 to 6.3%, for example from 6.3 to 6.4%, such as from 6.4 to 6.5%, for example from 6.5 to 6.6%, such as from 6.6 to 6.7%, for example from 6.7 to 6.8%, such as from 6.8 to 6.9%, for example from 6.9 to 7.0%, such as from 7.0 to 7.1%, for example from 7.1 to 7.2%, such as from 7.2 to 7.3%, for example from 7.3 to 7.4%, such as from 7.4 to 7.5%, for example from 7.5 to 7.6%, such as from 7.6 to 7.7%, for example from 7.7 to 7.8%, such as from 7.8 to 7.9%, for example from 7.9 to 8.0%.

The present invention relates in one embodiment to a fish feed composition wherein the percentage of Isoleucine of dietary protein is in the range from 0.5 to 20%, such as from 0.5 to 18%, for example from 0.5 to 16%, such as from 0.5 to 14%, for example from 0.5 to 12%, such as from 0.5 to 10%, for example from 0.5 to 8%, such as from 0.5 to 6%, for example from 0.5 to 4%, such as from 0.5 to 2%, for example from 2 to 20%, such as from 4 to 20%, for example from 6 to 20%, such as from 8 to 20%, for example from 10 to 20%, such as from 12 to 20%, for example from 14 to 20%, such as from 16 to 20%, for example from 18 to 20%, such as from 0.5 to 1%, for example from 1 to 1.5%, such as from 1.5 to 2%, for example from 2 to 2.5%, such as from 2 to 3%, for example from 3 to 4%, such as from 4 to 5%, for example from 5 to 6%, such as from 6 to 7%, for example from 7 to 8%, such as from 8 to 9%, for example from 9 to 10%, such as from 10 to 11%, for example from 11 to 12%, such as from 12 to 13%, for example from 13 to 14%, such as from 14 to 15%, for example from 15 to 16%, such as from 16 to 17%, for example from 17 to 18%, such as from 18 to 19%, for example from 19 to 20%, such as from 1.0 to 1.1%, for example from 1.1 to 1.2%, such as from 1.2 to 1.3%, for example from 1.3 to 1.4%, such as from 1.4 to 1.5%, for example from 1.5 to 1.6%, such as from 1.6 to 1.7%, for example from 1.7 to 1.8%, such as from 1.8 to 1.9%, for example from 1.9 to 2.0%, such as from 2.0 to 2.1%, for example from 2.1 to 2.2%, such as from 2.2 to 2.3%, for example from 2.3 to 2.4%, such as from 2.4 to 2.5%, for example from 2.5 to 2.6%, such as from 2.6 to 2.7%, for example from 2.7 to 2.8%, such as from 2.8 to 2.9%, for example from 2.9 to 3.0%, such as from 3.0 to 3.1%, for example from 3.1 to 3.2%, such as from 3.2 to 3.3%, for example from 3.3 to 3.4%, such as from 3.4 to 3.5%, for example from 3.5 to 3.6%, such as from 3.6 to 3.7%, for example from 3.7 to 3.8%, such as from 3.8 to 3.9%, for example from 3.9 to 4.0%, such as from 4.0 to 4.1%, for example from 4.1 to 4.2%, such as from 4.2 to 4.3%, for example from 4.3 to 4.4%, such as from 4.4 to 4.5%, for example from 4.5 to 4.6%, such as from 4.6 to 4.7%, for example from 4.7 to 4.8%, such as from 4.8 to 4.9%, for example from 4.9 to 5.0%, such as from 5.0 to 5.1%, for example from 5.1 to 5.2%, such as from 5.2 to 5.3%, for example from 5.3 to 5.4%, such as from 5.4 to 5.5%, for example from 5.5 to 5.6%, such as from 5.6 to 5.7%, for example from 5.7 to 5.8%, such as from 5.8 to 5.9%, for example from 5.9 to 6.0%, such as from 6.0 to 6.1%, for example from 6.1 to 6.2%, such as from 6.2 to 6.3%, for example from 6.3 to 6.4%, such as from 6.4 to 6.5%, for example from 6.5 to 6.6%, such as from 6.6 to 6.7%, for example from 6.7 to 6.8%, such as from 6.8 to 6.9%, for example from 6.9 to 7.0%, such as from 7.0 to 7.1%, for example from 7.1 to 7.2%, such as from 7.2 to 7.3%, for example from 7.3 to 7.4%, such as from 7.4 to 7.5%, for example from 7.5 to 7.6%, such as from 7.6 to 7.7%, for example from 7.7 to 7.8%, such as from 7.8 to 7.9%, for example from 7.9 to 8.0%.

The present invention relates in one embodiment to a fish feed composition wherein the percentage of Leucine of dietary protein is in the range from 0.5 to 20%, such as from 0.5 to 18%, for example from 0.5 to 16%, such as from 0.5 to 14%, for example from 0.5 to 12%, such as from 0.5 to 10%, for example from 0.5 to 8%, such as from 0.5 to 6%, for example from 0.5 to 4%, such as from 0.5 to 2%, for example from 2 to 20%, such as from 4 to 20%, for example from 6 to 20%, such as from 8 to 20%, for example from 10 to 20%, such as from 12 to 20%, for example from 14 to 20%, such as from 16 to 20%, for example from 18 to 20%, such as from 0.5 to 1%, for example from 1 to 1.5%, such as from 1.5 to 2%, for example from 2 to 2.5%, such as from 2 to 3%, for example from 3 to 4%, such as from 4 to 5%, for example from 5 to 6%, such as from 6 to 7%, for example from 7 to 8%, such as from 8 to 9%, for example from 9 to 10%, such as from 10 to 11%, for example from 11 to 12%, such as from 12 to 13%, for example from 13 to 14%, such as from 14 to 15%, for example from 15 to 16%, such as from 16 to 17%, for example from 17 to 18%, such as from 18 to 19%, for example from 19 to 20%, such as from 1.0 to 1.1%, for example from 1.1 to 1.2%, such as from 1.2 to 1.3%, for example from 1.3 to 1.4%, such as from 1.4 to 1.5%, for example from 1.5 to 1.6%, such as from 1.6 to 1.7%, for example from 1.7 to 1.8%, such as from 1.8 to 1.9%, for example from 1.9 to 2.0%, such as from 2.0 to 2.1%, for example from 2.1 to 2.2%, such as from 2.2 to 2.3%, for example from 2.3 to 2.4%, such as from 2.4 to 2.5%, for example from 2.5 to 2.6%, such as from 2.6 to 2.7%, for example from 2.7 to 2.8%, such as from 2.8 to 2.9%, for example from 2.9 to 3.0%, such as from 3.0 to 3.1%, for example from 3.1 to 3.2%, such as from 3.2 to 3.3%, for example from 3.3 to 3.4%, such as from 3.4 to 3.5%, for example from 3.5 to 3.6%, such as from 3.6 to 3.7%, for example from 3.7 to 3.8%, such as from 3.8 to 3.9%, for example from 3.9 to 4.0%, such as from 4.0 to 4.1%, for example from 4.1 to 4.2%, such as from 4.2 to 4.3%, for example from 4.3 to 4.4%, such as from 4.4 to 4.5%, for example from 4.5 to 4.6%, such as from 4.6 to 4.7%, for example from 4.7 to 4.8%, such as from 4.8 to 4.9%, for example from 4.9 to 5.0%, such as from 5.0 to 5.1%, for example from 5.1 to 5.2%, such as from 5.2 to 5.3%, for example from 5.3 to 5.4%, such as from 5.4 to 5.5%, for example from 5.5 to 5.6%, such as from 5.6 to 5.7%, for example from 5.7 to 5.8%, such as from 5.8 to 5.9%, for example from 5.9 to 6.0%, such as from 6.0 to 6.1%, for example from 6.1 to 6.2%, such as from 6.2 to 6.3%, for example from 6.3 to 6.4%, such as from 6.4 to 6.5%, for example from 6.5 to 6.6%, such as from 6.6 to 6.7%, for example from 6.7 to 6.8%, such as from 6.8 to 6.9%, for example from 6.9 to 7.0%, such as from 7.0 to 7.1%, for example from 7.1 to 7.2%, such as from 7.2 to 7.3%, for example from 7.3 to 7.4%, such as from 7.4 to 7.5%, for example from 7.5 to 7.6%, such as from 7.6 to 7.7%, for example from 7.7 to 7.8%, such as from 7.8 to 7.9%, for example from 7.9 to 8.0%.

The present invention relates in one embodiment to a fish feed composition wherein the percentage of Lysine of dietary protein is in the range from 0.5 to 20%, such as from 0.5 to 18%, for example from 0.5 to 16%, such as from 0.5 to 14%, for example from 0.5 to 12%, such as from 0.5 to 10%, for example from 0.5 to 8%, such as from 0.5 to 6%, for example from 0.5 to 4%, such as from 0.5 to 2%, for example from 2 to 20%, such as from 4 to 20%, for example from 6 to 20%, such as from 8 to 20%, for example from 10 to 20%, such as from 12 to 20%, for example from 14 to 20%, such as from 16 to 20%, for example from 18 to 20%, such as from 0.5 to 1%, for example from 1 to 1.5%, such as from 1.5 to 2%, for example from 2 to 2.5%, such as from 2 to 3%, for example from 3 to 4%, such as from 4 to 5%, for example from 5 to 6%, such as from 6 to 7%, for example from 7 to 8%, such as from 8 to 9%, for example from 9 to 10%, such as from 10 to 11%, for example from 11 to 12%, such as from 12 to 13%, for example from 13 to 14%, such as from 14 to 15%, for example from 15 to 16%, such as from 16 to 17%, for example from 17 to 18%, such as from 18 to 19%, for example from 19 to 20%, such as from 1.0 to 1.1%, for example from 1.1 to 1.2%, such as from 1.2 to 1.3%, for example from 1.3 to 1.4%, such as from 1.4 to 1.5%, for example from 1.5 to 1.6%, such as from 1.6 to 1.7%, for example from 1.7 to 1.8%, such as from 1.8 to 1.9%, for example from 1.9 to 2.0%, such as from 2.0 to 2.1%, for example from 2.1 to 2.2%, such as from 2.2 to 2.3%, for example from 2.3 to 2.4%, such as from 2.4 to 2.5%, for example from 2.5 to 2.6%, such as from 2.6 to 2.7%, for example from 2.7 to 2.8%, such as from 2.8 to 2.9%, for example from 2.9 to 3.0%, such as from 3.0 to 3.1%, for example from 3.1 to 3.2%, such as from 3.2 to 3.3%, for example from 3.3 to 3.4%, such as from 3.4 to 3.5%, for example from 3.5 to 3.6%, such as from 3.6 to 3.7%, for example from 3.7 to 3.8%, such as from 3.8 to 3.9%, for example from 3.9 to 4.0%, such as from 4.0 to 4.1%, for example from 4.1 to 4.2%, such as from 4.2 to 4.3%, for example from 4.3 to 4.4%, such as from 4.4 to 4.5%, for example from 4.5 to 4.6%, such as from 4.6 to 4.7%, for example from 4.7 to 4.8%, such as from 4.8 to 4.9%, for example from 4.9 to 5.0%, such as from 5.0 to 5.1%, for example from 5.1 to 5.2%, such as from 5.2 to 5.3%, for example from 5.3 to 5.4%, such as from 5.4 to 5.5%, for example from 5.5 to 5.6%, such as from 5.6 to 5.7%, for example from 5.7 to 5.8%, such as from 5.8 to 5.9%, for example from 5.9 to 6.0%, such as from 6.0 to 6.1%, for example from 6.1 to 6.2%, such as from 6.2 to 6.3%, for example from 6.3 to 6.4%, such as from 6.4 to 6.5%, for example from 6.5 to 6.6%, such as from 6.6 to 6.7%, for example from 6.7 to 6.8%, such as from 6.8 to 6.9%, for example from 6.9 to 7.0%, such as from 7.0 to 7.1%, for example from 7.1 to 7.2%, such as from 7.2 to 7.3%, for example from 7.3 to 7.4%, such as from 7.4 to 7.5%, for example from 7.5 to 7.6%, such as from 7.6 to 7.7%, for example from 7.7 to 7.8%, such as from 7.8 to 7.9%, for example from 7.9 to 8.0%.

The present invention relates in one embodiment to a fish feed composition wherein the percentage of Methionine of dietary protein is in the range from 0.5 to 20%, such as from 0.5 to 18%, for example from 0.5 to 16%, such as from 0.5 to 14%, for example from 0.5 to 12%, such as from 0.5 to 10%, for example from 0.5 to 8%, such as from 0.5 to 6%, for example from 0.5 to 4%, such as from 0.5 to 2%, for example from 2 to 20%, such as from 4 to 20%, for example from 6 to 20%, such as from 8 to 20%, for example from 10 to 20%, such as from 12 to 20%, for example from 14 to 20%, such as from 16 to 20%, for example from 18 to 20%, such as from 0.5 to 1%, for example from 1 to 1.5%, such as from 1.5 to 2%, for example from 2 to 2.5%, such as from 2 to 3%, for example from 3 to 4%, such as from 4 to 5%, for example from 5 to 6%, such as from 6 to 7%, for example from 7 to 8%, such as from 8 to 9%, for example from 9 to 10%, such as from 10 to 11%, for example from 11 to 12%, such as from 12 to 13%, for example from 13 to 14%, such as from 14 to 15%, for example from 15 to 16%, such as from 16 to 17%, for example from 17 to 18%, such as from 18 to 19%, for example from 19 to 20%, such as from 1.0 to 1.1%, for example from 1.1 to 1.2%, such as from 1.2 to 1.3%, for example from 1.3 to 1.4%, such as from 1.4 to 1.5%, for example from 1.5 to 1.6%, such as from 1.6 to 1.7%, for example from 1.7 to 1.8%, such as from 1.8 to 1.9%, for example from 1.9 to 2.0%, such as from 2.0 to 2.1%, for example from 2.1 to 2.2%, such as from 2.2 to 2.3%, for example from 2.3 to 2.4%, such as from 2.4 to 2.5%, for example from 2.5 to 2.6%, such as from 2.6 to 2.7%, for example from 2.7 to 2.8%, such as from 2.8 to 2.9%, for example from 2.9 to 3.0%, such as from 3.0 to 3.1%, for example from 3.1 to 3.2%, such as from 3.2 to 3.3%, for example from 3.3 to 3.4%, such as from 3.4 to 3.5%, for example from 3.5 to 3.6%, such as from 3.6 to 3.7%, for example from 3.7 to 3.8%, such as from 3.8 to 3.9%, for example from 3.9 to 4.0%, such as from 4.0 to 4.1%, for example from 4.1 to 4.2%, such as from 4.2 to 4.3%, for example from 4.3 to 4.4%, such as from 4.4 to 4.5%, for example from 4.5 to 4.6%, such as from 4.6 to 4.7%, for example from 4.7 to 4.8%, such as from 4.8 to 4.9%, for example from 4.9 to 5.0%, such as from 5.0 to 5.1%, for example from 5.1 to 5.2%, such as from 5.2 to 5.3%, for example from 5.3 to 5.4%, such as from 5.4 to 5.5%, for example from 5.5 to 5.6%, such as from 5.6 to 5.7%, for example from 5.7 to 5.8%, such as from 5.8 to 5.9%, for example from 5.9 to 6.0%, such as from 6.0 to 6.1%, for example from 6.1 to 6.2%, such as from 6.2 to 6.3%, for example from 6.3 to 6.4%, such as from 6.4 to 6.5%, for example from 6.5 to 6.6%, such as from 6.6 to 6.7%, for example from 6.7 to 6.8%, such as from 6.8 to 6.9%, for example from 6.9 to 7.0%, such as from 7.0 to 7.1%, for example from 7.1 to 7.2%, such as from 7.2 to 7.3%, for example from 7.3 to 7.4%, such as from 7.4 to 7.5%, for example from 7.5 to 7.6%, such as from 7.6 to 7.7%, for example from 7.7 to 7.8%, such as from 7.8 to 7.9%, for example from 7.9 to 8.0%.

The present invention relates in one embodiment to a fish feed composition wherein the percentage of Phenylalanine of dietary protein is in the range from 0.5 to 20%, such as from 0.5 to 18%, for example from 0.5 to 16%, such as from 0.5 to 14%, for example from 0.5 to 12%, such as from 0.5 to 10%, for example from 0.5 to 8%, such as from 0.5 to 6%, for example from 0.5 to 4%, such as from 0.5 to 2%, for example from 2 to 20%, such as from 4 to 20%, for example from 6 to 20%, such as from 8 to 20%, for example from 10 to 20%, such as from 12 to 20%, for example from 14 to 20%, such as from 16 to 20%, for example from 18 to 20%, such as from 0.5 to 1%, for example from 1 to 1.5%, such as from 1.5 to 2%, for example from 2 to 2.5%, such as from 2 to 3%, for example from 3 to 4%, such as from 4 to 5%, for example from 5 to 6%, such as from 6 to 7%, for example from 7 to 8%, such as from 8 to 9%, for example from 9 to 10%, such as from 10 to 11%, for example from 11 to 12%, such as from 12 to 13%, for example from 13 to 14%, such as from 14 to 15%, for example from 15 to 16%, such as from 16 to 17%, for example from 17 to 18%, such as from 18 to 19%, for example from 19 to 20%, such as from 1.0 to 1.1%, for example from 1.1 to 1.2%, such as from 1.2 to 1.3%, for example from 1.3 to 1.4%, such as from 1.4 to 1.5%, for example from 1.5 to 1.6%, such as from 1.6 to 1.7%, for example from 1.7 to 1.8%, such as from 1.8 to 1.9%, for example from 1.9 to 2.0%, such as from 2.0 to 2.1%, for example from 2.1 to 2.2%, such as from 2.2 to 2.3%, for example from 2.3 to 2.4%, such as from 2.4 to 2.5%, for example from 2.5 to 2.6%, such as from 2.6 to 2.7%, for example from 2.7 to 2.8%, such as from 2.8 to 2.9%, for example from 2.9 to 3.0%, such as from 3.0 to 3.1%, for example from 3.1 to 3.2%, such as from 3.2 to 3.3%, for example from 3.3 to 3.4%, such as from 3.4 to 3.5%, for example from 3.5 to 3.6%, such as from 3.6 to 3.7%, for example from 3.7 to 3.8%, such as from 3.8 to 3.9%, for example from 3.9 to 4.0%, such as from 4.0 to 4.1%, for example from 4.1 to 4.2%, such as from 4.2 to 4.3%, for example from 4.3 to 4.4%, such as from 4.4 to 4.5%, for example from 4.5 to 4.6%, such as from 4.6 to 4.7%, for example from 4.7 to 4.8%, such as from 4.8 to 4.9%, for example from 4.9 to 5.0%, such as from 5.0 to 5.1%, for example from 5.1 to 5.2%, such as from 5.2 to 5.3%, for example from 5.3 to 5.4%, such as from 5.4 to 5.5%, for example from 5.5 to 5.6%, such as from 5.6 to 5.7%, for example from 5.7 to 5.8%, such as from 5.8 to 5.9%, for example from 5.9 to 6.0%, such as from 6.0 to 6.1%, for example from 6.1 to 6.2%, such as from 6.2 to 6.3%, for example from 6.3 to 6.4%, such as from 6.4 to 6.5%, for example from 6.5 to 6.6%, such as from 6.6 to 6.7%, for example from 6.7 to 6.8%, such as from 6.8 to 6.9%, for example from 6.9 to 7.0%, such as from 7.0 to 7.1%, for example from 7.1 to 7.2%, such as from 7.2 to 7.3%, for example from 7.3 to 7.4%, such as from 7.4 to 7.5%, for example from 7.5 to 7.6%, such as from 7.6 to 7.7%, for example from 7.7 to 7.8%, such as from 7.8 to 7.9%, for example from 7.9 to 8.0%.

The present invention relates in one embodiment to a fish feed composition wherein the percentage of Threonine of dietary protein is in the range from 0.5 to 20%, such as from 0.5 to 18%, for example from 0.5 to 16%, such as from 0.5 to 14%, for example from 0.5 to 12%, such as from 0.5 to 10%, for example from 0.5 to 8%, such as from 0.5 to 6%, for example from 0.5 to 4%, such as from 0.5 to 2%, for example from 2 to 20%, such as from 4 to 20%, for example from 6 to 20%, such as from 8 to 20%, for example from 10 to 20%, such as from 12 to 20%, for example from 14 to 20%, such as from 16 to 20%, for example from 18 to 20%, such as from 0.5 to 1%, for example from 1 to 1.5%, such as from 1.5 to 2%, for example from 2 to 2.5%, such as from 2 to 3%, for example from 3 to 4%, such as from 4 to 5%, for example from 5 to 6%, such as from 6 to 7%, for example from 7 to 8%, such as from 8 to 9%, for example from 9 to 10%, such as from 10 to 11%, for example from 11 to 12%, such as from 12 to 13%, for example from 13 to 14%, such as from 14 to 15%, for example from 15 to 16%, such as from 16 to 17%, for example from 17 to 18%, such as from 18 to 19%, for example from 19 to 20%, such as from 1.0 to 1.1%, for example from 1.1 to 1.2%, such as from 1.2 to 1.3%, for example from 1.3 to 1.4%, such as from 1.4 to 1.5%, for example from 1.5 to 1.6%, such as from 1.6 to 1.7%, for example from 1.7 to 1.8%, such as from 1.8 to 1.9%, for example from 1.9 to 2.0%, such as from 2.0 to 2.1%, for example from 2.1 to 2.2%, such as from 2.2 to 2.3%, for example from 2.3 to 2.4%, such as from 2.4 to 2.5%, for example from 2.5 to 2.6%, such as from 2.6 to 2.7%, for example from 2.7 to 2.8%, such as from 2.8 to 2.9%, for example from 2.9 to 3.0%, such as from 3.0 to 3.1%, for example from 3.1 to 3.2%, such as from 3.2 to 3.3%, for example from 3.3 to 3.4%, such as from 3.4 to 3.5%, for example from 3.5 to 3.6%, such as from 3.6 to 3.7%, for example from 3.7 to 3.8%, such as from 3.8 to 3.9%, for example from 3.9 to 4.0%, such as from 4.0 to 4.1%, for example from 4.1 to 4.2%, such as from 4.2 to 4.3%, for example from 4.3 to 4.4%, such as from 4.4 to 4.5%, for example from 4.5 to 4.6%, such as from 4.6 to 4.7%, for example from 4.7 to 4.8%, such as from 4.8 to 4.9%, for example from 4.9 to 5.0%, such as from 5.0 to 5.1%, for example from 5.1 to 5.2%, such as from 5.2 to 5.3%, for example from 5.3 to 5.4%, such as from 5.4 to 5.5%, for example from 5.5 to 5.6%, such as from 5.6 to 5.7%, for example from 5.7 to 5.8%, such as from 5.8 to 5.9%, for example from 5.9 to 6.0%, such as from 6.0 to 6.1%, for example from 6.1 to 6.2%, such as from 6.2 to 6.3%, for example from 6.3 to 6.4%, such as from 6.4 to 6.5%, for example from 6.5 to 6.6%, such as from 6.6 to 6.7%, for example from 6.7 to 6.8%, such as from 6.8 to 6.9%, for example from 6.9 to 7.0%, such as from 7.0 to 7.1%, for example from 7.1 to 7.2%, such as from 7.2 to 7.3%, for example from 7.3 to 7.4%, such as from 7.4 to 7.5%, for example from 7.5 to 7.6%, such as from 7.6 to 7.7%, for example from 7.7 to 7.8%, such as from 7.8 to 7.9%, for example from 7.9 to 8.0%.

The present invention relates in one embodiment to a fish feed composition wherein the percentage of Tryptophan of dietary protein is in the range from 0.1 to 10%, such as from 0.1 to 9.5%, for example from 1.0 to 9.0%, such as from 0.1 to 8.5%, for example from 1.0 to 8.0%, such as from 0.1 to 7.5%, for example from 1.0 to 7.0%, such as from 0.1 to 6.5%, for example from 1.0 to 6.0%, such as from 0.1 to 5.5%, for example from 1.0 to 5.0%, such as from 0.1 to 4.5%, for example from 1.0 to 4.0%, such as from 0.1 to 3.5%, for example from 1.0 to 3.0%, such as from 0.1 to 2.5%, for example from 1.0 to 2.0%, such as from 0.1 to 1.5%, for example from 1.0 to 1.0%, such as from 0.1 to 0.5%, for example from 0.5 to 10%, such as from 1.0 to 10%, for example from 1.5 to 10%, such as from 2.0 to 10%, for example from 2.5 to 10%, such as from 3.0 to 10%, for example from 3.5 to 10%, such as from 4.0 to 10%, for example from 4.5 to 10%, such as from 5.0 to 10%, for example from 5.5 to 10%, such as from 6.0 to 10%, for example from 6.5 to 10%, such as from 7.0 to 10%, for example from 7.5 to 10%, such as from 8.0 to 10%, for example from 8.5 to 10%, such as from 9.0 to 10%, for example from 9.5 to 10%, such as from 0.1 to 1%, for example from 1 to 2%, such as from 2 to 3%, for example from 3 to 4%, such as from 4 to 5%, for example from 5 to 6%, such as from 6 to 7%, for example from 7 to 8%, such as from 8 to 9%, for example from 9 to 10%, such as from 0.1 to 0.2%, for example from 0.2 to 0.3%, such as from 0.3 to 0.4%, for example from 0.4 to 0.5%, such as from 0.5 to 0.6%, for example from 0.6 to 0.7%, such as from 0.7 to 0.8%, for example from 0.8 to 0.9%, such as from 0.9 to 1.0%, for example from 1.0 to 1.1%, such as from 1.1 to 1.2%, for example from 1.2 to 1.3%, such as from 1.3 to 1.4%, for example from 1.4 to 1.5%, such as from 1.5 to 1.6%, for example from 1.6 to 1.7%, such as from 1.7 to 1.8%, for example from 1.8 to 1.9%, such as from 1.9 to 2.0%, for example from 2.1 to 2.2%, such as from 2.2 to 2.3%, for example from 2.3 to 2.4%, such as from 2.4 to 2.5%, for example from 2.5 to 2.6%, such as from 2.6 to 2.7%, for example from 2.7 to 2.8%, such as from 2.8 to 2.9%, for example from 2.9 to 3.0%.

The present invention relates in one embodiment to a fish feed composition wherein the percentage of Valine of dietary protein is in the range from 0.5 to 20%, such as from 0.5 to 18%, for example from 0.5 to 16%, such as from 0.5 to 14%, for example from 0.5 to 12%, such as from 0.5 to 10%, for example from 0.5 to 8%, such as from 0.5 to 6%, for example from 0.5 to 4%, such as from 0.5 to 2%, for example from 2 to 20%, such as from 4 to 20%, for example from 6 to 20%, such as from 8 to 20%, for example from 10 to 20%, such as from 12 to 20%, for example from 14 to 20%, such as from 16 to 20%, for example from 18 to 20%, such as from 0.5 to 1%, for example from 1 to 1.5%, such as from 1.5 to 2%, for example from 2 to 2.5%, such as from 2 to 3%, for example from 3 to 4%, such as from 4 to 5%, for example from 5 to 6%, such as from 6 to 7%, for example from 7 to 8%, such as from 8 to 9%, for example from 9 to 10%, such as from 10 to 11%, for example from 11 to 12%, such as from 12 to 13%, for example from 13 to 14%, such as from 14 to 15%, for example from 15 to 16%, such as from 16 to 17%, for example from 17 to 18%, such as from 18 to 19%, for example from 19 to 20%, such as from 1.0 to 1.1%, for example from 1.1 to 1.2%, such as from 1.2 to 1.3%, for example from 1.3 to 1.4%, such as from 1.4 to 1.5%, for example from 1.5 to 1.6%, such as from 1.6 to 1.7%, for example from 1.7 to 1.8%, such as from 1.8 to 1.9%, for example from 1.9 to 2.0%, such as from 2.0 to 2.1%, for example from 2.1 to 2.2%, such as from 2.2 to 2.3%, for example from 2.3 to 2.4%, such as from 2.4 to 2.5%, for example from 2.5 to 2.6%, such as from 2.6 to 2.7%, for example from 2.7 to 2.8%, such as from 2.8 to 2.9%, for example from 2.9 to 3.0%, such as from 3.0 to 3.1%, for example from 3.1 to 3.2%, such as from 3.2 to 3.3%, for example from 3.3 to 3.4%, such as from 3.4 to 3.5%, for example from 3.5 to 3.6%, such as from 3.6 to 3.7%, for example from 3.7 to 3.8%, such as from 3.8 to 3.9%, for example from 3.9 to 4.0%, such as from 4.0 to 4.1%, for example from 4.1 to 4.2%, such as from 4.2 to 4.3%, for example from 4.3 to 4.4%, such as from 4.4 to 4.5%, for example from 4.5 to 4.6%, such as from 4.6 to 4.7%, for example from 4.7 to 4.8%, such as from 4.8 to 4.9%, for example from 4.9 to 5.0%, such as from 5.0 to 5.1%, for example from 5.1 to 5.2%, such as from 5.2 to 5.3%, for example from 5.3 to 5.4%, such as from 5.4 to 5.5%, for example from 5.5 to 5.6%, such as from 5.6 to 5.7%, for example from 5.7 to 5.8%, such as from 5.8 to 5.9%, for example from 5.9 to 6.0%, such as from 6.0 to 6.1%, for example from 6.1 to 6.2%, such as from 6.2 to 6.3%, for example from 6.3 to 6.4%, such as from 6.4 to 6.5%, for example from 6.5 to 6.6%, such as from 6.6 to 6.7%, for example from 6.7 to 6.8%, such as from 6.8 to 6.9%, for example from 6.9 to 7.0%, such as from 7.0 to 7.1%, for example from 7.1 to 7.2%, such as from 7.2 to 7.3%, for example from 7.3 to 7.4%, such as from 7.4 to 7.5%, for example from 7.5 to 7.6%, such as from 7.6 to 7.7%, for example from 7.7 to 7.8%, such as from 7.8 to 7.9%, for example from 7.9 to 8.0%.

The lipids of the feed composition according to the present invention constitute between 0.5 to 50% of the feed (by % inclusion) such as between 1 and 45%, such as between 1.5 and 40% such as between 2 and 35%, such as between 2.5 and 30%, such as between 3 and 25%, such as between 3.5 and 30%, such as between 4 and 25%, or between 4.5 and 20%, such as between 5 and 15%, such as between 5.5 and 10%, such as between 6 and 9%, such as between 6.5 and 8%, such as around 7%, or such as between 2 and 10%, such as between 3 and 9% such as between 4 and 8%, such as between 5 and 7% such as around 6% of the feed composition.

In another embodiment the lipids of the feed composition according to the present invention constitute from 5 to 25% of the feed, such as from 5 to 6%, for example from 6-7%, such as from 7 to 8%, for example from 8-9%, such as from 9 to 10%, for example from 10-11%, such as from 11 to 12%, for example from 12-13%, such as from 13 to 14%, for example from 14-15%, such as from 15 to 16%, for example from 16-17%, such as from 17 to 18%, for example from 18-19%, such as from 19 to 20%, for example from 20-21%, such as from 21 to 22%, for example from 22-23%, such as from 23 to 24%, for example from 24-25%.

The present invention relates in one embodiment to a fish feed composition wherein the percentage of EPA (eicosapentaenoic acid), 20:5(n-3) is in the range from 0.1 to 25%, such as from 0.1 to 20%, for example from 0.1 to 15%, such as from 0.1 to 10%, for example from 0.1 to 5%, such as from 1 to 25%, for example from 5 to 25%, such as from 10 to 25%, for example from 15 to 25%, such as from 20 to 25%, for example from 0.1 to 5%, such as from 5 to 10%, for example from 10 to 15%, such as from 15 to 20%, for example from 20 to 25%, such as from 0.1 to 0.2%, for example from 0.2 to 0.3%, such as from 0.3 to 0.4%, for example from 0.4 to 0.5%, such as from 0.5 to 0.6%, for example from 0.6 to 0.7%, such as from 0.7 to 0.8%, for example from 0.8 to 0.9%, such as from 0.9 to 1.0%, for example from 1.0 to 1.1%, such as from 1.1 to 1.2%, for example from 1.2 to 1.3%, such as from 1.3 to 1.4%, for example from 1.4 to 1.5%, such as from 1.5 to 1.6%, for example from 1.6 to 1.7%, such as from 1.7 to 1.8%, for example from 1.8 to 1.9%, such as from 1.9 to 2.0%, for example from 2.0 to 2.1%, such as from 2.1 to 2.2%, for example from 2.2 to 2.3%, such as from 2.3 to 2.4%, for example from 2.4 to 2.5%, such as from 2.5 to 2.6%, for example from 2.6 to 2.7%, such as from 2.7 to 2.8%, for example from 2.8 to 2.9%, such as from 2.9 to 3.0%, for example from 3.0 to 3.1%, such as from 3.1 to 3.2%, for example from 3.2 to 3.3%, such as from 3.3 to 3.4%, for example from 3.4 to 3.5%, such as from 3.5 to 3.6%, for example from 3.6 to 3.7%, such as from 3.7 to 3.8%, for example from 3.8 to 3.9%, such as from 3.9 to 4.0%.

The present invention relates in one embodiment to a fish feed composition wherein the percentage of Linolenic acid, 18:3(n-3) is in the range from 0.1 to 25%, such as from 0.1 to 20%, for example from 0.1 to 15%, such as from 0.1 to 10%, for example from 0.1 to 5%, such as from 1 to 25%, for example from 5 to 25%, such as from 10 to 25%, for example from 15 to 25%, such as from 20 to 25%, for example from 0.1 to 5%, such as from 5 to 10%, for example from 10 to 15%, such as from 15 to 20%, for example from 20 to 25%, such as from 0.1 to 0.2%, for example from 0.2 to 0.3%, such as from 0.3 to 0.4%, for example from 0.4 to 0.5%, such as from 0.5 to 0.6%, for example from 0.6 to 0.7%, such as from 0.7 to 0.8%, for example from 0.8 to 0.9%, such as from 0.9 to 1.0%, for example from 1.0 to 1.1%, such as from 1.1 to 1.2%, for example from 1.2 to 1.3%, such as from 1.3 to 1.4%, for example from 1.4 to 1.5%, such as from 1.5 to 1.6%, for example from 1.6 to 1.7%, such as from 1.7 to 1.8%, for example from 1.8 to 1.9%, such as from 1.9 to 2.0%, for example from 2.0 to 2.1%, such as from 2.1 to 2.2%, for example from 2.2 to 2.3%, such as from 2.3 to 2.4%, for example from 2.4 to 2.5%, such as from 2.5 to 2.6%, for example from 2.6 to 2.7%, such as from 2.7 to 2.8%, for example from 2.8 to 2.9%, such as from 2.9 to 3.0%, for example from 3.0 to 3.1%, such as from 3.1 to 3.2%, for example from 3.2 to 3.3%, such as from 3.3 to 3.4%, for example from 3.4 to 3.5%, such as from 3.5 to 3.6%, for example from 3.6 to 3.7%, such as from 3.7 to 3.8%, for example from 3.8 to 3.9%, such as from 3.9 to 4.0%.

The present invention relates in one embodiment to a fish feed composition wherein the percentage of DHA (docosahexaenoic acid), 22:6(n-3) is in the range from 0.1 to 25%, such as from 0.1 to 20%, for example from 0.1 to 15%, such as from 0.1 to 10%, for example from 0.1 to 5%, such as from 1 to 25%, for example from 5 to 25%, such as from 10 to 25%, for example from 15 to 25%, such as from 20 to 25%, for example from 0.1 to 5%, such as from 5 to 10%, for example from 10 to 15%, such as from 15 to 20%, for example from 20 to 25%, such as from 0.1 to 0.2%, for example from 0.2 to 0.3%, such as from 0.3 to 0.4%, for example from 0.4 to 0.5%, such as from 0.5 to 0.6%, for example from 0.6 to 0.7%, such as from 0.7 to 0.8%, for example from 0.8 to 0.9%, such as from 0.9 to 1.0%, for example from 1.0 to 1.1%, such as from 1.1 to 1.2%, for example from 1.2 to 1.3%, such as from 1.3 to 1.4%, for example from 1.4 to 1.5%, such as from 1.5 to 1.6%, for example from 1.6 to 1.7%, such as from 1.7 to 1.8%, for example from 1.8 to 1.9%, such as from 1.9 to 2.0%, for example from 2.0 to 2.1%, such as from 2.1 to 2.2%, for example from 2.2 to 2.3%, such as from 2.3 to 2.4%, for example from 2.4 to 2.5%, such as from 2.5 to 2.6%, for example from 2.6 to 2.7%, such as from 2.7 to 2.8%, for example from 2.8 to 2.9%, such as from 2.9 to 3.0%, for example from 3.0 to 3.1%, such as from 3.1 to 3.2%, for example from 3.2 to 3.3%, such as from 3.3 to 3.4%, for example from 3.4 to 3.5%, such as from 3.5 to 3.6%, for example from 3.6 to 3.7%, such as from 3.7 to 3.8%, for example from 3.8 to 3.9%, such as from 3.9 to 4.0%.

The present invention relates in one embodiment to a fish feed composition wherein the percentage of linoleic acid, 18:2(n-6) is in the range from 0.1 to 25%, such as from 0.1 to 20%, for example from 0.1 to 15%, such as from 0.1 to 10%, for example from 0.1 to 5%, such as from 1 to 25%, for example from 5 to 25%, such as from 10 to 25%, for example from 15 to 25%, such as from 20 to 25%, for example from 0.1 to 5%, such as from 5 to 10%, for example from 10 to 15%, such as from 15 to 20%, for example from 20 to 25%, such as from 0.1 to 0.2%, for example from 0.2 to 0.3%, such as from 0.3 to 0.4%, for example from 0.4 to 0.5%, such as from 0.5 to 0.6%, for example from 0.6 to 0.7%, such as from 0.7 to 0.8%, for example from 0.8 to 0.9%, such as from 0.9 to 1.0%, for example from 1.0 to 1.1%, such as from 1.1 to 1.2%, for example from 1.2 to 1.3%, such as from 1.3 to 1.4%, for example from 1.4 to 1.5%, such as from 1.5 to 1.6%, for example from 1.6 to 1.7%, such as from 1.7 to 1.8%, for example from 1.8 to 1.9%, such as from 1.9 to 2.0%, for example from 2.0 to 2.1%, such as from 2.1 to 2.2%, for example from 2.2 to 2.3%, such as from 2.3 to 2.4%, for example from 2.4 to 2.5%, such as from 2.5 to 2.6%, for example from 2.6 to 2.7%, such as from 2.7 to 2.8%, for example from 2.8 to 2.9%, such as from 2.9 to 3.0%, for example from 3.0 to 3.1%, such as from 3.1 to 3.2%, for example from 3.2 to 3.3%, such as from 3.3 to 3.4%, for example from 3.4 to 3.5%, such as from 3.5 to 3.6%, for example from 3.6 to 3.7%, such as from 3.7 to 3.8%, for example from 3.8 to 3.9%, such as from 3.9 to 4.0%.

The present invention relates in one embodiment to a fish feed composition wherein the percentage of arachidonic acid, 20:4(n-6) is in the range from 0.1 to 25%, such as from 0.1 to 20%, for example from 0.1 to 15%, such as from 0.1 to 10%, for example from 0.1 to 5%, such as from 1 to 25%, for example from 5 to 25%, such as from 10 to 25%, for example from 15 to 25%, such as from 20 to 25%, for example from 0.1 to 5%, such as from 5 to 10%, for example from 10 to 15%, such as from 15 to 20%, for example from 20 to 25%, such as from 0.1 to 0.2%, for example from 0.2 to 0.3%, such as from 0.3 to 0.4%, for example from 0.4 to 0.5%, such as from 0.5 to 0.6%, for example from 0.6 to 0.7%, such as from 0.7 to 0.8%, for example from 0.8 to 0.9%, such as from 0.9 to 1.0%, for example from 1.0 to 1.1%, such as from 1.1 to 1.2%, for example from 1.2 to 1.3%, such as from 1.3 to 1.4%, for example from 1.4 to 1.5%, such as from 1.5 to 1.6%, for example from 1.6 to 1.7%, such as from 1.7 to 1.8%, for example from 1.8 to 1.9%, such as from 1.9 to 2.0%, for example from 2.0 to 2.1%, such as from 2.1 to 2.2%, for example from 2.2 to 2.3%, such as from 2.3 to 2.4%, for example from 2.4 to 2.5%, such as from 2.5 to 2.6%, for example from 2.6 to 2.7%, such as from 2.7 to 2.8%, for example from 2.8 to 2.9%, such as from 2.9 to 3.0%, for example from 3.0 to 3.1%, such as from 3.1 to 3.2%, for example from 3.2 to 3.3%, such as from 3.3 to 3.4%, for example from 3.4 to 3.5%, such as from 3.5 to 3.6%, for example from 3.6 to 3.7%, such as from 3.7 to 3.8%, for example from 3.8 to 3.9%, such as from 3.9 to 4.0%.

In one embodiment the fish feed composition comprise one or more carbohydrates in the range from 0.5 to 30%, such as from 0.5 to 5%, for example from 5% to 10%, such as from 10 to 15%, for example from 15% to 20%, such as from 20 to 25%, for example from 25% to 30%, such as from 0.5 to 2%, for example from 2% to 4%, such as from 4 to 6%, for example from 6% to 8%, such as from 8 to 10%, for example from 10% to 12%, such as from 12 to 14%, for example from 14% to 16%, such as from 16 to 18%, for example from 18% to 20%, such as from 20 to 22%, for example from 22% to 24%, such as from 24 to 26%, for example from 26% to 28%, such as from 28 to 30%.

It is an object of the present invention to provide a feed composition which comprises at least one mineral source such as a commercial mineral mix optionally further supplemented by one or more minerals such as calcium phosphate and/or magnesium sulphate. In one embodiment the minerals of the feed composition according to the present invention constitute between 0.5 to 40% of the feed (by % inclusion) such as between 1 and 35%, such as between 1.5 and 30% such as between 2 and 25%, such as between 2.5 and 20%, such as between 3 and 15%, such as between 3.5 and 10%, such as between 4 and 9%, or between 4.5 and 8%, such as between 5 and 7%, such as around 6% or between 1 and 13% such as between 1.5 and 11%, such as between 2 and 9%, such as between 2.5 and 7%, such as between 3 and 5%, such as between 3.5 and 4.5%, such as around 4%, or around 3% or around 3.5% of the feed composition.

In one embodiment the fish feed composition comprise calcium in the range from 0.1 to 5% calcium, such as from 0.1 to 4%, for example from 0.1 to 3%, such as from 0.1 to 2%, for example from 0.1 to 1%, such as from 1 to 5%, for example from 2 to 5%, such as from 3 to 5%, for example from 4 to 5%, such as from 0.1 to 0.2%, for example from 0.2 to 0.3%, such as from 0.3 to 0.4%, for example from 0.4 to 0.5%, such as from 0.5 to 0.6%, for example from 0.6 to 0.7%, such as from 0.7 to 0.8%, for example from 0.8 to 0.9%, such as from 0.9 to 1.0%, for example from 1.0 to 1.1%, such as from 1.1 to 1.2%, for example from 1.2 to 1.3%, such as from 1.3 to 1.4%, for example from 1.4 to 1.5%, such as from 1.5 to 1.6%, for example from 1.6 to 1.7%, such as from 1.7 to 1.8%, for example from 1.8 to 1.9%, such as from 1.9 to 2.0%, for example from 2.0 to 2.1%, such as from 2.1 to 2.2%, for example from 2.2 to 2.3%, such as from 2.3 to 2.4%, for example from 2.4 to 2.5%, such as from 2.5 to 2.6%, for example from 2.6 to 2.7%, such as from 2.7 to 2.8%, for example from 2.8 to 2.9%, such as from 2.9 to 3.0%, for example from 3.0 to 3.1%, such as from 3.1 to 3.2%, for example from 3.2 to 3.3%, such as from 3.3 to 3.4%, for example from 3.4 to 3.3%, such as from 3.5 to 3.6%, for example from 3.6 to 3.3%, such as from 3.7 to 3.8%, for example from 3.8 to 3.3%, such as from 3.9 to 4.0%, for example from 4.0 to 3.3%, such as from 4.1 to 4.2%, for example from 4.2 to 4.3%, such as from 4.3 to 4.4%, for example from 4.4 to 4.5%, such as from 4.5 to 4.6%, for example from 4.6 to 4.7%, such as from 4.7 to 4.8%, for example from 4.8 to 4.9%, such as from 4.9 to 5.0%.

In one embodiment the fish feed composition comprise phosphorus in the range from 0.1 to 5% calcium, such as from 0.1 to 4%, for example from 0.1 to 3%, such as from 0.1 to 2%, for example from 0.1 to 1%, such as from 1 to 5%, for example from 2 to 5%, such as from 3 to 5%, for example from 4 to 5%, such as from 0.1 to 0.2%, for example from 0.2 to 0.3%, such as from 0.3 to 0.4%, for example from 0.4 to 0.5%, such as from 0.5 to 0.6%, for example from 0.6 to 0.7%, such as from 0.7 to 0.8%, for example from 0.8 to 0.9%, such as from 0.9 to 1.0%, for example from 1.0 to 1.1%, such as from 1.1 to 1.2%, for example from 1.2 to 1.3%, such as from 1.3 to 1.4%, for example from 1.4 to 1.5%, such as from 1.5 to 1.6%, for example from 1.6 to 1.7%, such as from 1.7 to 1.8%, for example from 1.8 to 1.9%, such as from 1.9 to 2.0%, for example from 2.0 to 2.1%, such as from 2.1 to 2.2%, for example from 2.2 to 2.3%, such as from 2.3 to 2.4%, for example from 2.4 to 2.5%, such as from 2.5 to 2.6%, for example from 2.6 to 2.7%, such as from 2.7 to 2.8%, for example from 2.8 to 2.9%, such as from 2.9 to 3.0%, for example from 3.0 to 3.1%, such as from 3.1 to 3.2%, for example from 3.2 to 3.3%, such as from 3.3 to 3.4%, for example from 3.4 to 3.3%, such as from 3.5 to 3.6%, for example from 3.6 to 3.3%, such as from 3.7 to 3.8%, for example from 3.8 to 3.3%, such as from 3.9 to 4.0%, for example from 4.0 to 3.3%, such as from 4.1 to 4.2%, for example from 4.2 to 4.3%, such as from 4.3 to 4.4%, for example from 4.4 to 4.5%, such as from 4.5 to 4.6%, for example from 4.6 to 4.7%, such as from 4.7 to 4.8%, for example from 4.8 to 4.9%, such as from 4.9 to 5.0%.

In one embodiment the fish feed composition comprise magnesium in the range from 0.01 to 0.5% of the diet, such as from 0.01 to 0.45%, for example from 0.01 to 0.4%, such as from 0.01 to 0.35%, for example from 0.01 to 0.3%, such as from 0.01 to 0.25%, for example from 0.01 to 0.2%, such as from 0.01 to 0.15%, for example from 0.01 to 0.1%, such as from 0.01 to 0.05%, for example from 0.05 to 0.5%, such as from 0.1 to 0.5%, for example from 0.15 to 0.5%, such as from 0.2 to 0.5%, for example from 0.25 to 0.5%, such as from 0.3 to 0.5%, for example from 0.35 to 0.5%, such as from 0.4 to 0.5%, for example from 0.45 to 0.5%, such as from 0.01 to 0.05%, for example from 0.05 to 0.1%, such as from 0.1 to 0.15%, for example from 0.15 to 0.2%, such as from 0.2 to 0.25%, for example from 0.25 to 0.3%, such as from 0.3 to 0.35%, for example from 0.35 to 0.4%, such as from 0.4 to 0.45%, for example from 0.45 to 0.5%, such as from 0.01 to 0.02%, for example from 0.02 to 0.03%, such as from 0.03 to 0.04%, for example from 0.04 to 0.05%, such as from 0.05 to 0.06%, for example from 0.06 to 0.07%, such as from 0.07 to 0.08%, for example from 0.08 to 0.09%, such as from 0.09 to 0.10%, for example from 0.10 to 0.11%, such as from 0.11 to 0.12%, for example from 0.12 to 0.13%, such as from 0.13 to 0.14%, for example from 0.14 to 0.15%, such as from 0.15 to 0.16%, for example from 0.16 to 0.17%, such as from 0.17 to 0.18%, for example from 0.18 to 0.19%, such as from 0.19 to 0.20%.

In one embodiment the present invention relates to a fish feed composition wherein the percentage of potassium is in the range from 0.1 to 5%, such as from 0.1 to 4%, for example from 0.1 to 3%, such as from 0.1 to 2%, for example from 0.1 to 1%, such as from 1 to 5%, for example from 2 to 5%, such as from 3 to 5%, for example from 4 to 5%, such as from 0.1 to 0.2%, for example from 0.2 to 0.3%, such as from 0.3 to 0.4%, for example from 0.4 to 0.5%, such as from 0.5 to 0.6%, for example from 0.6 to 0.7%, such as from 0.7 to 0.8%, for example from 0.8 to 0.9%, such as from 0.9 to 1.0%, for example from 1.0 to 1.1%, such as from 1.1 to 1.2%, for example from 1.2 to 1.3%, such as from 1.3 to 1.4%, for example from 1.4 to 1.5%, such as from 1.5 to 1.6%, for example from 1.6 to 1.7%, such as from 1.7 to 1.8%, for example from 1.8 to 1.9%, such as from 1.9 to 2.0%, for example from 2.0 to 2.1%, such as from 2.1 to 2.2%, for example from 2.2 to 2.3%, such as from 2.3 to 2.4%, for example from 2.4 to 2.5%, such as from 2.5 to 2.6%, for example from 2.6 to 2.7%, such as from 2.7 to 2.8%, for example from 2.8 to 2.9%, such as from 2.9 to 3.0%, for example from 3.0 to 3.1%, such as from 3.1 to 3.2%, for example from 3.2 to 3.3%, such as from 3.3 to 3.4%, for example from 3.4 to 3.3%, such as from 3.5 to 3.6%, for example from 3.6 to 3.3%, such as from 3.7 to 3.8%, for example from 3.8 to 3.3%, such as from 3.9 to 4.0%, for example from 4.0 to 3.3%, such as from 4.1 to 4.2%, for example from 4.2 to 4.3%, such as from 4.3 to 4.4%, for example from 4.4 to 4.5%, such as from 4.5 to 4.6%, for example from 4.6 to 4.7%, such as from 4.7 to 4.8%, for example from 4.8 to 4.9%, such as from 4.9 to 5.0%.

In another embodiment the fish feed composition according to the present invention comprises from 0.1 to 25% sodium, such as from 0.1 to 20%, for example from 0.1 to 15%, such as from 0.1 to 10%, for example from 0.1 to 5%, such as from 1 to 25%, for example from 5 to 25%, such as from 10 to 25%, for example from 15 to 25%, such as from 20 to 25%, for example from 0.1 to 5%, such as from 5 to 10%, for example from 10 to 15%, such as from 15 to 20%, for example from 20 to 25%, such as from 0.1 to 0.2%, for example from 0.2 to 0.3%, such as from 0.3 to 0.4%, for example from 0.4 to 0.5%, such as from 0.5 to 0.6%, for example from 0.6 to 0.7%, such as from 0.7 to 0.8%, for example from 0.8 to 0.9%, such as from 0.9 to 1.0%, for example from 1.0 to 1.1%, such as from 1.1 to 1.2%, for example from 1.2 to 1.3%, such as from 1.3 to 1.4%, for example from 1.4 to 1.5%, such as from 1.5 to 1.6%, for example from 1.6 to 1.7%, such as from 1.7 to 1.8%, for example from 1.8 to 1.9%, such as from 1.9 to 2.0%, for example from 2.0 to 2.1%, such as from 2.1 to 2.2%, for example from 2.2 to 2.3%, such as from 2.3 to 2.4%, for example from 2.4 to 2.5%, such as from 2.5 to 2.6%, for example from 2.6 to 2.7%, such as from 2.7 to 2.8%, for example from 2.8 to 2.9%, such as from 2.9 to 3.0%, for example from 3.0 to 3.1%, such as from 3.1 to 3.2%, for example from 3.2 to 3.3%, such as from 3.3 to 3.4%, for example from 3.4 to 3.5%, such as from 3.5 to 3.6%, for example from 3.6 to 3.7%, such as from 3.7 to 3.8%, for example from 3.8 to 3.9%, such as from 3.9 to 4.0%, such as from 1 to 3%, for example from 3% to 5%, such as from 5 to 7%, for example from 7% to 9%, such as from 9 to 11%, for example from 11% to 13%, such as from 13 to 15%, for example from 15% to 17%, such as from 17 to 19%, for example from 19% to 21%, such as from 17 to 19%, for example from 19% to 21%, such as from 19 to 21%, for example from 21% to 23%, such as from 23 to 25%.

In another embodiment the fish feed composition according to the present invention comprises from 0.1 to 25% chloride, such as from 0.1 to 20%, for example from 0.1 to 15%, such as from 0.1 to 10%, for example from 0.1 to 5%, such as from 1 to 25%, for example from 5 to 25%, such as from 10 to 25%, for example from 15 to 25%, such as from 20 to 25%, for example from 0.1 to 5%, such as from 5 to 10%, for example from 10 to 15%, such as from 15 to 20%, for example from 20 to 25%, such as from 0.1 to 0.2%, for example from 0.2 to 0.3%, such as from 0.3 to 0.4%, for example from 0.4 to 0.5%, such as from 0.5 to 0.6%, for example from 0.6 to 0.7%, such as from 0.7 to 0.8%, for example from 0.8 to 0.9%, such as from 0.9 to 1.0%, for example from 1.0 to 1.1%, such as from 1.1 to 1.2%, for example from 1.2 to 1.3%, such as from 1.3 to 1.4%, for example from 1.4 to 1.5%, such as from 1.5 to 1.6%, for example from 1.6 to 1.7%, such as from 1.7 to 1.8%, for example from 1.8 to 1.9%, such as from 1.9 to 2.0%, for example from 2.0 to 2.1%, such as from 2.1 to 2.2%, for example from 2.2 to 2.3%, such as from 2.3 to 2.4%, for example from 2.4 to 2.5%, such as from 2.5 to 2.6%, for example from 2.6 to 2.7%, such as from 2.7 to 2.8%, for example from 2.8 to 2.9%, such as from 2.9 to 3.0%, for example from 3.0 to 3.1%, such as from 3.1 to 3.2%, for example from 3.2 to 3.3%, such as from 3.3 to 3.4%, for example from 3.4 to 3.5%, such as from 3.5 to 3.6%, for example from 3.6 to 3.7%, such as from 3.7 to 3.8%, for example from 3.8 to 3.9%, such as from 3.9 to 4.0%, such as from 1 to 3%, for example from 3% to 5%, such as from 5 to 7%, for example from 7% to 9%, such as from 9 to 11%, for example from 11% to 13%, such as from 13 to 15%, for example from 15% to 17%, such as from 17 to 19%, for example from 19% to 21%, such as from 17 to 19%, for example from 19% to 21%, such as from 19 to 21%, for example from 21% to 23%, such as from 23 to 25%.

In one embodiment the present invention relates to a fish feed composition comprising from 5-500 mg/kg of iron, such as from 5 to 10 mg/kg, for example 10 to 15 mg/kg, such as from 15 to 20 mg/kg, for example 20 to 25 mg/kg, such as from 25 to 30 mg/kg, for example 30 to 35 mg/kg, such as from 35 to 40 mg/kg, for example 40 to 45 mg/kg, such as from 45 to 50 mg/kg, for example 50 to 55 mg/kg, such as from 55 to 60 mg/kg, for example 60 to 65 mg/kg, such as from 65 to 70 mg/kg, for example 70 to 75 mg/kg, such as from 75 to 80 mg/kg, for example 80 to 85 mg/kg, such as from 85 to 90 mg/kg, for example 90 to 95 mg/kg, such as from 95 to 100 mg/kg, for example 100 to 110 mg/kg, such as from 110 to 120 mg/kg, for example 120 to 130 mg/kg, such as from 130 to 140 mg/kg, for example 140 to 150 mg/kg, such as from 150 to 160 mg/kg, for example 160 to 170 mg/kg, such as from 170 to 180 mg/kg, for example 180 to 190 mg/kg, such as from 190 to 200 mg/kg, for example 200 to 220 mg/kg, such as from 190 to 200 mg/kg, for example 200 to 220 mg/kg, such as from 220 to 240 mg/kg, for example 240 to 260 mg/kg, such as from 260 to 280 mg/kg, for example 280 to 300 mg/kg, such as from 300 to 320 mg/kg, for example 320 to 340 mg/kg, such as from 340 to 360 mg/kg, for example 360 to 380 mg/kg, such as from 380 to 400 mg/kg, for example 400 to 420 mg/kg, such as from 420 to 440 mg/kg, for example 440 to 460 mg/kg, such as from 460 to 480 mg/kg, for example 480 to 500 mg/kg.

In one embodiment the present invention relates to a fish feed composition comprising from 1-1000 mg/kg copper, such as from 1-2 mg/kg, for example from 2-3 mg/kg, such as from 3-4 mg/kg, for example from 4-5 mg/kg, such as from 5-6 mg/kg, for example from 6-7 mg/kg, such as from 7-8 mg/kg, for example from 8-9 mg/kg, such as from 9-10 mg/kg, for example from 10-12 mg/kg, such as from 12-14 mg/kg, for example from 14-16 mg/kg, such as from 16-18 mg/kg, for example from 18-20 mg/kg, such as from 20-25 mg/kg, for example from 25-30 mg/kg, such as from 30-35 mg/kg, for example from 35-40 mg/kg, such as from 40-45 mg/kg, for example from 45-50 mg/kg, such as from 50-60 mg/kg, for example from 60-70 mg/kg, such as from 70-80 mg/kg, for example from 80-90 mg/kg, such as from 90-100 mg/kg, for example from 100-150 mg/kg, such as from 150-200 mg/kg, for example from 200-250 mg/kg, such as from 250-300 mg/kg, for example from 300-350 mg/kg, such as from 350-400 mg/kg, for example from 400-500 mg/kg, such as from 500-600 mg/kg, for example from 600-700 mg/kg, such as from 700-800 mg/kg, for example from 800-900 mg/kg, such as from 900-1000 mg/kg.

In one embodiment the present invention relates to a fish feed composition comprising from 1-1000 mg/kg zinc, such as from 1-2 mg/kg, for example from 2-3 mg/kg, such as from 3-4 mg/kg, for example from 4-5 mg/kg, such as from 5-6 mg/kg, for example from 6-7 mg/kg, such as from 7-8 mg/kg, for example from 8-9 mg/kg, such as from 9-10 mg/kg, for example from 10-12 mg/kg, such as from 12-14 mg/kg, for example from 14-16 mg/kg, such as from 16-18 mg/kg, for example from 18-20 mg/kg, such as from 20-25 mg/kg, for example from 25-30 mg/kg, such as from 30-35 mg/kg, for example from 35-40 mg/kg, such as from 40-45 mg/kg, for example from 45-50 mg/kg, such as from 50-60 mg/kg, for example from 60-70 mg/kg, such as from 70-80 mg/kg, for example from 80-90 mg/kg, such as from 90-100 mg/kg, for example from 100-150 mg/kg, such as from 150-200 mg/kg, for example from 200-250 mg/kg, such as from 250-300 mg/kg, for example from 300-350 mg/kg, such as from 350-400 mg/kg, for example from 400-500 mg/kg, such as from 500-600 mg/kg, for example from 600-700 mg/kg, such as from 700-800 mg/kg, for example from 800-900 mg/kg, such as from 900-1000 mg/kg.

In one embodiment the present invention relates to a fish feed composition comprising from 1-1000 mg/kg manganese, such as from 1-2 mg/kg, for example from 2-3 mg/kg, such as from 3-4 mg/kg, for example from 4-5 mg/kg, such as from 5-6 mg/kg, for example from 6-7 mg/kg, such as from 7-8 mg/kg, for example from 8-9 mg/kg, such as from 9-10 mg/kg, for example from 10-12 mg/kg, such as from 12-14 mg/kg, for example from 14-16 mg/kg, such as from 16-18 mg/kg, for example from 18-20 mg/kg, such as from 20-25 mg/kg, for example from 25-30 mg/kg, such as from 30-35 mg/kg, for example from 35-40 mg/kg, such as from 40-45 mg/kg, for example from 45-50 mg/kg, such as from 50-60 mg/kg, for example from 60-70 mg/kg, such as from 70-80 mg/kg, for example from 80-90 mg/kg, such as from 90-100 mg/kg, for example from 100-150 mg/kg, such as from 150-200 mg/kg, for example from 200-250 mg/kg, such as from 250-300 mg/kg, for example from 300-350 mg/kg, such as from 350-400 mg/kg, for example from 400-500 mg/kg, such as from 500-600 mg/kg, for example from 600-700 mg/kg, such as from 700-800 mg/kg, for example from 800-900 mg/kg, such as from 900-1000 mg/kg.

In one embodiment the present invention relates to a fish feed composition comprising from 0.01 to 20 mg/kg selenium, such as 0.01 to 0.05 mg/kg, for example 0.05 to 0.1 mg/kg, such as 0.1 to 0.15 mg/kg, for example 0.15 to 0.2 mg/kg, such as 0.2 to 0.25 mg/kg, for example 0.25 to 0.3 mg/kg, such as 0.3 to 0.35 mg/kg, for example 0.35 to 0.4 mg/kg, such as 0.4 to 0.45 mg/kg, for example 0.45 to 0.5 mg/kg, such as 0.5 to 0.55 mg/kg, for example 0.55 to 0.6 mg/kg, such as 0.6 to 0.65 mg/kg, for example 0.65 to 0.7 mg/kg, such as 0.7 to 0.75 mg/kg, for example 0.75 to 0.8 mg/kg, such as 0.8 to 0.85 mg/kg, for example 0.85 to 0.9 mg/kg, such as 0.9 to 0.95 mg/kg, for example 0.95 to 1.0 mg/kg, such as 1 to 2 mg/kg, for example 2 to 3 mg/kg, such as 3 to 4 mg/kg, for example 4 to 5 mg/kg, such as 5 to 6 mg/kg, for example 6 to 7 mg/kg, such as 7 to 8 mg/kg, for example 8 to 9 mg/kg, such as 9 to 10 mg/kg, for example 10 to 11 mg/kg, such as 11 to 12 mg/kg, for example 12 to 13 mg/kg, such as 13 to 14 mg/kg, for example 14 to 15 mg/kg, such as 15 to 16 mg/kg, for example 16 to 17 mg/kg, such as 17 to 18 mg/kg, for example 18 to 19 mg/kg, such as 19 to 20 mg/kg.

In one embodiment the present invention relates to a fish feed composition comprising from 1-1000 mg/kg iodine, such as from 1-2 mg/kg, for example from 2-3 mg/kg, such as from 3-4 mg/kg, for example from 4-5 mg/kg, such as from 5-6 mg/kg, for example from 6-7 mg/kg, such as from 7-8 mg/kg, for example from 8-9 mg/kg, such as from 9-10 mg/kg, for example from 10-12 mg/kg, such as from 12-14 mg/kg, for example from 14-16 mg/kg, such as from 16-18 mg/kg, for example from 18-20 mg/kg, such as from 20-25 mg/kg, for example from 25-30 mg/kg, such as from 30-35 mg/kg, for example from 35-40 mg/kg, such as from 40-45 mg/kg, for example from 45-50 mg/kg, such as from 50-60 mg/kg, for example from 60-70 mg/kg, such as from 70-80 mg/kg, for example from 80-90 mg/kg, such as from 90-100 mg/kg, for example from 100-150 mg/kg, such as from 150-200 mg/kg, for example from 200-250 mg/kg, such as from 250-300 mg/kg, for example from 300-350 mg/kg, such as from 350-400 mg/kg, for example from 400-500 mg/kg, such as from 500-600 mg/kg, for example from 600-700 mg/kg, such as from 700-800 mg/kg, for example from 800-900 mg/kg, such as from 900-1000 mg/kg.

In one embodiment the present invention relates to a fish feed composition comprising from 1-1000 mg/kg fluorine, such as from 1-2 mg/kg, for example from 2-3 mg/kg, such as from 3-4 mg/kg, for example from 4-5 mg/kg, such as from 5-6 mg/kg, for example from 6-7 mg/kg, such as from 7-8 mg/kg, for example from 8-9 mg/kg, such as from 9-10 mg/kg, for example from 10-12 mg/kg, such as from 12-14 mg/kg, for example from 14-16 mg/kg, such as from 16-18 mg/kg, for example from 18-20 mg/kg, such as from 20-25 mg/kg, for example from 25-30 mg/kg, such as from 30-35 mg/kg, for example from 35-40 mg/kg, such as from 40-45 mg/kg, for example from 45-50 mg/kg, such as from 50-60 mg/kg, for example from 60-70 mg/kg, such as from 70-80 mg/kg, for example from 80-90 mg/kg, such as from 90-100 mg/kg, for example from 100-150 mg/kg, such as from 150-200 mg/kg, for example from 200-250 mg/kg, such as from 250-300 mg/kg, for example from 300-350 mg/kg, such as from 350-400 mg/kg, for example from 400-500 mg/kg, such as from 500-600 mg/kg, for example from 600-700 mg/kg, such as from 700-800 mg/kg, for example from 800-900 mg/kg, such as from 900-1000 mg/kg.

In one embodiment the present invention relates to a fish feed composition comprising from 1-1000 mg/kg fluoride, such as from 1-2 mg/kg, for example from 2-3 mg/kg, such as from 3-4 mg/kg, for example from 4-5 mg/kg, such as from 5-6 mg/kg, for example from 6-7 mg/kg, such as from 7-8 mg/kg, for example from 8-9 mg/kg, such as from 9-10 mg/kg, for example from 10-12 mg/kg, such as from 12-14 mg/kg, for example from 14-16 mg/kg, such as from 16-18 mg/kg, for example from 18-20 mg/kg, such as from 20-25 mg/kg, for example from 25-30 mg/kg, such as from 30-35 mg/kg, for example from 35-40 mg/kg, such as from 40-45 mg/kg, for example from 45-50 mg/kg, such as from 50-60 mg/kg, for example from 60-70 mg/kg, such as from 70-80 mg/kg, for example from 80-90 mg/kg, such as from 90-100 mg/kg, for example from 100-150 mg/kg, such as from 150-200 mg/kg, for example from 200-250 mg/kg, such as from 250-300 mg/kg, for example from 300-350 mg/kg, such as from 350-400 mg/kg, for example from 400-500 mg/kg, such as from 500-600 mg/kg, for example from 600-700 mg/kg, such as from 700-800 mg/kg, for example from 800-900 mg/kg, such as from 900-1000 mg/kg.

In one embodiment the present invention relates to a fish feed composition comprising from 1-1000 mg/kg chromium, such as from 1-2 mg/kg, for example from 2-3 mg/kg, such as from 3-4 mg/kg, for example from 4-5 mg/kg, such as from 5-6 mg/kg, for example from 6-7 mg/kg, such as from 7-8 mg/kg, for example from 8-9 mg/kg, such as from 9-10 mg/kg, for example from 10-12 mg/kg, such as from 12-14 mg/kg, for example from 14-16 mg/kg, such as from 16-18 mg/kg, for example from 18-20 mg/kg, such as from 20-25 mg/kg, for example from 25-30 mg/kg, such as from 30-35 mg/kg, for example from 35-40 mg/kg, such as from 40-45 mg/kg, for example from 45-50 mg/kg, such as from 50-60 mg/kg, for example from 60-70 mg/kg, such as from 70-80 mg/kg, for example from 80-90 mg/kg, such as from 90-100 mg/kg, for example from 100-150 mg/kg, such as from 150-200 mg/kg, for example from 200-250 mg/kg, such as from 250-300 mg/kg, for example from 300-350 mg/kg, such as from 350-400 mg/kg, for example from 400-500 mg/kg, such as from 500-600 mg/kg, for example from 600-700 mg/kg, such as from 700-800 mg/kg, for example from 800-900 mg/kg, such as from 900-1000 mg/kg.

In one embodiment the present invention relates to a fish feed composition comprising one or more vitamins such as the fat-soluble vitamins A , D, E, and K.

The present invention relates in one embodiment to a fish feed composition comprising any amount of one or more of the compounds selected from the group consisting of Thiamin, Riboflavin, Vitamin B6 (Pyridoxine), Pantothenic acid, Niacin, Biotin, Folate, Vitamin B12, Choline, Myoinositol and Vitamin C.

### Binders

A binder is a material used to bind together two or more other materials in mixtures. Its two principal properties are adhesion and cohesion. Generally binders may be of protein (see the above section on protein), carbohydrate (see in the below) or gum base.

Non-limiting examples of binders include wax, linseed oil, gum arabic, gum tragacanth, methyl cellulose, gums, or protein such as egg white or casein, flours of corn, wheat or other “breakfast” cereals, polysaccharide binders, plant gum agar-agar, tapioca flour (starch) cassava flour, lactose, sucrose, microcrystalline cellulose, pectin, poly vinyl pyrrolidone and various other starches, gelatinized starch, wheat, gum binders, plant gum, gum arabic, locust bean, agar carageenin and other alginates and gelatine from any animal source, preferably porcine and/or bovine, and beef heart.

In an embodiment, the binders constitute between 1 and 60% of the feed composition, such as between 3 and 55% of the feed composition, such as between 5 and 50%, such as between 7 and 45%, such as between 9 and 40%, such as between 10 and 35%, such as between 11 and 30%, or between 13 and 25%, such as between 15 and 20%, or such as between 20 and 30%, such as between 22 and 28%, such as between 24 and 26%, such as around 25% of the feed composition.

In another embodiment the protein based binder fraction of the feed composition including stickwater constitutes between 1 and 40%, such as between 3 and 35%, such as between 5 and 30% such as between 7 and 25%, such as between 9 and 23%, such as between 11 and 22%, such as between 13 and 21%, such as between 15 and 19%, such as around 17%, such as 16 or 18% of the feed composition. Likewise, the carbohydrate based binders such as gelatinized starch, corn flour and wheat constitute between 0.5 and 30%, such as between 1 and 25%, such as between 2 and 20% such as between 3 and 18%, such as between 4 and 16%, such as between 5 and 14%, such as between 6 and 12%, such as between 8 and 10%, such as around 9%, 8% or 10% of the feed composition.

Other common additives incorporated into feeds for aquatic species are chemoattractants and flavorings, such as fish hydrosylates and condensed fish solubles (typically added at 5% of the diet). The amino acids glycine and alanine, and the chemicals betaine and betafin (choline) are also known to stimulate strong feeding behavior in fish. In an embodiment, the attractants constitute between 0 and 20%, such as between 0.5 and 18%, such as between 1 and 16%, such as between 1.5 and 14%, such as between 2 and 12%, or between 2.5 and 10%, such as between 3 and 8%, such as between 3.5 and 8% such as between 4 and 6% such as around 5%, such as 4% or 6% of the feed composition or between 0.5 and 10%, such as between 2.5 and 7.5% of the feed composition.

The pigments of the feed composition comprise in one embodiment between 0 and 7% of the feed composition such as between 0.5 and 6%, such as between 1 and 5%, such as between 1.5 and 4% such as between 2 and 3%, such as around 2.5%, or such around 0.5%, 1%, 1.5 or 2% of the feed composition.

An embodiment of the present invention comprises between 0 and 2% of preservatives such as between 0.05 to 1.5% such as between 0.1 to 1%, such as between 0.15 to 0.5%, such as between 0.2 to 0.25% or such as between 0.05 and 0.2% such as around 0.1% of the feed composition.

In one embodiment the present invention relates to a fish feed composition with 1-15% water, such as from 1-2%, for example from 2-3%, such as from 3-4%, for example from 4-5%, such as from 5-6%, for example from 6-7%, such as from 7-8%, for example from 8-9%, such as from 9-10%, for example from 10-11%, such as from 11-12%, for example from 12-13%, such as from 13-14%, for example from 14-15%.

In one embodiment the present invention relates to fish feed composition comprising from 1-15% fiber, such as from 1-2%, for example 2-3%, such as from 3-4%, for example 4-5%, such as from 5-6%, for example 6-7%, such as from 7-8%, for example 8-9%, such as from 9-10%, for example 10-11%, such as from 11-12%, for example 12-13%, such as from 13-14%, for example 14-15%.

The present invention relates in one embodiment to a fish feed composition comprising one or more hormones.

The present invention relates in one embodiment to a fish feed composition comprising one or more antibiotics.

The present invention relates in one embodiment to a fish feed composition comprising one or more antioxidants.

Feeding Rate, Frequency, and Methods

The present invention relates in one embodiment to a fish feed composition comprising Hydroxyproline. This fish feed composition can be used for feeding fish at any feeding rate and by any feeding method including the ones mentioned herein below.

Feeding rates and frequencies are in part a function of fish size. Small larval fish and fry need to be fed a high protein diet frequently and usually in excess. Small fish have a high energy demand and must eat nearly continuously and be fed almost hourly. Feeding small fish in excess is not as much of a problem as overfeeding larger fish because small fish require only a small amount of feed relative to the volume of water in the culture system.

As fish grow, feeding rates and frequencies should be lowered, and protein content reduced. However, rather than switching to a lower protein diet, feeding less allows the grower to use the same feed (protein level) throughout the grow-out period, thereby simplifying feed inventory and storage.

Feeding fish is labour-intensive and expensive. Feeding frequency is dependent on labour availability, farm size, and the fish species and sizes grown. Large catfish farms with many ponds usually feed only once per day because of time and labour limitations, while smaller farms may feed twice per day. Generally, growth and feed conversion increase with feeding frequency. In indoor, intensive fish culture systems, fish may be fed as many as 5 times per day in order to maximize growth at optimum temperatures.

Many factors affect the feeding rates of fish. These include time of day, season, water temperature, dissolved oxygen levels, and other water quality variables. For example, feeding fish grown in ponds early in the morning when the lowest dissolved oxygen levels occur is not advisable. In contrast, in recirculating aquaculture systems where oxygen is continuously supplied, fish can be fed at nearly any time. During the winter and at low water temperatures, feeding rates of warmwater fishes in ponds decline and feeding rates should decrease proportionally.

Feed acceptability, palatability and digestibility vary with the ingredients and feed quality. Fish farmers pay careful attention to feeding activity in order to help determine feed acceptance, calculate feed conversion ratios and feed efficiencies, monitor feed costs, and track feed demand throughout the year.

Farmers can calculate optimum feeding rates based on the average size in length or weight and the number of fish in the tank, raceway, or pond. Farmed fish typically are fed 1-4% of their body weight per day.

Fish can be fed by hand, by automatic feeders, and by demand feeders. Many fish farmers like to hand feed their fish each day to assure that the fish are healthy, feeding vigorously, and exhibiting no problems. Large catfish farms often drive feed trucks with compressed air blowers to distribute (toss) feed uniformly throughout the pond.

There are a variety of automatic (timed) feeders ranging in design from belt feeders that work on wind-up springs, to electric vibrating feeders, to timed feeders that can be programmed to feed hourly and for extended periods. Demand feeders do not require electricity or batteries. They usually are suspended above fish tanks and raceways and work by allowing the fish to trigger feed release by striking a moving rod that extends into the water. Whenever a fish strikes the trigger, a small amount of feed is released into the tank. Some growers use night lights and bug zappers to attract and kill flying insects and bugs to provide a supplemental source of natural food for their fish.

Feed Conversion and Efficiency Calculations:

Because feed is expensive, feed conversion ratio (FCR) or feed efficiency (FE) are important calculations for the grower. They can be used to determine if feed is being used as efficiently as possible.

FCR is calculated as the weight of the feed fed to the fish divided by the weight of fish growth. For example, if fish are fed 10 pounds of feed and then exhibit a 5 pound weight gain, the FCR is 10/5=2.0. FCRs of <1.5 are considered good growth for most species.

FE is simply the reciprocal of FCRs (1/FCR). FEs greater than 50% are considered good growth.

Types of Fish

The present fish food can be used for feeding of any fish species including herbivorous fish (plant eating), omnivorous fish (plant-animal eaters) and carnivorous (flesh-eating) fish. Examples of fish suitable for the invention are Albacore Tuna (Thunnus alalunga), Arrowtooth Flounder (Atheresthes stomias), Atlantic Cod (Gadus morhua), Atlantic Cutlassfish (Trichiurus lepturus), Atlantic Salmon (Salmo salar), Atlantic Wolffish (Anarhichas lupus), Black Drum (Pogonias cromis), Black Pomfret (Parastromateus niger), Blackback Flounder (Sole, Pleuronectes americanus), Blacktip Shark (Carcharhinus limbatus), Catfish (Ictalurus furcatus), Crab, Blue (Callinectes sapidus), Marlin (Makaira nigricans), Rockfish (Sebastes auriculatus), Puffer (Sphoeroides annulatus), Scorpionfish (Scorpaena guttata), Sheephead (Semicossyphus pulcher), Rockfish (Sebastes pinniger), Snapper (Lutjanus purpureus), Catfish (Ictalurus punctatus), Rockfish (Sebastes goodei, Sebastes nebulosus), Chinook (Oncorhynchus tshawytscha), Chub Mackerel (Scomber japonicus), Coho Salmon (Silver, Medium Red) (Oncorhynchus kisutch), Thresher Shark (Alopias vulpinus), Grouper (Epinephelus fulva), Cusk (Brosme brosme), Mahi-mahi (Coryphaena hippurus), Sole (Microstomus pacificus), Sole (Pleuronectes vetulus), Escolar (Lepidocybium flavobrunneum), Dory (Zeus faber), Ocean Perch (Sebastes norvegicus), Snapper (Lutjanus griseus), Sole (Flounder) (Glyptocephalus cynoglossus), Barracuda (Sphyraena barracuda), Haddock (Melanogrammus aeglefinus), Tuna (Euthynnus affinis), Snapper (Lutjanus synagris), Lingcod (Ophiodon elongatus), Milkfish (Chanos chanos), Tilapia (Tilapia mossambica), Nile Tilapia (Tilapia nilotica), Puffer (Sphoeroides maculatus), Tilefish (Caulolatilus princeps), Oilfish (Ruvettus pretiosus), Orange Roughy (Hoplostethus atlanticus), Barracuda (Sphyraena argentea), (Bonito(Sarda chiliensis), Cod (Alaska Cod, Gadus macrocephalus), Jack (Caranx caninus), Jack (Selene peruviana), Ocean Perch (Sebastes alutus), Mackerel (Scomber scombrus), Spanish (Scomberomorus sierra), Snapper (Lutjanus peru), Patagonian Toothfish (Dissostichus eleginoides), Sole (Flounder, Eopsetta jordani), Pink Salmon (Humpback) (Oncorhynchus gorbuscha), Pollock (Pollachius virens), Rockfish (Sebastes maliger), Trout, Rainbow (Steelhead) (Oncorhynchus mykiss), Drum (Redfish) (Sciaenops ocellatus), Porgy (Chrysophrys auratus), Snapper (Lutjanus campechanus), Rockfish (Sebastes proriger), Sole (Flounder, Errex zachirus), Rockfish (Sebastes aleutianus), Schoolmaster (Lutjanus apodus), Sheepshead (Archosargus probatocephalus), Shark, Mako (Isurus oxyrinchus), Snapper (Lutjanus vivanus), Butterfish (Pampus argenteus), Rockfish (Sebastes brevispinis), Skipjack Tuna (Katsuwonus pelamis), Spinefoot (Siganus favus), Croaker or Corvina (Roncador steamsi), Flounder (Platichthys stellatus), Marlin (Tetrapturus audax), Bass (Morone chrysops x saxatilis), Swordfish (Xiphias gladius), Carp (Barbodes schwanefeldi), Pollock (Alaska Pollock, Theragra chalcogramma), Hake (Urophycis tenuis), Rockfish (Sebastes entomelas), Flounder (Scophthalmus aquosus), Croaker (Yellowfish, Pseudosciaena manchurica), Rockfish (Sebastes ruberrimus), Tuna (Thunnus albacares), Yellowstripe Scad (Selaroides leptolepis), Yellowtail (Seriola lalandei), Flounder (Limanda ferruginea), Rockfish (Sebastes flavidus) and Snapper (Ocyurus chrysurus) Arctic char (Salvelinus alpinus), Turbot, Greenland halibut (Reinhartdius hippoglossoides) Halibut (Hippoglossus hippoglossus)

Non-limiting examples of the most popular fish species being raised and including both marine and freshwater fish, and thus species that fall within the scope of the present invention are: Arctic Char, Atlantic Salmon, Atlantic Cod, Bass, such as Sea bass, Australian sea bass, black sea bass, giant sea bass, Chilean sea bass and European sea bass, striped bass (Morone saxatilis), white bass (M. chrysops), black basses such as the largemouth bass (Micropterus salmoides), smallmouth bass (M. dolomieu), spotted bass (M. punctulatus), Guadalupe bass (M. treculii) and rock bass (Ambloplites rupestris), Barramundi, Blue Gill, Brook Trout, Brown Trout, Carps, Catfish, Chinook Salmon, Crappie, Drum, Halibut, Flounders, Rainbow Trout, Hybrid Striped Bass, Tilapias, Tunas, Walleye, Perch and Grouper. Perch and grouper are of special interest in connection with the present feed composition and examples of these include, but are not limited to: Grouper: Black grouper (Mycteroperca bonaci), Comet grouper (Epinephelus morrhua), Gag grouper (Mycteroperca microlepis), Giant grouper (Epinephelus lanceolatus), Goliath grouper (Epinephelus itajara), Nassau grouper (Epinephelus striatus), Saddletail grouper (Epinephelus daemelii), White grouper (Epinephelus aeneus), Queensland Groper (Epinephelus lanceolatus) and Yellowfin grouper (Myceroperca venenosa); Perch: Balkhash perch (Perca schrenkii), European perch (Perca fluviatilis), Yellow perch (Perca flavescens), fish of the family Percichthyidae are known as the Temperate perch and several other species of fish, including Estuary perch (Macquaria colonorum), Golden perch (Macquaria ambigua), Macquarie perch (Macquaria australasica), Nile perch (Lates niloticus), Pirate perch (Aphredoderus sayanus), White perch (Morone americana), Rock Perch (Ambloplites rupestris), Sacramento perch (Archoplites interruptus), shiner perch (Cymatogaster aggregata), Silver Perch (Bidyanus bidyanus), American silver perch (Bairdiella chrysoura), Cod, Turbot, Halibut, Sea bass, Sea bream, Perch, Grouper, Sturgeon, White fish and the Climbing perch family (Climbing gourami, Anabantidae).

In one embodiment the fish can be selected from, but is not limited to, the group consisting of Albacore Tuna (Thunnus alalunga), Arrowtooth Flounder (Atheresthes stomias), Atlantic Cod (Gadus morhua), Atlantic Cutlassfish (Trichiurus lepturus), Atlantic Salmon (Salmo salar), Atlantic Wolffish (Anarhichas lupus), Black Drum (Pogonias cromis), Black Pomfret (Parastromateus niger), Blackback Flounder (Sole, Pleuronectes americanus), Blacktip Shark (Carcharhinus limbatus), Catfish (Ictalurus furcatus), Crab, Blue (Callinectes sapidus), Marlin (Makaira nigricans), Rockfish (Sebastes auriculatus), Puffer (Sphoeroides annulatus), Scorpionfish (Scorpaena guttata), Sheephead (Semicossyphus pulcher), Rockfish (Sebastes pinniger), Snapper (Lutjanus purpureus), Catfish (Ictalurus punctatus), Rockfish (Sebastes goodei, Sebastes nebulosus), Chinook (Oncorhynchus tshawytscha), Chub Mackerel (Scomber japonicus), Coho Salmon (Silver, Medium Red) (Oncorhynchus kisutch), Thresher Shark (Alopias vulpinus), Grouper (Epinephelus fulva), Cusk (Brosme brosme), Mahi-mahi (Coryphaena hippurus), Sole (Microstomus pacificus), Sole (Pleuronectes vetulus), Escolar (Lepidocybium flavobrunneum), Dory (Zeus faber), Ocean Perch (Sebastes norvegicus), Snapper (Lutjanus griseus), Sole (Flounder) (Glyptocephalus cynoglossus), Barracuda (Sphyraena barracuda), Haddock (Melanogrammus aeglefinus), Tuna (Euthynnus affinis), Snapper (Lutjanus synagris), Lingcod (Ophiodon elongatus), Milkfish (Chanos chanos), Tilapia (Tilapia mossambica), Nile Tilapia (Tilapia nilotica), Puffer (Sphoeroides maculatus), Tilefish (Caulolatilus princeps), Oilfish (Ruvettus pretiosus), Orange Roughy (Hoplostethus atlanticus), Barracuda (Sphyraena argentea), (Bonito(Sarda chiliensis), Cod (Alaska Cod, Gadus macrocephalus), Jack (Caranx caninus), Jack (Selene peruviana), Ocean Perch (Sebastes alutus), Mackerel (Scomber scombrus), Spanish (Scomberomorus sierra), Snapper (Lutjanus peru), Patagonian Toothfish (Dissostichus eleginoides), Sole (Flounder, Eopsetta jordani), Pink Salmon (Humpback) (Oncorhynchus gorbuscha), Pollock (Pollachius virens), Rockfish (Sebastes maliger), Trout, Rainbow (Steelhead) (Oncorhynchus mykiss), Drum (Redfish) (Sciaenops ocellatus), Porgy (Chrysophrys auratus), Snapper (Lutjanus campechanus), Rockfish (Sebastes proriger), Sole (Flounder, Errex zachirus), Rockfish (Sebastes aleutianus), Schoolmaster (Lutjanus apodus), Sheepshead (Archosargus probatocephalus), Shark, Mako (Isurus oxyrinchus), Snapper (Lutjanus vivanus), Butterfish (Pampus argenteus), Rockfish (Sebastes brevispinis), Skipjack Tuna (Katsuwonus pelamis), Spinefoot (Siganus favus), Croaker or Corvina (Roncador steamsi), Flounder (Platichthys stellatus), Marlin (Tetrapturus audax), Bass (Morone chrysops x saxatilis), Swordfish (Xiphias gladius), Carp (Barbodes schwanefeldi), Pollock (Alaska Pollock, Theragra chalcogramma), Hake (Urophycis tenuis), Rockfish (Sebastes entomelas), Flounder (Scophthalmus aquosus), Croaker (Yellowfish, Pseudosciaena manchurica), Rockfish (Sebastes ruberrimus), Tuna (Thunnus albacares), Yellowstripe Scad (Selaroides leptolepis), Yellowtail (Seriola lalandei), Flounder (Limanda ferruginea), Rockfish (Sebastes flavidus) and Snapper (Ocyurus chrysurus) Arctic char (Salvelinus alpinus), Turbot, Greenland halibut (Reinhartdius hippoglossoides) and Halibut (Hippoglossus hippoglossus).

In one embodiment the fish can be selected from, but is not limited to, the group consisting of Ablennes hians, Acanthopagrus berda, Acanthophthalmus semicinctus, Acanthuridae, Acanthurus lineatus, Acanthurus sp., Acanthurus triostegus, Acentrogobius janthinopterus, Alectis sp., Ambassidae, Ambassis buruensis, Ambassis commersoni auctorum, Ambassis miops, Ambassis miops, Amblygaster sirm, Amphacanthus oramin, Siganus canaliculatus, Anabantidae, Anabas testudineus, Anguilla bicolor pacifica, Anguilla celebensis, Anguilla marmorata, Anguilla mauritiana, Anguilla marmorata, Anguilla sp., Anguillidae, Anodontostoma chacunda, Anomalopidae, Anomalops katoptron, Anyperodon leucogrammicus, Apogon ellioti, Apogon sp. auctorum, Hypseleotris bipartite, Apogonichthys ellioti, Apogon ellioti, Apogonidae, Apolectus niger, Parastromateus niger, Ariidae, Aristichthys nobilis, Arius manillensis, Arius sp., Arius thallassinus, Netuma thalassina, Arothron manilensis, Arothron mappa, Atherina balabacensis, Atherinidae, Atule mate, Bagridae, Balistes flavimarginatus, Pseudobalistes flavimarginatus, Balistidae, “banak”, Belonidae, Belontiidae, Berycidae, Beryx splendens, Bodianus mesothorax, “boloan”, Botia macracanthus, Butts amboinensis, Caesio cuning, Caesio erythrogaster, Caesio cuning, Caesio lunaris, Caesionidae, Capoeta tetrazona, Puntius tetrazona, Carangidae, Carangoides armatus, Caranx affinis, Atule mate, Caranx armatus, Carangoides armatus, Caranx sexfasciatus, Caranx sp., Caranx speciosus, Gnathanodon speciosus, Carassius auratus, Carassius carassius, Carassius sp., “catfish”, Centropomidae, Cephalopholis sonnerati, Chaetodon collare, Chaetodontidae, Chanidae, Channa striata, Channidae, Chanos chanos, Characidae, Cheilinus diagrammus, Oxycheilinus diagrammus, Cheilio inermis, Choerodon anchorago, Chonophorus melanocephalus, Cichlidae, Cirrhina mrigala, Cirrhinus mrigala, Cirrhinus mrigala, Clarias batrachus, Clarias macrocephalus, Clariidae, Clupeidae, Cobitidae, Colisa lalia, Trichogaster lalia “colored tilapia”, Creisson validus, Acentrogobius janthinopterus, Ctenopharyngodon idella, Cymbacephalus nematophthalmus, Cyprinidae, Cyprinus carpio, Cyprinus carpio carpio, Cyprinus carpio carpio, Cyprinus sp., Decapterus macrosoma, Decapterus sp., Dipterygonotus balteatus, Dischistodus fasciatus, “eel”, Eleotridae, Eleotris fusca, Eleotris melanosoma, Eleutheronema tetradactylum, Engraulidae, Epalzeorhynchos bicolour, Epalzeorhynchos frenatum, Ephippidae, Epinephelus bleekeri, Epinephelus malabaricus, Epinephelus sp., Epinephelus undulosus, Erythrichthys leucogrammicus, Dipterygonotus balteatus, Euthynnus affinis, Euthunnus yaito, Euthynnus affinis, Gazza minuta, Gerreidae, Gerres filamentosus, Gerres kapas, Glossogobius biocellatus, Glossogobius giuris, Gnathanodon speciosus, Gobiidae, “grouper”, Gymnocranius griseus, Haemulidae, Harengula dispilonotus, Herklotsichthys dispilonotus, Hemiramphidae, Hemiramphus dussumieri, Hyporhamphus dussumieri, Hemiramphus georgii, Rhynchorhamphus georgii, Hepsetia balabacensis, Atherina balabacensis, Herklotsichthys dispilonotus, Holocentridae, Holocentrus samara, Neoniphon samara, Holocentrus violaceus, Sargocentron violaceum, Hypophthalmichthys molitrix, Hyporhamphus dussumieri, Hypseleotris bipartite, “igat”, lniistius sp., Xyrichtys sp., Labeo bicolour, Epalzeorhynchos bicolour, Labeo frenatus, Epalzeorhynchos frenatum, Labridae, Lactariidae, Lactarius lactarius, Lates calcarifer, Leiocassis siamensis, Leiognathidae, Leiognathus equulus, Leiognathus sp., Lepidaplois mesothorax, Bodianus mesothorax, Lethrinidae, Lethrinus erythropterus, Lethrinus hypselopterus, Lethrinus erythropterus, Lethrinus nebulosus, Liza subviridis, Lutjanidae, Lutjanus bohar, Lutjanus dodecacanthus, Lutjanus malabaricus, Lutjanus fulviflamma, Lutjanus gibbus, Lutjanus malabaricus, Lutjanus rangus, Lutjanus bohar, Lutjanus sp., Lutjanus vitta, Megalaspis cordyla, Megalopidae, Megalops cyprinoides, Mene maculate, Menidae, Moenkhausia collettii, Mollienesia latipinna, Poecilia latipinna, Monocentridae, Monocentris sp., Monopterus albus, Mugil cephalus, Mugil dussumieri, Liza subviridis, Mugil sp., Mugilidae, “mullet”, Mullidae, Muraenesocidae, Muraenesox cinereus, Mylopharyngodon piceus, Naso hexacanthus, Naso unicornis, Naso vlamingii, Nematabramis verecundus, Nemipteridae, Nemipterus sp., Neoniphon samara, Netuma thalassina, Ophicephalus striatus, Channa striata, Ophieleotris aporos, Ophiocara aporos, Ophieleotris aporos, Oreochromis mossambicus, Oreochromis mossambicus x O. niloticus, Oreochromis niloticus niloticus, Otolithes rubber, Oxycheilinus diagrammus, Oxyurichthys microlepis, Pangasiidae, Pangasius sutchi, Pantodon buchholzi, Pantodontidae, Parachaetodon ocellatus, Paracheirodon simulans, Paralichthyidae, Parastromateus niger, Parupeneus barberinus, Parupeneus indicus, Parupeneus macronema, Pelates quadrilineatus, Pennahia anea, Petrochromis orthognathus, Pinjalo pinjalo, Platax orbicularis, Platybelone sp., Platycephalidae, Platycephalus indicus, Plotosidae, Plotosus anguillaris, Plotosus lineatus, Plotosus lineatus, Poecilia latipinna, Poecilia reticulate, Poeciliidae, Polymixia japonica, Polymixiidae, Polynemidae, Pomacentridae, Pomacentrus fasciatus, Dischistodus fasciatus, Pomadasys argenteus, Pomadasys hasta, Pomadasys argenteus, Priacanthidae, Priacanthus tayenus, Pristipoma hasta, Pomadasys argenteus, Psettodes erumei, Psettodidae, Pseudobalistes flavimarginatus, Pseudorhombus javanicus, Pseudosciaena anea, Pennahia anea, Puntius binotatus, Puntius manalak, Puntius palata, Spratellicypris palata, Puntius tetrazona, Rasbora kalochroma, Rastrelliger brachysoma, Rastrelliger chrysozonus, Rastrelliger kanagurta, Rastrelliger faughni, Rastrelliger kanagurta, Rhynchorhamphus georgii, Sardinella albella, Sardinella longiceps, Sardinella perforate, Sardinella albella, Sardinella sirm, Amblygaster sirm, Sargocentron violaceum, Saurida tumbil, Scaridae, Scarus festivus, Scarus lunula, Scarus festivus, Scatophagidae, Scatophagus argus, Sciaenidae, Scolopsis sp., Scomberoides lysan, Scomberoides sp., Scomberomorus commerson, Scombridae, Selar crumenophthalmus, Selaroides leptolepsis, Seriola nigrofasciata, Seriolina nigrofasciata, Seriolina nigrofasciata, Serranidae, Sicyopterus sp., Siganidae, Siganus argenteus, Siganus canaliculatus, Siganus fuscescens, Siganus guttatus, Siganus javus, Siganus oramin, Siganus canaliculatus, Siganus sp., Siganus spinus, Siganus striolatus, Siganus spinus, Siganus virgatus, Sillaginidae, Sillago maculate, Sillago sihama, “snapper”, Soleidae, Sparidae, Sparus berda, Acanthopagrus berda, Sphaerichthys osphromenoides, Sphyraena jello, Sphyraena langsar, Sphyraena sp., Sphyraenidae, Spratellicypris palata, Stolephorus commerson, Stolephorus sp., Strongylura incise, Strongylura leiura, Strongylura strongylura, Strongylura urviffii, Synaptura sorsogonensis, Synbranchidae, Synodontidae, “talilong” , Tanichthys albonubes, Terapon argenteus auctorum, Terapon plumbeus, Terapon jarbua, Terapon plumbeus, Terapon puta, Terapontidae, Tetrodon manilensis, Arothron manilensis, Tetraodontidae, Teuthis concatenate, Siganus guttatus, Teuthis javus, Siganus javus, Teuthis rostrata, Siganus argenteus, Teuthis sp., Acanthurus sp., Teuthis virgata, Siganus virgatus, Thysanophrys nematophthalmus, Cymbacephalus nematophthalmus “tilapia”, Tilapia mossambica, Oreochromis mossambicus, Tilapia nilotica, Oreochromis niloticus niloticus, Tilapia sp., Tilapia ziffii, “top minnow”, Trichiuridae, Trichiurus haumela, Trichiurus lepturus, Trichiurus lepturus, Trichogaster lalia, Trichogaster pectoralis, Trichogaster trichopterus, Tylosurus acus, Tylosurus acus acus, Tylosurus acus melanotus, Tylosurus crocodilus, Tylosurus crocodilus crocodilus, Tylosurus crocodilus crocodilus, Tylosurus punctulatus, Tylosurus sp., Xiphophorus maculates, Xiphophorus sp., Xyrichtys sp.

Preparation of Fish Food

The fish feed composition of the present invention can be prepared by any method including any method comprising one or more of the steps mentioned herein below.

The conventional processes for preparing such types of fish feed normally include an extrusion pelleting process whereby the carbohydrate-containing feed mixture is pre-cooked at temperatures above the starch gelatinization temperature followed by an extrusion step wherein the feed mixture is cooked together with the other feed ingredients at a temperature above 100° C., resulting in solid, dry feed pellets. In the known methods of manufacturing dry fish feed, carbohydrates in amounts from about 8 to about 30 wt % is an ingredient which in the industry is considered as required to obtain a fish feed having a sufficient long term stability towards disintegration and deformation during storage and distribution.

In a further aspect, the invention provides, a method of preparing a fish feed composition, the method comprising as a first step the mixing of a lipid and an emulsifying agent. If it is required to melt the emulsifying agent, the lipid is pre-heated to a temperature above the melting point of the emulsifying agent e.g. to a temperature in the range of about 55-65° C. In a subsequent step, the protein source as defined above is added to the resulting mixture in the form of an aqueous suspension and/or solution or as a slurry of protein. The protein content of the aqueous suspension and/or solution may vary, but is preferably in the range of 10-90 wt %, such as 20 to 80 wt % protein, including a content of 30 to 70 wt % and e.g. from 40 to 60 wt % protein.

The mixture resulting from the above subsequent step is then subjected to emulsifying conditions using a conventional homogenising or an emulsifying apparatus to obtain a water-in-lipid or a lipid-in-water emulsion, depending on the type of emulsifying agent used. Subsequently, the resulting emulsion is formed into particles by a forming process e.g. pelleting at a temperature not exceeding 100° C. and, if required, the particles are dried e.g. using air drying, hot air drying, freeze drying or drum drying to obtain the feed composition. However, when a high melting emulsifying agent is used a drying step is generally not required to obtain solid and coherent feed particles.

In a still further aspect, the invention relates to an alternative method for preparing a fish feed composition which method comprises as a first step the preparation of an emulsion comprising a lipid, an aqueous phase and at least one emulsifying agent. The aqueous phase may optionally contain protein in an amounts as mentioned above, i.e. in the range of 10-90 wt %, such as 20 to 80 wt % protein, including a content of 30 to 70 wt % and e.g. from 40 to 60 wt % protein.

In a subsequent step, this emulsion is combined with at least one dry protein-containing ingredient to obtain a fish feed mixture which is subsequently formed and, if required, dried to obtain the fish feed composition.

In either of the above methods, the aqueous suspension and/or solution may also contain at least one further structurizing or consistency controlling ingredient selected from a thickening agent, a viscosity enhancing agent and a stabilizing agent including as an example, a non-protein hydrocolloid such as a polysaccharide of vegetable or microbial origin, preferably a carrageenan or a gum, a wetting agent such as a ketone or an alcohol, preferably ethanol, a flavouring agent such as betaine, a mineral compound and/or a vitamin.

In particular it has been found that the addition of a wetting agent including an alcohol or a ketone to the emulsion mixture facilitates an effective emulsification and subsequent mixing with the other ingredients of the feed composition. A suitable amount of an alcohol or ketone is 5-15 wt % of the emulsion and 1-5 wt %, calculated on the feed composition after mixing. Evidently, part of an added alcohol or ketone will evaporate from the feed composition during mixing and storage of the finished feed composition.

It is an important aspect of the methods according to the invention that the process temperature is low, whereby a deterioration of the dietary quality of the protein and lipid content is avoided and the energy consumption is low. Thus, the processes are preferably carried out at temperatures which are in the range of 10-100° C., preferably at the lowest possible temperatures where efficient mixing of the component can still be attained. Thus, the process temperature may preferably be within a range of 15-80° C. such as a range of 20-60° C.

Clinical Applications:

Inclusion of clinical application of Hydroxyproline is relevant because the documentation observed for fish may be relevant for other animals and humans with specific conditions. Extreme nutrition such as vegetarians might lack Hydroxyproline. In other individuals the metabolism of Hydroxyproline may be limiting e.g. due to genetic reasons or reduced production by elderly people (as for Vitamin B12). This could e.g. be due to one or several enzyme limitations in the metabolism of Hydroxyproline or to lack of co-factors or other.

Hydroxyproline is a single component naturally present in the organism. Hydroxyproline is a major component of the protein collagen. Hydroxyproline and Proline play key roles for collagen stability. They permit the sharp twisting of the collagen helix. It helps provide stability to the triple-helical structure of collagen by forming hydrogen bonds.

Hydroxyproline is found in few proteins other than collagen such as elastin. Proline hydroxylation requires ascorbic acid. The most obvious, first effects (gum and hair problems) of absence of ascorbic acid in humans come from the resulting defect in hydroxylation of Proline residues of collagen, with reduced stability of the collagen molecule causing scurvy. The composition comprising Hydroxyproline can be used for stimulation of hair growth in human beings if hydroxyproline synthesis for some reason is limiting.

If dietary supplement of Hydroxyproline is beneficial, it is reasonable to believe that this affect tissues that are rich in Hydroxyproline such as skin, bone, gills and the gastrointestinal tract.

Gingivitis

The composition comprising Hydroxyproline can be used for treatment of gingivitis e.g. in human beings. Gingivitis is the inflammation of the gums around the teeth. Gingivitis is caused by the long-term effects of plaque deposits. Plaque is a sticky material that develops on the exposed portions of the teeth, consisting of bacteria, mucus, and food debris. Gingivitis is a major cause of tooth decay. Unremoved plaque mineralizes into a hard deposit called tartar that becomes trapped at the base of the tooth. Plaque and tartar irritate and inflame the gingiva. Bacteria, and the toxins bacteria produce, cause the gums to become infected, swollen, and tender.

Injury or trauma to the gums from any cause, including overly vigorous brushing or flossing of the teeth, can also cause gingivitis. The risks for developing gingivitis include uncontrolled diabetes, pregnancy (because of hormonal changes that increase the sensitivity of the gingiva), general illness, and poor dental hygiene. Malocclusion of teeth (misaligned teeth), rough edges of fillings, and ill fitting or unclean mouth appliances (such as orthodontic appliances, dentures, bridges, and crowns) can irritate the gums and increase the risk of gingivitis. Medications such as phenytoin and birth control pills, and ingestion of heavy metals such as lead and bismuth are also associated with gingivitis. Many people experience gingivitis to a varying degree. It usually develops during puberty or early adulthood due to hormonal changes and may persist or recur frequently, depending on how healthy your teeth and gums are.

Symptoms of gingivitis include mouth sores, swollen gums, bright red or red-purple appearance to gums or shiny appearance to gums, bleeding gum and tender gums.

The condition is generally reversible. Gingivitis can be prevented through regular oral hygiene that includes daily brushing and flossing. A dentist or dental hygienist will perform a thorough cleaning of the teeth and gums; following this, persistent oral hygiene is necessary. The removal of plaque is usually not painful, and the inflammation of the gums should be gone between one and two weeks. Oral hygiene including proper brushing and flossing is required to prevent the recurrence of gingivitis. Anti-bacterial rinses or mouthwash, in particular Chlorhexidine digluconate 0.2% solution, may reduce the swelling and local mouth gels which are usually antiseptic and an aesthetic can also help. Warm salt water or antibacterial rinses can reduce the puffiness. Over-the-counter anti-inflammatory medications will ease any discomfort from a rigorous cleaning.

One important nutrient that can help heal red, swollen gums is vitamin C. This antioxidant boosts your immune system, brings down inflammation, and speeds up wound healing. In fact, a deficiency of vitamin C can lead to gingivitis. Vitamin C has proven to be vital for the production of collagen, the basic protein building block for the fibrous framework of all tissues, including gums. The vitamin strengthens gum tissue and helps the gum lining resist bacteria. Lack of available hydroxyproline may have the same effect and supplementation of hydroxyproline will improve the development and healing of gingivitis.

The conversion of proline to hydroxyproline requires vitamin C (ascorbic acid). Vitamin C deficiency results in the poor conversion of proline to hydroxyproline.

Osteoporosis

The clinical application of Hydroxyproline is supported by the documentation seen for fish showing for the first time that Hydroxyproline may be limiting. Several connective and bone disorders lack explanations in spite of intensive scientific research. We are not aware of any research that has tested the effect of dietary Hydroxyproline supplementation. In a recent review for osteoporosis the lack of explanation is concluded as well as suggestion that this may be due to an unidentified micronutrient. This micronutrient could be Hydroxyproline.

The functional properties and mechanical strength of the connective tissue in the bone and other tissues are dependent on the amount of hydroxylated proline and lysine for optimal formation of the triple helix structure of collagen (Blanck and Peterkofsky, 1975; Tucker 1983; Brinckmann et al., 2005).). During bone growth, the organic matrix which is mainly comprised by collagen fibres is first formed as osteoid, and is subsequently mineralised (Meunier, 2002). Several determinants of mineralisation such as pinealectomy, photoperiod and growth rate (Fjelldal 2005) as well as phosphorus deficiency (Albrektsen et al., 2006) have been demonstrated in Atlantic salmon. The mineral content in the bone has been found to be an important determinant of the mechanical strength of the vertebral bone (Fjelldal 2005; Albrektsen et al., 2006).

The composition comprising Hydroxyproline can be used for stimulation of bone growth and bone strength in animals including human beings, fish and birds. The composition can be used for in treatment of osteoporosis e.g. in human beings.

Bone is hard tissue that is in a constant state of flux, being built up by bone-forming cells called osteoblasts while also being broken down or resorbed by cells known as osteoclasts. During childhood and adolescence, bone formation is dominant; bone length and girth increase with age, ending at early adulthood when peak bone mass is attained.

Osteoporosis is a disease characterized by low bone mass and structural deterioration of bone tissue, leading to bone fragility and an increased susceptibility to fractures, especially of the hip, spine, and wrist. Osteoporosis occurs primarily as a result of normal ageing, but can arise as a result of impaired development of peak bone mass (e.g. due to delayed puberty or malnutrition) or excessive bone loss during adulthood (e.g. due to estrogen deficiency in women, malnutrition, or corticosteriod use). Osteoporosis is much more common in women than men and it is also far more common in Caucasians than in oriental or black people. Women are particularly vulnerable to osteoporosis after the menopause, especially if this occurs before the age of 45, because their ovaries no longer produce oestrogen which helps to maintain bone mass. Removal of the ovaries has the same effect.

Predisposing factors and causes of osteoporosis include thin stature, calcium deficiency, kidney failure and certain hormonal disorders such as Cushing's syndrome or prolonged corticosteroid therapy. Drugs such as Tamoxifen and antacids are implicated too. In men, osteoporosis is sometimes due to lack of testosterone, but excess consumption of alcohol is the main cause. Prolonged immobility and sedentary lifestyle is also a factor. Osteoporosis is more common in smokers and heavy drinkers and is often associated with chronic bronchitis and emphysema. Other factors to bear in mind are a history of over-dieting, anorexia nervosa and a family history of osteoporosis. Diabetes mellitus can lead to secondary osteoporosis. Vitamin D deficiency and over-prescription of thyroxine for hypothyroidism also predisposes towards the disease. The incidence of osteoporosis is lower in vegetarians, while it has been established that high meat consumption can inhibit calcium metabolism.

Other forms of primary osteoporosis are idiopathic osteoporosis, an osteoporotic condition where there is no identifiable cause for the bone loss. Idiopathic osteoporosis can affect children and adults. Juvenile osteoporosis is osteoporosis occurring in children between the ages of about 8 and about 14 years of age. In juvenile osteoporosis, impairment of bone growth occurs over a period of several years, and in most cases, goes into remission. However, the period of impaired bone growth and remodeling can lead to skeletal deformations, such as curvature of the spine and short stature. Moreover, inadequate accumulation of bone mass could lead to an increased risk of fractures later in life.

In many cases, osteoporosis is a ‘silent disease’, without any particular symptoms, and often the first sign is a fracture after a fall that would not have caused any damage in a young person. Typical fracture sites are at the top of the femur and just above the wrist. There may also be spontaneous fracture of one or more vertebrae, causing the bones to crumble and leading to a progressive loss of height, or pain due to spinal nerve compression. Hip fractures are the most serious, as they nearly always result in hospitalization, are fatal about 20% of the time, and produce permanent disability about half the time. Fracture rates increase rapidly with age and the lifetime risk of fracture in 50 year-old women is about 40%.

Treatment of Osteoporosis

Bone tissue that has already been lost cannot easily be replaced, and preventative measures should be taken early on. The primary goal of treatment of osteoporosis is to reduce the risk of pathologic fractures. The three main forms of treatment are:

1) Weight-Bearing Exercise:

Exercise is an important treatment for osteoporosis to maintain healthy bones. Activities that stress bones have been shown to have a positive effect in maintaining and increasing bone mass and preventing osteoporosis.

2) Nutrition Supplementation:

Calcium supplementation is an important treatment. Recommended oral intake is at least 1200 to 1500 mg every day. Patients at-risk for osteoporosis should take a calcium supplement each day (1200 to 1500 mg) in combination with Vitamin D (400-800 Units) to help increase the absorption. Calcium intake can be increased with milk products, lots of leafy green vegetables, nuts, Soya products and sardines. Cod liver oil increases bone elasticity. Also, caffeinated substances (e.g. coffee, soda, etc.) decrease calcium absorption, and should be avoided. Black cohosh, which attaches to oestrogen receptors in the bones, should help maintain bone density and delay or slow the progression of osteoporosis. The herb Fenugreek also has known osteoprotective effects. If you are seriously overweight, a sensible diet regime should be introduced.

Bran and wholemeal bread should not be consumed as they contain calcium-binding phytic acid. Salt intake should be reduced as it can increase calcium and phosphate loss through the kidneys.

3) Medications:

There are several available medications used for osteoporosis treatment. These include:

Bisphosphanates

Bisphosphanates e.g. Fosamax (Alendronate sodium), Boniva (ibandronate sodium) or Actonel (risedronate sodium) are a type of medication that helps to regulate calcium and prevent bone breakdown. Bone turnover, or replacement of old bone with new bone, is a normal process within our body. In patients with osteoporosis, the replacement with new bone does not maintain pace with the breakdown of old bone. Bisphosphanates slow the rate of bone breakdown to help maintain bone mass by inhibiting the osteoclast, the cell responsible for bone breakdown.

Strontium ranelate (Protelos) is a drug which stimulates new bone formation, and is used if you do not tolerate bisphosphonates well. Strontium is one of many trace minerals essential for bone health. Strontium supports healthy osteoblast differentiation and helps to keep osteoclast activity in balance. Additionally, strontium supplement supports healthy collagen formation by osteoblasts, enhancing bone tensile strength.

Calcitonin (Miacalcin)

Calcitonin is a naturally occurring hormone produced by the thyroid gland that can be given as an injection or taken as a nasal spray. Sold under the trade name Miacalcin, calcitonin also inhibits the function of the osteoclasts that break down bone. Calcitonin has long been known to be beneficial in patients with osteoporosis, but the injections were difficult to administer, and had unpleasant side-effects. The nasal spray has greatly improved the use of calcitonin, and it is much more commonly used today. Calcitonin has been shown to slow bone loss, and also decrease pain associated with osteoporosis fractures.

Raloxifene

Raloxifene is a newer medication that has been developed to provide some of the same advantages of estrogen (HRT), without the potential side-effects. Raloxifene is a type of medication called a Selective Estrogen Receptor Modulator, or SERM. The effects of Raloxifene have been shown to be similar to estrogen, including an increase in bone mass and lower cholesterol. However, the SERMs do not have the same effects on the uterine lining, and therefore do not need to be combined with progesterone. Furthermore, there is evidence that Raloxifene may decrease the risk of breast cancer.

Estrogen

Hormonal replacement therapy, or HRT, not only helps maintain, but it can increase bone mass after menopause. Multiple studies have shown the benefits of estrogen therapy, including a lower risk of osteoporosis and fractured bones. In addition, other benefits of estrogen replacement in the postmenopausal patient include lower cholesterol, decreased risk of colon cancer, and fewer postmenopausal symptoms. HRT was shown to increase the risk of uterine cancer, but this risk is eliminated when the estrogen is combined with progesterone. There have been studies showing an increase risk of breast cancer in some study populations. Patients on HRT have also shown a slightly increased risk of developing blood clots and strokes.

Parathyroid hormone (PTH) Produced by the parathyroid glands, PTH, stimulates both resorption and new bone formation. Intermittent administration stimulates formation more than resorption. Clinical trials to date suggest that PTH therapy is effective in both the prevention and treatment of osteoporosis, and a preparation called Forteo, given by daily injection, is now FDA approved for the treatment of severe osteoporosis. Forteo is more effective at building spine bone density than any other treatment. However, because it requires daily injection, and because of its expense, it is usually reserved for patients with very severe spine osteoporosis.

Didronel (etidronate disodium) acts primarily on bone. It can inhibit the formation, growth, and dissolution of hydroxyapatite crystals and their amorphous precursors by chemisorption to calcium phosphate surfaces. Inhibition of crystal resorption occurs at lower doses than are required to inhibit crystal growth.

The present invention relates in one embodiment to treatment of osteoporosis with

Hydroxyproline in combination with one or more other treatments of osteoporosis including the ones mentioned herein above.

Diagnosis

The present invention relates to treatment of osteoporosis after diagnosis of osteoporosis by any method including the methods mentioned herein below.

Women who have had a fracture due to fragility are diagnosed with severe osteoporosis and have a high risk for another fracture. Early diagnosis can prevent fractures and slow deterioration of bone tissue. By measuring bone mineral density (BMD) physicians determine if a patient has osteoporosis and/or is at risk for bone fracture. This testing also helps to monitor the effectiveness of treatment. BMD testing is a quick, noninvasive, and painless procedure.

Screening Methods

1) Dual-energy x-ray absorptiometry (DEXA): The current method of diagnosis of osteoporosis is by means of dual energy x-ray absorptiometry (DEXA), which provides a quantitative measurement of the amount of mineral present in bone and allows determination of fracture risk at a measured site. DEXA is used to scan the entire body and measures BMD. From the results, physicians assess the risk for fracture in the hip, spine, and wrist. DEXA produces a high resolution image, resulting in a precise BMD measurement. The level of radiation is low and the test takes less than 5 minutes. DEXA effectively monitors changes in bone density during treatment.

2) Quantitative computed tomography (QCT): QCT measures bone density in the hip and spine and produces a three-dimensional image that shows true volume density. QCT has the capacity to isolate an area for testing. The radiation level in QCT is 10 times higher than in DEXA.

3) Peripheral bone density testing uses ultrasound to identify bone loss in a localized area such as the heel or hand.

4) X-rays can only verify advanced bone loss, vertebral collapse, and bone fractures.

The World Health Organization (WHO) has established criteria for making the diagnosis of osteoporosis, as well as determining levels which predict higher chances of fractures. These criteria are based on comparing bone mineral density (BMD) in a particular patient with those of a 25 year old female. BMD values which fall well below the average for the 25 year old female (stated statistically as 2.5 standard deviations below the average) are diagnosed as “osteoporotic”. If a patient has a BMD value less than the normal 25 year old female, but not 2.5 standard deviations below the average, the bone is said to be “osteopenic” (osteopenic means decreased bone mineral density, but not as sever as osteoporosis). Interestingly, although these criteria are widely used, they were devised in a Caucasian female so there will be some differences when these levels are applied to non Caucasian females or to males in general. Despite this flaw, measurement of BMD is used daily and has proven to be very helpful in all groups. Some men will be subject to increased fracture rates when they have significantly less BMD than the predicted fracture level for women. In other words, some men will be at increased risk for fracture even when they have osteopenia.

The Z-score is used to classify the type of osteoporosis. A score below 1.5 indicates primary osteoporosis, which is age related. A score of 1.5 and higher indicates secondary osteoporosis, which is associated with calcitonin imbalance, malabsorption conditions (e.g., celiac disease, cystic fibrosis, lactose intolerance), alcohol abuse, smoking, and the use of certain medications.

These methods mentioned above for determination of bone mineral density can not provide detailed information about the mechanical properties of bone, such as elasticity and strength. In fact, the loss with age of the biomechanical strength of bone is more pronounced than the loss of bone mass, and, at any given age, there is greater inter-individual variation in the mechanical properties of bone than in the bone mass. Therefore, it is desirable to develop a clinically relevant method for determining the mechanical properties of bone.

The U.S. Pat. No. 4,976,267 describes a method of determining in vivo an estimate of the modulus of elasticity of a bone. The method including the steps of applying an ultrasound beam to a limb from a transmitting transducer to a receiving transducer, and then measuring the flight times, T.sub.s for an ultrasound beam along a first path through the cortex of the bone, and T.sub.m along a second path through the cortex and medulla of said bone. After performing these steps, the method calculates the diameter X of the bone from the external diameter of the limb minus a predetermined constant allowing for skin and flesh, calculates the apparent velocity C.sub.a from X divided by T.sub.s, calculates the mean velocity V from X divided by T.sub.m, and calculates the cortical bone velocity C.sub.bE from a predetermined function f multiplied by C.sub.a. Next, the method determines a ratio R for the diameter of the medulla to the diameter of the cortex and then estimates the cross-sectional area CSA.sub.E of the bone from the values of the diameter X, the ratio R and from predetermined constants. From these derivations, the bone mineral content, BMC, the estimated compact bone density, CBD.sub.E, and an estimate of the modulus of elasticity from the product of CBD.sub.E and C.sub.bE are determined.

This is also supported by advice given by WHO (www.who.int/nutrition/topics/5population_nutrient/en/index25.html). The clinical effects may be the same as observed for Hydroxyproline limitation due to Vitamin C deficiency, osteoporosis, wounds, etc.

The total picture for the reason for osteoporosis is not clear. This invention shows that hydroxyproline supplementation is beneficial for bone growth, and that lack of hydroxyproline may be one reason for bone malfunction. Supplementation of hydroxyproline will therefore improve the bone structure and prevent and heal bone malfunction such as osteoporosis.

A possible effect of Hydroxyproline is supported by several scientific observations. For instance that collagen hydrolysate support bone formation, metabolism and strength as well as cartilage production (Moskowitz, 2000. Seminars in Arthritis and Rheumatism, 30, pp 87-99). The beneficial effect of collagen hydrolysate on cartilage and bone structure is well described. Hydroxyproline could also be relevant in cancer treatment because it has been described that hydroxylation of Proline has an impact on bone cancer (Nissi et al., 2004. Acta Histochemica, 106, 111-121).

The hydroxyproline supplementation can be used for treatment of wounds in animals including human beings, fish and birds. A wound is defined as a type of physical trauma wherein the skin is torn, cut or punctured (an open wound), or where blunt force trauma causes a contusion (a closed wound). A wound is also defined as a condition where the connective tissue is weak and vulnerable due to lack of hydroxyproline and thus the protective layer is disrupted.

Enteritis

The hydroxyproline supplementation can in another embodiment be used for treatment of enteritis in animals including human beings, fish and birds. Enteritis has been describes in fish living of a diet with high Soya content. The general assumption in the field is that this enteritis is due to compounds in Soya. However, our data show is that enteritis in Soya fed fish may be caused completely or partly by lack of Hydroxyproline.

Enteritis is an inflammation of the small intestine caused by a bacterial or viral infection. The inflammation frequently also involves the stomach and large intestine. Enteritis is usually caused by eating or drinking substances contaminated with bacteria or viruses. The organism settles in the small intestine and causes inflammation and swelling that can cause abdominal pain, cramping, vomiting, diarrhea, fever, and dehydration. The symptoms may begin soon after exposure, or there can be a delay of several days. Risk factors include recent family illness with intestinal symptoms, recent travel, or exposure to untreated or contaminated water.

Types of enteritis include: Food poisoning, Salmonella enteritis, Shigella enteritis, Staph aureus food poisoning, Campylobacter enteritis, E. coli enteritis, Bacterial gastroenteritis, Radiation enteritis, Crohn's disease and regional enteritis

Mild cases usually need no treatment. Antidiarrhea medication may delay the elimination of the organism from the digestive tract, and therefore may not be recommended. Rehydration with electrolyte solutions may be necessary if dehydration from diarrhea occurs. People with diarrhea (especially young children) who are unable to take oral fluids because of nausea may need medical attention and intravenous fluids. People taking diuretics need to be cautious with diarrhea, and may need to stop taking the diuretic during the acute episode, as directed by the health care provider.

The hydroxyproline supplementation can be used for treatment of enteritis in animals including human beings, fish and birds. Enteritis may occur due to a condition where the connective tissue is weak and vulnerable due to lack of hydroxyproline and thus the protective layer is disrupted.

Psoriasis

In yet another embodiment Hydoxyproline supplementation can be used to treat psoriasis e.g. in human beings. The Hydoxyproline supplementation can be used to treat any type of psoriasis including the ones mentioned herein below.

The severity, location, and appearance of psoriasis depend on which kind you have. Plaque psoriasis is the most common form. Other kinds of psoriasis are much less common, including guttate, pustular, inverse, and a very inflammatory but rare form known as erythrodermic psoriasis.

Psoriasis affects both men and women of all ages although most people develop psoriasis as adults. The most significant risk factor for psoriasis is a family history of the condition. Environmental factors also are important. People who live in colder climates are more likely to develop psoriasis conditions and often develop the condition earlier in life. Other factors that may influence your psoriasis risk include ethnicity. Psoriasis is less common in African-Americans and is rare in native ethnic groups from North and South America.

Symptoms of Psoriasis

The Hydoxyproline supplementation can be used to ameliorate one or more symptoms of psoriasis including the ones mentioned herein below.

Dry, red patches of skin are the most well-known symptoms of psoriasis. The skin replaces itself more quickly than normal, causing excess skin cells to pile up in thick layers. Roughly half of people with psoriasis also have irregular fingernail growth that results in pitting, crumbling, or discoloration of the nail. A smaller proportion of people also experience arthritis symptoms.

Psoriasis may begin with small red bumps that eventually grow into large patches of dry skin that flake off in scales. Patches of affected skin often are symmetrical, meaning that they are present in the same place on both sides of the body. Some areas of the body are more likely to be affected, including the scalp, face, arms, hands, legs, groin, feet, and places where the skin folds.

Treatment

The Hydoxyproline supplementation can be used to treat psoriasis in combination with one or more other treatments of psoriasis including the ones mentioned herein below.

Initial treatment of psoriasis focuses on clearing away lesions during outbreaks of the disease, followed by lower-dose maintenance medications that prevent lesions from returning. Different treatments are often most effective when used in combination and may include creams and other topical (external) medications, light therapy, and more powerful systemic (internal) medications. Topical treatments are medications that are applied directly to the affected skin and that can effectively control many mild cases. These are all available by prescription.

Classical treatments of psoriasis include calcipotriene (a vitamin D.sub.3 derivative), topical coal tar preparations, systemic antimitotic agents such as methotrexate, and retinoids, particularly etretinate.

Corticosteroids (known by the generic name cortisone) have long been used to treat psoriasis by clearing the skin for a limited time. Cortisone is available in creams, ointments, lotions, shampoos, oils, and foams convenient for scalp treatment or other special applications. A dermatologist also may provide low-dose cortisone injections. Depending on which part of the body is being treated, different strengths can be applied; weaker formulas are used for the face and other sensitive areas. Calcipotriene can be used in small amounts for psoriasis lesions that are confined to less than 20% of the body's surface. The prescription ointment known as calcipotriene (Dovonex®) is different from regular vitamin D that is available over the counter. Other compounds related to vitamin D, including calcitrol, are currently being investigated as alternative treatments.

Extensive psoriasis can be treated by photosensitization with oral 8-methoxypsoralen, followed by ultraviolet A. Corticosteroids are given for psoriatic arthritis and acute attacks of pustular psoriasis. More recently, cyclosporin A has been tested in clinical trials at doses of 3-7 mg/kg with promising results, but associated with the risk of renal toxicity.

Current biotechnology approaches to psoriasis treatment relate to a direct pharmaceutical-mediated attack, either on cell proliferation or on the immune component of the disease. Japanese patent application JP 6145069 describes angiogenesis inhibitors comprising ganglioside GM3 or a GM3 analogue as an active agent. U.S. Pat. No. 5,339,977 describes n-deacetyl-lysoganglioside derivatives for use as phospholipase A2 inhibitors for the treatment of proliferative and autoimmune diseases, including various forms of cancer, psoriasis, and rheumatoid arthritis.

An IL-2 fusion toxin has been developed (Seragen, Inc.) that is designed to selectively destroy activated T cells in psoriatic plaques, leaving normal cells alone. The objective is to destroy activated T cells, and thereby clear the psoriasis. Comparable improvement was observed in patients with moderate to severe psoriasis. However, in order to obtain this response, the compound was administered three days per week for four weeks.

Various formulations containing the compound BCX-34 for psoriasis, cutaneous T cell lymphoma, and HIV infection have been tested (Bioworld Today, Sep. 29, 1997; see also WO 95/01355; WO 93/21187; WO 90/10631; U.S. Pat. Nos. 5,008,270, 5,008,265, and 4,985,434). BCX-34 is a small molecule drug that inhibits purine nucleoside phosphorylase, a human enzyme believed to be involved in the proliferation of T cells. An oral formulation is being tested in an ongoing Phase I/II trial. A topical formulation advanced to the Phase III stage for both lymphoma and psoriasis. The Phase III psoriasis study showed only a 14% greater improvement in mean lesion scores in the treated group compared to placebos, which for these studies was not statistically significant.

Several drugs have been designed to treat psoriasis by targeting specific cells, specific cytokines, or specific interactions between ligands and receptors. The main advantage of these biological agents over cyclosporin and methotrexate is the absence of nephrotoxicity and hepatotoxicity, but toxic effects can take years to develop. Candidates for biological therapy will be those who cannot comply with the rigours of a phototherapy regimen or have received too much PUVA and are at risk of hepatotoxicity and nephrotoxicity.

Another product available in the art consists in a chimeric human tumor necrosis factor a monoclonal antibody derived from mouse. It is made of the human constant and mouse variable regions of the IgG antibody. This agent is administered by intravenous infusion over at least 2 h and neutralizes soluble tumour necrosis factor alpha bound to cell membranes. Results of several case reports attest to the efficacy of this agent in treatment of psoriasis. Drawbacks of the drug include the need for slow intravenous infusion. A small but real proportion of patients have infusion reactions, including potentially serious reactions such as hypotension, rigors, and allergic reactions. These reactions can often be prevented by slowing down the infusion or pre-treating with antihistamines or, for some conditions, systemic corticosteroids. Neutralizing antibodies can develop, making the treatment less effective. Worse, patient who develop neutralizing antibodies are more likely to develop infusion reactions. One of the greatest concerns is the potential for infection with this tumour necrosis factor alpha inhibitor.

Alternatively, recombinant tumour necrosis factor alpha receptor fusion proteins were developed, which consist of two extracellular ligand-binding domains of the human p75 tumour necrosis factor alpha receptor fused to the Fc portion of human IgG1. These fusion proteins are likely effective for psoriatic arthritis and seems to be safer than cyclosporine or methotrexate with no nephrotoxicity or hepatotoxicity. Non-neutralising antibodies occur in less than 5% of patients. Antinuclear antibodies and antibodies to double-stranded DNA have been reported, but full cases of systemic lupus erythematosus are rare. Anticardiolipin antibodies also develop but have been attributed to minor concomitant infections. Because this agent is a tumour necrosis factor alpha inhibitor, concern has been raised about the potential for immunosuppression leading to infection.

Humanised anti-CD11a monoclonal antibody was also developed for the treatment for moderate-to-severe plaque psoriasis and moderate-to-severe rheumatoid arthritis. It is normally administered as a subcutaneous (under the skin) injection, and is designed to inhibit the binding of immune system T-cells to other cell types and targets three key processes in the cascade of events that lead to autoimmune symptoms.

Another family of products is known in the art to prevent T cell activation by blocking the LFA-3/CD-2 pathway through binding to the CD2 receptor. It is an IV/IM administered product. Since this family of products may cause reduction in CD4+ and CD8+ T lymphocyte counts, it may not be appropriate for all individuals.

A fully human fusion protein consisting of a binding site of LFA-3 fused to the Fe portion of IgGI CD45Ro+ memory T cells, which have a major role in development of psoriasis, maximally express CD2, a natural ligand of LFA-3 has been developed. By binding CD2, it prevents T-cell activation. Moreover, the Fe portion of the molecule engages Fe receptors on macrophages and NK cells, which results in apoptosis of the CD45RO+ T cells.

Another humanised monoclonal antibody to CD11A was developed, which is a component of LFA-1 on T cells and ICAM-1 on antigen-presenting cells is an important co-stimulatory signal resulting in T-cell activation. ICAM-1 on endothelial cells also interacts with LFA-1 on circulating T cells, a necessary step for migration of T cells into inflamed skin. It can thus interfere with development of psoriasis by blocking T-cell migration into the skin and by preventing T-cell activation. In clinical trials, some patients developed flu-like symptoms including headache, chills, fever, nausea, vomiting, or myalgias. Symptoms arose on the day of injection or the following 2 days. These acute adverse events subsided by the third dose.

Systemic treatment has been used in patients with physically, socially, or economically disabling psoriasis that has not responded to topical treatment. The choices to date have been phototherapy or systemic drug therapy. Generally, systemic treatment has employed phototherapy with Ultraviolet B irradiation, photo chemotherapy which combines the photosensitizing drug methoxsalen with Ultraviolet A phototherapy (PVA), methotrexate, etretinate, systemic corticosteroids, and cyclosporine. Each of these systemic treatments has variable efficacy and undesired side effects, and some of them are very toxic and present frequent relapses of the disease.

The hydroxyproline supplementation can be used for “survival enhancement” in animals including human beings, fish and birds.

The above mentioned applications of the hydroxyproline can be used for treatment, prophylactic treatment, ameliorating treatment and curative treatment.

The composition can be administrated either orally, sublingually, nasally, by inhalation, intravenously, intramuscularly, intrathecally, subcutaneously, by implantation, rectally, vaginally, by the ocular route, cutaneously or transdermally using either tablets, capsulated, micorincapsulated or liquid form of the compound.

The use of the Hydroxyproline for pharmaceutical application is probably relevant for pure product or pure product as a basis for pharmaceutical product.

The administrative level is probably as for the fish, although higher dosages may be relevant in clinical disorders.

### EXAMPLES

The following examples, wherein Fish were fed a diet with varying levels of hydroxyproline, and assessed for retention of hydroxyproline, levels of hydroxyproline in blood and tissue, level of incorporation of hydroxyproline in connective tissue are provided to illustrate the invention.

Materials and Methods

Diet Composition

The diets used were composed such that they are equal in protein, lipid and energy, and designed to vary only in level of Hydroxyproline. The small amounts of Hydroxyproline added (L-4-Hydroksyproline, >99.0% pure, Fluka BioChemika. Sigma-Aldrich Chemie GmbH) were exchanged with fish meal, and the diets were otherwise adjusted with the content of raw wheat to secure equal chemical composition. Five different levels of Hydroxyproline were used in a regression design with two replicates for each level, except the diet without Hydroxyproline addition that was run in triplicate tanks. The levels used of hydroxyproline were 0, 0.7 gkg⁻¹, 1.4 gkg⁻¹, 2.8 gkg⁻¹ and 5.6 gkg⁻¹. As a negative control, Proline (L-Proline, (Hydroxy-L-Proline Free) Sigma grade, Sigma Chemical Company) was added to a level of 2.8 gkg⁻¹.

The composition of the diets are given in Tables 1.A, 1.B and 1.C.

The chemical composition was found to be about equal for all diets (Table 1.A).

The diets were high in plant protein sources and moderate level of fish meal (185 gkg⁻¹), corresponding to 31% of total dietary protein. The mix of plant protein sources was corn gluten, soy protein concentrate, full fat soy bean meal and extracted soy bean meal (Table 1.B). All diets were supplemented with crystalline synthetic L-lysine HCl (0.4 gkg⁻¹) and monocalcium phosphate (2.2 gkg¹).

TABLE 1.A
Chemical composition of experimental diets.

| | Diet no | | | | |
|---|---|---|---|---|---|
| | 1 | 2 | 3 | 4 | 5 |
| Added | 0 | 0.7 | 1.4 | 2.8 | 5.6 |
| hydroxyproline gkg⁻¹ | | | | | |
| Moisture | 57 | 52 | 52 | 58 | 58 |
| Crude protein | 380 | 377 | 378 | 379 | 375 |
| Lipid | 301 | 303 | 300 | 294 | 301 |
| Ash | 65 | 64 | 65 | 64 | 64 |
| Gross energy^{a} MJ | 24.2 | 24.3 | 24.3 | 24.1 | 24.2 |
| kg⁻¹ | | | | | |

^{a}Calculated using the following combustion values (MJ kg⁻¹): protein: 23.64; lipid 39.45; carbohydrate: 17.15.

TABLE 1.B
Composition of experimental diets (gkg−1)

| | Diet no | | | | |
|---|---|---|---|---|---|
| Ingredients | 1 | 2 | 3 | 4 | 5 |
| Hydroxyproline | 0 | 0.7 | 1.4 | 2.8 | 5.6 |
| Fishmeal^{a} | 185 | 185 | 185 | 185 | 183 |
| Soy protein concentrate | 67 | 67 | 67 | 67 | 67 |
| Full fat soy bean meal | 70 | 70 | 70 | 70 | 70 |
| Extracted soy bean meal | 50 | 50 | 50 | 50 | 50 |
| Wheat gluten | 30 | 30 | 30 | 30 | 30 |
| Corn gluten | 160 | 160 | 160 | 160 | 160 |
| Raw wheat^{b} | 103 | 102 | 102 | 101 | 100 |
| Fish oil^{c} | 290 | 290 | 290 | 290 | 290 |
| Vitamins^{d} | 10 | 10 | 10 | 10 | 10 |
| Minerals^{e} | 8 | 8 | 8 | 8 | 8 |
| Carophyll Pink (8%) | 0.6 | 0.6 | 0.6 | 0.6 | 0.6 |
| Lysine HCL | 4.0 | 4.0 | 4.0 | 4.0 | 4.0 |
| Monocalcium phosphate | 22 | 22 | 22 | 22 | 22 |

^{a}Norse-LT 94, Norsildmel, Norway
^{b}Whole wheat, Norgesmøllene, Bergen, Norway
^{c}NorSeaOil, Norsildmel, Norway
^{d}Provided per kg of feed: vitamin D₃, 3000 I.E.; vitamin E, 160 mg; thiamin, 20 mg; riboflavin, 30 mg; pyrodoxine-HCl, 25 mg; vitamin C, 200 mg;; calcium pantothenate, 60 mg; biotin. 1 mg; folic acid, 10 mg; niacin, 200 mg; vitamin B_{12,} 0.05 mg; menadion bisulphite, 20 mg
^{e}Provided per kg of feed: magnesium 600 mg; potassium, 480 mg; zinc, 96 mg; iron, 60 mg; manganese, 12 mg; copper, 6 mg.

TABLE 1.C
Dietary level of amino acids (gkg⁻¹ protein)

| | Diet | | | | |
|---|---|---|---|---|---|
| | 1 | 2 | 3 | 4 | 5 |
| 1. Added | 0 | 0.7 | 1.4 | 2.8 | 5.6 |
| hydroxyproline (gkg⁻¹ | | | | | |
| diet) | | | | | |
| Aspartic acid | 82 | 83 | 83 | 81 | 79 |
| Glutamic acid | 183 | 186 | 187 | 183 | 183 |
| Hydroxyproline | 7 | 8 | 10 | 13 | 21 |
| Serine | 47 | 48 | 48 | 47 | 49 |
| Glycine | 46 | 46 | 47 | 46 | 47 |
| Histidine | 21 | 21 | 21 | 21 | 22 |
| Arginine | 55 | 55 | 56 | 55 | 59 |
| Threonine | 34 | 34 | 35 | 34 | 37 |
| Alanine | 59 | 59 | 59 | 58 | 59 |
| Proline | 61 | 60 | 61 | 59 | 60 |
| Tyrosine | 32 | 32 | 31 | 32 | 37 |
| Valine | 44 | 45 | 45 | 44 | 46 |
| Methionine | 21 | 20 | 20 | 20 | 21 |
| Isoleucine | 44 | 45 | 46 | 44 | 43 |
| Leucine | 100 | 101 | 100 | 99 | 99 |
| Phenylalanine | 48 | 49 | 48 | 48 | 50 |
| Lysine | 59 | 60 | 61 | 60 | 61 |

Methods of Preparation of the Diet

The diets were extruded (4.5 mm pellet) in a Wenger twin screw extruder at fixed and defined conditions. No differences were seen in any diets with regard to physical quality or sinking properties (14-17 seconds per meter for all diets).

Experimental Conditions for Growth of Fish

Atlantic salmon (Salmo salar) was obtained from Salmobreed May 2006. The fish was prevaccinated with Trippel Forte and was acclimated for 56 days before the trial started. The fish was given EWOS Opal Boost 50 3A, 3 mm pellets during this period. The fish was randomly distributed among 14 fiberglass tanks (1.5×1.5×1 m³) with 80 fish per tank. Diet 1 and Diet 6 were run in triplicate tanks. Diet 2, Diet 3, Diet 4 and Diet 5 was run in duplicate tanks. Data are given also for a fish meal diet with no plant protein included. This diet was run simultaneously with fish of the same size, origin and treatment. This diet (Diet 7) was run in 2 m×2 m×1 m tanks in triplicate in a parallel experiment. The tanks were supplied with 75 I min⁻¹ of seawater taken from a depth of 50 m with average salinity of 3.2-3.3%. Water temperature was kept constant at 10.4±0.4° C. during the acclimatization and the experimental period, using a heat exchanger to adjust the sea water temperature. Throughout the experimental period of 88 days, the fish were exposed to 24 h artificial light. Great care was taken to reduce disturbance from the environment. Automatic feeders were used to add feed from 05.00 to 08.00 hrs and from 14.00 to 03.00 hrs. The level of feeding was regulated in such a way that offered feed was 110% of expected consumption based on daily monitoring of wasted feed. Uneaten feed was continuously removed by automatic collection and separated from feces using an Excess Fishfeed Collector (Holland Teknologi, Norway). Feed intake was measured daily as the difference between total feed given and total uneaten feed collected. Feed intake for the total experimental period was measured as the sum of the daily feed intakes, divided by feeding days and average fish weight during the experimental period; (start weight+final weight)/2. Feeding was sustained until the fish was sampled for analyses.

The six experimental diets were randomly assigned to the 14 tanks, with 3 replicate tanks for Diet 1 and Diet 6 and to duplicate tanks for the other experimental diets. Initial fish weights and lengths were recorded. Twenty fish representing the weight ranges were sampled, killed by a blow on the head, frozen and kept at −80° C. pending analyses of fish at start. Final weights were determined for each fish in all tanks. At the end of the feeding period, the fish were anaesthetized with Eugonol (iso-eugunol was diluted 1:1 with ethanol and the diluted solution was added 15 ml per m³ water in the tanks). After about 15 min, 10 fish from each tank were sampled, killed by a blow in the head, gutted and weight of slaughtered fish, liver and viscera were noted. Five other fish from each tank were pooled and frozen for pending analyses.

Feed Consumption, Growth and Feed Efficiency.

The 88 days growth experiment was run successfully with a thermal growth coefficient (TGC) of 2.5 for the fish meal control diet. Some loss of fish occurred during the experimental period and some fish seemed not to develop normally. The number of fish that did not develop and that was lost, did not however differ significantly between experimental groups or even for the diet containing the fish meal control, which generally performed better than the experimental diets low in fish meal for growth and feed efficiency (Table 2). Statistical evaluation did not differ if the undeveloped fish were excluded or included in the calculations, and no statistical conclusions were different. The results and statistical evaluation given below are based on data excluding the undeveloped fish both for evaluation of growth and feed efficiency and for evaluation of HIS, DOP and vertebral data.

Calculations and Statistical Methods.

Specific growth rate (SGR) for weight was calculated as SGR (%)=(ln w₂−ln w₁)×100/feeding days, where w₁ and w₂ are start and final fish weights, respectively and ln the natural logarithm. Thermal growth coefficient (TGC) was calculated as described by Cho (1992):

TGC=(w₂^{1/3}−w₁^{1/3})×1000/Σ(t×feeding days), where Σ(t×feeding days) is the sum of water temperatures (° C.) for every feeding day in the experiment. The content of carbohydrate in the diets was calculated as: Carbohydrate (g kg⁻¹)=1000−(protein+lipid+moisture+ash).

Condition factor was calculated as fish weight (g)×fish length⁻³×1000. Hepatosomatic index (HSI) was defined as liver weight/fish weight×100. Dressing out percentage (DOP) was calculated as weight of viscera in percentage of total fish weight. Biological and analytical data obtained from Diet 1, Diet 2, Diet 3, Diet 4 and Diet 5 were subjected to regression analyses (ANOVA) using SYSTAT 5.0 for Windows (Wilkinson et al., 1992). Statistical calculation to make comparisons between Diet 1 and Diet 4 and Diet 1 and Diet 6 were achieved by the similar program in a t-test.

Chemical Analyses

Crude protein (N×6.25) in feed ingredients, diets, feces and fish tissues, was determined by the Kjeldahl method (ISO5983-1997), moisture (ISO 6496-1999) and ash (ISO 5984-2002) gravimetrically after drying for 4 h at 105° C. and after combustion for 16 h at 550° C., respectively. Total amino acids were analyzed after hydrolysis in 6M HCl for 22 hours at 110° C. and analyzed by HPLC using a fluorescence technique for detection (Cohen and Michaud, 1993).

All analyses were carried out in duplicates except amino acid analyses in the vertebra column. If differences between parallels exceeded standardised values, new duplicate analyses were performed according to accredited procedures. Vitamin C content in Diet 1 and Diet 4 (after 6 months storage at arbitrary temperature) was analysed by the method of Keck and Schüeb (1992).

Results

The chemical composition was found to be about equal for all diets (Table 1), except for the dietary level of Hydroxyproline and Proline that were found to be about as the levels that can be calculated for the respective added amounts (Table 3). Dietary levels of all the other amino acids were about equal in the experimental Diets 1 to Diet 6. Vitamin C content in Diet 1 and Diet 4 was 170 mgkg-1 and 150 mgkg-1 diet, respectively.

### Example 1

Trout were fed fish hydrolysate and fractions thereof to identify bioactive compounds (see Table 2).

TABLE 2

| | PP | LH | LR | HH | HR | FM |
|---|---|---|---|---|---|---|
| Dietary plant protein - gkg⁻¹- | 353 | 288 | 291 | 223 | 232 | 223 |
| % of total protein | 90.6 | 73.9 | 74.9 | 57.2 | 59.4 | 57.0 |
| Free amino acids | 3.5 | 9.8 | 8.2 | 14.6 | 11.3 | 3.5 |
| (% of total protein) | | | | | | |
| Taurine | 0.8 | 2.8 | 2.3 | 4.3 | 2.8 | 3.3 |
| Anserine | 0.3 | 3.3 | 2.3 | 5.0 | 3.0 | 1.3 |
| Aspartic acid | 87 | 83 | 84 | 81 | 83 | 82 |
| Glutamic acid | 213 | 194 | 196 | 184 | 192 | 187 |
| Hydroxyproline | 2 | 8 | 8 | 12 | 14 | 5 |
| Serine | 53 | 49 | 50 | 48 | 49 | 50 |
| Glycine | 42 | 54 | 52 | 68 | 72 | 51 |
| Histidine | 24 | 23 | 23 | 22 | 22 | 22 |
| Arginine | 61 | 62 | 61 | 65 | 60 | 65 |
| Threonine | 36 | 35 | 34 | 35 | 34 | 37 |
| Alanine | 58 | 57 | 57 | 58 | 59 | 57 |
| Proline | 78 | 74 | 75 | 70 | 79 | 67 |
| Tyrosine | 43 | 32 | 32 | 31 | 33 | 37 |
| Valine | 48 | 43 | 43 | 41 | 41 | 47 |
| Methionine | 17 | 18 | 18 | 20 | 20 | 21 |
| Isoleucine | 45 | 40 | 39 | 37 | 37 | 43 |
| Leucine | 104 | 88 | 88 | 78 | 78 | 89 |
| Phenylalanine | 55 | 48 | 48 | 43 | 43 | 49 |
| Lysine | 52 | 62 | 56 | 62 | 58 | 51 |

PP = Diet high in plant protein, LH, HH, LR, HR and FM = Diet with marine protein source varying in hydroxyproline content.

### Example 2

### Increased Levels of Hydroxyproline in the Diet Lead to Less Retention of Hydroxyproline

The experiments where fish were fed a diet including varying levels of hydroxyproline were performed as described above.

The retention of hydroxyproline is statistically correlated to the dietary level. Low dietary levels (0.8 g per kg diet) give retention of close to 100% while higher dietary levels (>4.5 g per kg diet) gives retention of 20-30%. This indicates that the component has some biological impact and that the detrimental effect is observed at dietary levels of less 3-4 g per kg diet (FIG. 1).

### Example 3

### Increased Levels of Hydroxyproline in the Diet Lead to Increased Levels of Hydroxyproline in the Plasma as Well as in the Tissue

The blood plasma levels of the component shown in FIG. 2. The y axis is plasma levels in micromoles per 100 ml and the x-axis is the dietary level of component in g per kg diet. At low levels (0.8 g per kg) the plasma levels are much lower than earlier published (normal values) and indicate that dietary supplement is too low and that the component easily retrieved from the plasma and into the tissues. Dietary levels >4 gives very high plasma levels (highest described in the literature) and indicate that you have a surplus (FIG. 2).

FIG. 3 demonstrated the data for filet and whole fish. These show the same tendency and conclusion as for plasma levels.

The data therefore indicate that there is a nutritional requirement for hydroxyproline and that the negative effects are eliminated at dietary levels of higher than 3-4 g per kg diet.

### Example 4

### Increased Levels of Hydroxyproline in the Diet Lead to Increased Weight Gain in Fish

Experiments where fish were fed a diet including varying levels of hydroxyproline were performed as described above.

Dietary inclusion of hydroxyproline showed a growth promoting effect on final weight, weight gain, specific growth rate (SGR, see above) and thermal growth coefficient (TGC, see above). The increase in weight gain is shown in FIG. 1 and Table 3 (below). The growth curve indicates that the component is essential and that the nutritional requirement is about 2.5-3 g per kg diet. This conclusion is supported by positive changes in the vertebra chemical composition and structure (see examples below).

All the growth parameters significantly (p<0.01) fit a polynomial regression with dietary inclusion level of Hydroxyproline. The corresponding regression equations were: Weight gain=−2.07(Hydroxyproline)2+12.1 (Hydroxyproline)+137 (n=11, r2=0.57), SGR=−0.009(Hydroxyproline)2+0.051 (Hydroxyproline)+0.93 (n=11, r2=0.48), TGC=−0.018(Hydroxyproline)2+0.105 (Hydroxyproline)+1.62 (n=11, r2=0.52), where Hydroxyproline is added Hydroxyproline in gkg-1 diet.

The equation for weight gain corresponds to a maximum added value of Hydroxyproline of 2.9 gkg−1 giving a weight gain of 155 g, or an increase of 14.0% compared with no Hydroxyproline added (Diet 1). The data obtained for weight gain are also shown in FIG. 1. No significant difference was observed in growth parameters comparing Diet 1 with no addition and Diet 6 with added 2.8 gkg−1 Proline. No significant differences or regressions where observed for feed intake or feed efficiency for any groups except the fish meal diet showing higher feed intake and better feed efficiency.

The analyses of fish showed no difference or significant regressions with dietary Hydroxyproline on HIS, DOP or condition factor.

TABLE 3
Growth and feed performance. (Values are mean ± SD for three replicates).

| | | | | | | Probability of |
|---|---|---|---|---|---|---|
| | 1 | 2 | 3 | 4 | 5 | regression |
| N | 3 | 2 | 2 | 2 | 2 | 11 |
| Body weight at start, | 110 ± 0.2 | 108 ± 2.0 | 110 ± 0.4 | 111 ± 0.5 | 110 ± 0 | Ns |
| g | | | | | | |
| Body weight at end, g | 246 ± 7 | 256 ± 9 | 264 ± 2 | 262 ± 3 | 251 ± 8 | <0.01 |
| Weight gain, g/fish | 136 ± 7 | 147 ± 11 | 155 ± 2 | 151 ± 2 | 141 ± 8 | <0.01 |
| SGR | 0.92 ± 0.03 | 0.97 ± 0.06 | 1.00 ± 0.01 | 0.98 ± 0.0 | 0.94 ± 0.04 | <0.01 |
| TGC | 1.60 ± 0.06 | 1.71 ± 0.11 | 1.76 ± 0.02 | 1.73 ± 0.01 | 1.64 ± 0.07 | <0.01 |
| Feed consumption, | 0.96 ± 0.05 | 1.05 ± 0.08 | 1.08 ± 0.15 | 0.98 ± 0.03 | 1.04 ± 0.10 | Ns |
| % of av. fish | | | | | | |
| weight/day | | | | | | |
| FER, g fish growth/g | 0.91 ± 0.05 | 0.88 ± 0.11 | 0.88 ± 0.13 | 0.94 ± 0.02 | 0.86 ± 0.11 | Ns |
| feed eaten | | | | | | |
| Condition factor^{a} | 1.13 ± 0.08 | 1.16 ± 0.08 | 1.17 ± 0.09 | 1.10 ± 0.14 | 1.17 ± 0.09 | Ns |
| HSI^{ab} | 1.52 ± 0.31 | 1.51 ± 0.28 | 1.45 ± 0.26 | 1.45 ± 0.29 | 1.38 ± 0.22 | p = 0.07 |
| DOP^{ac} | 17.7 ± 2.5 | 18.1 ± 2.0 | 17.3 ± 2.6 | 17.7 ± 1.8 | 17.9 ± 1.8 | Ns |
| Undeveloped fish | 14.2 ± 2.6 | 15.6 ± 9.7 | 14.4 ± 9.7 | 9.4 ± 2.6 | 18.1 ± 2.7 | Ns |
| (% of initial number) | | | | | | |
| Dead fish (% per day) | 0.18 ± 0.03 | 0.20 ± 0.12 | 0.18 ± 0.12 | 0.12 ± 0.03 | 0.23 ± 0.03 | Ns |
| Wounds on skin^{ad} | 1.07 ± 1.05 | 1.20 ± 0.77 | 0.70 ± 0.65 | 1.20 ± 1.15 | 0.60 ± 0.75 | p = 0.10 |
| Damage on dorsal fin^{ad} | 0.53 ± 0.90 | 1.40 ± 1.23 | 0.95 ± 1.15 | 1.35 ± 1.27 | 0.40 ± 1.00 | ns |
| Loss of shells^{ad} | 1.13 ± 0.90 | 1.20 ± 0.52 | 0.90 ± 0.64 | 0.70 ± 0.66 | 0.85 ± 0.59 | p = 0.06 |

### Example 5

### Increased Levels of Hydroxyproline in the Diet Increased Vertebrate Growth in Fish

The experiments where fish were fed a diet including varying levels of hydroxyproline were performed as described above.

Increased vertebral growth was measured as follows. Five gutted fish from the six tanks representing Diet 1 and Diet 4 (0 gkg-1 hydroxyproline and 2.6 gkg−1 hydroxyproline respectively) that were available after determination of HSI and DOP percentage were also used to obtain more data on the vertebra.

The fish were thawed overnight at about 10° C. and each fish was measured for length and weight. The fish was filleted with head and tail remaining, and the gills were removed. The fish was dipped in boiling water for 1 min and coagulated muscle was removed by cold water flushing. Remaining muscle and neural tissue was thoroughly removed by hand. Head and tail were removed from the vertebral column and side bones were cut at the vertebral base.

The vertebral column free from surface water was weighed and the length determined. Individual lengths (anterior to posterior) and minimum (dorsal-ventral) and maximum (side to side) width in the middle of vertebrae no 40 were determined with a slide caliper. The vertebral columns were lyophilized and the chemical composition was determined in the column from each specific fish. The amino acid composition was determined in the total vertebral column following a similar procedure with individual fish samples.

Analyses of Vertebral Column.

Analytical data for the vertebral column and specifically for vertebrae no 40 of fish fed Diet 1 (no addition) and Diet 4 (added 2.8 g Hydroxyproline per kg diet) are shown in Table 4 below. And was shown to contain significant lower level of ash in the vertebral column (in lipid free dry matter), greater length of vertebrae no 40, greater length of vertebrae no 40 related to total vertebrae length and close to significant greater length of vertebrae 40 related to maximum width (side to side) of vertebrae no 40. The amino acid composition of the total vertebral column showed significant higher level of Hydroxyproline in fish given Diet 4 compared to fish given Diet no 1 (Table 4). No other amino acids were different in the vertebral column from the two groups.

TABLE 4
Physical and chemical data for vertebral column and vertebrae no 40.

| | Diet | | |
|---|---|---|---|
| | 1 | 4 | Probability |
| Added hydroxyproline | 0 | 2.8 | |
| (gkg⁻¹ diet) | | | |
| Fish weight g | 200 ± 39 | 226 ± 44 | |
| Fish length cm | 25.8 ± 1.2 | 26.7 ± 1.3 | |
| Vertebra length, cm | 19.9 ± 1.0 | 20.4 ± 1.2 | |
| Vertebra length/fish | 77.3 ± 1.3 | 76.3 ± 1.5 | |
| length, % | | | |
| Minimum dorso-ventral | 3.5 ± 0.3 | 3.7 ± 0.4 | |
| height, vertebrae no. | | | |
| 40, mm | | | |
| Maximum dorso-ventral | 4.7 ± 0.4 | 4.7 ± 0.4 | |
| width, vertebrae no | | | |
| 40, mm | | | |
| Length, vertebrae no | 3.8 ± 0.2^{a} | 4.2 ± 0.4^{b} | <0.05 |
| 40, mm | | | |
| Length vertebrae 40/ | 1.93 ± 0.10^{a} | 2.04 ± 0.14^{b} | <0.05 |
| total vertebrae | | | |
| length, % | | | |
| Length vertebrae 40/ | 81.7 ± 7.8 | 88.6 ± 9.3 | 0.07 |
| V40, max dorso-ventral | | | |
| height, % | | | |
| Wet weight of vertebrae, | 2.49 ± 0.3 | 2.85 ± 0.6 | |
| g | | | |
| Wet weight of vertebrae/ | 1.27 ± 0.17 | 1.26 ± 0.12 | |
| fish weight, % | | | |
| Wet weight of vertebrae/ | 9.65 ± 0.85 | 10.63 ± 1.78 | |
| fish length, % | | | |
| Dry weight of vertebrae, | 1.17 ± 0.17 | 1.29 ± 0.25 | |
| g | | | |
| Vertebrae dry matter | 47.0 ± 4.4 | 45.7 ± 3.6 | |
| Ash in vertebrae lipid | 52.8 ± 1.33^{a} | 51.4 ± 1.41^{b} | <0.05 |
| free dry matter | | | |
| Protein in vertebrae | 45.8 ± 1.2 | 45.6 ± 0.9 | |
| lipid free dry matter | | | |
| Ash/protein in vertebrae | 1.16 ± 0.04 | 1.13 ± 0.05 | |

Values are mean ± SD (n = 30 for Diet 1; n = 20 for Diet 4).

### Example 6

### Increased Levels of Hydroxyproline in the Diet Lead to Increased Levels of Hydroxyproline Incorporated in the Skeleton

The experiments where fish were fed a diet including varying levels of hydroxyproline were performed as described above.

TABLE 5
Amino acid composition in vertebral column (gkg−1 protein (Nx6.25).

| | Diet | | |
|---|---|---|---|
| | 1 | 4 | Probability |
| 1. Added (gkg⁻¹ diet) | 0 | 2.8 | |
| Aspartic acid | 6.77 ± 0.43 | 6.62 ± 0.45 | |
| Glutamic acid | 10.7 ± 0.5 | 10.6 ± 0.5 | |
| Hydroxyproline | 6.45 ± 0.13^{a} | 6.63 ± 0.10^{b} | <0.01 |
| Serine | 4.57 ± 0.10 | 4.64 ± 0.03 | |
| Glycine | 18.5 ± 0.5 | 18.8 ± 0.3 | |
| Histidine | 2.18 ± 0.04 | 2.21 ± 0.01 | |
| Arginine | 7.83 ± 0.08 | 7.86 ± 0.14 | |
| Threonine | 3.43 ± 0.10 | 3.37 ± 0.01 | |
| Alanine | 7.47 ± 0.17 | 7.47 ± 0.07 | |
| Proline | 8.58 ± 0.17 | 8.68 ± 0.03 | |
| Tyrosine | 1.59 ± 0.17 | 1.62 ± 0.20 | |
| Valine | 2.93 ± 0.05 | 2.93 ± 0.01 | |
| Methionine | 3.03 ± 0.05 | 3.06 ± 0.06 | |
| Isoleucine | 2.21 ± 0.04 | 2.19 ± 0.01 | |
| Leucine | 4.03 ± 0.06 | 4.01 ± 0.04 | |
| Phenylalanine | 2.53 ± 0.03 | 2.53 ± 0.04 | |
| Lysine | 3.99 ± 0.19 | 3.98 ± 0.25 | |
| hydroxyproline/proline | 0.63 ± 0.01^{a} | 0.65 ± 0.01^{b} | <0.01 |
| ratio | | | |
| Sum amino acids | 95.8 ± 2.2 | 96.2 ± 0.4 | |

Values are mean ± SD (n = 15 for diet 1; n = 10 for Diet 4).

### Example 7

### Increased Levels of Hydroxyproline have Beneficial Effects on Wound Healing, Shell Loss, and Damage to Dorsal Fin

The experiments where fish were fed a diet including varying levels of hydroxyproline were performed as described above.

Wounds on skin, damages on dorsal fin and loss of shells were evaluated in the 10 salmons from each tank. The measure was subjectively based on a scale from 0 (no damage) to 5 (severe damages). The data and statistics are included in the table below (based on 110 measures) and shown in the figure based on average values for each tank). The statistics given are based on linear regression. Loss of shells was significantly correlated to dietary content in a squared regression. (Table 3 and FIGS. 5, 6 and 7)

1. A composition comprising hydroxyproline and optionally a protein source.

2. The composition according to claim 1, wherein the hydroxyproline is present in the form of a free amino acid, or a physiologically acceptable salt thereof

3. The composition according to claim 1, wherein the hydroxyproline is a hydroxyproline residue forming part of a hydrolysed oligopeptide.

4. (canceled)

5. The composition according to claim 1, wherein the protein source is selected from the group consisting of a plant protein source and animal protein source, wherein said protein source is optionally in hydrolysed form.

6. The composition according to claim 5, wherein the composition comprises both a plant protein source and an animal protein source.

7-24. (canceled)

25. The composition according to claim 1, wherein the composition is a dried composition.

26-45. (canceled)

46. A pharmaceutical composition comprising a therapeutically effective amount of hydroxyproline or hydroxyprolyl.

47-49. (canceled)

50. A method for treatment of degenerative bone disorder comprising the steps of administering to an individual an effective amount of the composition according to claim 46 to said individual.

51-147. (canceled)

148. A method for the treatment of a wound in an animal, said method comprising the steps of:
i) providing the composition according to claim 1,
ii) feeding said animal an effective amount of said composition, and
iii) treating said wound,
thereby obtaining healing of said wound or accelerated healing of said wound in said animal.

149-157. (canceled)

158. A method for promoting bone formation in an animal, said method comprising the steps of:
i) providing the composition according to claim 1,
ii) feeding said animal an effective amount of said composition,
thereby promoting bone formation in said animal.

159-162. (canceled)

163. A method for promoting bone strength in an animal, said method comprising the steps of:
i) providing the composition according to claim 1, p1 feeding said animal an effective amount of said composition,
thereby promoting bone strength in said animal.

164-167. (canceled)

168. A method for promoting growth or survival enhancement of an animal, said method comprising the steps of:
i) providing the composition according to claim 1
ii) feeding said animal an effective amount of said composition, thereby promoting growth or survival enhancement of said animal.

169-175. (canceled)

176. A method for the treatment of a wound in an animal, said treatment comprising the steps of:
i) providing the composition according to claim 46,
ii) administering to said animal an effective amount of said composition, and
iii) treating said wound,
thereby obtaining healing of said wound or accelerated healing of said wound in said animal.

177-195. (canceled)

196. A method for promoting growth or survival enhancement of an animal, said method comprising the steps of:
i) providing of the composition according to claim 46,
ii) administering to said animal an effective amount of said composition,
thereby promoting growth or survival enhancement of said animal.

197-220. (canceled)

221. A method for supplementing or increasing hydroxyproline levels in an individual in need thereof, said method comprising the steps of administering to said individual the composition according to any of claims 1 or 46.

222-226. (canceled)
